SEQUENCE LISTING (862 sequences)

SEQ ID NO: 1 - RNA, 19 nt, synthetic construct
  source 1..19: mol_type = other RNA
cggctcaccttgaaggaca

SEQ ID NO: 2 - RNA, 19 nt, synthetic construct
  source 1..19: mol_type = other RNA
ggctcaccttgaaggacac

SEQ ID NO: 3 - RNA, 19 nt, synthetic construct
  source 1..19: mol_type = other RNA
gctcaccttgaaggacacc

SEQ ID NO: 4 - RNA, 19 nt, synthetic construct
  source 1..19: mol_type = other RNA
ctcaccttgaaggacacct

SEQ ID NO: 5 - RNA, 19 nt, synthetic construct
  source 1..19: mol_type = other RNA
tcaccttgaaggacacctc

SEQ ID NO: 6 - RNA, 19 nt, synthetic construct
  source 1..19: mol_type = other RNA
gaggccatgctgtcctcct

SEQ ID NO: 7 - RNA, 19 nt, synthetic construct
  source 1..19: mol_type = other RNA
aggccatgctgtcctcctg

SEQ ID NO: 8 - RNA, 19 nt, synthetic construct
  source 1..19: mol_type = other RNA
ggccatgctgtcctcctgg

SEQ ID NO: 9 - RNA, 19 nt, synthetic construct
  source 1..19: mol_type = other RNA
gccatgctgtcctcctgga

SEQ ID NO: 10 - RNA, 19 nt, synthetic construct
  source 1..19: mol_type = other RNA
ccatgctgtcctcctggaa

SEQ ID NO: 11 - RNA, 19 nt, synthetic construct
  source 1..19: mol_type = other RNA
catgctgtcctcctggaag

SEQ ID NO: 12 - RNA, 19 nt, synthetic construct
  source 1..19: mol_type = other RNA
atgctgtcctcctggaagc

SEQ ID NO: 13 - RNA, 19 nt, synthetic construct
  source 1..19: mol_type = other RNA
tgctgtcctcctggaagca

SEQ ID NO: 14 - RNA, 19 nt, synthetic construct
  source 1..19: mol_type = other RNA
acccagcggtcagcgatga

SEQ ID NO: 15 - RNA, 19 nt, synthetic construct
  source 1..19: mol_type = other RNA
cccagcggtcagcgatgag

SEQ ID NO: 16 - RNA, 19 nt, synthetic construct
  source 1..19: mol_type = other RNA
cctggaggccacagtcaca

SEQ ID NO: 17 - RNA, 19 nt, synthetic construct
  source 1..19: mol_type = other RNA
tggaggccacagtcacagt

SEQ ID NO: 18 - RNA, 19 nt, synthetic construct
  source 1..19: mol_type = other RNA
ggaggccacagtcacagtg

SEQ ID NO: 19 - RNA, 19 nt, synthetic construct
  source 1..19: mol_type = other RNA
gtgagggagatctgggagg

SEQ ID NO: 20 - RNA, 19 nt, synthetic construct
  source 1..19: mol_type = other RNA
agggagatctgggaggtga

SEQ ID NO: 21 - RNA, 19 nt, synthetic construct
  source 1..19: mol_type = other RNA
gggagatctgggaggtgaa

SEQ ID NO: 22 - RNA, 19 nt, synthetic construct
  source 1..19: mol_type = other RNA
ttgtaccctaggaaatacc

SEQ ID NO: 23 - RNA, 19 nt, synthetic construct
  source 1..19: mol_type = other RNA
tgtaccctaggaaatacca

SEQ ID NO: 24 - RNA, 19 nt, synthetic construct
  source 1..19: mol_type = other RNA
ttcttgcccttgcggtagc

SEQ ID NO: 25 - RNA, 19 nt, synthetic construct
  source 1..19: mol_type = other RNA
tcttgcccttgcggtagcc

SEQ ID NO: 26 - RNA, 19 nt, synthetic construct
  source 1..19: mol_type = other RNA
tggaggctggcctgccatg

SEQ ID NO: 27 - RNA, 19 nt, synthetic construct
  source 1..19: mol_type = other RNA
tgccatggccactcaccct

SEQ ID NO: 28 - RNA, 19 nt, synthetic construct
  source 1..19: mol_type = other RNA
ccatggccactcaccctcg

SEQ ID NO: 29 - RNA, 19 nt, synthetic construct
  source 1..19: mol_type = other RNA
ccactcaccctcggaggac

SEQ ID NO: 30 - RNA, 19 nt, synthetic construct
  source 1..19: mol_type = other RNA
cactcaccctcggaggaca

SEQ ID NO: 31 - RNA, 19 nt, synthetic construct
  source 1..19: mol_type = other RNA
actcaccctcggaggacac

SEQ ID NO: 32 - RNA, 19 nt, synthetic construct
  source 1..19: mol_type = other RNA
cagtttctctcatccaggc

SEQ ID NO: 33 - RNA, 19 nt, synthetic construct
  source 1..19: mol_type = other RNA
agtttctctcatccaggcc

SEQ ID NO: 34 - RNA, 19 nt, synthetic construct
  source 1..19: mol_type = other RNA
gccaagccgtagtccagag

SEQ ID NO: 35 - RNA, 19 nt, synthetic construct
  source 1..19: mol_type = other RNA
ccaagccgtagtccagaga

SEQ ID NO: 36 - RNA, 19 nt, synthetic construct
  source 1..19: mol_type = other RNA
aaccagaagaagcaggtga

SEQ ID NO: 37 - RNA, 19 nt, synthetic construct
  source 1..19: mol_type = other RNA
accagaagaagcaggtgag

SEQ ID NO: 38 - RNA, 19 nt, synthetic construct
  source 1..19: mol_type = other RNA
gagcatcttctgggctttg

SEQ ID NO: 39 - RNA, 19 nt, synthetic construct
  source 1..19: mol_type = other RNA
agcatcttctgggctttgg

SEQ ID NO: 40 - RNA, 19 nt, synthetic construct
  source 1..19: mol_type = other RNA
gcatcttctgggctttggc

SEQ ID NO: 41 - RNA, 19 nt, synthetic construct
  source 1..19: mol_type = other RNA
caggcagctttattccaaa

SEQ ID NO: 42 - RNA, 19 nt, synthetic construct
  source 1..19: mol_type = other RNA
aggcagctttattccaaag

SEQ ID NO: 43 - RNA, 19 nt, synthetic construct
  source 1..19: mol_type = other RNA
gcagctttattccaaaggg

SEQ ID NO: 44 - RNA, 19 nt, synthetic construct
  source 1..19: mol_type = other RNA
cagctttattccaaagggc

SEQ ID NO: 45 - RNA, 19 nt, synthetic construct
  source 1..19: mol_type = other RNA
agctttattccaaagggca

SEQ ID NO: 46 - RNA, 19 nt, synthetic construct
  source 1..19: mol_type = other RNA
tttattccaaagggcagct

SEQ ID NO: 47 - RNA, 19 nt, synthetic construct
  source 1..19: mol_type = other RNA
ttattccaaagggcagctg

SEQ ID NO: 48 - RNA, 19 nt, synthetic construct
  source 1..19: mol_type = other RNA
caaagggcagctgagctca

SEQ ID NO: 49 - RNA, 19 nt, synthetic construct
  source 1..19: mol_type = other RNA
aaagggcagctgagctcac

SEQ ID NO: 50 - RNA, 19 nt, synthetic construct
  source 1..19: mol_type = other RNA
accaggggcttccgaagct

SEQ ID NO: 51 - RNA, 19 nt, synthetic construct
  source 1..19: mol_type = other RNA
ccagcattcttgctgctga

SEQ ID NO: 52 - RNA, 19 nt, synthetic construct
  source 1..19: mol_type = other RNA
cagcattcttgctgctgag

SEQ ID NO: 53 - RNA, 19 nt, synthetic construct
  source 1..19: mol_type = other RNA
agcattcttgctgctgagc

SEQ ID NO: 54 - RNA, 19 nt, synthetic construct
  source 1..19: mol_type = other RNA
cattcttgctgctgagcca

SEQ ID NO: 55 - RNA, 19 nt, synthetic construct
  source 1..19: mol_type = other RNA
attcttgctgctgagccac

SEQ ID NO: 56 - RNA, 19 nt, synthetic construct
  source 1..19: mol_type = other RNA
catcactggagcagacatc

SEQ ID NO: 57 - RNA, 19 nt, synthetic construct
  source 1..19: mol_type = other RNA
atcactggagcagacatca

SEQ ID NO: 58 - RNA, 19 nt, synthetic construct
  source 1..19: mol_type = other RNA
tcactggagcagacatcag

SEQ ID NO: 59 - RNA, 19 nt, synthetic construct
  source 1..19: mol_type = other RNA
actggagcagacatcaggg

SEQ ID NO: 60 - RNA, 19 nt, synthetic construct
  source 1..19: mol_type = other RNA
gttcctcaggtcaccactt

SEQ ID NO: 61 - RNA, 19 nt, synthetic construct
  source 1..19: mol_type = other RNA
ttcctcaggtcaccacttg

SEQ ID NO: 62 - RNA, 19 nt, synthetic construct
  source 1..19: mol_type = other RNA
tcctcaggtcaccacttgc

SEQ ID NO: 63 - RNA, 19 nt, synthetic construct
  source 1..19: mol_type = other RNA
cctcaggtcaccacttgct

SEQ ID NO: 64 - RNA, 19 nt, synthetic construct
  source 1..19: mol_type = other RNA
ctcaggtcaccacttgctg

SEQ ID NO: 65 - RNA, 19 nt, synthetic construct
  source 1..19: mol_type = other RNA
tcaggtcaccacttgctgg

SEQ ID NO: 66 - RNA, 19 nt, synthetic construct
  source 1..19: mol_type = other RNA
aggtcaccacttgctggat

SEQ ID NO: 67 - RNA, 19 nt, synthetic construct
  source 1..19: mol_type = other RNA
tcaccacttgctggatcca

SEQ ID NO: 68 - RNA, 19 nt, synthetic construct
  source 1..19: mol_type = other RNA
caccacttgctggatccag

SEQ ID NO: 69 - RNA, 19 nt, synthetic construct
  source 1..19: mol_type = other RNA
atcacacctgtgatgcggg

SEQ ID NO: 70 - RNA, 19 nt, synthetic construct
  source 1..19: mol_type = other RNA
tcacacctgtgatgcgggt

SEQ ID NO: 71 - RNA, 19 nt, synthetic construct
  source 1..19: mol_type = other RNA
tgtagacgccgaagtagtt

SEQ ID NO: 72 - RNA, 19 nt, synthetic construct
  source 1..19: mol_type = other RNA
acaggtcctgtgggatcaa

SEQ ID NO: 73 - RNA, 19 nt, synthetic construct
  source 1..19: mol_type = other RNA
tcctgtgggatcaactgca

SEQ ID NO: 74 - RNA, 19 nt, synthetic construct
  source 1..19: mol_type = other RNA
caccttgaaggacacctct

SEQ ID NO: 75 - RNA, 19 nt, synthetic construct
  source 1..19: mol_type = other RNA
accttgaaggacacctctc

SEQ ID NO: 76 - RNA, 19 nt, synthetic construct
  source 1..19: mol_type = other RNA
ttgaaggacacctctccag

SEQ ID NO: 77 - RNA, 19 nt, synthetic construct
  source 1..19: mol_type = other RNA
atgtgtcgaccccgaacct

SEQ ID NO: 78 - RNA, 19 nt, synthetic construct
  source 1..19: mol_type = other RNA
ctcacactggaaggtgaat

SEQ ID NO: 79 - RNA, 19 nt, synthetic construct
  source 1..19: mol_type = other RNA
tcacactggaaggtgaatg

SEQ ID NO: 80 - RNA, 19 nt, synthetic construct
  source 1..19: mol_type = other RNA
cacactggaaggtgaatgt

SEQ ID NO: 81 - RNA, 19 nt, synthetic construct
  source 1..19: mol_type = other RNA
acactggaaggtgaatgtc

SEQ ID NO: 82 - RNA, 19 nt, synthetic construct
  source 1..19: mol_type = other RNA
cactggaaggtgaatgtcc

SEQ ID NO: 83 - RNA, 19 nt, synthetic construct
  source 1..19: mol_type = other RNA
cactggaatgtggctctgc

SEQ ID NO: 84 - RNA, 19 nt, synthetic construct
  source 1..19: mol_type = other RNA
actggaatgtggctctgca

SEQ ID NO: 85 - RNA, 19 nt, synthetic construct
  source 1..19: mol_type = other RNA
ctggaatgtggctctgcaa

SEQ ID NO: 86 - RNA, 19 nt, synthetic construct
  source 1..19: mol_type = other RNA
tggaatgtggctctgcaaa

SEQ ID NO: 87 - RNA, 19 nt, synthetic construct
  source 1..19: mol_type = other RNA
ggctctgcaaacgcagttt

SEQ ID NO: 88 - RNA, 19 nt, synthetic construct
  source 1..19: mol_type = other RNA
gctctgcaaacgcagtttc

SEQ ID NO: 89 - RNA, 19 nt, synthetic construct
  source 1..19: mol_type = other RNA
ctctgcaaacgcagtttct

SEQ ID NO: 90 - RNA, 19 nt, synthetic construct
  source 1..19: mol_type = other RNA
tctgcaaacgcagtttctc

SEQ ID NO: 91 - RNA, 19 nt, synthetic construct
  source 1..19: mol_type = other RNA
ctgcaaacgcagtttctct

SEQ ID NO: 92 - RNA, 19 nt, synthetic construct
  source 1..19: mol_type = other RNA
tgcaaacgcagtttctctc

SEQ ID NO: 93 - RNA, 19 nt, synthetic construct
  source 1..19: mol_type = other RNA
gcaaacgcagtttctctca

SEQ ID NO: 94 - RNA, 19 nt, synthetic construct
  source 1..19: mol_type = other RNA
caaacgcagtttctctcat

SEQ ID NO: 95 - RNA, 19 nt, synthetic construct
  source 1..19: mol_type = other RNA
aaacgcagtttctctcatc

SEQ ID NO: 96 - RNA, 19 nt, synthetic construct
  source 1..19: mol_type = other RNA
aacgcagtttctctcatcc

SEQ ID NO: 97 - RNA, 19 nt, synthetic construct
  source 1..19: mol_type = other RNA
acgcagtttctctcatcca

SEQ ID NO: 98 - RNA, 19 nt, synthetic construct
  source 1..19: mol_type = other RNA
cgcagtttctctcatccag

SEQ ID NO: 99 - RNA, 19 nt, synthetic construct
  source 1..19: mol_type = other RNA
gcagtttctctcatccagg

SEQ ID NO: 100 - RNA, 19 nt, synthetic construct
  source 1..19: mol_type = other RNA
tccttgaccccatcacagg

SEQ ID NO: 101 - RNA, 19 nt, synthetic construct
  source 1..19: mol_type = other RNA
ggtccgactggttgtacaa

SEQ ID NO: 102 - RNA, 19 nt, synthetic construct
  source 1..19: mol_type = other RNA
gtccgactggttgtacaag

SEQ ID NO: 103 - RNA, 19 nt, synthetic construct
  source 1..19: mol_type = other RNA
tccgactggttgtacaagc

SEQ ID NO: 104 - RNA, 19 nt, synthetic construct
  source 1..19: mol_type = other RNA
cgactggttgtacaagcca

SEQ ID NO: 105 - RNA, 19 nt, synthetic construct
  source 1..19: mol_type = other RNA
gactggttgtacaagccat

SEQ ID NO: 106 - RNA, 19 nt, synthetic construct
  source 1..19: mol_type = other RNA
actggttgtacaagccata

SEQ ID NO: 107 - RNA, 19 nt, synthetic construct
  source 1..19: mol_type = other RNA
ctggttgtacaagccatag

SEQ ID NO: 108 - RNA, 19 nt, synthetic construct
  source 1..19: mol_type = other RNA
tggttgtacaagccatagt

SEQ ID NO: 109 - RNA, 19 nt, synthetic construct
  source 1..19: mol_type = other RNA
ggttgtacaagccatagtg

SEQ ID NO: 110 - RNA, 19 nt, synthetic construct
  source 1..19: mol_type = other RNA
gttgtacaagccatagtgc

SEQ ID NO: 111 - RNA, 19 nt, synthetic construct
  source 1..19: mol_type = other RNA
ttgtacaagccatagtgca

SEQ ID NO: 112 - RNA, 19 nt, synthetic construct
  source 1..19: mol_type = other RNA
tgtacaagccatagtgcac

SEQ ID NO: 113 - RNA, 19 nt, synthetic construct
  source 1..19: mol_type = other RNA
gtacaagccatagtgcacc

SEQ ID NO: 114 - RNA, 19 nt, synthetic construct
  source 1..19: mol_type = other RNA
tacaagccatagtgcaccc

SEQ ID NO: 115 - RNA, 19 nt, synthetic construct
  source 1..19: mol_type = other RNA
acaagccatagtgcacccg

SEQ ID NO: 116 - RNA, 19 nt, synthetic construct
  source 1..19: mol_type = other RNA
aagccatagtgcacccgca

SEQ ID NO: 117 - RNA, 19 nt, synthetic construct
  source 1..19: mol_type = other RNA
ccatagtgcacccgcacac

SEQ ID NO: 118 - RNA, 19 nt, synthetic construct
  source 1..19: mol_type = other RNA
tcctgttctggatcgtcca

SEQ ID NO: 119 - RNA, 19 nt, synthetic construct
  source 1..19: mol_type = other RNA
tgttctggatcgtccactg

SEQ ID NO: 120 - RNA, 19 nt, synthetic construct
  source 1..19: mol_type = other RNA
tgcacggcaaatcatactt

SEQ ID NO: 121 - RNA, 19 nt, synthetic construct
  source 1..19: mol_type = other RNA
gcacggcaaatcatacttc

SEQ ID NO: 122 - RNA, 19 nt, synthetic construct
  source 1..19: mol_type = other RNA
cacggcaaatcatacttct

SEQ ID NO: 123 - RNA, 19 nt, synthetic construct
  source 1..19: mol_type = other RNA
acggcaaatcatacttctg

SEQ ID NO: 124 - RNA, 19 nt, synthetic construct
  source 1..19: mol_type = other RNA
ggcaaatcatacttctgcc

SEQ ID NO: 125 - RNA, 19 nt, synthetic construct
  source 1..19: mol_type = other RNA
gcaaatcatacttctgcct

SEQ ID NO: 126 - RNA, 19 nt, synthetic construct
  source 1..19: mol_type = other RNA
caaatcatacttctgcctc

SEQ ID NO: 127 - RNA, 19 nt, synthetic construct
  source 1..19: mol_type = other RNA
aaatcatacttctgcctcc

SEQ ID NO: 128 - RNA, 19 nt, synthetic construct
  source 1..19: mol_type = other RNA
gagtagtagctggggaagt

SEQ ID NO: 129 - RNA, 19 nt, synthetic construct
  source 1..19: mol_type = other RNA
tagtagctggggaagtacg

SEQ ID NO: 130 - RNA, 19 nt, synthetic construct
  source 1..19: mol_type = other RNA
gggtcgtagtagctgtgca

SEQ ID NO: 131 - RNA, 19 nt, synthetic construct
  source 1..19: mol_type = other RNA
tagctgtgcaggcccttct

SEQ ID NO: 132 - RNA, 19 nt, synthetic construct
  source 1..19: mol_type = other RNA
tcatacatggccagtcggt

SEQ ID NO: 133 - RNA, 19 nt, synthetic construct
  source 1..19: mol_type = other RNA
tagctgtagcggtaacaac

SEQ ID NO: 134 - RNA, 19 nt, synthetic construct
  source 1..19: mol_type = other RNA
gctgtagcggtaacaaccc

SEQ ID NO: 135 - RNA, 19 nt, synthetic construct
  source 1..19: mol_type = other RNA
ctgtagcggtaacaaccca

SEQ ID NO: 136 - RNA, 19 nt, synthetic construct
  source 1..19: mol_type = other RNA
tgtagcggtaacaacccag

SEQ ID NO: 137 - RNA, 19 nt, synthetic construct
  source 1..19: mol_type = other RNA
taacaacccagcgtggaat

SEQ ID NO: 138 - RNA, 19 nt, synthetic construct
  source 1..19: mol_type = other RNA
aacaacccagcgtggaatt

SEQ ID NO: 139 - RNA, 19 nt, synthetic construct
  source 1..19: mol_type = other RNA
tgcagctatgtctttcaca

SEQ ID NO: 140 - RNA, 19 nt, synthetic construct
  source 1..19: mol_type = other RNA
gcagctatgtctttcacac

SEQ ID NO: 141 - RNA, 19 nt, synthetic construct
  source 1..19: mol_type = other RNA
cagctatgtctttcacact

SEQ ID NO: 142 - RNA, 19 nt, synthetic construct
  source 1..19: mol_type = other RNA
agctatgtctttcacactg

SEQ ID NO: 143 - RNA, 19 nt, synthetic construct
  source 1..19: mol_type = other RNA
gctatgtctttcacactgg

SEQ ID NO: 144 - RNA, 19 nt, synthetic construct
  source 1..19: mol_type = other RNA
ctatgtctttcacactggc

SEQ ID NO: 145 - RNA, 19 nt, synthetic construct
  source 1..19: mol_type = other RNA
tatgtctttcacactggct

SEQ ID NO: 146 - RNA, 19 nt, synthetic construct
  source 1..19: mol_type = other RNA
atgtctttcacactggctt

SEQ ID NO: 147 - RNA, 19 nt, synthetic construct
  source 1..19: mol_type = other RNA
tgtctttcacactggcttc

SEQ ID NO: 148 - RNA, 19 nt, synthetic construct
  source 1..19: mol_type = other RNA
tgttgactgtggacagcag

SEQ ID NO: 149 - RNA, 19 nt, synthetic construct
  source 1..19: mol_type = other RNA
ttgactgtggacagcagct

SEQ ID NO: 150 - RNA, 19 nt, synthetic construct
  source 1..19: mol_type = other RNA
ggggatttggagaatgaac

SEQ ID NO: 151 - RNA, 19 nt, synthetic construct
  source 1..19: mol_type = other RNA
gggatttggagaatgaacc

SEQ ID NO: 152 - RNA, 19 nt, synthetic construct
  source 1..19: mol_type = other RNA
ggatttggagaatgaacca

SEQ ID NO: 153 - RNA, 19 nt, synthetic construct
  source 1..19: mol_type = other RNA
gatttggagaatgaaccag

SEQ ID NO: 154 - RNA, 19 nt, synthetic construct
  source 1..19: mol_type = other RNA
atttggagaatgaaccaga

SEQ ID NO: 155 - RNA, 19 nt, synthetic construct
  source 1..19: mol_type = other RNA
tttggagaatgaaccagaa

SEQ ID NO: 156 - RNA, 19 nt, synthetic construct
  source 1..19: mol_type = other RNA
ttggagaatgaaccagaag

SEQ ID NO: 157 - RNA, 19 nt, synthetic construct
  source 1..19: mol_type = other RNA
gagaatgaaccagaagaag

SEQ ID NO: 158 - RNA, 19 nt, synthetic construct
  source 1..19: mol_type = other RNA
agaatgaaccagaagaagc

SEQ ID NO: 159 - RNA, 19 nt, synthetic construct
  source 1..19: mol_type = other RNA
gaatgaaccagaagaagca

SEQ ID NO: 160 - RNA, 19 nt, synthetic construct
  source 1..19: mol_type = other RNA
aatgaaccagaagaagcag

SEQ ID NO: 161 - RNA, 19 nt, synthetic construct
  source 1..19: mol_type = other RNA
atgaaccagaagaagcagg

SEQ ID NO: 162 - RNA, 19 nt, synthetic construct
  source 1..19: mol_type = other RNA
tgaaccagaagaagcaggt

SEQ ID NO: 163 - RNA, 19 nt, synthetic construct
  source 1..19: mol_type = other RNA
gaaccagaagaagcaggtg

SEQ ID NO: 164 - RNA, 19 nt, synthetic construct
  source 1..19: mol_type = other RNA
aaaggaatagacggagctg

SEQ ID NO: 165 - RNA, 19 nt, synthetic construct
  source 1..19: mol_type = other RNA
gaatagacggagctggagt

SEQ ID NO: 166 - RNA, 19 nt, synthetic construct
  source 1..19: mol_type = other RNA
aatagacggagctggagtt

SEQ ID NO: 167 - RNA, 19 nt, synthetic construct
  source 1..19: mol_type = other RNA
atgagctccttgagcatct

SEQ ID NO: 168 - RNA, 19 nt, synthetic construct
  source 1..19: mol_type = other RNA
tgagctccttgagcatctt

SEQ ID NO: 169 - RNA, 19 nt, synthetic construct
  source 1..19: mol_type = other RNA
gagctccttgagcatcttc

SEQ ID NO: 170 - RNA, 19 nt, synthetic construct
  source 1..19: mol_type = other RNA
agctccttgagcatcttct

SEQ ID NO: 171 - RNA, 19 nt, synthetic construct
  source 1..19: mol_type = other RNA
gctccttgagcatcttctg

SEQ ID NO: 172 - RNA, 19 nt, synthetic construct
  source 1..19: mol_type = other RNA
ctccttgagcatcttctgg

SEQ ID NO: 173 - RNA, 19 nt, synthetic construct
  source 1..19: mol_type = other RNA
tccttgagcatcttctggg

SEQ ID NO: 174 - RNA, 19 nt, synthetic construct
  source 1..19: mol_type = other RNA
ttgagcatcttctgggctt

SEQ ID NO: 175 - RNA, 19 nt, synthetic construct
  source 1..19: mol_type = other RNA
tgagcatcttctgggcttt

SEQ ID NO: 176 - RNA, 19 nt, synthetic construct
  source 1..19: mol_type = other RNA
ctgagtacacctggctgac

SEQ ID NO: 177 - RNA, 19 nt, synthetic construct
  source 1..19: mol_type = other RNA
tgagtacacctggctgacc

SEQ ID NO: 178 - RNA, 19 nt, synthetic construct
  source 1..19: mol_type = other RNA
ccctaggaaataccagagt

SEQ ID NO: 179 - RNA, 19 nt, synthetic construct
  source 1..19: mol_type = other RNA
cctaggaaataccagagta

SEQ ID NO: 180 - RNA, 19 nt, synthetic construct
  source 1..19: mol_type = other RNA
ctaggaaataccagagtag

SEQ ID NO: 181 - RNA, 19 nt, synthetic construct
  source 1..19: mol_type = other RNA
taggaaataccagagtagc

SEQ ID NO: 182 - RNA, 19 nt, synthetic construct
  source 1..19: mol_type = other RNA
aggaaataccagagtagca

SEQ ID NO: 183 - RNA, 19 nt, synthetic construct
  source 1..19: mol_type = other RNA
ggaaataccagagtagcac

SEQ ID NO: 184 - RNA, 19 nt, synthetic construct
  source 1..19: mol_type = other RNA
gaaataccagagtagcacc

SEQ ID NO: 185 - RNA, 19 nt, synthetic construct
  source 1..19: mol_type = other RNA
aaataccagagtagcaccc

SEQ ID NO: 186 - RNA, 19 nt, synthetic construct
  source 1..19: mol_type = other RNA
ggcttttctcttggagtcc

SEQ ID NO: 187 - RNA, 19 nt, synthetic construct
  source 1..19: mol_type = other RNA
gcttttctcttggagtcct

SEQ ID NO: 188 - RNA, 19 nt, synthetic construct
  source 1..19: mol_type = other RNA
ttttctcttggagtcctca

SEQ ID NO: 189 - RNA, 19 nt, synthetic construct
  source 1..19: mol_type = other RNA
tttctcttggagtcctcac

SEQ ID NO: 190 - RNA, 19 nt, synthetic construct
  source 1..19: mol_type = other RNA
ttctcttggagtcctcaca

SEQ ID NO: 191 - RNA, 19 nt, synthetic construct
  source 1..19: mol_type = other RNA
tctcttggagtcctcacag

SEQ ID NO: 192 - RNA, 19 nt, synthetic construct
  source 1..19: mol_type = other RNA
ctcttggagtcctcacagg

SEQ ID NO: 193 - DNA, 25 bp, synthetic construct
  misc_feature 1..23: note = RNA
  misc_feature 24..25: note = DNA
  source 1..25: mol_type = other DNA
tgtccttcaaggtgagccgcctgct

SEQ ID NO: 194 - DNA, 25 bp, synthetic construct
  misc_feature 1..23: note = RNA
  misc_feature 24..25: note = DNA
  source 1..25: mol_type = other DNA
gtgtccttcaaggtgagccgcctgc

SEQ ID NO: 195 - DNA, 25 bp, synthetic construct
  misc_feature 1..23: note = RNA
  misc_feature 24..25: note = DNA
  source 1..25: mol_type = other DNA
ggtgtccttcaaggtgagccgcctg

SEQ ID NO: 196 - DNA, 25 bp, synthetic construct
  misc_feature 1..23: note = RNA
  misc_feature 24..25: note = DNA
  source 1..25: mol_type = other DNA
aggtgtccttcaaggtgagccgcct

SEQ ID NO: 197 - DNA, 25 bp, synthetic construct
  misc_feature 1..23: note = RNA
  misc_feature 24..25: note = DNA
  source 1..25: mol_type = other DNA
gaggtgtccttcaaggtgagccgcc

SEQ ID NO: 198 - DNA, 25 bp, synthetic construct
  misc_feature 1..23: note = RNA
  misc_feature 24..25: note = DNA
  source 1..25: mol_type = other DNA
aggaggacagcatggcctccacggt

SEQ ID NO: 199 - DNA, 25 bp, synthetic construct
  misc_feature 1..23: note = RNA
  misc_feature 24..25: note = DNA
  source 1..25: mol_type = other DNA
caggaggacagcatggcctccacgg

SEQ ID NO: 200 - DNA, 25 bp, synthetic construct
  misc_feature 1..23: note = RNA
  misc_feature 24..25: note = DNA
  source 1..25: mol_type = other DNA
ccaggaggacagcatggcctccacg

SEQ ID NO: 201 - DNA, 25 bp, synthetic construct
  misc_feature 1..23: note = RNA
  misc_feature 24..25: note = DNA
  source 1..25: mol_type = other DNA
tccaggaggacagcatggcctccac

SEQ ID NO: 202 - DNA, 25 bp, synthetic construct
  misc_feature 1..23: note = RNA
  misc_feature 24..25: note = DNA
  source 1..25: mol_type = other DNA
ttccaggaggacagcatggcctcca

SEQ ID NO: 203 - DNA, 25 bp, synthetic construct
  misc_feature 1..23: note = RNA
  misc_feature 24..25: note = DNA
  source 1..25: mol_type = other DNA
cttccaggaggacagcatggcctcc

SEQ ID NO: 204 - DNA, 25 bp, synthetic construct
  misc_feature 1..23: note = RNA
  misc_feature 24..25: note = DNA
  source 1..25: mol_type = other DNA
gcttccaggaggacagcatggcctc

SEQ ID NO: 205 - DNA, 25 bp, synthetic construct
  misc_feature 1..23: note = RNA
  misc_feature 24..25: note = DNA
  source 1..25: mol_type = other DNA
tgcttccaggaggacagcatggcct

SEQ ID NO: 206 - DNA, 25 bp, synthetic construct
  misc_feature 1..23: note = RNA
  misc_feature 24..25: note = DNA
  source 1..25: mol_type = other DNA
tcatcgctgaccgctgggtgataac

SEQ ID NO: 207 - DNA, 25 bp, synthetic construct
  misc_feature 1..23: note = RNA
  misc_feature 24..25: note = DNA
  source 1..25: mol_type = other DNA
ctcatcgctgaccgctgggtgataa

SEQ ID NO: 208 - DNA, 25 bp, synthetic construct
  misc_feature 1..23: note = RNA
  misc_feature 24..25: note = DNA
  source 1..25: mol_type = other DNA
tgtgactgtggcctccagggcccct

SEQ ID NO: 209 - DNA, 25 bp, synthetic construct
  misc_feature 1..23: note = RNA
  misc_feature 24..25: note = DNA
  source 1..25: mol_type = other DNA
actgtgactgtggcctccagggccc

SEQ ID NO: 210 - DNA, 25 bp, synthetic construct
  misc_feature 1..23: note = RNA
  misc_feature 24..25: note = DNA
  source 1..25: mol_type = other DNA
cactgtgactgtggcctccagggcc

SEQ ID NO: 211 - DNA, 25 bp, synthetic construct
  misc_feature 1..23: note = RNA
  misc_feature 24..25: note = DNA
  source 1..25: mol_type = other DNA
cctcccagatctccctcaccgggcc

SEQ ID NO: 212 - DNA, 25 bp, synthetic construct
  misc_feature 1..23: note = RNA
  misc_feature 24..25: note = DNA
  source 1..25: mol_type = other DNA
tcacctcccagatctccctcaccgg

SEQ ID NO: 213 - DNA, 25 bp, synthetic construct
  misc_feature 1..23: note = RNA
  misc_feature 24..25: note = DNA
  source 1..25: mol_type = other DNA
ttcacctcccagatctccctcaccg

SEQ ID NO: 214 - DNA, 25 bp, synthetic construct
  misc_feature 1..23: note = RNA
  misc_feature 24..25: note = DNA
  source 1..25: mol_type = other DNA
ggtatttcctagggtacaaggcgga

SEQ ID NO: 215 - DNA, 25 bp, synthetic construct
  misc_feature 1..23: note = RNA
  misc_feature 24..25: note = DNA
  source 1..25: mol_type = other DNA
tggtatttcctagggtacaaggcgg

SEQ ID NO: 216 - DNA, 25 bp, synthetic construct
  misc_feature 1..23: note = RNA
  misc_feature 24..25: note = DNA
  source 1..25: mol_type = other DNA
gctaccgcaagggcaagaaggatgc

SEQ ID NO: 217 - DNA, 25 bp, synthetic construct
  misc_feature 1..23: note = RNA
  misc_feature 24..25: note = DNA
  source 1..25: mol_type = other DNA
ggctaccgcaagggcaagaaggatg

SEQ ID NO: 218 - DNA, 25 bp, synthetic construct
  misc_feature 1..23: note = RNA
  misc_feature 24..25: note = DNA
  source 1..25: mol_type = other DNA
catggcaggccagcctccaggttcg

SEQ ID NO: 219 - DNA, 25 bp, synthetic construct
  misc_feature 1..23: note = RNA
  misc_feature 24..25: note = DNA
  source 1..25: mol_type = other DNA
agggtgagtggccatggcaggccag

SEQ ID NO: 220 - DNA, 25 bp, synthetic construct
  misc_feature 1..23: note = RNA
  misc_feature 24..25: note = DNA
  source 1..25: mol_type = other DNA
cgagggtgagtggccatggcaggcc

SEQ ID NO: 221 - DNA, 25 bp, synthetic construct
  misc_feature 1..23: note = RNA
  misc_feature 24..25: note = DNA
  source 1..25: mol_type = other DNA
gtcctccgagggtgagtggccatgg

SEQ ID NO: 222 - DNA, 25 bp, synthetic construct
  misc_feature 1..23: note = RNA
  misc_feature 24..25: note = DNA
  source 1..25: mol_type = other DNA
tgtcctccgagggtgagtggccatg

SEQ ID NO: 223 - DNA, 25 bp, synthetic construct
  misc_feature 1..23: note = RNA
  misc_feature 24..25: note = DNA
  source 1..25: mol_type = other DNA
gtgtcctccgagggtgagtggccat

SEQ ID NO: 224 - DNA, 25 bp, synthetic construct
  misc_feature 1..23: note = RNA
  misc_feature 24..25: note = DNA
  source 1..25: mol_type = other DNA
gcctggatgagagaaactgcgtttg

SEQ ID NO: 225 - DNA, 25 bp, synthetic construct
  misc_feature 1..23: note = RNA
  misc_feature 24..25: note = DNA
  source 1..25: mol_type = other DNA
ggcctggatgagagaaactgcgttt

SEQ ID NO: 226 - DNA, 25 bp, synthetic construct
  misc_feature 1..23: note = RNA
  misc_feature 24..25: note = DNA
  source 1..25: mol_type = other DNA
ctctggactacggcttggccctctg

SEQ ID NO: 227 - DNA, 25 bp, synthetic construct
  misc_feature 1..23: note = RNA
  misc_feature 24..25: note = DNA
  source 1..25: mol_type = other DNA
tctctggactacggcttggccctct

SEQ ID NO: 228 - DNA, 25 bp, synthetic construct
  misc_feature 1..23: note = RNA
  misc_feature 24..25: note = DNA
  source 1..25: mol_type = other DNA
tcacctgcttcttctggttcattct

SEQ ID NO: 229 - DNA, 25 bp, synthetic construct
  misc_feature 1..23: note = RNA
  misc_feature 24..25: note = DNA
  source 1..25: mol_type = other DNA
ctcacctgcttcttctggttcattc

SEQ ID NO: 230 - DNA, 25 bp, synthetic construct
  misc_feature 1..23: note = RNA
  misc_feature 24..25: note = DNA
  source 1..25: mol_type = other DNA
caaagcccagaagatgctcaaggag

SEQ ID NO: 231 - DNA, 25 bp, synthetic construct
  misc_feature 1..23: note = RNA
  misc_feature 24..25: note = DNA
  source 1..25: mol_type = other DNA
ccaaagcccagaagatgctcaagga

SEQ ID NO: 232 - DNA, 25 bp, synthetic construct
  misc_feature 1..23: note = RNA
  misc_feature 24..25: note = DNA
  source 1..25: mol_type = other DNA
gccaaagcccagaagatgctcaagg

SEQ ID NO: 233 - DNA, 25 bp, synthetic construct
  misc_feature 1..23: note = RNA
  misc_feature 24..25: note = DNA
  source 1..25: mol_type = other DNA
tttggaataaagctgcctgatccaa

SEQ ID NO: 234 - DNA, 25 bp, synthetic construct
  misc_feature 1..23: note = RNA
  misc_feature 24..25: note = DNA
  source 1..25: mol_type = other DNA
ctttggaataaagctgcctgatcca

SEQ ID NO: 235 - DNA, 25 bp, synthetic construct
  misc_feature 1..23: note = RNA
  misc_feature 24..25: note = DNA
  source 1..25: mol_type = other DNA
ccctttggaataaagctgcctgatc

SEQ ID NO: 236 - DNA, 25 bp, synthetic construct
  misc_feature 1..23: note = RNA
  misc_feature 24..25: note = DNA
  source 1..25: mol_type = other DNA
gccctttggaataaagctgcctgat

SEQ ID NO: 237 - DNA, 25 bp, synthetic construct
  misc_feature 1..23: note = RNA
  misc_feature 24..25: note = DNA
  source 1..25: mol_type = other DNA
tgccctttggaataaagctgcctga

SEQ ID NO: 238 - DNA, 25 bp, synthetic construct
  misc_feature 1..23: note = RNA
  misc_feature 24..25: note = DNA
  source 1..25: mol_type = other DNA
agctgccctttggaataaagctgcc

SEQ ID NO: 239 - DNA, 25 bp, synthetic construct
  misc_feature 1..23: note = RNA
  misc_feature 24..25: note = DNA
  source 1..25: mol_type = other DNA
cagctgccctttggaataaagctgc

SEQ ID NO: 240 - DNA, 25 bp, synthetic construct
  misc_feature 1..23: note = RNA
  misc_feature 24..25: note = DNA
  source 1..25: mol_type = other DNA
tgagctcagctgccctttggaataa

SEQ ID NO: 241 - DNA, 25 bp, synthetic construct
  misc_feature 1..23: note = RNA
  misc_feature 24..25: note = DNA
  source 1..25: mol_type = other DNA
gtgagctcagctgccctttggaata

SEQ ID NO: 242 - DNA, 25 bp, synthetic construct
  misc_feature 1..23: note = RNA
  misc_feature 24..25: note = DNA
  source 1..25: mol_type = other DNA
agcttcggaagcccctggtctaact

SEQ ID NO: 243 - DNA, 25 bp, synthetic construct
  misc_feature 1..23: note = RNA
  misc_feature 24..25: note = DNA
  source 1..25: mol_type = other DNA
tcagcagcaagaatgctggttctac

SEQ ID NO: 244 - DNA, 25 bp, synthetic construct
  misc_feature 1..23: note = RNA
  misc_feature 24..25: note = DNA
  source 1..25: mol_type = other DNA
ctcagcagcaagaatgctggttcta

SEQ ID NO: 245 - DNA, 25 bp, synthetic construct
  misc_feature 1..23: note = RNA
  misc_feature 24..25: note = DNA
  source 1..25: mol_type = other DNA
gctcagcagcaagaatgctggttct

SEQ ID NO: 246 - DNA, 25 bp, synthetic construct
  misc_feature 1..23: note = RNA
  misc_feature 24..25: note = DNA
  source 1..25: mol_type = other DNA
tggctcagcagcaagaatgctggtt

SEQ ID NO: 247 - DNA, 25 bp, synthetic construct
  misc_feature 1..23: note = RNA
  misc_feature 24..25: note = DNA
  source 1..25: mol_type = other DNA
gtggctcagcagcaagaatgctggt

SEQ ID NO: 248 - DNA, 25 bp, synthetic construct
  misc_feature 1..23: note = RNA
  misc_feature 24..25: note = DNA
  source 1..25: mol_type = other DNA
gatgtctgctccagtgatggcagga

SEQ ID NO: 249 - DNA, 25 bp, synthetic construct
  misc_feature 1..23: note = RNA
  misc_feature 24..25: note = DNA
  source 1..25: mol_type = other DNA
tgatgtctgctccagtgatggcagg

SEQ ID NO: 250 - DNA, 25 bp, synthetic construct
  misc_feature 1..23: note = RNA
  misc_feature 24..25: note = DNA
  source 1..25: mol_type = other DNA
ctgatgtctgctccagtgatggcag

SEQ ID NO: 251 - DNA, 25 bp, synthetic construct
  misc_feature 1..23: note = RNA
  misc_feature 24..25: note = DNA
  source 1..25: mol_type = other DNA
ccctgatgtctgctccagtgatggc

SEQ ID NO: 252 - DNA, 25 bp, synthetic construct
  misc_feature 1..23: note = RNA
  misc_feature 24..25: note = DNA
  source 1..25: mol_type = other DNA
aagtggtgacctgaggaactgcccc

SEQ ID NO: 253 - DNA, 25 bp, synthetic construct
  misc_feature 1..23: note = RNA
  misc_feature 24..25: note = DNA
  source 1..25: mol_type = other DNA
caagtggtgacctgaggaactgccc

SEQ ID NO: 254 - DNA, 25 bp, synthetic construct
  misc_feature 1..23: note = RNA
  misc_feature 24..25: note = DNA
  source 1..25: mol_type = other DNA
gcaagtggtgacctgaggaactgcc

SEQ ID NO: 255 - DNA, 25 bp, synthetic construct
  misc_feature 1..23: note = RNA
  misc_feature 24..25: note = DNA
  source 1..25: mol_type = other DNA
agcaagtggtgacctgaggaactgc

SEQ ID NO: 256 - DNA, 25 bp, synthetic construct
  misc_feature 1..23: note = RNA
  misc_feature 24..25: note = DNA
  source 1..25: mol_type = other DNA
cagcaagtggtgacctgaggaactg

SEQ ID NO: 257 - DNA, 25 bp, synthetic construct
  misc_feature 1..23: note = RNA
  misc_feature 24..25: note = DNA
  source 1..25: mol_type = other DNA
ccagcaagtggtgacctgaggaact

SEQ ID NO: 258 - DNA, 25 bp, synthetic construct
  misc_feature 1..23: note = RNA
  misc_feature 24..25: note = DNA
  source 1..25: mol_type = other DNA
atccagcaagtggtgacctgaggaa

SEQ ID NO: 259 - DNA, 25 bp, synthetic construct
  misc_feature 1..23: note = RNA
  misc_feature 24..25: note = DNA
  source 1..25: mol_type = other DNA
tggatccagcaagtggtgacctgag

SEQ ID NO: 260 - DNA, 25 bp, synthetic construct
  misc_feature 1..23: note = RNA
  misc_feature 24..25: note = DNA
  source 1..25: mol_type = other DNA
ctggatccagcaagtggtgacctga

SEQ ID NO: 261 - DNA, 25 bp, synthetic construct
  misc_feature 1..23: note = RNA
  misc_feature 24..25: note = DNA
  source 1..25: mol_type = other DNA
cccgcatcacaggtgtgatcagctg

SEQ ID NO: 262 - DNA, 25 bp, synthetic construct
  misc_feature 1..23: note = RNA
  misc_feature 24..25: note = DNA
  source 1..25: mol_type = other DNA
acccgcatcacaggtgtgatcagct

SEQ ID NO: 263 - DNA, 25 bp, synthetic construct
  misc_feature 1..23: note = RNA
  misc_feature 24..25: note = DNA
  source 1..25: mol_type = other DNA
aactacttcggcgtctacacccgca

SEQ ID NO: 264 - DNA, 25 bp, synthetic construct
  misc_feature 1..23: note = RNA
  misc_feature 24..25: note = DNA
  source 1..25: mol_type = other DNA
ttgatcccacaggacctgtgcagcg

SEQ ID NO: 265 - DNA, 25 bp, synthetic construct
  misc_feature 1..23: note = RNA
  misc_feature 24..25: note = DNA
  source 1..25: mol_type = other DNA
tgcagttgatcccacaggacctgtg

SEQ ID NO: 266 - DNA, 25 bp, synthetic construct
  misc_feature 1..23: note = RNA
  misc_feature 24..25: note = DNA
  source 1..25: mol_type = other DNA
agaggtgtccttcaaggtgagccgc

SEQ ID NO: 267 - DNA, 25 bp, synthetic construct
  misc_feature 1..23: note = RNA
  misc_feature 24..25: note = DNA
  source 1..25: mol_type = other DNA
gagaggtgtccttcaaggtgagccg

SEQ ID NO: 268 - DNA, 25 bp, synthetic construct
  misc_feature 1..23: note = RNA
  misc_feature 24..25: note = DNA
  source 1..25: mol_type = other DNA
ctggagaggtgtccttcaaggtgag

SEQ ID NO: 269 - DNA, 25 bp, synthetic construct
  misc_feature 1..23: note = RNA
  misc_feature 24..25: note = DNA
  source 1..25: mol_type = other DNA
aggttcggggtcgacacatctgtgg

SEQ ID NO: 270 - DNA, 25 bp, synthetic construct
  misc_feature 1..23: note = RNA
  misc_feature 24..25: note = DNA
  source 1..25: mol_type = other DNA
attcaccttccagtgtgaggaccgg

SEQ ID NO: 271 - DNA, 25 bp, synthetic construct
  misc_feature 1..23: note = RNA
  misc_feature 24..25: note = DNA
  source 1..25: mol_type = other DNA
cattcaccttccagtgtgaggaccg

SEQ ID NO: 272 - DNA, 25 bp, synthetic construct
  misc_feature 1..23: note = RNA
  misc_feature 24..25: note = DNA
  source 1..25: mol_type = other DNA
acattcaccttccagtgtgaggacc

SEQ ID NO: 273 - DNA, 25 bp, synthetic construct
  misc_feature 1..23: note = RNA
  misc_feature 24..25: note = DNA
  source 1..25: mol_type = other DNA
gacattcaccttccagtgtgaggac

SEQ ID NO: 274 - DNA, 25 bp, synthetic construct
  misc_feature 1..23: note = RNA
  misc_feature 24..25: note = DNA
  source 1..25: mol_type = other DNA
ggacattcaccttccagtgtgagga

SEQ ID NO: 275 - DNA, 25 bp, synthetic construct
  misc_feature 1..23: note = RNA
  misc_feature 24..25: note = DNA
  source 1..25: mol_type = other DNA
gcagagccacattccagtgcaaaga

SEQ ID NO: 276 - DNA, 25 bp, synthetic construct
  misc_feature 1..23: note = RNA
  misc_feature 24..25: note = DNA
  source 1..25: mol_type = other DNA
tgcagagccacattccagtgcaaag

SEQ ID NO: 277 - DNA, 25 bp, synthetic construct
  misc_feature 1..23: note = RNA
  misc_feature 24..25: note = DNA
  source 1..25: mol_type = other DNA
ttgcagagccacattccagtgcaaa

SEQ ID NO: 278 - DNA, 25 bp, synthetic construct
  misc_feature 1..23: note = RNA
  misc_feature 24..25: note = DNA
  source 1..25: mol_type = other DNA
tttgcagagccacattccagtgcaa

SEQ ID NO: 279 - DNA, 25 bp, synthetic construct
  misc_feature 1..23: note = RNA
  misc_feature 24..25: note = DNA
  source 1..25: mol_type = other DNA
aaactgcgtttgcagagccacattc

SEQ ID NO: 280 - DNA, 25 bp, synthetic construct
  misc_feature 1..23: note = RNA
  misc_feature 24..25: note = DNA
  source 1..25: mol_type = other DNA
gaaactgcgtttgcagagccacatt

SEQ ID NO: 281 - DNA, 25 bp, synthetic construct
  misc_feature 1..23: note = RNA
  misc_feature 24..25: note = DNA
  source 1..25: mol_type = other DNA
agaaactgcgtttgcagagccacat

SEQ ID NO: 282 - DNA, 25 bp, synthetic construct
  misc_feature 1..23: note = RNA
  misc_feature 24..25: note = DNA
  source 1..25: mol_type = other DNA
gagaaactgcgtttgcagagccaca

SEQ ID NO: 283 - DNA, 25 bp, synthetic construct
  misc_feature 1..23: note = RNA
  misc_feature 24..25: note = DNA
  source 1..25: mol_type = other DNA
agagaaactgcgtttgcagagccac

SEQ ID NO: 284 - DNA, 25 bp, synthetic construct
  misc_feature 1..23: note = RNA
  misc_feature 24..25: note = DNA
  source 1..25: mol_type = other DNA
gagagaaactgcgtttgcagagcca

SEQ ID NO: 285 - DNA, 25 bp, synthetic construct
  misc_feature 1..23: note = RNA
  misc_feature 24..25: note = DNA
  source 1..25: mol_type = other DNA
tgagagaaactgcgtttgcagagcc

SEQ ID NO: 286 - DNA, 25 bp, synthetic construct
  misc_feature 1..23: note = RNA
  misc_feature 24..25: note = DNA
  source 1..25: mol_type = other DNA
atgagagaaactgcgtttgcagagc

SEQ ID NO: 287 - DNA, 25 bp, synthetic construct
  misc_feature 1..23: note = RNA
  misc_feature 24..25: note = DNA
  source 1..25: mol_type = other DNA
gatgagagaaactgcgtttgcagag

SEQ ID NO: 288 - DNA, 25 bp, synthetic construct
  misc_feature 1..23: note = RNA
  misc_feature 24..25: note = DNA
  source 1..25: mol_type = other DNA
ggatgagagaaactgcgtttgcaga

SEQ ID NO: 289 - DNA, 25 bp, synthetic construct
  misc_feature 1..23: note = RNA
  misc_feature 24..25: note = DNA
  source 1..25: mol_type = other DNA
tggatgagagaaactgcgtttgcag

SEQ ID NO: 290 - DNA, 25 bp, synthetic construct
  misc_feature 1..23: note = RNA
  misc_feature 24..25: note = DNA
  source 1..25: mol_type = other DNA
ctggatgagagaaactgcgtttgca

SEQ ID NO: 291 - DNA, 25 bp, synthetic construct
  misc_feature 1..23: note = RNA
  misc_feature 24..25: note = DNA
  source 1..25: mol_type = other DNA
cctggatgagagaaactgcgtttgc

SEQ ID NO: 292 - DNA, 25 bp, synthetic construct
  misc_feature 1..23: note = RNA
  misc_feature 24..25: note = DNA
  source 1..25: mol_type = other DNA
cctgtgatggggtcaaggactgccc

SEQ ID NO: 293 - DNA, 25 bp, synthetic construct
  misc_feature 1..23: note = RNA
  misc_feature 24..25: note = DNA
  source 1..25: mol_type = other DNA
ttgtacaaccagtcggacccctgcc

SEQ ID NO: 294 - DNA, 25 bp, synthetic construct
  misc_feature 1..23: note = RNA
  misc_feature 24..25: note = DNA
  source 1..25: mol_type = other DNA
cttgtacaaccagtcggacccctgc

SEQ ID NO: 295 - DNA, 25 bp, synthetic construct
  misc_feature 1..23: note = RNA
  misc_feature 24..25: note = DNA
  source 1..25: mol_type = other DNA
gcttgtacaaccagtcggacccctg

SEQ ID NO: 296 - DNA, 25 bp, synthetic construct
  misc_feature 1..23: note = RNA
  misc_feature 24..25: note = DNA
  source 1..25: mol_type = other DNA
tggcttgtacaaccagtcggacccc

SEQ ID NO: 297 - DNA, 25 bp, synthetic construct
  misc_feature 1..23: note = RNA
  misc_feature 24..25: note = DNA
  source 1..25: mol_type = other DNA
atggcttgtacaaccagtcggaccc

SEQ ID NO: 298 - DNA, 25 bp, synthetic construct
  misc_feature 1..23: note = RNA
  misc_feature 24..25: note = DNA
  source 1..25: mol_type = other DNA
tatggcttgtacaaccagtcggacc

SEQ ID NO: 299 - DNA, 25 bp, synthetic construct
  misc_feature 1..23: note = RNA
  misc_feature 24..25: note = DNA
  source 1..25: mol_type = other DNA
ctatggcttgtacaaccagtcggac

SEQ ID NO: 300 - DNA, 25 bp, synthetic construct
  misc_feature 1..23: note = RNA
  misc_feature 24..25: note = DNA
  source 1..25: mol_type = other DNA
actatggcttgtacaaccagtcgga

SEQ ID NO: 301 - DNA, 25 bp, synthetic construct
  misc_feature 1..23: note = RNA
  misc_feature 24..25: note = DNA
  source 1..25: mol_type = other DNA
cactatggcttgtacaaccagtcgg

SEQ ID NO: 302 - DNA, 25 bp, synthetic construct
  misc_feature 1..23: note = RNA
  misc_feature 24..25: note = DNA
  source 1..25: mol_type = other DNA
gcactatggcttgtacaaccagtcg

SEQ ID NO: 303 - DNA, 25 bp, synthetic construct
  misc_feature 1..23: note = RNA
  misc_feature 24..25: note = DNA
  source 1..25: mol_type = other DNA
tgcactatggcttgtacaaccagtc

SEQ ID NO: 304 - DNA, 25 bp, synthetic construct
  misc_feature 1..23: note = RNA
  misc_feature 24..25: note = DNA
  source 1..25: mol_type = other DNA
gtgcactatggcttgtacaaccagt

SEQ ID NO: 305 - DNA, 25 bp, synthetic construct
  misc_feature 1..23: note = RNA
  misc_feature 24..25: note = DNA
  source 1..25: mol_type = other DNA
ggtgcactatggcttgtacaaccag

SEQ ID NO: 306 - DNA, 25 bp, synthetic construct
  misc_feature 1..23: note = RNA
  misc_feature 24..25: note = DNA
  source 1..25: mol_type = other DNA
gggtgcactatggcttgtacaacca

SEQ ID NO: 307 - DNA, 25 bp, synthetic construct
  misc_feature 1..23: note = RNA
  misc_feature 24..25: note = DNA
  source 1..25: mol_type = other DNA
cgggtgcactatggcttgtacaacc

SEQ ID NO: 308 - DNA, 25 bp, synthetic construct
  misc_feature 1..23: note = RNA
  misc_feature 24..25: note = DNA
  source 1..25: mol_type = other DNA
tgcgggtgcactatggcttgtacaa

SEQ ID NO: 309 - DNA, 25 bp, synthetic construct
  misc_feature 1..23: note = RNA
  misc_feature 24..25: note = DNA
  source 1..25: mol_type = other DNA
gtgtgcgggtgcactatggcttgta

SEQ ID NO: 310 - DNA, 25 bp, synthetic construct
  misc_feature 1..23: note = RNA
  misc_feature 24..25: note = DNA
  source 1..25: mol_type = other DNA
tggacgatccagaacaggaggctgt

SEQ ID NO: 311 - DNA, 25 bp, synthetic construct
  misc_feature 1..23: note = RNA
  misc_feature 24..25: note = DNA
  source 1..25: mol_type = other DNA
cagtggacgatccagaacaggaggc

SEQ ID NO: 312 - DNA, 25 bp, synthetic construct
  misc_feature 1..23: note = RNA
  misc_feature 24..25: note = DNA
  source 1..25: mol_type = other DNA
aagtatgatttgccgtgcacccagg

SEQ ID NO: 313 - DNA, 25 bp, synthetic construct
  misc_feature 1..23: note = RNA
  misc_feature 24..25: note = DNA
  source 1..25: mol_type = other DNA
gaagtatgatttgccgtgcacccag

SEQ ID NO: 314 - DNA, 25 bp, synthetic construct
  misc_feature 1..23: note = RNA
  misc_feature 24..25: note = DNA
  source 1..25: mol_type = other DNA
agaagtatgatttgccgtgcaccca

SEQ ID NO: 315 - DNA, 25 bp, synthetic construct
  misc_feature 1..23: note = RNA
  misc_feature 24..25: note = DNA
  source 1..25: mol_type = other DNA
cagaagtatgatttgccgtgcaccc

SEQ ID NO: 316 - DNA, 25 bp, synthetic construct
  misc_feature 1..23: note = RNA
  misc_feature 24..25: note = DNA
  source 1..25: mol_type = other DNA
ggcagaagtatgatttgccgtgcac

SEQ ID NO: 317 - DNA, 25 bp, synthetic construct
  misc_feature 1..23: note = RNA
  misc_feature 24..25: note = DNA
  source 1..25: mol_type = other DNA
aggcagaagtatgatttgccgtgca

SEQ ID NO: 318 - DNA, 25 bp, synthetic construct
  misc_feature 1..23: note = RNA
  misc_feature 24..25: note = DNA
  source 1..25: mol_type = other DNA
gaggcagaagtatgatttgccgtgc

SEQ ID NO: 319 - DNA, 25 bp, synthetic construct
  misc_feature 1..23: note = RNA
  misc_feature 24..25: note = DNA
  source 1..25: mol_type = other DNA
ggaggcagaagtatgatttgccgtg

SEQ ID NO: 320 - DNA, 25 bp, synthetic construct
  misc_feature 1..23: note = RNA
  misc_feature 24..25: note = DNA
  source 1..25: mol_type = other DNA
acttccccagctactactcgcccca

SEQ ID NO: 321 - DNA, 25 bp, synthetic construct
  misc_feature 1..23: note = RNA
  misc_feature 24..25: note = DNA
  source 1..25: mol_type = other DNA
cgtacttccccagctactactcgcc

SEQ ID NO: 322 - DNA, 25 bp, synthetic construct
  misc_feature 1..23: note = RNA
  misc_feature 24..25: note = DNA
  source 1..25: mol_type = other DNA
tgcacagctactacgaccccttcgt

SEQ ID NO: 323 - DNA, 25 bp, synthetic construct
  misc_feature 1..23: note = RNA
  misc_feature 24..25: note = DNA
  source 1..25: mol_type = other DNA
agaagggcctgcacagctactacga

SEQ ID NO: 324 - DNA, 25 bp, synthetic construct
  misc_feature 1..23: note = RNA
  misc_feature 24..25: note = DNA
  source 1..25: mol_type = other DNA
accgactggccatgtatgacgtggc

SEQ ID NO: 325 - DNA, 25 bp, synthetic construct
  misc_feature 1..23: note = RNA
  misc_feature 24..25: note = DNA
  source 1..25: mol_type = other DNA
gttgttaccgctacagctacgtggg

SEQ ID NO: 326 - DNA, 25 bp, synthetic construct
  misc_feature 1..23: note = RNA
  misc_feature 24..25: note = DNA
  source 1..25: mol_type = other DNA
gggttgttaccgctacagctacgtg

SEQ ID NO: 327 - DNA, 25 bp, synthetic construct
  misc_feature 1..23: note = RNA
  misc_feature 24..25: note = DNA
  source 1..25: mol_type = other DNA
tgggttgttaccgctacagctacgt

SEQ ID NO: 328 - DNA, 25 bp, synthetic construct
  misc_feature 1..23: note = RNA
  misc_feature 24..25: note = DNA
  source 1..25: mol_type = other DNA
ctgggttgttaccgctacagctacg

SEQ ID NO: 329 - DNA, 25 bp, synthetic construct
  misc_feature 1..23: note = RNA
  misc_feature 24..25: note = DNA
  source 1..25: mol_type = other DNA
attccacgctgggttgttaccgcta

SEQ ID NO: 330 - DNA, 25 bp, synthetic construct
  misc_feature 1..23: note = RNA
  misc_feature 24..25: note = DNA
  source 1..25: mol_type = other DNA
aattccacgctgggttgttaccgct

SEQ ID NO: 331 - DNA, 25 bp, synthetic construct
  misc_feature 1..23: note = RNA
  misc_feature 24..25: note = DNA
  source 1..25: mol_type = other DNA
tgtgaaagacatagctgcattgaat

SEQ ID NO: 332 - DNA, 25 bp, synthetic construct
  misc_feature 1..23: note = RNA
  misc_feature 24..25: note = DNA
  source 1..25: mol_type = other DNA
gtgtgaaagacatagctgcattgaa

SEQ ID NO: 333 - DNA, 25 bp, synthetic construct
  misc_feature 1..23: note = RNA
  misc_feature 24..25: note = DNA
  source 1..25: mol_type = other DNA
agtgtgaaagacatagctgcattga

SEQ ID NO: 334 - DNA, 25 bp, synthetic construct
  misc_feature 1..23: note = RNA
  misc_feature 24..25: note = DNA
  source 1..25: mol_type = other DNA
cagtgtgaaagacatagctgcattg

SEQ ID NO: 335 - DNA, 25 bp, synthetic construct
  misc_feature 1..23: note = RNA
  misc_feature 24..25: note = DNA
  source 1..25: mol_type = other DNA
ccagtgtgaaagacatagctgcatt

SEQ ID NO: 336 - DNA, 25 bp, synthetic construct
  misc_feature 1..23: note = RNA
  misc_feature 24..25: note = DNA
  source 1..25: mol_type = other DNA
gccagtgtgaaagacatagctgcat

SEQ ID NO: 337 - DNA, 25 bp, synthetic construct
  misc_feature 1..23: note = RNA
  misc_feature 24..25: note = DNA
  source 1..25: mol_type = other DNA
agccagtgtgaaagacatagctgca

SEQ ID NO: 338 - DNA, 25 bp, synthetic construct
  misc_feature 1..23: note = RNA
  misc_feature 24..25: note = DNA
  source 1..25: mol_type = other DNA
aagccagtgtgaaagacatagctgc

SEQ ID NO: 339 - DNA, 25 bp, synthetic construct
  misc_feature 1..23: note = RNA
  misc_feature 24..25: note = DNA
  source 1..25: mol_type = other DNA
gaagccagtgtgaaagacatagctg

SEQ ID NO: 340 - DNA, 25 bp, synthetic construct
  misc_feature 1..23: note = RNA
  misc_feature 24..25: note = DNA
  source 1..25: mol_type = other DNA
ctgctgtccacagtcaacagctcgg

SEQ ID NO: 341 - DNA, 25 bp, synthetic construct
  misc_feature 1..23: note = RNA
  misc_feature 24..25: note = DNA
  source 1..25: mol_type = other DNA
agctgctgtccacagtcaacagctc

SEQ ID NO: 342 - DNA, 25 bp, synthetic construct
  misc_feature 1..23: note = RNA
  misc_feature 24..25: note = DNA
  source 1..25: mol_type = other DNA
gttcattctccaaatccccgagcac

SEQ ID NO: 343 - DNA, 25 bp, synthetic construct
  misc_feature 1..23: note = RNA
  misc_feature 24..25: note = DNA
  source 1..25: mol_type = other DNA
ggttcattctccaaatccccgagca

SEQ ID NO: 344 - DNA, 25 bp, synthetic construct
  misc_feature 1..23: note = RNA
  misc_feature 24..25: note = DNA
  source 1..25: mol_type = other DNA
tggttcattctccaaatccccgagc

SEQ ID NO: 345 - DNA, 25 bp, synthetic construct
  misc_feature 1..23: note = RNA
  misc_feature 24..25: note = DNA
  source 1..25: mol_type = other DNA
ctggttcattctccaaatccccgag

SEQ ID NO: 346 - DNA, 25 bp, synthetic construct
  misc_feature 1..23: note = RNA
  misc_feature 24..25: note = DNA
  source 1..25: mol_type = other DNA
tctggttcattctccaaatccccga

SEQ ID NO: 347 - DNA, 25 bp, synthetic construct
  misc_feature 1..23: note = RNA
  misc_feature 24..25: note = DNA
  source 1..25: mol_type = other DNA
ttctggttcattctccaaatccccg

SEQ ID NO: 348 - DNA, 25 bp, synthetic construct
  misc_feature 1..23: note = RNA
  misc_feature 24..25: note = DNA
  source 1..25: mol_type = other DNA
cttctggttcattctccaaatcccc

SEQ ID NO: 349 - DNA, 25 bp, synthetic construct
  misc_feature 1..23: note = RNA
  misc_feature 24..25: note = DNA
  source 1..25: mol_type = other DNA
cttcttctggttcattctccaaatc

SEQ ID NO: 350 - DNA, 25 bp, synthetic construct
  misc_feature 1..23: note = RNA
  misc_feature 24..25: note = DNA
  source 1..25: mol_type = other DNA
gcttcttctggttcattctccaaat

SEQ ID NO: 351 - DNA, 25 bp, synthetic construct
  misc_feature 1..23: note = RNA
  misc_feature 24..25: note = DNA
  source 1..25: mol_type = other DNA
tgcttcttctggttcattctccaaa

SEQ ID NO: 352 - DNA, 25 bp, synthetic construct
  misc_feature 1..23: note = RNA
  misc_feature 24..25: note = DNA
  source 1..25: mol_type = other DNA
ctgcttcttctggttcattctccaa

SEQ ID NO: 353 - DNA, 25 bp, synthetic construct
  misc_feature 1..23: note = RNA
  misc_feature 24..25: note = DNA
  source 1..25: mol_type = other DNA
cctgcttcttctggttcattctcca

SEQ ID NO: 354 - DNA, 25 bp, synthetic construct
  misc_feature 1..23: note = RNA
  misc_feature 24..25: note = DNA
  source 1..25: mol_type = other DNA
acctgcttcttctggttcattctcc

SEQ ID NO: 355 - DNA, 25 bp, synthetic construct
  misc_feature 1..23: note = RNA
  misc_feature 24..25: note = DNA
  source 1..25: mol_type = other DNA
cacctgcttcttctggttcattctc

SEQ ID NO: 356 - DNA, 25 bp, synthetic construct
  misc_feature 1..23: note = RNA
  misc_feature 24..25: note = DNA
  source 1..25: mol_type = other DNA
cagctccgtctattcctttggggag

SEQ ID NO: 357 - DNA, 25 bp, synthetic construct
  misc_feature 1..23: note = RNA
  misc_feature 24..25: note = DNA
  source 1..25: mol_type = other DNA
actccagctccgtctattcctttgg

SEQ ID NO: 358 - DNA, 25 bp, synthetic construct
  misc_feature 1..23: note = RNA
  misc_feature 24..25: note = DNA
  source 1..25: mol_type = other DNA
aactccagctccgtctattcctttg

SEQ ID NO: 359 - DNA, 25 bp, synthetic construct
  misc_feature 1..23: note = RNA
  misc_feature 24..25: note = DNA
  source 1..25: mol_type = other DNA
agatgctcaaggagctcatcaccag

SEQ ID NO: 360 - DNA, 25 bp, synthetic construct
  misc_feature 1..23: note = RNA
  misc_feature 24..25: note = DNA
  source 1..25: mol_type = other DNA
aagatgctcaaggagctcatcacca

SEQ ID NO: 361 - DNA, 25 bp, synthetic construct
  misc_feature 1..23: note = RNA
  misc_feature 24..25: note = DNA
  source 1..25: mol_type = other DNA
gaagatgctcaaggagctcatcacc

SEQ ID NO: 362 - DNA, 25 bp, synthetic construct
  misc_feature 1..23: note = RNA
  misc_feature 24..25: note = DNA
  source 1..25: mol_type = other DNA
agaagatgctcaaggagctcatcac

SEQ ID NO: 363 - DNA, 25 bp, synthetic construct
  misc_feature 1..23: note = RNA
  misc_feature 24..25: note = DNA
  source 1..25: mol_type = other DNA
cagaagatgctcaaggagctcatca

SEQ ID NO: 364 - DNA, 25 bp, synthetic construct
  misc_feature 1..23: note = RNA
  misc_feature 24..25: note = DNA
  source 1..25: mol_type = other DNA
ccagaagatgctcaaggagctcatc

SEQ ID NO: 365 - DNA, 25 bp, synthetic construct
  misc_feature 1..23: note = RNA
  misc_feature 24..25: note = DNA
  source 1..25: mol_type = other DNA
cccagaagatgctcaaggagctcat

SEQ ID NO: 366 - DNA, 25 bp, synthetic construct
  misc_feature 1..23: note = RNA
  misc_feature 24..25: note = DNA
  source 1..25: mol_type = other DNA
aagcccagaagatgctcaaggagct

SEQ ID NO: 367 - DNA, 25 bp, synthetic construct
  misc_feature 1..23: note = RNA
  misc_feature 24..25: note = DNA
  source 1..25: mol_type = other DNA
aaagcccagaagatgctcaaggagc

SEQ ID NO: 368 - DNA, 25 bp, synthetic construct
  misc_feature 1..23: note = RNA
  misc_feature 24..25: note = DNA
  source 1..25: mol_type = other DNA
gtcagccaggtgtactcaggcagtc

SEQ ID NO: 369 - DNA, 25 bp, synthetic construct
  misc_feature 1..23: note = RNA
  misc_feature 24..25: note = DNA
  source 1..25: mol_type = other DNA
ggtcagccaggtgtactcaggcagt

SEQ ID NO: 370 - DNA, 25 bp, synthetic construct
  misc_feature 1..23: note = RNA
  misc_feature 24..25: note = DNA
  source 1..25: mol_type = other DNA
actctggtatttcctagggtacaag

SEQ ID NO: 371 - DNA, 25 bp, synthetic construct
  misc_feature 1..23: note = RNA
  misc_feature 24..25: note = DNA
  source 1..25: mol_type = other DNA
tactctggtatttcctagggtacaa

SEQ ID NO: 372 - DNA, 25 bp, synthetic construct
  misc_feature 1..23: note = RNA
  misc_feature 24..25: note = DNA
  source 1..25: mol_type = other DNA
ctactctggtatttcctagggtaca

SEQ ID NO: 373 - DNA, 25 bp, synthetic construct
  misc_feature 1..23: note = RNA
  misc_feature 24..25: note = DNA
  source 1..25: mol_type = other DNA
gctactctggtatttcctagggtac

SEQ ID NO: 374 - DNA, 25 bp, synthetic construct
  misc_feature 1..23: note = RNA
  misc_feature 24..25: note = DNA
  source 1..25: mol_type = other DNA
tgctactctggtatttcctagggta

SEQ ID NO: 375 - DNA, 25 bp, synthetic construct
  misc_feature 1..23: note = RNA
  misc_feature 24..25: note = DNA
  source 1..25: mol_type = other DNA
gtgctactctggtatttcctagggt

SEQ ID NO: 376 - DNA, 25 bp, synthetic construct
  misc_feature 1..23: note = RNA
  misc_feature 24..25: note = DNA
  source 1..25: mol_type = other DNA
ggtgctactctggtatttcctaggg

SEQ ID NO: 377 - DNA, 25 bp, synthetic construct
  misc_feature 1..23: note = RNA
  misc_feature 24..25: note = DNA
  source 1..25: mol_type = other DNA
gggtgctactctggtatttcctagg

SEQ ID NO: 378 - DNA, 25 bp, synthetic construct
  misc_feature 1..23: note = RNA
  misc_feature 24..25: note = DNA
  source 1..25: mol_type = other DNA
ggactccaagagaaaagcccggggc

SEQ ID NO: 379 - DNA, 25 bp, synthetic construct
  misc_feature 1..23: note = RNA
  misc_feature 24..25: note = DNA
  source 1..25: mol_type = other DNA
aggactccaagagaaaagcccgggg

SEQ ID NO: 380 - DNA, 25 bp, synthetic construct
  misc_feature 1..23: note = RNA
  misc_feature 24..25: note = DNA
  source 1..25: mol_type = other DNA
tgaggactccaagagaaaagcccgg

SEQ ID NO: 381 - DNA, 25 bp, synthetic construct
  misc_feature 1..23: note = RNA
  misc_feature 24..25: note = DNA
  source 1..25: mol_type = other DNA
gtgaggactccaagagaaaagcccg

SEQ ID NO: 382 - DNA, 25 bp, synthetic construct
  misc_feature 1..23: note = RNA
  misc_feature 24..25: note = DNA
  source 1..25: mol_type = other DNA
tgtgaggactccaagagaaaagccc

SEQ ID NO: 383 - DNA, 25 bp, synthetic construct
  misc_feature 1..23: note = RNA
  misc_feature 24..25: note = DNA
  source 1..25: mol_type = other DNA
ctgtgaggactccaagagaaaagcc

SEQ ID NO: 384 - DNA, 25 bp, synthetic construct
  misc_feature 1..23: note = RNA
  misc_feature 24..25: note = DNA
  source 1..25: mol_type = other DNA
cctgtgaggactccaagagaaaagc

SEQ ID NO: 385 - RNA, 27 nt, synthetic construct
  source 1..27: mol_type = other RNA
agcaggcggctcaccttgaaggacacc

SEQ ID NO: 386 - RNA, 27 nt, synthetic construct
  source 1..27: mol_type = other RNA
gcaggcggctcaccttgaaggacacct

SEQ ID NO: 387 - RNA, 27 nt, synthetic construct
  source 1..27: mol_type = other RNA
caggcggctcaccttgaaggacacctc

SEQ ID NO: 388 - RNA, 27 nt, synthetic construct
  source 1..27: mol_type = other RNA
aggcggctcaccttgaaggacacctct

SEQ ID NO: 389 - RNA, 27 nt, synthetic construct
  source 1..27: mol_type = other RNA
ggcggctcaccttgaaggacacctctc

SEQ ID NO: 390 - RNA, 27 nt, synthetic construct
  source 1..27: mol_type = other RNA
accgtggaggccatgctgtcctcctgg

SEQ ID NO: 391 - RNA, 27 nt, synthetic construct
  source 1..27: mol_type = other RNA
ccgtggaggccatgctgtcctcctgga

SEQ ID NO: 392 - RNA, 27 nt, synthetic construct
  source 1..27: mol_type = other RNA
cgtggaggccatgctgtcctcctggaa

SEQ ID NO: 393 - RNA, 27 nt, synthetic construct
  source 1..27: mol_type = other RNA
gtggaggccatgctgtcctcctggaag

SEQ ID NO: 394 - RNA, 27 nt, synthetic construct
  source 1..27: mol_type = other RNA
tggaggccatgctgtcctcctggaagc

SEQ ID NO: 395 - RNA, 27 nt, synthetic construct
  source 1..27: mol_type = other RNA
ggaggccatgctgtcctcctggaagca

SEQ ID NO: 396 - RNA, 27 nt, synthetic construct
  source 1..27: mol_type = other RNA
gaggccatgctgtcctcctggaagcag

SEQ ID NO: 397 - RNA, 27 nt, synthetic construct
  source 1..27: mol_type = other RNA
aggccatgctgtcctcctggaagcagt

SEQ ID NO: 398 - RNA, 27 nt, synthetic construct
  source 1..27: mol_type = other RNA
gttatcacccagcggtcagcgatgagg

SEQ ID NO: 399 - RNA, 27 nt, synthetic construct
  source 1..27: mol_type = other RNA
ttatcacccagcggtcagcgatgaggg

SEQ ID NO: 400 - RNA, 27 nt, synthetic construct
  source 1..27: mol_type = other RNA
aggggccctggaggccacagtcacagt

SEQ ID NO: 401 - RNA, 27 nt, synthetic construct
  source 1..27: mol_type = other RNA
gggccctggaggccacagtcacagtgc

SEQ ID NO: 402 - RNA, 27 nt, synthetic construct
  source 1..27: mol_type = other RNA
ggccctggaggccacagtcacagtgct

SEQ ID NO: 403 - RNA, 27 nt, synthetic construct
  source 1..27: mol_type = other RNA
ggcccggtgagggagatctgggaggtg

SEQ ID NO: 404 - RNA, 27 nt, synthetic construct
  source 1..27: mol_type = other RNA
ccggtgagggagatctgggaggtgaag

SEQ ID NO: 405 - RNA, 27 nt, synthetic construct
  source 1..27: mol_type = other RNA
cggtgagggagatctgggaggtgaagt

SEQ ID NO: 406 - RNA, 27 nt, synthetic construct
  source 1..27: mol_type = other RNA
tccgccttgtaccctaggaaataccag

SEQ ID NO: 407 - RNA, 27 nt, synthetic construct
  source 1..27: mol_type = other RNA
ccgccttgtaccctaggaaataccaga

SEQ ID NO: 408 - RNA, 27 nt, synthetic construct
  source 1..27: mol_type = other RNA
gcatccttcttgcccttgcggtagccg

SEQ ID NO: 409 - RNA, 27 nt, synthetic construct
  source 1..27: mol_type = other RNA
catccttcttgcccttgcggtagccgg

SEQ ID NO: 410 - RNA, 27 nt, synthetic construct
  source 1..27: mol_type = other RNA
cgaacctggaggctggcctgccatggc

SEQ ID NO: 411 - RNA, 27 nt, synthetic construct
  source 1..27: mol_type = other RNA
ctggcctgccatggccactcaccctcg

SEQ ID NO: 412 - RNA, 27 nt, synthetic construct
  source 1..27: mol_type = other RNA
ggcctgccatggccactcaccctcgga

SEQ ID NO: 413 - RNA, 27 nt, synthetic construct
  source 1..27: mol_type = other RNA
ccatggccactcaccctcggaggacac

SEQ ID NO: 414 - RNA, 27 nt, synthetic construct
  source 1..27: mol_type = other RNA
catggccactcaccctcggaggacaca

SEQ ID NO: 415 - RNA, 27 nt, synthetic construct
  source 1..27: mol_type = other RNA
atggccactcaccctcggaggacacag

SEQ ID NO: 416 - RNA, 27 nt, synthetic construct
  source 1..27: mol_type = other RNA
caaacgcagtttctctcatccaggccg

SEQ ID NO: 417 - RNA, 27 nt, synthetic construct
  source 1..27: mol_type = other RNA
aaacgcagtttctctcatccaggccgt

SEQ ID NO: 418 - RNA, 27 nt, synthetic construct
  source 1..27: mol_type = other RNA
cagagggccaagccgtagtccagagag

SEQ ID NO: 419 - RNA, 27 nt, synthetic construct
  source 1..27: mol_type = other RNA
agagggccaagccgtagtccagagagg

SEQ ID NO: 420 - RNA, 27 nt, synthetic construct
  source 1..27: mol_type = other RNA
agaatgaaccagaagaagcaggtgagg

SEQ ID NO: 421 - RNA, 27 nt, synthetic construct
  source 1..27: mol_type = other RNA
gaatgaaccagaagaagcaggtgaggg

SEQ ID NO: 422 - RNA, 27 nt, synthetic construct
  source 1..27: mol_type = other RNA
ctccttgagcatcttctgggctttggc

SEQ ID NO: 423 - RNA, 27 nt, synthetic construct
  source 1..27: mol_type = other RNA
tccttgagcatcttctgggctttggcg

SEQ ID NO: 424 - RNA, 27 nt, synthetic construct
  source 1..27: mol_type = other RNA
ccttgagcatcttctgggctttggcgg

SEQ ID NO: 425 - RNA, 27 nt, synthetic construct
  source 1..27: mol_type = other RNA
ttggatcaggcagctttattccaaagg

SEQ ID NO: 426 - RNA, 27 nt, synthetic construct
  source 1..27: mol_type = other RNA
tggatcaggcagctttattccaaaggg

SEQ ID NO: 427 - RNA, 27 nt, synthetic construct
  source 1..27: mol_type = other RNA
gatcaggcagctttattccaaagggca

SEQ ID NO: 428 - RNA, 27 nt, synthetic construct
  source 1..27: mol_type = other RNA
atcaggcagctttattccaaagggcag

SEQ ID NO: 429 - RNA, 27 nt, synthetic construct
  source 1..27: mol_type = other RNA
tcaggcagctttattccaaagggcagc

SEQ ID NO: 430 - RNA, 27 nt, synthetic construct
  source 1..27: mol_type = other RNA
ggcagctttattccaaagggcagctga

SEQ ID NO: 431 - RNA, 27 nt, synthetic construct
  source 1..27: mol_type = other RNA
gcagctttattccaaagggcagctgag

SEQ ID NO: 432 - RNA, 27 nt, synthetic construct
  source 1..27: mol_type = other RNA
ttattccaaagggcagctgagctcacc

SEQ ID NO: 433 - RNA, 27 nt, synthetic construct
  source 1..27: mol_type = other RNA
tattccaaagggcagctgagctcacct

SEQ ID NO: 434 - RNA, 27 nt, synthetic construct
  source 1..27: mol_type = other RNA
agttagaccaggggcttccgaagctgg

SEQ ID NO: 435 - RNA, 27 nt, synthetic construct
  source 1..27: mol_type = other RNA
gtagaaccagcattcttgctgctgagc

SEQ ID NO: 436 - RNA, 27 nt, synthetic construct
  source 1..27: mol_type = other RNA
tagaaccagcattcttgctgctgagcc

SEQ ID NO: 437 - RNA, 27 nt, synthetic construct
  source 1..27: mol_type = other RNA
agaaccagcattcttgctgctgagcca

SEQ ID NO: 438 - RNA, 27 nt, synthetic construct
  source 1..27: mol_type = other RNA
aaccagcattcttgctgctgagccact

SEQ ID NO: 439 - RNA, 27 nt, synthetic construct
  source 1..27: mol_type = other RNA
accagcattcttgctgctgagccactg

SEQ ID NO: 440 - RNA, 27 nt, synthetic construct
  source 1..27: mol_type = other RNA
tcctgccatcactggagcagacatcag

SEQ ID NO: 441 - RNA, 27 nt, synthetic construct
  source 1..27: mol_type = other RNA
cctgccatcactggagcagacatcagg

SEQ ID NO: 442 - RNA, 27 nt, synthetic construct
  source 1..27: mol_type = other RNA
ctgccatcactggagcagacatcaggg

SEQ ID NO: 443 - RNA, 27 nt, synthetic construct
  source 1..27: mol_type = other RNA
gccatcactggagcagacatcagggac

SEQ ID NO: 444 - RNA, 27 nt, synthetic construct
  source 1..27: mol_type = other RNA
ggggcagttcctcaggtcaccacttgc

SEQ ID NO: 445 - RNA, 27 nt, synthetic construct
  source 1..27: mol_type = other RNA
gggcagttcctcaggtcaccacttgct

SEQ ID NO: 446 - RNA, 27 nt, synthetic construct
  source 1..27: mol_type = other RNA
ggcagttcctcaggtcaccacttgctg

SEQ ID NO: 447 - RNA, 27 nt, synthetic construct
  source 1..27: mol_type = other RNA
gcagttcctcaggtcaccacttgctgg

SEQ ID NO: 448 - RNA, 27 nt, synthetic construct
  source 1..27: mol_type = other RNA
cagttcctcaggtcaccacttgctgga

SEQ ID NO: 449 - RNA, 27 nt, synthetic construct
  source 1..27: mol_type = other RNA
agttcctcaggtcaccacttgctggat

SEQ ID NO: 450 - RNA, 27 nt, synthetic construct
  source 1..27: mol_type = other RNA
ttcctcaggtcaccacttgctggatcc

SEQ ID NO: 451 - RNA, 27 nt, synthetic construct
  source 1..27: mol_type = other RNA
ctcaggtcaccacttgctggatccagc

SEQ ID NO: 452 - RNA, 27 nt, synthetic construct
  source 1..27: mol_type = other RNA
tcaggtcaccacttgctggatccagct

SEQ ID NO: 453 - RNA, 27 nt, synthetic construct
  source 1..27: mol_type = other RNA
cagctgatcacacctgtgatgcgggtg

SEQ ID NO: 454 - RNA, 27 nt, synthetic construct
  source 1..27: mol_type = other RNA
agctgatcacacctgtgatgcgggtgt

SEQ ID NO: 455 - RNA, 27 nt, synthetic construct
  source 1..27: mol_type = other RNA
tgcgggtgtagacgccgaagtagttag

SEQ ID NO: 456 - RNA, 27 nt, synthetic construct
  source 1..27: mol_type = other RNA
cgctgcacaggtcctgtgggatcaact

SEQ ID NO: 457 - RNA, 27 nt, synthetic construct
  source 1..27: mol_type = other RNA
cacaggtcctgtgggatcaactgcaca

SEQ ID NO: 458 - RNA, 27 nt, synthetic construct
  source 1..27: mol_type = other RNA
gcggctcaccttgaaggacacctctcc

SEQ ID NO: 459 - RNA, 27 nt, synthetic construct
  source 1..27: mol_type = other RNA
cggctcaccttgaaggacacctctcca

SEQ ID NO: 460 - RNA, 27 nt, synthetic construct
  source 1..27: mol_type = other RNA
ctcaccttgaaggacacctctccaggc

SEQ ID NO: 461 - RNA, 27 nt, synthetic construct
  source 1..27: mol_type = other RNA
ccacagatgtgtcgaccccgaacctgg

SEQ ID NO: 462 - RNA, 27 nt, synthetic construct
  source 1..27: mol_type = other RNA
ccggtcctcacactggaaggtgaatgt

SEQ ID NO: 463 - RNA, 27 nt, synthetic construct
  source 1..27: mol_type = other RNA
cggtcctcacactggaaggtgaatgtc

SEQ ID NO: 464 - RNA, 27 nt, synthetic construct
  source 1..27: mol_type = other RNA
ggtcctcacactggaaggtgaatgtcc

SEQ ID NO: 465 - RNA, 27 nt, synthetic construct
  source 1..27: mol_type = other RNA
gtcctcacactggaaggtgaatgtccc

SEQ ID NO: 466 - RNA, 27 nt, synthetic construct
  source 1..27: mol_type = other RNA
tcctcacactggaaggtgaatgtccca

SEQ ID NO: 467 - RNA, 27 nt, synthetic construct
  source 1..27: mol_type = other RNA
tctttgcactggaatgtggctctgcaa

SEQ ID NO: 468 - RNA, 27 nt, synthetic construct
  source 1..27: mol_type = other RNA
ctttgcactggaatgtggctctgcaaa

SEQ ID NO: 469 - RNA, 27 nt, synthetic construct
  source 1..27: mol_type = other RNA
tttgcactggaatgtggctctgcaaac

SEQ ID NO: 470 - RNA, 27 nt, synthetic construct
  source 1..27: mol_type = other RNA
ttgcactggaatgtggctctgcaaacg

SEQ ID NO: 471 - RNA, 27 nt, synthetic construct
  source 1..27: mol_type = other RNA
gaatgtggctctgcaaacgcagtttct

SEQ ID NO: 472 - RNA, 27 nt, synthetic construct
  source 1..27: mol_type = other RNA
aatgtggctctgcaaacgcagtttctc

SEQ ID NO: 473 - RNA, 27 nt, synthetic construct
  source 1..27: mol_type = other RNA
atgtggctctgcaaacgcagtttctct

SEQ ID NO: 474 - RNA, 27 nt, synthetic construct
  source 1..27: mol_type = other RNA
tgtggctctgcaaacgcagtttctctc

SEQ ID NO: 475 - RNA, 27 nt, synthetic construct
  source 1..27: mol_type = other RNA
gtggctctgcaaacgcagtttctctca

SEQ ID NO: 476 - RNA, 27 nt, synthetic construct
  source 1..27: mol_type = other RNA
tggctctgcaaacgcagtttctctcat

SEQ ID NO: 477 - RNA, 27 nt, synthetic construct
  source 1..27: mol_type = other RNA
ggctctgcaaacgcagtttctctcatc

SEQ ID NO: 478 - RNA, 27 nt, synthetic construct
  source 1..27: mol_type = other RNA
gctctgcaaacgcagtttctctcatcc

SEQ ID NO: 479 - RNA, 27 nt, synthetic construct
  source 1..27: mol_type = other RNA
ctctgcaaacgcagtttctctcatcca

SEQ ID NO: 480 - RNA, 27 nt, synthetic construct
  source 1..27: mol_type = other RNA
tctgcaaacgcagtttctctcatccag

SEQ ID NO: 481 - RNA, 27 nt, synthetic construct
  source 1..27: mol_type = other RNA
ctgcaaacgcagtttctctcatccagg

SEQ ID NO: 482 - RNA, 27 nt, synthetic construct
  source 1..27: mol_type = other RNA
tgcaaacgcagtttctctcatccaggc

SEQ ID NO: 483 - RNA, 27 nt, synthetic construct
  source 1..27: mol_type = other RNA
gcaaacgcagtttctctcatccaggcc

SEQ ID NO: 484 - RNA, 27 nt, synthetic construct
  source 1..27: mol_type = other RNA
gggcagtccttgaccccatcacaggca

SEQ ID NO: 485 - RNA, 27 nt, synthetic construct
  source 1..27: mol_type = other RNA
ggcaggggtccgactggttgtacaagc

SEQ ID NO: 486 - RNA, 27 nt, synthetic construct
  source 1..27: mol_type = other RNA
gcaggggtccgactggttgtacaagcc

SEQ ID NO: 487 - RNA, 27 nt, synthetic construct
  source 1..27: mol_type = other RNA
caggggtccgactggttgtacaagcca

SEQ ID NO: 488 - RNA, 27 nt, synthetic construct
  source 1..27: mol_type = other RNA
ggggtccgactggttgtacaagccata

SEQ ID NO: 489 - RNA, 27 nt, synthetic construct
  source 1..27: mol_type = other RNA
gggtccgactggttgtacaagccatag

SEQ ID NO: 490 - RNA, 27 nt, synthetic construct
  source 1..27: mol_type = other RNA
ggtccgactggttgtacaagccatagt

SEQ ID NO: 491 - RNA, 27 nt, synthetic construct
  source 1..27: mol_type = other RNA
gtccgactggttgtacaagccatagtg

SEQ ID NO: 492 - RNA, 27 nt, synthetic construct
  source 1..27: mol_type = other RNA
tccgactggttgtacaagccatagtgc

SEQ ID NO: 493 - RNA, 27 nt, synthetic construct
  source 1..27: mol_type = other RNA
ccgactggttgtacaagccatagtgca

SEQ ID NO: 494 - RNA, 27 nt, synthetic construct
  source 1..27: mol_type = other RNA
cgactggttgtacaagccatagtgcac

SEQ ID NO: 495 - RNA, 27 nt, synthetic construct
  source 1..27: mol_type = other RNA
gactggttgtacaagccatagtgcacc

SEQ ID NO: 496 - RNA, 27 nt, synthetic construct
  source 1..27: mol_type = other RNA
actggttgtacaagccatagtgcaccc

SEQ ID NO: 497 - RNA, 27 nt, synthetic construct
  source 1..27: mol_type = other RNA
ctggttgtacaagccatagtgcacccg

SEQ ID NO: 498 - RNA, 27 nt, synthetic construct
  source 1..27: mol_type = other RNA
tggttgtacaagccatagtgcacccgc

SEQ ID NO: 499 - RNA, 27 nt, synthetic construct
  source 1..27: mol_type = other RNA
ggttgtacaagccatagtgcacccgca

SEQ ID NO: 500 - RNA, 27 nt, synthetic construct
  source 1..27: mol_type = other RNA
ttgtacaagccatagtgcacccgcaca

SEQ ID NO: 501 - RNA, 27 nt, synthetic construct
  source 1..27: mol_type = other RNA
tacaagccatagtgcacccgcacaccg

SEQ ID NO: 502 - RNA, 27 nt, synthetic construct
  source 1..27: mol_type = other RNA
acagcctcctgttctggatcgtccact

SEQ ID NO: 503 - RNA, 27 nt, synthetic construct
  source 1..27: mol_type = other RNA
gcctcctgttctggatcgtccactggc

SEQ ID NO: 504 - RNA, 27 nt, synthetic construct
  source 1..27: mol_type = other RNA
cctgggtgcacggcaaatcatacttct

SEQ ID NO: 505 - RNA, 27 nt, synthetic construct
  source 1..27: mol_type = other RNA
ctgggtgcacggcaaatcatacttctg

SEQ ID NO: 506 - RNA, 27 nt, synthetic construct
  source 1..27: mol_type = other RNA
tgggtgcacggcaaatcatacttctgc

SEQ ID NO: 507 - RNA, 27 nt, synthetic construct
  source 1..27: mol_type = other RNA
gggtgcacggcaaatcatacttctgcc

SEQ ID NO: 508 - RNA, 27 nt, synthetic construct
  source 1..27: mol_type = other RNA
gtgcacggcaaatcatacttctgcctc

SEQ ID NO: 509 - RNA, 27 nt, synthetic construct
  source 1..27: mol_type = other RNA
tgcacggcaaatcatacttctgcctcc

SEQ ID NO: 510 - RNA, 27 nt, synthetic construct
  source 1..27: mol_type = other RNA
gcacggcaaatcatacttctgcctcct

SEQ ID NO: 511 - RNA, 27 nt, synthetic construct
  source 1..27: mol_type = other RNA
cacggcaaatcatacttctgcctcctc

SEQ ID NO: 512 - RNA, 27 nt, synthetic construct
  source 1..27: mol_type = other RNA
tggggcgagtagtagctggggaagtac

SEQ ID NO: 513 - RNA, 27 nt, synthetic construct
  source 1..27: mol_type = other RNA
ggcgagtagtagctggggaagtacggg

SEQ ID NO: 514 - RNA, 27 nt, synthetic construct
  source 1..27: mol_type = other RNA
acgaaggggtcgtagtagctgtgcagg

SEQ ID NO: 515 - RNA, 27 nt, synthetic construct
  source 1..27: mol_type = other RNA
tcgtagtagctgtgcaggcccttcttc

SEQ ID NO: 516 - RNA, 27 nt, synthetic construct
  source 1..27: mol_type = other RNA
gccacgtcatacatggccagtcggtcc

SEQ ID NO: 517 - RNA, 27 nt, synthetic construct
  source 1..27: mol_type = other RNA
cccacgtagctgtagcggtaacaaccc

SEQ ID NO: 518 - RNA, 27 nt, synthetic construct
  source 1..27: mol_type = other RNA
cacgtagctgtagcggtaacaacccag

SEQ ID NO: 519 - RNA, 27 nt, synthetic construct
  source 1..27: mol_type = other RNA
acgtagctgtagcggtaacaacccagc

SEQ ID NO: 520 - RNA, 27 nt, synthetic construct
  source 1..27: mol_type = other RNA
cgtagctgtagcggtaacaacccagcg

SEQ ID NO: 521 - RNA, 27 nt, synthetic construct
  source 1..27: mol_type = other RNA
tagcggtaacaacccagcgtggaattc

SEQ ID NO: 522 - RNA, 27 nt, synthetic construct
  source 1..27: mol_type = other RNA
agcggtaacaacccagcgtggaattca

SEQ ID NO: 523 - RNA, 27 nt, synthetic construct
  source 1..27: mol_type = other RNA
attcaatgcagctatgtctttcacact

SEQ ID NO: 524 - RNA, 27 nt, synthetic construct
  source 1..27: mol_type = other RNA
ttcaatgcagctatgtctttcacactg

SEQ ID NO: 525 - RNA, 27 nt, synthetic construct
  source 1..27: mol_type = other RNA
tcaatgcagctatgtctttcacactgg

SEQ ID NO: 526 - RNA, 27 nt, synthetic construct
  source 1..27: mol_type = other RNA
caatgcagctatgtctttcacactggc

SEQ ID NO: 527 - RNA, 27 nt, synthetic construct
  source 1..27: mol_type = other RNA
aatgcagctatgtctttcacactggct

SEQ ID NO: 528 - RNA, 27 nt, synthetic construct
  source 1..27: mol_type = other RNA
atgcagctatgtctttcacactggctt

SEQ ID NO: 529 - RNA, 27 nt, synthetic construct
  source 1..27: mol_type = other RNA
tgcagctatgtctttcacactggcttc

SEQ ID NO: 530 - RNA, 27 nt, synthetic construct
  source 1..27: mol_type = other RNA
gcagctatgtctttcacactggcttcc

SEQ ID NO: 531 - RNA, 27 nt, synthetic construct
  source 1..27: mol_type = other RNA
cagctatgtctttcacactggcttcca

SEQ ID NO: 532 - RNA, 27 nt, synthetic construct
  source 1..27: mol_type = other RNA
ccgagctgttgactgtggacagcagct

SEQ ID NO: 533 - RNA, 27 nt, synthetic construct
  source 1..27: mol_type = other RNA
gagctgttgactgtggacagcagctcc

SEQ ID NO: 534 - RNA, 27 nt, synthetic construct
  source 1..27: mol_type = other RNA
gtgctcggggatttggagaatgaacca

SEQ ID NO: 535 - RNA, 27 nt, synthetic construct
  source 1..27: mol_type = other RNA
tgctcggggatttggagaatgaaccag

SEQ ID NO: 536 - RNA, 27 nt, synthetic construct
  source 1..27: mol_type = other RNA
gctcggggatttggagaatgaaccaga

SEQ ID NO: 537 - RNA, 27 nt, synthetic construct
  source 1..27: mol_type = other RNA
ctcggggatttggagaatgaaccagaa

SEQ ID NO: 538 - RNA, 27 nt, synthetic construct
  source 1..27: mol_type = other RNA
tcggggatttggagaatgaaccagaag

SEQ ID NO: 539 - RNA, 27 nt, synthetic construct
  source 1..27: mol_type = other RNA
cggggatttggagaatgaaccagaaga

SEQ ID NO: 540 - RNA, 27 nt, synthetic construct
  source 1..27: mol_type = other RNA
ggggatttggagaatgaaccagaagaa

SEQ ID NO: 541 - RNA, 27 nt, synthetic construct
  source 1..27: mol_type = other RNA
gatttggagaatgaaccagaagaagca

SEQ ID NO: 542 - RNA, 27 nt, synthetic construct
  source 1..27: mol_type = other RNA
atttggagaatgaaccagaagaagcag

SEQ ID NO: 543 - RNA, 27 nt, synthetic construct
  source 1..27: mol_type = other RNA
tttggagaatgaaccagaagaagcagg

SEQ ID NO: 544 - RNA, 27 nt, synthetic construct
  source 1..27: mol_type = other RNA
ttggagaatgaaccagaagaagcaggt

SEQ ID NO: 545 - RNA, 27 nt, synthetic construct
  source 1..27: mol_type = other RNA
tggagaatgaaccagaagaagcaggtg

SEQ ID NO: 546 - RNA, 27 nt, synthetic construct
  source 1..27: mol_type = other RNA
ggagaatgaaccagaagaagcaggtga

SEQ ID NO: 547 - RNA, 27 nt, synthetic construct
  source 1..27: mol_type = other RNA
gagaatgaaccagaagaagcaggtgag

SEQ ID NO: 548 - RNA, 27 nt, synthetic construct
  source 1..27: mol_type = other RNA
ctccccaaaggaatagacggagctgga

SEQ ID NO: 549 - RNA, 27 nt, synthetic construct
  source 1..27: mol_type = other RNA
ccaaaggaatagacggagctggagttg

SEQ ID NO: 550 - RNA, 27 nt, synthetic construct
  source 1..27: mol_type = other RNA
caaaggaatagacggagctggagttgt

SEQ ID NO: 551 - RNA, 27 nt, synthetic construct
  source 1..27: mol_type = other RNA
ctggtgatgagctccttgagcatcttc

SEQ ID NO: 552 - RNA, 27 nt, synthetic construct
  source 1..27: mol_type = other RNA
tggtgatgagctccttgagcatcttct

SEQ ID NO: 553 - RNA, 27 nt, synthetic construct
  source 1..27: mol_type = other RNA
ggtgatgagctccttgagcatcttctg

SEQ ID NO: 554 - RNA, 27 nt, synthetic construct
  source 1..27: mol_type = other RNA
gtgatgagctccttgagcatcttctgg

SEQ ID NO: 555 - RNA, 27 nt, synthetic construct
  source 1..27: mol_type = other RNA
tgatgagctccttgagcatcttctggg

SEQ ID NO: 556 - RNA, 27 nt, synthetic construct
  source 1..27: mol_type = other RNA
gatgagctccttgagcatcttctgggc

SEQ ID NO: 557 - RNA, 27 nt, synthetic construct
  source 1..27: mol_type = other RNA
atgagctccttgagcatcttctgggct

SEQ ID NO: 558 - RNA, 27 nt, synthetic construct
  source 1..27: mol_type = other RNA
agctccttgagcatcttctgggctttg

SEQ ID NO: 559 - RNA, 27 nt, synthetic construct
  source 1..27: mol_type = other RNA
gctccttgagcatcttctgggctttgg

SEQ ID NO: 560 - RNA, 27 nt, synthetic construct
  source 1..27: mol_type = other RNA
gactgcctgagtacacctggctgacca

SEQ ID NO: 561 - RNA, 27 nt, synthetic construct
  source 1..27: mol_type = other RNA
actgcctgagtacacctggctgaccat

SEQ ID NO: 562 - RNA, 27 nt, synthetic construct
  source 1..27: mol_type = other RNA
cttgtaccctaggaaataccagagtag

SEQ ID NO: 563 - RNA, 27 nt, synthetic construct
  source 1..27: mol_type = other RNA
ttgtaccctaggaaataccagagtagc

SEQ ID NO: 564 - RNA, 27 nt, synthetic construct
  source 1..27: mol_type = other RNA
tgtaccctaggaaataccagagtagca

SEQ ID NO: 565 - RNA, 27 nt, synthetic construct
  source 1..27: mol_type = other RNA
gtaccctaggaaataccagagtagcac

SEQ ID NO: 566 - RNA, 27 nt, synthetic construct
  source 1..27: mol_type = other RNA
taccctaggaaataccagagtagcacc

SEQ ID NO: 567 - RNA, 27 nt, synthetic construct
  source 1..27: mol_type = other RNA
accctaggaaataccagagtagcaccc

SEQ ID NO: 568 - RNA, 27 nt, synthetic construct
  source 1..27: mol_type = other RNA
ccctaggaaataccagagtagcacccc

SEQ ID NO: 569 - RNA, 27 nt, synthetic construct
  source 1..27: mol_type = other RNA
cctaggaaataccagagtagcaccccc

SEQ ID NO: 570 - RNA, 27 nt, synthetic construct
  source 1..27: mol_type = other RNA
gccccgggcttttctcttggagtcctc

SEQ ID NO: 571 - RNA, 27 nt, synthetic construct
  source 1..27: mol_type = other RNA
ccccgggcttttctcttggagtcctca

SEQ ID NO: 572 - RNA, 27 nt, synthetic construct
  source 1..27: mol_type = other RNA
ccgggcttttctcttggagtcctcaca

SEQ ID NO: 573 - RNA, 27 nt, synthetic construct
  source 1..27: mol_type = other RNA
cgggcttttctcttggagtcctcacag

SEQ ID NO: 574 - RNA, 27 nt, synthetic construct
  source 1..27: mol_type = other RNA
gggcttttctcttggagtcctcacagg

SEQ ID NO: 575 - RNA, 27 nt, synthetic construct
  source 1..27: mol_type = other RNA
ggcttttctcttggagtcctcacaggc

SEQ ID NO: 576 - RNA, 27 nt, synthetic construct
  source 1..27: mol_type = other RNA
gcttttctcttggagtcctcacaggcc

SEQ ID NO: 577 - RNA, 36 nt, synthetic construct
  source 1..36: mol_type = other RNA
gtgaggactccaagagaaaagcagccgaaaggctgc

SEQ ID NO: 578 - RNA, 36 nt, synthetic construct
  source 1..36: mol_type = other RNA
gggtgctactctggtatttagcagccgaaaggctgc

SEQ ID NO: 579 - RNA, 36 nt, synthetic construct
  source 1..36: mol_type = other RNA
ggtgctactctggtatttcagcagccgaaaggctgc

SEQ ID NO: 580 - RNA, 36 nt, synthetic construct
  source 1..36: mol_type = other RNA
gctactctggtatttcctaagcagccgaaaggctgc

SEQ ID NO: 581 - RNA, 36 nt, synthetic construct
  source 1..36: mol_type = other RNA
ggtatttcctagggtacaaagcagccgaaaggctgc

SEQ ID NO: 582 - RNA, 36 nt, synthetic construct
  source 1..36: mol_type = other RNA
cacctgcttcttctggttcagcagccgaaaggctgc

SEQ ID NO: 583 - RNA, 36 nt, synthetic construct
  source 1..36: mol_type = other RNA
gaagccagtgtgaaagacaagcagccgaaaggctgc

SEQ ID NO: 584 - RNA, 36 nt, synthetic construct
  source 1..36: mol_type = other RNA
cgtacttccccagctactaagcagccgaaaggctgc

SEQ ID NO: 585 - RNA, 36 nt, synthetic construct
  source 1..36: mol_type = other RNA
ggaggcagaagtatgatttagcagccgaaaggctgc

SEQ ID NO: 586 - RNA, 36 nt, synthetic construct
  source 1..36: mol_type = other RNA
gggtgcactatggcttgtaagcagccgaaaggctgc

SEQ ID NO: 587 - RNA, 36 nt, synthetic construct
  source 1..36: mol_type = other RNA
gcactatggcttgtacaacagcagccgaaaggctgc

SEQ ID NO: 588 - RNA, 36 nt, synthetic construct
  source 1..36: mol_type = other RNA
gatgagagaaactgcgtttagcagccgaaaggctgc

SEQ ID NO: 589 - RNA, 36 nt, synthetic construct
  source 1..36: mol_type = other RNA
gaggtgtccttcaaggtgaagcagccgaaaggctgc

SEQ ID NO: 590 - RNA, 36 nt, synthetic construct
  source 1..36: mol_type = other RNA
ctcacctgcttcttctggtagcagccgaaaggctgc

SEQ ID NO: 591 - RNA, 36 nt, synthetic construct
  source 1..36: mol_type = other RNA
tcacctgcttcttctggttagcagccgaaaggctgc

SEQ ID NO: 592 - RNA, 36 nt, synthetic construct
  source 1..36: mol_type = other RNA
tctggttcattctccaaatagcagccgaaaggctgc

SEQ ID NO: 593 - RNA, 36 nt, synthetic construct
  source 1..36: mol_type = other RNA
gttcattctccaaatccccagcagccgaaaggctgc

SEQ ID NO: 594 - RNA, 36 nt, synthetic construct
  source 1..36: mol_type = other RNA
aaagtggatgtgcagttgaagcagccgaaaggctgc

SEQ ID NO: 595 - RNA, 36 nt, synthetic construct
  source 1..36: mol_type = other RNA
aactccagctccgtctattagcagccgaaaggctgc

SEQ ID NO: 596 - RNA, 36 nt, synthetic construct
  source 1..36: mol_type = other RNA
acctgcttcttctggttcaagcagccgaaaggctgc

SEQ ID NO: 597 - RNA, 36 nt, synthetic construct
  source 1..36: mol_type = other RNA
agtgtgaaagacatagctgagcagccgaaaggctgc

SEQ ID NO: 598 - RNA, 22 nt, synthetic construct
  source 1..22: mol_type = other RNA
ttttctcttggagtcctcacgg

SEQ ID NO: 599 - RNA, 22 nt, synthetic construct
  source 1..22: mol_type = other RNA
taaataccagagtagcacccgg

SEQ ID NO: 600 - RNA, 22 nt, synthetic construct
  source 1..22: mol_type = other RNA
tgaaataccagagtagcaccgg

SEQ ID NO: 601 - RNA, 22 nt, synthetic construct
  source 1..22: mol_type = other RNA
ttaggaaataccagagtagcgg

SEQ ID NO: 602 - RNA, 22 nt, synthetic construct
  source 1..22: mol_type = other RNA
tttgtaccctaggaaataccgg

SEQ ID NO: 603 - RNA, 22 nt, synthetic construct
  source 1..22: mol_type = other RNA
tgaaccagaagaagcaggtggg

SEQ ID NO: 604 - RNA, 22 nt, synthetic construct
  source 1..22: mol_type = other RNA
ttgtctttcacactggcttcgg

SEQ ID NO: 605 - RNA, 22 nt, synthetic construct
  source 1..22: mol_type = other RNA
ttagtagctggggaagtacggg

SEQ ID NO: 606 - RNA, 22 nt, synthetic construct
  source 1..22: mol_type = other RNA
taaatcatacttctgcctccgg

SEQ ID NO: 607 - RNA, 22 nt, synthetic construct
  source 1..22: mol_type = other RNA
ttacaagccatagtgcacccgg

SEQ ID NO: 608 - RNA, 22 nt, synthetic construct
  source 1..22: mol_type = other RNA
tgttgtacaagccatagtgcgg

SEQ ID NO: 609 - RNA, 22 nt, synthetic construct
  source 1..22: mol_type = other RNA
taaacgcagtttctctcatcgg

SEQ ID NO: 610 - RNA, 22 nt, synthetic construct
  source 1..22: mol_type = other RNA
ttcaccttgaaggacacctcgg

SEQ ID NO: 611 - RNA, 22 nt, synthetic construct
  source 1..22: mol_type = other RNA
taccagaagaagcaggtgaggg

SEQ ID NO: 612 - RNA, 22 nt, synthetic construct
  source 1..22: mol_type = other RNA
taaccagaagaagcaggtgagg

SEQ ID NO: 613 - RNA, 22 nt, synthetic construct
  source 1..22: mol_type = other RNA
tatttggagaatgaaccagagg

SEQ ID NO: 614 - RNA, 22 nt, synthetic construct
  source 1..22: mol_type = other RNA
tggggatttggagaatgaacgg

SEQ ID NO: 615 - RNA, 22 nt, synthetic construct
  source 1..22: mol_type = other RNA
ttcaactgcacatccactttgg

SEQ ID NO: 616 - RNA, 22 nt, synthetic construct
  source 1..22: mol_type = other RNA
taatagacggagctggagttgg

SEQ ID NO: 617 - RNA, 22 nt, synthetic construct
  source 1..22: mol_type = other RNA
ttgaaccagaagaagcaggtgg

SEQ ID NO: 618 - RNA, 22 nt, synthetic construct
  source 1..22: mol_type = other RNA
tcagctatgtctttcacactgg

SEQ ID NO: 619 - RNA, 36 nt, synthetic construct; identical to SEQ ID NO: 577
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl uridine
  modified_base 3: mod_base = OTHER; note = 2'-O-methyl guanosine
  modified_base 4: mod_base = OTHER; note = 2'-O-methyl adenosine
  modified_base 5: mod_base = OTHER; note = 2'-O-methyl guanosine
  modified_base 6: mod_base = OTHER; note = 2'-O-methyl guanosine
  modified_base 7: mod_base = OTHER; note = 2'-O-methyl adenosine
  modified_base 8: mod_base = OTHER; note = 2'-fluoro cytidine
  modified_base 9: mod_base = OTHER; note = 2'-fluoro uridine
  modified_base 10: mod_base = OTHER; note = 2'-fluoro cytidine
  modified_base 11: mod_base = OTHER; note = 2'-fluoro cytidine
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl guanosine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl adenosine
  modified_base 18: mod_base = OTHER; note = 2'-O-methyl adenosine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl mcytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 620 - RNA, 36 nt, synthetic construct; identical to SEQ ID NO: 578
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl guanosine
  modified_base 4: mod_base = OTHER; note = 2'-O-methyl uridine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl uridine
  modified_base 8: mod_base = OTHER; note = 2'-fluoro adenosine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro cytidine
  modified_base 12: mod_base = OTHER; note = 2'-O-methyl uridine
  modified_base 13: mod_base = OTHER; note = 2'-O-methyl guanosine
  modified_base 14: mod_base = OTHER; note = 2'-O-methyl guanosine
  modified_base 15: mod_base = OTHER; note = 2'-O-methyl uridine
  modified_base 16: mod_base = OTHER; note = 2'-O-methyl adenosine
  modified_base 17: mod_base = OTHER; note = 2'-O-methyl uridine
  modified_base 18: mod_base = OTHER; note = 2'-O-methyl uridine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 621 - RNA, 36 nt, synthetic construct; identical to SEQ ID NO: 579
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl cytidine
  modified_base 6: mod_base = OTHER; note = 2'-O-methyl uridine
  modified_base 7: mod_base = OTHER; note = 2'-O-methyl adenosine
  modified_base 8: mod_base = OTHER; note = 2'-fluoro cytidine
  modified_base 9: mod_base = OTHER; note = 2'-fluoro uridine
  modified_base 10: mod_base = OTHER; note = 2'-fluoro cytidine
  modified_base 11: mod_base = OTHER; note = 2'-fluoro uridine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-O-methyl uridine
  modified_base 15: mod_base = OTHER; note = 2'-O-methyl adenosine
  modified_base 16: mod_base = OTHER; note = 2'-O-methyl uridine
  modified_base 17: mod_base = OTHER; note = 2'-O-methyl uridine
  modified_base 18: mod_base = OTHER; note = 2'-O-methyl uridine
  modified_base 19: mod_base = OTHER; note = 2'-O-methyl cytidine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 622 - RNA, 36 nt, synthetic construct; identical to SEQ ID NO: 580
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl cytidine
  modified_base 3: mod_base = OTHER; note = 2'-O-methyl uridine
  modified_base 4: mod_base = OTHER; note = 2'-O-methyl adenosine
  modified_base 5: mod_base = OTHER; note = 2'-O-methyl cytidine
  modified_base 6: mod_base = OTHER; note = 2'-O-methyl uridine
  modified_base 7: mod_base = OTHER; note = 2'-O-methyl cytidine
  modified_base 8: mod_base = OTHER; note = 2'-fluoro uridine
  modified_base 9: mod_base = OTHER; note = 2'-fluoro guanosine
  modified_base 10: mod_base = OTHER; note = 2'-fluoro guanosine
  modified_base 11: mod_base = OTHER; note = 2'-fluoro uridine
  modified_base 12: mod_base = OTHER; note = 2'-O-methyl adenosine
  modified_base 13: mod_base = OTHER; note = 2'-O-methyl uridine
  modified_base 14: mod_base = OTHER; note = 2'-O-methyl uridine
  modified_base 15: mod_base = OTHER; note = 2'-O-methyl uridine
  modified_base 16: mod_base = OTHER; note = 2'-O-methyl cytidine
  modified_base 17: mod_base = OTHER; note = 2'-O-methyl cytidine
  modified_base 18: mod_base = OTHER; note = 2'-O-methyl uridine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 623 - RNA, 36 nt, synthetic construct; identical to SEQ ID NO: 581
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl uridine
  modified_base 4: mod_base = OTHER; note = 2'-O-methyl adenosine
  modified_base 5: mod_base = OTHER; note = 2'-O-methyl uridine
  modified_base 6: mod_base = OTHER; note = 2'-O-methyl uridine
  modified_base 7: mod_base = OTHER; note = 2'-O-methyl uridine
  modified_base 8: mod_base = OTHER; note = 2'-fluoro cytidine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro adensosine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-O-methyl guanosine
  modified_base 15: mod_base = OTHER; note = 2'-O-methyl uridine
  modified_base 16: mod_base = OTHER; note = 2'-O-methyl adenosine
  modified_base 17: mod_base = OTHER; note = 2'-O-methyl cytidine
  modified_base 18: mod_base = OTHER; note = 2'-O-methyl adenosine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 624 - RNA, 36 nt, synthetic construct; identical to SEQ ID NO: 582
  misc_feature 1..36: note = synthetic construct
  misc_feature 1..2: note = linked via phosphorothioate
  modified_base 1: mod_base = OTHER; note = 2'-O-methyl cytidine
  modified_base 2: mod_base = OTHER; note = 2'-O-methyl adenosine
  modified_base 3: mod_base = OTHER; note = 2'-O-methyl cytidine
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl cytidine
  modified_base 8: mod_base = OTHER; note = 2'-fluoro uridine
  modified_base 9: mod_base = OTHER; note = 2'-fluoro uridine
  modified_base 10: mod_base = OTHER; note = 2'-fluoro cytidine
  modified_base 11: mod_base = OTHER; note = 2'-fluoro uridine
  modified_base 12: mod_base = OTHER; note = 2'-O-methyl uridine
  modified_base 13: mod_base = OTHER; note = 2'-O-methyl cytidine
  modified_base 14: mod_base = OTHER; note = 2'-O-methyl uridine
  modified_base 15: mod_base = OTHER; note = 2'-O-methyl guanosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl uridine
  modified_base 19: mod_base = OTHER; note = 2'-O-methyl cytidine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 625 - RNA, 36 nt, synthetic construct; identical to SEQ ID NO: 583
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl adensosine
  modified_base 3: mod_base = OTHER; note = 2'-O-methyl adenosine
  modified_base 4: mod_base = OTHER; note = 2'-O-methyl guanosine
  modified_base 5: mod_base = OTHER; note = 2'-O-methyl cytidine
  modified_base 6: mod_base = OTHER; note = 2'-O-methyl cytidine
  modified_base 7: mod_base = OTHER; note = 2'-O-methyl adenosine
  modified_base 8: mod_base = OTHER; note = 2'-fluoro guanosine
  modified_base 9: mod_base = OTHER; note = 2'-fluoro uridine
  modified_base 10: mod_base = OTHER; note = 2'-fluoro guanosine
  modified_base 11: mod_base = OTHER; note = 2'-fluoro uridine
  modified_base 12: mod_base = OTHER; note = 2'-O-methyl guanosine
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl adenosine
  modified_base 18: mod_base = OTHER; note = 2'-O-methyl cytidine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 626 - RNA, 36 nt, synthetic construct; identical to SEQ ID NO: 584
  misc_feature 1..36: note = synthetic construct
  misc_feature 1..2: note = linked via phosphorothioate
  modified_base 1: mod_base = OTHER; note = 2'-O-methyl cytidine
  modified_base 2: mod_base = OTHER; note = 2'-O-methyl guanosine
  modified_base 3: mod_base = OTHER; note = 2'-O-methyl uridine
  modified_base 4: mod_base = OTHER; note = 2'-O-methyl adenosine
  modified_base 5: mod_base = OTHER; note = 2'-O-methyl cytidine
  modified_base 6: mod_base = OTHER; note = 2'-O-methyl uridine
  modified_base 7: mod_base = OTHER; note = 2'-O-methyl uridine
  modified_base 8: mod_base = OTHER; note = 2'-fluoro cytidine
  modified_base 9: mod_base = OTHER; note = 2'-fluoro cytidine
  modified_base 10: mod_base = OTHER; note = 2'-fluoro cytidine
  modified_base 11: mod_base = OTHER; note = 2'-fluoro cytidine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-O-methyl cytidine
  modified_base 15: mod_base = OTHER; note = 2'-O-methyl uridine
  modified_base 16: mod_base = OTHER; note = 2'-O-methyl adenosine
  modified_base 17: mod_base = OTHER; note = 2'-O-methyl cytidine
  modified_base 18: mod_base = OTHER; note = 2'-O-methyl uridine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 627 - RNA, 36 nt, synthetic construct; identical to SEQ ID NO: 585
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl adenosine
  modified_base 4: mod_base = OTHER; note = 2'-O-methyl guanosine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl adenosine
  modified_base 8: mod_base = OTHER; note = 2'-fluoro guanosine
  modified_base 9: mod_base = OTHER; note = 2'-fluoro adenosine
  modified_base 10: mod_base = OTHER; note = 2'-fluoro adenosine
  modified_base 11: mod_base = OTHER; note = 2'-fluoro guanosine
  modified_base 12: mod_base = OTHER; note = 2'-O-methyl uridine
  modified_base 13: mod_base = OTHER; note = 2'-O-methyl adenosine
  modified_base 14: mod_base = OTHER; note = 2'-O-methyl uridine
  modified_base 15: mod_base = OTHER; note = 2'-O-methyl guanosine
  modified_base 16: mod_base = OTHER; note = 2'-O-methyl adenosine
  modified_base 17: mod_base = OTHER; note = 2'-O-methyl uridine
  modified_base 18: mod_base = OTHER; note = 2'-O-methyl uridine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 628 - RNA, 36 nt, synthetic construct; identical to SEQ ID NO: 586
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl guanosine
  modified_base 3: mod_base = OTHER; note = 2'-O-methyl guanosine
  modified_base 4: mod_base = OTHER; note = 2'-O-methyl uridine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl adenosine
  modified_base 8: mod_base = OTHER; note = 2'-fluoro cytidine
  modified_base 9: mod_base = OTHER; note = 2'-fluoro uridine
  modified_base 10: mod_base = OTHER; note = 2'-fluoro adenosine
  modified_base 11: mod_base = OTHER; note = 2'-fluoro uridine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-O-methyl cytidine
  modified_base 15: mod_base = OTHER; note = 2'-O-methyl uridine
  modified_base 16: mod_base = OTHER; note = 2'-O-methyl uridine
  modified_base 17: mod_base = OTHER; note = 2'-O-methyl guanosine
  modified_base 18: mod_base = OTHER; note = 2'-O-methyl uridine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 629 - RNA, 36 nt, synthetic construct; identical to SEQ ID NO: 587
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl cytidine
  modified_base 3: mod_base = OTHER; note = 2'-O-methyl adenosine
  modified_base 4: mod_base = OTHER; note = 2'-O-methyl cytidine
  modified_base 5: mod_base = OTHER; note = 2'-O-methyl uridine
  modified_base 6: mod_base = OTHER; note = 2'-O-methyl adenosine
  modified_base 7: mod_base = OTHER; note = 2'-O-methyl uridine
  modified_base 8: mod_base = OTHER; note = 2'-fluoro guanosine
  modified_base 9: mod_base = OTHER; note = 2'-fluoro guanosine
  modified_base 10: mod_base = OTHER; note = 2'-fluoro cytidine
  modified_base 11: mod_base = OTHER; note = 2'-fluoro uridine
  modified_base 12: mod_base = OTHER; note = 2'-O-methyl uridine
  modified_base 13: mod_base = OTHER; note = 2'-O-methyl guanosine
  modified_base 14: mod_base = OTHER; note = 2'-O-methyl uridine
  modified_base 15: mod_base = OTHER; note = 2'-O-methyl adenosine
  modified_base 16: mod_base = OTHER; note = 2'-O-methyl cytidine
  modified_base 17: mod_base = OTHER; note = 2'-O-methyl adenosine
  modified_base 18: mod_base = OTHER; note = 2'-O-methyl adenosine
  modified_base 19: mod_base = OTHER; note = 2'-O-methyl cytidine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 630 - RNA, 36 nt, synthetic construct; identical to SEQ ID NO: 588
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl adenosine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl adenosine
  modified_base 6: mod_base = OTHER; note = 2'-O-methyl guanosine
  modified_base 7: mod_base = OTHER; note = 2'-O-methyl adenosine
  modified_base 8: mod_base = OTHER; note = 2'-fluoro guanosine
  modified_base 9: mod_base = OTHER; note = 2'-fluoro adenosine
  modified_base 10: mod_base = OTHER; note = 2'-fluoro adenosine
  modified_base 11: mod_base = OTHER; note = 2'-fluoro adenosine
  modified_base 12: mod_base = OTHER; note = 2'-O-methyl cytidine
  modified_base 13: mod_base = OTHER; note = 2'-O-methyl uridine
  modified_base 14: mod_base = OTHER; note = 2'-O-methyl guanosine
  modified_base 15: mod_base = OTHER; note = 2'-O-methyl cytidine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl uridine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 631 - RNA, 36 nt, synthetic construct; identical to SEQ ID NO: 589
  misc_feature 1..36: note = synthetic construct
  misc_feature 1..2: note = linked via phosphorothioate
  modified_base 1: mod_base = OTHER; note = 2'-O-methyl guanosine
  modified_base 2: mod_base = OTHER; note = 2'-O-methyl adenosine
  modified_base 3: mod_base = OTHER; note = 2'-O-methyl guanosine
  modified_base 4: mod_base = OTHER; note = 2'-O-methyl guanosine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl uridine
  modified_base 8: mod_base = OTHER; note = 2'-fluoro cytidine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro uridine
  modified_base 12: mod_base = OTHER; note = 2'-O-methyl cytidine
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl guanosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl guanosine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 632 - RNA, 36 nt, synthetic construct; identical to SEQ ID NO: 590
  misc_feature 1..36: note = synthetic construct
  misc_feature 1..2: note = linked via phosphorothioate
  modified_base 1: mod_base = OTHER; note = 2'-O-methyl cytidine
  modified_base 2: mod_base = OTHER; note = 2'-O-methyl uridine
  modified_base 3: mod_base = OTHER; note = 2'-O-methyl cytidine
  modified_base 4: mod_base = OTHER; note = 2'-O-methyl adenosine
  modified_base 5: mod_base = OTHER; note = 2'-O-methyl cytidine
  modified_base 6: mod_base = OTHER; note = 2'-O-methyl cytidine
  modified_base 7: mod_base = OTHER; note = 2'-O-methyl uridine
  modified_base 8: mod_base = OTHER; note = 2'-fluoro guanosine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro uridine
  modified_base 12: mod_base = OTHER; note = 2'-O-methyl cytidine
  modified_base 13: mod_base = OTHER; note = 2'-O-methyl uridine
  modified_base 14: mod_base = OTHER; note = 2'-O-methyl uridine
  modified_base 15: mod_base = OTHER; note = 2'-O-methyl cytidine
  modified_base 16: mod_base = OTHER; note = 2'-O-methyl uridine
  modified_base 17: mod_base = OTHER; note = 2'-O-methyl guanosine
  modified_base 18: mod_base = OTHER; note = 2'-O-methyl guanosine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 633 - RNA, 36 nt, synthetic construct; identical to SEQ ID NO: 591
  misc_feature 1..36: note = synthetic construct
  misc_feature 1..2: note = linked via phosphorothioate
  modified_base 1: mod_base = OTHER; note = 2'-O-methyl uridine
  modified_base 2: mod_base = OTHER; note = 2'-O-methyl cytidine
  modified_base 3: mod_base = OTHER; note = 2'-O-methyl adenosine
  modified_base 4: mod_base = OTHER; note = 2'-O-methyl cytidine
  modified_base 5: mod_base = OTHER; note = 2'-O-methyl cytidine
  modified_base 6: mod_base = OTHER; note = 2'-O-methyl uridine
  modified_base 7: mod_base = OTHER; note = 2'-O-methyl guanosine
  modified_base 8: mod_base = OTHER; note = 2'-fluoro cytidine
  modified_base 9: mod_base = OTHER; note = 2'-fluoro uridine
  modified_base 10: mod_base = OTHER; note = 2'-fluoro uridine
  modified_base 11: mod_base = OTHER; note = 2'-fluoro cytidine
  modified_base 12: mod_base = OTHER; note = 2'-O-methyl uridine
  modified_base 13: mod_base = OTHER; note = 2'-O-methyl uridine
  modified_base 14: mod_base = OTHER; note = 2'-O-methyl cytidine
  modified_base 15: mod_base = OTHER; note = 2'-O-methyl uridine
  modified_base 16: mod_base = OTHER; note = 2'-O-methyl guanosine
  modified_base 17: mod_base = OTHER; note = 2'-O-methyl guanosine
  modified_base 18: mod_base = OTHER; note = 2'-O-methyl uridine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 634 - RNA, 36 nt, synthetic construct; identical to SEQ ID NO: 592
  misc_feature 1..36: note = synthetic construct
  misc_feature 1..2: note = linked via phosphorothioate
  modified_base 1: mod_base = OTHER; note = 2'-O-methyl uridine
  modified_base 2: mod_base = OTHER; note = 2'-O-methyl cytidine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl guanosine
  modified_base 6: mod_base = OTHER; note = 2'-O-methyl uridine
  modified_base 7: mod_base = OTHER; note = 2'-O-methyl uridine
  modified_base 8: mod_base = OTHER; note = 2'-fluoro cytidine
  modified_base 9: mod_base = OTHER; note = 2'-fluoro adenosine
  modified_base 10: mod_base = OTHER; note = 2'-fluoro uridine
  modified_base 11: mod_base = OTHER; note = 2'-fluoro uridine
  modified_base 12: mod_base = OTHER; note = 2'-O-methyl cytidine
  modified_base 13: mod_base = OTHER; note = 2'-O-methyl uridine
  modified_base 14: mod_base = OTHER; note = 2'-O-methyl cytidine
  modified_base 15: mod_base = OTHER; note = 2'-O-methyl cytidine
  modified_base 16: mod_base = OTHER; note = 2'-O-methyl adenosine
  modified_base 17: mod_base = OTHER; note = 2'-O-methyl adenosine
  modified_base 18: mod_base = OTHER; note = 2'-O-methyl adenosine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 635 - RNA, 36 nt, synthetic construct; identical to SEQ ID NO: 593
  misc_feature 1..36: note = synthetic construct
  misc_feature 1..2: note = linked via phosphorothioate
  modified_base 1: mod_base = OTHER; note = 2'-O-methyl guonosine
  modified_base 2: mod_base = OTHER; note = 2'-O-methyl uridine
  modified_base 3: mod_base = OTHER; note = 2'-O-methyl uridine
  modified_base 4: mod_base = OTHER; note = 2'-O-methyl cytidine
  modified_base 5: mod_base = OTHER; note = 2'-O-methyl adenosine
  modified_base 6: mod_base = OTHER; note = 2'-O-methyl uridine
  modified_base 7: mod_base = OTHER; note = 2'-O-methyl uridine
  modified_base 8: mod_base = OTHER; note = 2'-fluoro cytidine
  modified_base 9: mod_base = OTHER; note = 2'-fluoro uridine
  modified_base 10: mod_base = OTHER; note = 2'-fluoro cytidine
  modified_base 11: mod_base = OTHER; note = 2'-fluoro cytidine
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-O-methyl adenosine
  modified_base 15: mod_base = OTHER; note = 2'-O-methyl uridine
  modified_base 16: mod_base = OTHER; note = 2'-O-methyl cytidine
  modified_base 17: mod_base = OTHER; note = 2'-O-methyl cytidine
  modified_base 18: mod_base = OTHER; note = 2'-O-methyl cytidine
  modified_base 19: mod_base = OTHER; note = 2'-O-methyl cytidine
  modified_base 20: mod_base = OTHER; note = 2'-O-methyl adenosine
  modified_base 21: mod_base = OTHER; note = 2'-O-methyl guonosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guonosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guonosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guonosine
  modified_base 32: mod_base = OTHER; note = 2'-O-methyl guonosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guonosine
  modified_base 36: mod_base = OTHER; note = 2'-O-methyl cytidine
  source 1..36: mol_type = other RNA

SEQ ID NO: 636 - RNA, 36 nt, synthetic construct; identical to SEQ ID NO: 594
  misc_feature 1..36: note = synthetic construct
  misc_feature 1..2: note = linked via phosphorothioate
  modified_base 1: mod_base = OTHER; note = 2'-O-methyl adenosine
  modified_base 2: mod_base = OTHER; note = 2'-O-methyl adenosine
  modified_base 3: mod_base = OTHER; note = 2'-O-methyl adenosine
  modified_base 4: mod_base = OTHER; note = 2'-O-methyl guanosine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl guanosine
  modified_base 8: mod_base = OTHER; note = 2'-fluoro adenosine
  modified_base 9: mod_base = OTHER; note = 2'-fluoro uridine
  modified_base 10: mod_base = OTHER; note = 2'-fluoro guanosine
  modified_base 11: mod_base = OTHER; note = 2'-fluoro uridine
  modified_base 12: mod_base = OTHER; note = 2'-O-methyl guanosine
  modified_base 13: mod_base = OTHER; note = 2'-O-methyl cytidine
  modified_base 14: mod_base = OTHER; note = 2'-O-methyl adenosine
  modified_base 15: mod_base = OTHER; note = 2'-O-methyl guanosine
  modified_base 16: mod_base = OTHER; note = 2'-O-methyl uridine
  modified_base 17: mod_base = OTHER; note = 2'-O-methyl uridine
  modified_base 18: mod_base = OTHER; note = 2'-O-methyl guanosine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 637 - RNA, 36 nt, synthetic construct; identical to SEQ ID NO: 595
  misc_feature 1..36: note = synthetic construct
  misc_feature 1..2: note = linked via phosphorothioate
  modified_base 1: mod_base = OTHER; note = 2'-O-methyl adenosine
  modified_base 2: mod_base = OTHER; note = 2'-O-methyl adenosine
  modified_base 3: mod_base = OTHER; note = 2'-O-methyl cytidine
  modified_base 4: mod_base = OTHER; note = 2'-O-methyl uridine
  modified_base 5: mod_base = OTHER; note = 2'-O-methyl cytidine
  modified_base 6: mod_base = OTHER; note = 2'-O-methyl cytidine
  modified_base 7: mod_base = OTHER; note = 2'-O-methyl adenosine
  modified_base 8: mod_base = OTHER; note = 2'-fluoro guanosine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro cytidine
  modified_base 12: mod_base = OTHER; note = 2'-O-methyl cytidine
  modified_base 13: mod_base = OTHER; note = 2'-O-methyl guanosine
  modified_base 14: mod_base = OTHER; note = 2'-O-methyl uridine
  modified_base 15: mod_base = OTHER; note = 2'-O-methyl cytidine
  modified_base 16: mod_base = OTHER; note = 2'-O-methyl uridine
  modified_base 17: mod_base = OTHER; note = 2'-O-methyl adenosine
  modified_base 18: mod_base = OTHER; note = 2'-O-methyl uridine
  modified_base 19: mod_base = OTHER; note = 2'-O-methyl uridine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 638 - RNA, 36 nt, synthetic construct; identical to SEQ ID NO: 596
  misc_feature 1..36: note = synthetic construct
  misc_feature 1..2: note = linked via phosphorothioate
  modified_base 1: mod_base = OTHER; note = 2'-O-methyl adenosine
  modified_base 2: mod_base = OTHER; note = 2'-O-methyl cytidine
  modified_base 3: mod_base = OTHER; note = 2'-O-methyl cytidine
  modified_base 4: mod_base = OTHER; note = 2'-O-methyl uridine
  modified_base 5: mod_base = OTHER; note = 2'-O-methyl guanosine
  modified_base 6: mod_base = OTHER; note = 2'-O-methyl cytidine
  modified_base 7: mod_base = OTHER; note = 2'-O-methyl uridine
  modified_base 8: mod_base = OTHER; note = 2'-fluoro uridine
  modified_base 9: mod_base = OTHER; note = 2'-fluoro cytidine
  modified_base 10: mod_base = OTHER; note = 2'-fluoro uridine
  modified_base 11: mod_base = OTHER; note = 2'-fluoro uridine
  modified_base 12: mod_base = OTHER; note = 2'-O-methyl cytidine
  modified_base 13: mod_base = OTHER; note = 2'-O-methyl uridine
  modified_base 14: mod_base = OTHER; note = 2'-O-methyl guanosine
  modified_base 15: mod_base = OTHER; note = 2'-O-methyl guanosine
  modified_base 16: mod_base = OTHER; note = 2'-O-methyl uridine
  modified_base 17: mod_base = OTHER; note = 2'-O-methyl uridine
  modified_base 18: mod_base = OTHER; note = 2'-O-methyl cytidine
  modified_base 19: mod_base = OTHER; note = 2'-O-methyl adenosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 639 - RNA, 36 nt, synthetic construct; identical to SEQ ID NO: 597
  misc_feature 1..36: note = synthetic construct
  misc_feature 1..2: note = linked via phosphorothioate
  modified_base 1: mod_base = OTHER; note = 2'-O-methyl adenosine
  modified_base 2: mod_base = OTHER; note = 2'-O-methyl guanosine
  modified_base 3: mod_base = OTHER; note = 2'-O-methyl uridine
  modified_base 4: mod_base = OTHER; note = 2'-O-methyl guanosine
  modified_base 5: mod_base = OTHER; note = 2'-O-methyl uridine
  modified_base 6: mod_base = OTHER; note = 2'-O-methyl guanosine
  modified_base 7: mod_base = OTHER; note = 2'-O-methyl adenosine
  modified_base 8: mod_base = OTHER; note = 2'-fluoro adenosine
  modified_base 9: mod_base = OTHER; note = 2'-fluoro adenosine
  modified_base 10: mod_base = OTHER; note = 2'-fluoro guanosine
  modified_base 11: mod_base = OTHER; note = 2'-fluoro adenosine
  modified_base 12: mod_base = OTHER; note = 2'-O-methyl cytidine
  modified_base 13: mod_base = OTHER; note = 2'-O-methyl adenosine
  modified_base 14: mod_base = OTHER; note = 2'-O-methyl uridine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl cytidine
  modified_base 18: mod_base = OTHER; note = 2'-O-methyl uridine
  modified_base 19: mod_base = OTHER; note = 2'-O-methyl guanosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl cytidine
  modified_base 23: mod_base = OTHER; note = 2'-O-methyl adenosine
  modified_base 24: mod_base = OTHER; note = 2'-O-methyl guanosine
  modified_base 25: mod_base = OTHER; note = 2'-O-methyl cytidine
  modified_base 26: mod_base = OTHER; note = 2'-O-methyl cytidine
  modified_base 27: mod_base = OTHER; note = 2'-O-methyl guanosine
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = OTHER; note = 2'-O-methyl guanosine
  modified_base 32: mod_base = OTHER; note = 2'-O-methyl guanosine
  modified_base 33: mod_base = OTHER; note = 2'-O-methyl cytidine
  modified_base 34: mod_base = OTHER; note = 2'-O-methyl uridine
  modified_base 35: mod_base = OTHER; note = 2'-O-methyl guanosine
  modified_base 36: mod_base = OTHER; note = 2'-O-methyl cytidine
  source 1..36: mol_type = other RNA

SEQ ID NO: 640 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro uridine
  modified_base 4: mod_base = OTHER; note = 2'-fluoro uridine
  modified_base 5: mod_base = OTHER; note = 2'-fluoro cytidine
  modified_base 6: mod_base = OTHER; note = 2'-O-methyl uridine
  modified_base 7: mod_base = OTHER; note = 2'-fluoro cytidine
  modified_base 8: mod_base = OTHER; note = 2'-O-methyl uridine
  modified_base 9: mod_base = OTHER; note = 2'-O-methyl uridine
  modified_base 10: mod_base = OTHER; note = 2'-fluoro guanosine
  modified_base 11: mod_base = OTHER; note = 2'-O-methyl guanosine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-fluoro uridine
  modified_base 15: mod_base = OTHER; note = 2'-O-methyl cytidine
  modified_base 16: mod_base = OTHER; note = 2'-O-methyl cytidine
  modified_base 17: mod_base = OTHER; note = 2'-O-methyl uridine
  modified_base 18: mod_base = OTHER; note = 2'-O-methyl modified
  modified_base 18: mod_base = OTHER; note = 2'-O-methyl cytidine
  misc_feature 18..20: note = linked via phosphorothioate
  modified_base 19: mod_base = OTHER; note = 2'-O-methyl adenosine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
ttttctcttggagtcctcacgg

SEQ ID NO: 641 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro adenosine
  modified_base 4: mod_base = OTHER; note = 2'-fluoro adenosine
  modified_base 5: mod_base = OTHER; note = 2'-fluoro uridine
  modified_base 6: mod_base = OTHER; note = 2'-O-methyl adenosine
  modified_base 7: mod_base = OTHER; note = 2'-fluoro cytidine
  modified_base 8: mod_base = OTHER; note = 2'-O-methyl cytidine
  modified_base 9: mod_base = OTHER; note = 2'-O-methyl adenosine
  modified_base 10: mod_base = OTHER; note = 2'-fluoro guanosine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl uridine
  modified_base 14: mod_base = OTHER; note = 2'-fluoro adenosine
  modified_base 15: mod_base = OTHER; note = 2'-O-methyl guanosine
  modified_base 16: mod_base = OTHER; note = 2'-O-methyl cytidine
  modified_base 17: mod_base = OTHER; note = 2'-O-methyl adenosine
  modified_base 18: mod_base = OTHER; note = 2'-O-methyl cytidine
  misc_feature 18..20: note = linked via phosphorothioate
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
taaataccagagtagcacccgg

SEQ ID NO: 642 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro guanosine
  modified_base 3: mod_base = OTHER; note = 2'-fluoro adenosine
  modified_base 4: mod_base = OTHER; note = 2'-fluoro adenosine
  modified_base 5: mod_base = OTHER; note = 2'-fluoro adenosine
  modified_base 6: mod_base = OTHER; note = 2'-O-methyl uridine
  modified_base 7: mod_base = OTHER; note = 2'-fluoro adenosine
  modified_base 8: mod_base = OTHER; note = 2'-O-methyl cytidine
  modified_base 9: mod_base = OTHER; note = 2'-O-methyl cytidine
  modified_base 10: mod_base = OTHER; note = 2'-fluoro adenosine
  modified_base 11: mod_base = OTHER; note = 2'-O-methyl guanosine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-fluoro uridine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl cytidine
  modified_base 18: mod_base = OTHER; note = 2'-O-methyl adenosine
  misc_feature 18..20: note = linked via phosphorothioate
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
tgaaataccagagtagcaccgg

SEQ ID NO: 643 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro adenosine
  modified_base 4: mod_base = OTHER; note = 2'-fluoro guanosine
  modified_base 5: mod_base = OTHER; note = 2'-fluoro guanosine
  modified_base 6: mod_base = OTHER; note = 2'-O-methyl adenosine
  modified_base 7: mod_base = OTHER; note = 2'-fluoro adenosine
  modified_base 8: mod_base = OTHER; note = 2'-O-methyl adenosine
  modified_base 9: mod_base = OTHER; note = 2'-O-methyl uridine
  modified_base 10: mod_base = OTHER; note = 2'-fluoro adenosine
  modified_base 11: mod_base = OTHER; note = 2'-O-methyl cytidine
  modified_base 12: mod_base = OTHER; note = 2'-O-methyl cytidine
  modified_base 13: mod_base = OTHER; note = 2'-O-methyl adenosine
  modified_base 14: mod_base = OTHER; note = 2'-fluoro guanosine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl adenosine
  misc_feature 18..20: note = linked via phosphorothioate
  modified_base 19: mod_base = OTHER; note = 2'-O-methyl guanosine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
ttaggaaataccagagtagcgg

SEQ ID NO: 644 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro uridine
  modified_base 4: mod_base = OTHER; note = 2'-fluoro guanosine
  modified_base 5: mod_base = OTHER; note = 2'-fluoro uridine
  modified_base 6: mod_base = OTHER; note = 2'-O-methyl adenosine
  modified_base 7: mod_base = OTHER; note = 2'-fluoro cytidine
  modified_base 8: mod_base = OTHER; note = 2'-O-methyl cytidine
  modified_base 9: mod_base = OTHER; note = 2'-O-methyl cytidine
  modified_base 10: mod_base = OTHER; note = 2'-fluoro uridine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-fluoro adenosine
  modified_base 15: mod_base = OTHER; note = 2'-O-methyl adenosine
  modified_base 16: mod_base = OTHER; note = 2'-O-methyl adenosine
  modified_base 17: mod_base = OTHER; note = 2'-O-methyl uridine
  modified_base 18: mod_base = OTHER; note = 2'-O-methyl adenosine
  misc_feature 18..20: note = linked via phosphorothioate
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
tttgtaccctaggaaataccgg

SEQ ID NO: 645 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro guanosine
  modified_base 3: mod_base = OTHER; note = 2'-fluoro adenosine
  modified_base 4: mod_base = OTHER; note = 2'-fluoro adenosine
  modified_base 5: mod_base = OTHER; note = 2'-fluoro cytidine
  modified_base 6: mod_base = OTHER; note = 2'-O-methyl cytidine
  modified_base 7: mod_base = OTHER; note = 2'-fluoro adenosine
  modified_base 8: mod_base = OTHER; note = 2'-O-methyl guanosine
  modified_base 9: mod_base = OTHER; note = 2'-O-methyl adenosine
  modified_base 10: mod_base = OTHER; note = 2'-fluoro adenosine
  modified_base 11: mod_base = OTHER; note = 2'-O-methyl guanosine
  modified_base 12: mod_base = OTHER; note = 2'-O-methyl adenosine
  modified_base 13: mod_base = OTHER; note = 2'-O-methyl adenosine
  modified_base 14: mod_base = OTHER; note = 2'-fluoro guanosine
  modified_base 15: mod_base = OTHER; note = 2'-O-methyl cytidine
  modified_base 16: mod_base = OTHER; note = 2'-O-methyl adenosine
  modified_base 17: mod_base = OTHER; note = 2'-O-methyl guanosine
  modified_base 18: mod_base = OTHER; note = 2'-O-methyl guanosine
  misc_feature 18..20: note = linked via phosphorothioate
  modified_base 19: mod_base = OTHER; note = 2'-O-methyl uridine
  modified_base 20: mod_base = OTHER; note = 2'-O-methyl guanosine
  modified_base 21: mod_base = OTHER; note = 2'-O-methyl guanosine
  modified_base 22: mod_base = OTHER; note = 2'-O-methyl guanosine
  source 1..22: mol_type = other RNA
tgaaccagaagaagcaggtggg

SEQ ID NO: 646 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro guanosine
  modified_base 4: mod_base = OTHER; note = 2'-fluoro uridine
  modified_base 5: mod_base = OTHER; note = 2'-fluoro cytidine
  modified_base 6: mod_base = OTHER; note = 2'-O-methyl uridine
  modified_base 7: mod_base = OTHER; note = 2'-fluoro uridine
  modified_base 8: mod_base = OTHER; note = 2'-O-methyl uridine
  modified_base 9: mod_base = OTHER; note = 2'-O-methyl cytidine
  modified_base 10: mod_base = OTHER; note = 2'-fluoro adenosine
  modified_base 11: mod_base = OTHER; note = 2'-O-methyl cytidine
  modified_base 12: mod_base = OTHER; note = 2'-O-methyl adenosine
  modified_base 13: mod_base = OTHER; note = 2'-O-methyl cytidine
  modified_base 14: mod_base = OTHER; note = 2'-fluoro uridine
  modified_base 15: mod_base = OTHER; note = 2'-O-methyl guanosine
  modified_base 16: mod_base = OTHER; note = 2'-O-methyl guanosine
  modified_base 17: mod_base = OTHER; note = 2'-O-methyl cytidine
  modified_base 18: mod_base = OTHER; note = 2'-O-methyl uridine
  misc_feature 18..20: note = linked via phosphorothioate
  modified_base 19: mod_base = OTHER; note = 2'-O-methyl uridine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
ttgtctttcacactggcttcgg

SEQ ID NO: 647 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro adenosine
  modified_base 4: mod_base = OTHER; note = 2'-fluoro guanosine
  modified_base 5: mod_base = OTHER; note = 2'-fluoro uridine
  modified_base 6: mod_base = OTHER; note = 2'-O-methyl adenosine
  modified_base 7: mod_base = OTHER; note = 2'-fluoro guanosine
  modified_base 8: mod_base = OTHER; note = 2'-O-methyl cytidine
  modified_base 9: mod_base = OTHER; note = 2'-O-methyl uridine
  modified_base 10: mod_base = OTHER; note = 2'-fluoro guanosine
  modified_base 11: mod_base = OTHER; note = 2'-O-methyl guanosine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-fluoro adenosine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl adenosine
  misc_feature 18..20: note = linked via phosphorothioate
  modified_base 19: mod_base = OTHER; note = 2'-O-methyl cytidine
  modified_base 20: mod_base = OTHER; note = 2'-O-methyl guanosine
  modified_base 21: mod_base = OTHER; note = 2'-O-methyl guanosine
  modified_base 22: mod_base = OTHER; note = 2'-O-methyl guanosine
  source 1..22: mol_type = other RNA
ttagtagctggggaagtacggg

SEQ ID NO: 648 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro adenosine
  modified_base 4: mod_base = OTHER; note = 2'-fluoro adenosine
  modified_base 5: mod_base = OTHER; note = 2'-fluoro uridine
  modified_base 6: mod_base = OTHER; note = 2'-O-methyl cytidine
  modified_base 7: mod_base = OTHER; note = 2'-fluoro adenosine
  modified_base 8: mod_base = OTHER; note = 2'-O-methyl uridine
  modified_base 9: mod_base = OTHER; note = 2'-O-methyl adenosine
  modified_base 10: mod_base = OTHER; note = 2'-fluoro cytidine
  modified_base 11: mod_base = OTHER; note = 2'-O-methyl uridine
  modified_base 12: mod_base = OTHER; note = 2'-O-methyl uridine
  modified_base 13: mod_base = OTHER; note = 2'-O-methyl cytidine
  modified_base 14: mod_base = OTHER; note = 2'-fluoro uridine
  modified_base 15: mod_base = OTHER; note = 2'-O-methyl guanosine
  modified_base 16: mod_base = OTHER; note = 2'-O-methyl cytidine
  modified_base 17: mod_base = OTHER; note = 2'-O-methyl cytidine
  modified_base 18: mod_base = OTHER; note = 2'-O-methyl uridine
  misc_feature 18..20: note = linked via phosphorothioate
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
taaatcatacttctgcctccgg

SEQ ID NO: 649 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro adenosine
  modified_base 4: mod_base = OTHER; note = 2'-fluoro cytidine
  modified_base 5: mod_base = OTHER; note = 2'-fluoro adenosine
  modified_base 6: mod_base = OTHER; note = 2'-O-methyl adenosine
  modified_base 7: mod_base = OTHER; note = 2'-fluoro guanosine
  modified_base 8: mod_base = OTHER; note = 2'-O-methyl cytidine
  modified_base 9: mod_base = OTHER; note = 2'-O-methyl cytidine
  modified_base 10: mod_base = OTHER; note = 2'-fluoro adenosine
  modified_base 11: mod_base = OTHER; note = 2'-O-methyl uridine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-fluoro uridine
  modified_base 15: mod_base = OTHER; note = 2'-O-methyl guanosine
  modified_base 16: mod_base = OTHER; note = 2'-O-methyl cytidine
  modified_base 17: mod_base = OTHER; note = 2'-O-methyl adenosine
  modified_base 18: mod_base = OTHER; note = 2'-O-methyl cytidine
  misc_feature 18..20: note = linked via phosphorothioate
  modified_base 19: mod_base = OTHER; note = 2'-O-methyl cytidine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
ttacaagccatagtgcacccgg

SEQ ID NO: 650 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro guanosine
  modified_base 3: mod_base = OTHER; note = 2'-fluoro uridine
  modified_base 4: mod_base = OTHER; note = 2'-fluoro uridine
  modified_base 5: mod_base = OTHER; note = 2'-fluoro guanosine
  modified_base 6: mod_base = OTHER; note = 2'-O-methyl uridine
  modified_base 7: mod_base = OTHER; note = 2'-fluoro adenosine
  modified_base 8: mod_base = OTHER; note = 2'-O-methyl cytidine
  modified_base 9: mod_base = OTHER; note = 2'-O-methyl adenosine
  modified_base 10: mod_base = OTHER; note = 2'-fluoro adenosine
  modified_base 11: mod_base = OTHER; note = 2'-O-methyl guanosine
  modified_base 12: mod_base = OTHER; note = 2'-O-methyl cytidine
  modified_base 13: mod_base = OTHER; note = 2'-O-methyl cytidine
  modified_base 14: mod_base = OTHER; note = 2'-fluoro adenosine
  modified_base 15: mod_base = OTHER; note = 2'-O-methyl uridine
  modified_base 16: mod_base = OTHER; note = 2'-O-methyl adenosine
  modified_base 17: mod_base = OTHER; note = 2'-O-methyl guanosine
  modified_base 18: mod_base = OTHER; note = 2'-O-methyl uridine
  misc_feature 18..20: note = linked via phosphorothioate
  modified_base 19: mod_base = OTHER; note = 2'-O-methyl guanosine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
tgttgtacaagccatagtgcgg

SEQ ID NO: 651 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro adenosine
  modified_base 4: mod_base = OTHER; note = 2'-fluoro adenosine
  modified_base 5: mod_base = OTHER; note = 2'-fluoro cytidine
  modified_base 6: mod_base = OTHER; note = 2'-O-methyl guanosine
  modified_base 7: mod_base = OTHER; note = 2'-fluoro cytidine
  modified_base 8: mod_base = OTHER; note = 2'-O-methyl adenosine
  modified_base 9: mod_base = OTHER; note = 2'-O-methyl guanosine
  modified_base 10: mod_base = OTHER; note = 2'-fluoro uridine
  modified_base 11: mod_base = OTHER; note = 2'-O-methyl uridine
  modified_base 12: mod_base = OTHER; note = 2'-O-methyl uridine
  modified_base 13: mod_base = OTHER; note = 2'-O-methyl cytidine
  modified_base 14: mod_base = OTHER; note = 2'-fluoro uridine
  modified_base 15: mod_base = OTHER; note = 2'-O-methyl cytidine
  modified_base 16: mod_base = OTHER; note = 2'-O-methyl uridine
  modified_base 17: mod_base = OTHER; note = 2'-O-methyl cytidine
  modified_base 18: mod_base = OTHER; note = 2'-O-methyl adenosine
  misc_feature 18..20: note = linked via phosphorothioate
  modified_base 19: mod_base = OTHER; note = 2'-O-methyl uridine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
taaacgcagtttctctcatcgg

SEQ ID NO: 652 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro cytidine
  modified_base 4: mod_base = OTHER; note = 2'-fluoro adenosine
  modified_base 5: mod_base = OTHER; note = 2'-fluoro cytidine
  modified_base 6: mod_base = OTHER; note = 2'-O-methyl cytidine
  modified_base 7: mod_base = OTHER; note = 2'-fluoro uridine
  modified_base 8: mod_base = OTHER; note = 2'-O-methyl uridine
  modified_base 9: mod_base = OTHER; note = 2'-O-methyl guanosine
  modified_base 10: mod_base = OTHER; note = 2'-fluoro adenosine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl guanosine
  modified_base 14: mod_base = OTHER; note = 2'-fluoro adenosine
  modified_base 15: mod_base = OTHER; note = 2'-O-methyl cytidine
  modified_base 16: mod_base = OTHER; note = 2'-O-methyl adenosine
  modified_base 17: mod_base = OTHER; note = 2'-O-methyl cytidine
  modified_base 18: mod_base = OTHER; note = 2'-O-methyl cytidine
  misc_feature 18..20: note = linked via phosphorothioate
  modified_base 19: mod_base = OTHER; note = 2'-O-methyl uridine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
ttcaccttgaaggacacctcgg

SEQ ID NO: 653 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro cytidine
  modified_base 4: mod_base = OTHER; note = 2'-fluoro cytidine
  modified_base 5: mod_base = OTHER; note = 2'-fluoro adenosine
  modified_base 6: mod_base = OTHER; note = 2'-O-methyl guanosine
  modified_base 7: mod_base = OTHER; note = 2'-fluoro adenosine
  modified_base 8: mod_base = OTHER; note = 2'-O-methyl adenosine
  modified_base 9: mod_base = OTHER; note = 2'-O-methyl guanosine
  modified_base 10: mod_base = OTHER; note = 2'-fluoro adenosine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl cytidine
  modified_base 14: mod_base = OTHER; note = 2'-fluoro adenosine
  modified_base 15: mod_base = OTHER; note = 2'-O-methyl guanosine
  modified_base 16: mod_base = OTHER; note = 2'-O-methyl guanosine
  modified_base 17: mod_base = OTHER; note = 2'-O-methyl uridine
  modified_base 18: mod_base = OTHER; note = 2'-O-methyl guanosine
  misc_feature 18..20: note = linked via phosphorothioate
  modified_base 19: mod_base = OTHER; note = 2'-O-methyl adenosine
  modified_base 20: mod_base = OTHER; note = 2'-O-methyl guanosine
  modified_base 21: mod_base = OTHER; note = 2'-O-methyl guanosine
  modified_base 22: mod_base = OTHER; note = 2'-O-methyl guanosine
  source 1..22: mol_type = other RNA
taccagaagaagcaggtgaggg

SEQ ID NO: 654 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro adenosine
  modified_base 4: mod_base = OTHER; note = 2'-fluoro cytidine
  modified_base 5: mod_base = OTHER; note = 2'-fluoro cytidine
  modified_base 6: mod_base = OTHER; note = 2'-O-methyl adenosine
  modified_base 7: mod_base = OTHER; note = 2'-fluoro guanosine
  modified_base 8: mod_base = OTHER; note = 2'-O-methyl adenosine
  modified_base 9: mod_base = OTHER; note = 2'-O-methyl adenosine
  modified_base 10: mod_base = OTHER; note = 2'-fluoro guanosine
  modified_base 11: mod_base = OTHER; note = 2'-O-methyl adenosine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-fluoro cytidine
  modified_base 15: mod_base = OTHER; note = 2'-O-methyl adenosine
  modified_base 16: mod_base = OTHER; note = 2'-O-methyl guanosine
  modified_base 17: mod_base = OTHER; note = 2'-O-methyl guanosine
  modified_base 18: mod_base = OTHER; note = 2'-O-methyl uridine
  misc_feature 18..20: note = linked via phosphorothioate
  modified_base 19: mod_base = OTHER; note = 2'-O-methyl guanosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl guanosine
  source 1..22: mol_type = other RNA
taaccagaagaagcaggtgagg

SEQ ID NO: 655 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro uridine
  modified_base 4: mod_base = OTHER; note = 2'-fluoro uridine
  modified_base 5: mod_base = OTHER; note = 2'-fluoro uridine
  modified_base 6: mod_base = OTHER; note = 2'-O-methyl guanosine
  modified_base 7: mod_base = OTHER; note = 2'-fluoro guanosine
  modified_base 8: mod_base = OTHER; note = 2'-O-methyl adenosine
  modified_base 9: mod_base = OTHER; note = 2'-O-methyl guanosine
  modified_base 10: mod_base = OTHER; note = 2'-fluoro adenosine
  modified_base 11: mod_base = OTHER; note = 2'-O-methyl adenosine
  modified_base 12: mod_base = OTHER; note = 2'-O-methyl uridine
  modified_base 13: mod_base = OTHER; note = 2'-O-methyl guanosine
  modified_base 14: mod_base = OTHER; note = 2'-fluoro adenosine
  modified_base 15: mod_base = OTHER; note = 2'-O-methyl adenosine
  modified_base 16: mod_base = OTHER; note = 2'-O-methyl cytidine
  modified_base 17: mod_base = OTHER; note = 2'-O-methyl cytidine
  modified_base 18: mod_base = OTHER; note = 2'-O-methyl adenosine
  misc_feature 18..20: note = linked via phosphorothioate
  modified_base 19: mod_base = OTHER; note = 2'-O-methyl guanosine
  modified_base 20: mod_base = OTHER; note = 2'-O-methyl adenosine
  modified_base 21: mod_base = OTHER; note = 2'-O-methyl guanosine
  modified_base 22: mod_base = OTHER; note = 2'-O-methyl guanosine
  source 1..22: mol_type = other RNA
tatttggagaatgaaccagagg

SEQ ID NO: 656 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro guanosine
  modified_base 3: mod_base = OTHER; note = 2'-fluoro guanosine
  modified_base 4: mod_base = OTHER; note = 2'-fluoro guanosine
  modified_base 5: mod_base = OTHER; note = 2'-fluoro guanosine
  modified_base 6: mod_base = OTHER; note = 2'-O-methyl adenosine
  modified_base 7: mod_base = OTHER; note = 2'-fluoro uridine
  modified_base 8: mod_base = OTHER; note = 2'-O-methyl uridine
  modified_base 9: mod_base = OTHER; note = 2'-O-methyl uridine
  modified_base 10: mod_base = OTHER; note = 2'-fluoro guanosine
  modified_base 11: mod_base = OTHER; note = 2'-O-methyl guanosine
  modified_base 12: mod_base = OTHER; note = 2'-O-methyl adenosine
  modified_base 13: mod_base = OTHER; note = 2'-O-methyl guanosine
  modified_base 14: mod_base = OTHER; note = 2'-fluoro adenosine
  modified_base 15: mod_base = OTHER; note = 2'-O-methyl adenosine
  modified_base 16: mod_base = OTHER; note = 2'-O-methyl uridine
  modified_base 17: mod_base = OTHER; note = 2'-O-methyl guanosine
  modified_base 18: mod_base = OTHER; note = 2'-O-methyl adenosine
  misc_feature 18..20: note = linked via phosphorothioate
  modified_base 19: mod_base = OTHER; note = 2'-O-methyl adenosine
  modified_base 20: mod_base = OTHER; note = 2'-O-methyl cytidine
  modified_base 21: mod_base = OTHER; note = 2'-O-methyl guanosine
  modified_base 22: mod_base = OTHER; note = 2'-O-methyl guanosine
  source 1..22: mol_type = other RNA
tggggatttggagaatgaacgg

SEQ ID NO: 657 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro cytidine
  modified_base 4: mod_base = OTHER; note = 2'-fluoro adenosine
  modified_base 5: mod_base = OTHER; note = 2'-fluoro adenosine
  modified_base 6: mod_base = OTHER; note = 2'-O-methyl cytidine
  modified_base 7: mod_base = OTHER; note = 2'-fluoro uridine
  modified_base 8: mod_base = OTHER; note = 2'-O-methyl guanosine
  modified_base 9: mod_base = OTHER; note = 2'-O-methyl cytidine
  modified_base 10: mod_base = OTHER; note = 2'-fluoro adenosine
  modified_base 11: mod_base = OTHER; note = 2'-O-methyl cytidine
  modified_base 12: mod_base = OTHER; note = 2'-O-methyl adenosine
  modified_base 13: mod_base = OTHER; note = 2'-O-methyl uridine
  modified_base 14: mod_base = OTHER; note = 2'-fluoro cytidine
  modified_base 15: mod_base = OTHER; note = 2'-O-methyl cytidine
  modified_base 16: mod_base = OTHER; note = 2'-O-methyl adenosine
  modified_base 17: mod_base = OTHER; note = 2'-O-methyl cytidine
  modified_base 18: mod_base = OTHER; note = 2'-O-methyl uridine
  misc_feature 18..20: note = linked via phosphorothioate
  modified_base 19: mod_base = OTHER; note = 2'-O-methyl uridine
  modified_base 20: mod_base = OTHER; note = 2'-O-methyl uridine
  modified_base 21: mod_base = OTHER; note = 2'-O-methyl guanosine
  modified_base 22: mod_base = OTHER; note = 2'-O-methyl guanosine
  source 1..22: mol_type = other RNA
ttcaactgcacatccactttgg

SEQ ID NO: 658 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro adenosine
  modified_base 3: mod_base = OTHER; note = 2'-fluoro adenosine
  modified_base 4: mod_base = OTHER; note = 2'-fluoro uridine
  modified_base 5: mod_base = OTHER; note = 2'-fluoro adenosine
  modified_base 6: mod_base = OTHER; note = 2'-O-methyl guanosine
  modified_base 7: mod_base = OTHER; note = 2'-fluoro adenosine
  modified_base 8: mod_base = OTHER; note = 2'-O-methyl cytidine
  modified_base 9: mod_base = OTHER; note = 2'-O-methyl guanosine
  modified_base 10: mod_base = OTHER; note = 2'-fluoro guanosine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl cytidine
  modified_base 14: mod_base = OTHER; note = 2'-fluoro uridine
  modified_base 15: mod_base = OTHER; note = 2'-O-methyl guanosine
  modified_base 16: mod_base = OTHER; note = 2'-O-methyl guanosine
  modified_base 17: mod_base = OTHER; note = 2'-O-methyl adenosine
  modified_base 18: mod_base = OTHER; note = 2'-O-methyl guanosine
  misc_feature 18..20: note = linked via phosphorothioate
  modified_base 19: mod_base = OTHER; note = 2'-O-methyl uridine
  modified_base 20: mod_base = OTHER; note = 2'-O-methyl uridine
  modified_base 21: mod_base = OTHER; note = 2'-O-methyl guanosine
  modified_base 22: mod_base = OTHER; note = 2'-O-methyl guanosine
  source 1..22: mol_type = other RNA
taatagacggagctggagttgg

SEQ ID NO: 659 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro uridine
  modified_base 3: mod_base = OTHER; note = 2'-fluoro guanosine
  modified_base 4: mod_base = OTHER; note = 2'-fluoro adenosine
  modified_base 5: mod_base = OTHER; note = 2'-fluoro adenosine
  modified_base 6: mod_base = OTHER; note = 2'-O-methyl cytidine
  modified_base 7: mod_base = OTHER; note = 2'-fluoro cytidine
  modified_base 8: mod_base = OTHER; note = 2'-O-methyl adenosine
  modified_base 9: mod_base = OTHER; note = 2'-O-methyl guanosine
  modified_base 10: mod_base = OTHER; note = 2'-fluoro adenosine
  modified_base 11: mod_base = OTHER; note = 2'-O-methyl adenosine
  modified_base 12: mod_base = OTHER; note = 2'-O-methyl guanosine
  modified_base 13: mod_base = OTHER; note = 2'-O-methyl adenosine
  modified_base 14: mod_base = OTHER; note = 2'-fluoro adenosine
  modified_base 15: mod_base = OTHER; note = 2'-O-methyl guanosine
  modified_base 16: mod_base = OTHER; note = 2'-O-methyl cytidine
  modified_base 17: mod_base = OTHER; note = 2'-O-methyl adenosine
  modified_base 18: mod_base = OTHER; note = 2'-O-methyl guanosine
  misc_feature 18..20: note = linked via phosphorothioate
  modified_base 19: mod_base = OTHER; note = 2'-O-methyl guanosine
  modified_base 20: mod_base = OTHER; note = 2'-O-methyl uridine
  modified_base 21: mod_base = OTHER; note = 2'-O-methyl guanosine
  modified_base 22: mod_base = OTHER; note = 2'-O-methyl guanosine
  source 1..22: mol_type = other RNA
ttgaaccagaagaagcaggtgg

SEQ ID NO: 660 - RNA, 22 nt, synthetic construct
  misc_feature 1..22: note = synthetic construct
  misc_feature 1..4: note = linked via phosphorothioate
  modified_base 1: mod_base = OTHER; note = 5'-methoxyphosphonate-4-oxy uridine
  modified_base 2: mod_base = OTHER; note = 2'-fluoro cytidine
  modified_base 3: mod_base = OTHER; note = 2'-fluoro adenosine
  modified_base 4: mod_base = OTHER; note = 2'-fluoro guanosine
  modified_base 5: mod_base = OTHER; note = 2'-fluoro cytidine
  modified_base 6: mod_base = OTHER; note = 2'-O-methyl uridine
  modified_base 7: mod_base = OTHER; note = 2'-fluoro adenosine
  modified_base 8: mod_base = OTHER; note = 2'-O-methyl uridine
  modified_base 9: mod_base = OTHER; note = 2'-O-methyl guanosine
  modified_base 10: mod_base = OTHER; note = 2'-fluoro uridine
  modified_base 11: mod_base = OTHER; note = 2'-O-methyl cytidine
  modified_base 12: mod_base = OTHER; note = 2'-O-methyl uridine
  modified_base 13: mod_base = OTHER; note = 2'-O-methyl uridine
  modified_base 14: mod_base = OTHER; note = 2'-fluoro uridine
  modified_base 15: mod_base = OTHER; note = 2'-O-methyl cytidine
  modified_base 16: mod_base = OTHER; note = 2'-O-methyl adenosine
  modified_base 17: mod_base = OTHER; note = 2'-O-methyl cytidine
  modified_base 18: mod_base = OTHER; note = 2'-O-methyl adenosine
  misc_feature 18..20: note = linked via phosphorothioate
  modified_base 19: mod_base = OTHER; note = 2'-O-methyl cytidine
  modified_base 20: mod_base = OTHER; note = 2'-O-methyl uridine
  modified_base 21: mod_base = OTHER; note = 2'-O-methyl guanosine
  modified_base 22: mod_base = OTHER; note = 2'-O-methyl guanosine
  source 1..22: mol_type = other RNA
tcagctatgtctttcacactgg

SEQ ID NO: 661 - RNA, 19 nt, synthetic construct
  source 1..19: mol_type = other RNA
tgtccttcaaggtgagccg

SEQ ID NO: 662 - RNA, 19 nt, synthetic construct
  source 1..19: mol_type = other RNA
gtgtccttcaaggtgagcc

SEQ ID NO: 663 - RNA, 19 nt, synthetic construct
  source 1..19: mol_type = other RNA
ggtgtccttcaaggtgagc

SEQ ID NO: 664 - RNA, 19 nt, synthetic construct
  source 1..19: mol_type = other RNA
aggtgtccttcaaggtgag

SEQ ID NO: 665 - RNA, 19 nt, synthetic construct
  source 1..19: mol_type = other RNA
gaggtgtccttcaaggtga

SEQ ID NO: 666 - RNA, 19 nt, synthetic construct
  source 1..19: mol_type = other RNA
aggaggacagcatggcctc

SEQ ID NO: 667 - RNA, 19 nt, synthetic construct
  source 1..19: mol_type = other RNA
caggaggacagcatggcct

SEQ ID NO: 668 - RNA, 19 nt, synthetic construct
  source 1..19: mol_type = other RNA
ccaggaggacagcatggcc

SEQ ID NO: 669 - RNA, 19 nt, synthetic construct
  source 1..19: mol_type = other RNA
tccaggaggacagcatggc

SEQ ID NO: 670 - RNA, 19 nt, synthetic construct
  source 1..19: mol_type = other RNA
ttccaggaggacagcatgg

SEQ ID NO: 671 - RNA, 19 nt, synthetic construct
  source 1..19: mol_type = other RNA
cttccaggaggacagcatg

SEQ ID NO: 672 - RNA, 19 nt, synthetic construct
  source 1..19: mol_type = other RNA
gcttccaggaggacagcat

SEQ ID NO: 673 - RNA, 19 nt, synthetic construct
  source 1..19: mol_type = other RNA
tgcttccaggaggacagca

SEQ ID NO: 674 - RNA, 19 nt, synthetic construct
  source 1..19: mol_type = other RNA
tcatcgctgaccgctgggt

SEQ ID NO: 675 - RNA, 19 nt, synthetic construct
  source 1..19: mol_type = other RNA
ctcatcgctgaccgctggg

SEQ ID NO: 676 - RNA, 19 nt, synthetic construct
  source 1..19: mol_type = other RNA
tgtgactgtggcctccagg

SEQ ID NO: 677 - RNA, 19 nt, synthetic construct
  source 1..19: mol_type = other RNA
actgtgactgtggcctcca

SEQ ID NO: 678 - RNA, 19 nt, synthetic construct
  source 1..19: mol_type = other RNA
cactgtgactgtggcctcc

SEQ ID NO: 679 - RNA, 19 nt, synthetic construct
  source 1..19: mol_type = other RNA
cctcccagatctccctcac

SEQ ID NO: 680 - RNA, 19 nt, synthetic construct
  source 1..19: mol_type = other RNA
tcacctcccagatctccct

SEQ ID NO: 681 - RNA, 19 nt, synthetic construct
  source 1..19: mol_type = other RNA
ttcacctcccagatctccc

SEQ ID NO: 682 - RNA, 19 nt, synthetic construct
  source 1..19: mol_type = other RNA
ggtatttcctagggtacaa

SEQ ID NO: 683 - RNA, 19 nt, synthetic construct
  source 1..19: mol_type = other RNA
tggtatttcctagggtaca

SEQ ID NO: 684 - RNA, 19 nt, synthetic construct
  source 1..19: mol_type = other RNA
gctaccgcaagggcaagaa

SEQ ID NO: 685 - RNA, 19 nt, synthetic construct
  source 1..19: mol_type = other RNA
ggctaccgcaagggcaaga

SEQ ID NO: 686 - RNA, 19 nt, synthetic construct
  source 1..19: mol_type = other RNA
catggcaggccagcctcca

SEQ ID NO: 687 - RNA, 19 nt, synthetic construct
  source 1..19: mol_type = other RNA
agggtgagtggccatggca

SEQ ID NO: 688 - RNA, 19 nt, synthetic construct
  source 1..19: mol_type = other RNA
cgagggtgagtggccatgg

SEQ ID NO: 689 - RNA, 19 nt, synthetic construct
  source 1..19: mol_type = other RNA
gtcctccgagggtgagtgg

SEQ ID NO: 690 - RNA, 19 nt, synthetic construct
  source 1..19: mol_type = other RNA
tgtcctccgagggtgagtg

SEQ ID NO: 691 - RNA, 19 nt, synthetic construct
  source 1..19: mol_type = other RNA
gtgtcctccgagggtgagt

SEQ ID NO: 692 - RNA, 19 nt, synthetic construct
  source 1..19: mol_type = other RNA
gcctggatgagagaaactg

SEQ ID NO: 693 - RNA, 19 nt, synthetic construct
  source 1..19: mol_type = other RNA
ggcctggatgagagaaact

SEQ ID NO: 694 - RNA, 19 nt, synthetic construct
  source 1..19: mol_type = other RNA
ctctggactacggcttggc

SEQ ID NO: 695 - RNA, 19 nt, synthetic construct
  source 1..19: mol_type = other RNA
tctctggactacggcttgg

SEQ ID NO: 696 - RNA, 19 nt, synthetic construct
  source 1..19: mol_type = other RNA
tcacctgcttcttctggtt

SEQ ID NO: 697 - RNA, 19 nt, synthetic construct
  source 1..19: mol_type = other RNA
ctcacctgcttcttctggt

SEQ ID NO: 698 - RNA, 19 nt, synthetic construct
  source 1..19: mol_type = other RNA
caaagcccagaagatgctc

SEQ ID NO: 699 - RNA, 19 nt, synthetic construct
  source 1..19: mol_type = other RNA
ccaaagcccagaagatgct

SEQ ID NO: 700 - RNA, 19 nt, synthetic construct
  source 1..19: mol_type = other RNA
gccaaagcccagaagatgc

SEQ ID NO: 701 - RNA, 19 nt, synthetic construct
  source 1..19: mol_type = other RNA
tttggaataaagctgcctg

SEQ ID NO: 702 - RNA, 19 nt, synthetic construct
  source 1..19: mol_type = other RNA
ctttggaataaagctgcct

SEQ ID NO: 703 - RNA, 19 nt, synthetic construct
  source 1..19: mol_type = other RNA
ccctttggaataaagctgc

SEQ ID NO: 704 - RNA, 19 nt, synthetic construct
  source 1..19: mol_type = other RNA
gccctttggaataaagctg

SEQ ID NO: 705 - RNA, 19 nt, synthetic construct
  source 1..19: mol_type = other RNA
tgccctttggaataaagct

SEQ ID NO: 706 - RNA, 19 nt, synthetic construct
  source 1..19: mol_type = other RNA
agctgccctttggaataaa

SEQ ID NO: 707 - RNA, 19 nt, synthetic construct
  source 1..19: mol_type = other RNA
cagctgccctttggaataa

SEQ ID NO: 708 - RNA, 19 nt, synthetic construct
  source 1..19: mol_type = other RNA
tgagctcagctgccctttg

SEQ ID NO: 709 - RNA, 19 nt, synthetic construct
  source 1..19: mol_type = other RNA
gtgagctcagctgcccttt

SEQ ID NO: 710 - RNA, 19 nt, synthetic construct
  source 1..19: mol_type = other RNA
agcttcggaagcccctggt

SEQ ID NO: 711 - RNA, 19 nt, synthetic construct
  source 1..19: mol_type = other RNA
tcagcagcaagaatgctgg

SEQ ID NO: 712 - RNA, 19 nt, synthetic construct
  source 1..19: mol_type = other RNA
ctcagcagcaagaatgctg

SEQ ID NO: 713 - RNA, 19 nt, synthetic construct
  source 1..19: mol_type = other RNA
gctcagcagcaagaatgct

SEQ ID NO: 714 - RNA, 19 nt, synthetic construct
  source 1..19: mol_type = other RNA
tggctcagcagcaagaatg

SEQ ID NO: 715 - RNA, 19 nt, synthetic construct
  source 1..19: mol_type = other RNA
gtggctcagcagcaagaat

SEQ ID NO: 716 - RNA, 19 nt, synthetic construct
  source 1..19: mol_type = other RNA
gatgtctgctccagtgatg

SEQ ID NO: 717 - RNA, 19 nt, synthetic construct
  source 1..19: mol_type = other RNA
tgatgtctgctccagtgat

SEQ ID NO: 718 - RNA, 19 nt, synthetic construct
  source 1..19: mol_type = other RNA
ctgatgtctgctccagtga

SEQ ID NO: 719 - RNA, 19 nt, synthetic construct
  source 1..19: mol_type = other RNA
ccctgatgtctgctccagt

SEQ ID NO: 720 - RNA, 19 nt, synthetic construct
  source 1..19: mol_type = other RNA
aagtggtgacctgaggaac

SEQ ID NO: 721 - RNA, 19 nt, synthetic construct
  source 1..19: mol_type = other RNA
caagtggtgacctgaggaa

SEQ ID NO: 722 - RNA, 19 nt, synthetic construct
  source 1..19: mol_type = other RNA
gcaagtggtgacctgagga

SEQ ID NO: 723 - RNA, 19 nt, synthetic construct
  source 1..19: mol_type = other RNA
agcaagtggtgacctgagg

SEQ ID NO: 724 - RNA, 19 nt, synthetic construct
  source 1..19: mol_type = other RNA
cagcaagtggtgacctgag

SEQ ID NO: 725 - RNA, 19 nt, synthetic construct
  source 1..19: mol_type = other RNA
ccagcaagtggtgacctga

SEQ ID NO: 726 - RNA, 19 nt, synthetic construct
  source 1..19: mol_type = other RNA
atccagcaagtggtgacct

SEQ ID NO: 727 - RNA, 19 nt, synthetic construct
  source 1..19: mol_type = other RNA
tggatccagcaagtggtga

SEQ ID NO: 728 - RNA, 19 nt, synthetic construct
  source 1..19: mol_type = other RNA
ctggatccagcaagtggtg

SEQ ID NO: 729 - RNA, 19 nt, synthetic construct
  source 1..19: mol_type = other RNA
cccgcatcacaggtgtgat

SEQ ID NO: 730 - RNA, 19 nt, synthetic construct
  source 1..19: mol_type = other RNA
acccgcatcacaggtgtga

SEQ ID NO: 731 - RNA, 19 nt, synthetic construct
  source 1..19: mol_type = other RNA
aactacttcggcgtctaca

SEQ ID NO: 732 - RNA, 19 nt, synthetic construct
  source 1..19: mol_type = other RNA
ttgatcccacaggacctgt

SEQ ID NO: 733 - RNA, 19 nt, synthetic construct
  source 1..19: mol_type = other RNA
tgcagttgatcccacagga

SEQ ID NO: 734 - RNA, 19 nt, synthetic construct
  source 1..19: mol_type = other RNA
agaggtgtccttcaaggtg

SEQ ID NO: 735 - RNA, 19 nt, synthetic construct
  source 1..19: mol_type = other RNA
gagaggtgtccttcaaggt

SEQ ID NO: 736 - RNA, 19 nt, synthetic construct
  source 1..19: mol_type = other RNA
ctggagaggtgtccttcaa

SEQ ID NO: 737 - RNA, 19 nt, synthetic construct
  source 1..19: mol_type = other RNA
aggttcggggtcgacacat

SEQ ID NO: 738 - RNA, 19 nt, synthetic construct
  source 1..19: mol_type = other RNA
attcaccttccagtgtgag

SEQ ID NO: 739 - RNA, 19 nt, synthetic construct
  source 1..19: mol_type = other RNA
cattcaccttccagtgtga

SEQ ID NO: 740 - RNA, 19 nt, synthetic construct
  source 1..19: mol_type = other RNA
acattcaccttccagtgtg

SEQ ID NO: 741 - RNA, 19 nt, synthetic construct
  source 1..19: mol_type = other RNA
gacattcaccttccagtgt

SEQ ID NO: 742 - RNA, 19 nt, synthetic construct
  source 1..19: mol_type = other RNA
ggacattcaccttccagtg

SEQ ID NO: 743 - RNA, 19 nt, synthetic construct
  source 1..19: mol_type = other RNA
gcagagccacattccagtg

SEQ ID NO: 744 - RNA, 19 nt, synthetic construct
  source 1..19: mol_type = other RNA
tgcagagccacattccagt

SEQ ID NO: 745 - RNA, 19 nt, synthetic construct
  source 1..19: mol_type = other RNA
ttgcagagccacattccag

SEQ ID NO: 746 - RNA, 19 nt, synthetic construct
  source 1..19: mol_type = other RNA
tttgcagagccacattcca

SEQ ID NO: 747 - RNA, 19 nt, synthetic construct
  source 1..19: mol_type = other RNA
aaactgcgtttgcagagcc

SEQ ID NO: 748 - RNA, 19 nt, synthetic construct
  source 1..19: mol_type = other RNA
gaaactgcgtttgcagagc

SEQ ID NO: 749 - RNA, 19 nt, synthetic construct
  source 1..19: mol_type = other RNA
agaaactgcgtttgcagag

SEQ ID NO: 750 - RNA, 19 nt, synthetic construct
  source 1..19: mol_type = other RNA
gagaaactgcgtttgcaga

SEQ ID NO: 751 - RNA, 19 nt, synthetic construct
  source 1..19: mol_type = other RNA
agagaaactgcgtttgcag

SEQ ID NO: 752 - RNA, 19 nt, synthetic construct
  source 1..19: mol_type = other RNA
gagagaaactgcgtttgca

SEQ ID NO: 753 - RNA, 19 nt, synthetic construct
  source 1..19: mol_type = other RNA
tgagagaaactgcgtttgc

SEQ ID NO: 754 - RNA, 19 nt, synthetic construct
  source 1..19: mol_type = other RNA
atgagagaaactgcgtttg

SEQ ID NO: 755 - RNA, 19 nt, synthetic construct
  source 1..19: mol_type = other RNA
gatgagagaaactgcgttt

SEQ ID NO: 756 - RNA, 19 nt, synthetic construct
  source 1..19: mol_type = other RNA
ggatgagagaaactgcgtt

SEQ ID NO: 757 - RNA, 19 nt, synthetic construct
  source 1..19: mol_type = other RNA
tggatgagagaaactgcgt

SEQ ID NO: 758 - RNA, 19 nt, synthetic construct
  source 1..19: mol_type = other RNA
ctggatgagagaaactgcg

SEQ ID NO: 759 - RNA, 19 nt, synthetic construct
  source 1..19: mol_type = other RNA
cctggatgagagaaactgc

SEQ ID NO: 760 - RNA, 19 nt, synthetic construct
  source 1..19: mol_type = other RNA
cctgtgatggggtcaagga

SEQ ID NO: 761 - RNA, 19 nt, synthetic construct
  source 1..19: mol_type = other RNA
ttgtacaaccagtcggacc

SEQ ID NO: 762 - RNA, 19 nt, synthetic construct
  source 1..19: mol_type = other RNA
cttgtacaaccagtcggac

SEQ ID NO: 763 - RNA, 19 nt, synthetic construct
  source 1..19: mol_type = other RNA
gcttgtacaaccagtcgga

SEQ ID NO: 764 - RNA, 19 nt, synthetic construct
  source 1..19: mol_type = other RNA
tggcttgtacaaccagtcg

SEQ ID NO: 765 - RNA, 19 nt, synthetic construct
  source 1..19: mol_type = other RNA
atggcttgtacaaccagtc

SEQ ID NO: 766 - RNA, 19 nt, synthetic construct
  source 1..19: mol_type = other RNA
tatggcttgtacaaccagt

SEQ ID NO: 767 - RNA, 19 nt, synthetic construct
  source 1..19: mol_type = other RNA
ctatggcttgtacaaccag

SEQ ID NO: 768 - RNA, 19 nt, synthetic construct
  source 1..19: mol_type = other RNA
actatggcttgtacaacca

SEQ ID NO: 769 - RNA, 19 nt, synthetic construct
  source 1..19: mol_type = other RNA
cactatggcttgtacaacc

SEQ ID NO: 770 - RNA, 19 nt, synthetic construct
  source 1..19: mol_type = other RNA
gcactatggcttgtacaac

SEQ ID NO: 771 - RNA, 19 nt, synthetic construct
  source 1..19: mol_type = other RNA
tgcactatggcttgtacaa

SEQ ID NO: 772 - RNA, 19 nt, synthetic construct
  source 1..19: mol_type = other RNA
gtgcactatggcttgtaca

SEQ ID NO: 773 - RNA, 19 nt, synthetic construct
  source 1..19: mol_type = other RNA
ggtgcactatggcttgtac

SEQ ID NO: 774 - RNA, 19 nt, synthetic construct
  source 1..19: mol_type = other RNA
gggtgcactatggcttgta

SEQ ID NO: 775 - RNA, 19 nt, synthetic construct
  source 1..19: mol_type = other RNA
cgggtgcactatggcttgt

SEQ ID NO: 776 - RNA, 19 nt, synthetic construct
  source 1..19: mol_type = other RNA
tgcgggtgcactatggctt

SEQ ID NO: 777 - RNA, 19 nt, synthetic construct
  source 1..19: mol_type = other RNA
gtgtgcgggtgcactatgg

SEQ ID NO: 778 - RNA, 19 nt, synthetic construct
  source 1..19: mol_type = other RNA
tggacgatccagaacagga

SEQ ID NO: 779 - RNA, 19 nt, synthetic construct
  source 1..19: mol_type = other RNA
cagtggacgatccagaaca

SEQ ID NO: 780 - RNA, 19 nt, synthetic construct
  source 1..19: mol_type = other RNA
aagtatgatttgccgtgca

SEQ ID NO: 781 - RNA, 19 nt, synthetic construct
  source 1..19: mol_type = other RNA
gaagtatgatttgccgtgc

SEQ ID NO: 782 - RNA, 19 nt, synthetic construct
  source 1..19: mol_type = other RNA
agaagtatgatttgccgtg

SEQ ID NO: 783 - RNA, 19 nt, synthetic construct
  source 1..19: mol_type = other RNA
cagaagtatgatttgccgt

SEQ ID NO: 784 - RNA, 19 nt, synthetic construct
  source 1..19: mol_type = other RNA
ggcagaagtatgatttgcc

SEQ ID NO: 785 - RNA, 19 nt, synthetic construct
  source 1..19: mol_type = other RNA
aggcagaagtatgatttgc

SEQ ID NO: 786 - RNA, 19 nt, synthetic construct
  source 1..19: mol_type = other RNA
gaggcagaagtatgatttg

SEQ ID NO: 787 - RNA, 19 nt, synthetic construct
  source 1..19: mol_type = other RNA
ggaggcagaagtatgattt

SEQ ID NO: 788 - RNA, 19 nt, synthetic construct
  source 1..19: mol_type = other RNA
acttccccagctactactc

SEQ ID NO: 789 - RNA, 19 nt, synthetic construct
  source 1..19: mol_type = other RNA
cgtacttccccagctacta

SEQ ID NO: 790 - RNA, 19 nt, synthetic construct
  source 1..19: mol_type = other RNA
tgcacagctactacgaccc

SEQ ID NO: 791 - RNA, 19 nt, synthetic construct
  source 1..19: mol_type = other RNA
agaagggcctgcacagcta

SEQ ID NO: 792 - RNA, 19 nt, synthetic construct
  source 1..19: mol_type = other RNA
accgactggccatgtatga

SEQ ID NO: 793 - RNA, 19 nt, synthetic construct
  source 1..19: mol_type = other RNA
gttgttaccgctacagcta

SEQ ID NO: 794 - RNA, 19 nt, synthetic construct
  source 1..19: mol_type = other RNA
gggttgttaccgctacagc

SEQ ID NO: 795 - RNA, 19 nt, synthetic construct
  source 1..19: mol_type = other RNA
tgggttgttaccgctacag

SEQ ID NO: 796 - RNA, 19 nt, synthetic construct
  source 1..19: mol_type = other RNA
ctgggttgttaccgctaca

SEQ ID NO: 797 - RNA, 19 nt, synthetic construct
  source 1..19: mol_type = other RNA
attccacgctgggttgtta

SEQ ID NO: 798 - RNA, 19 nt, synthetic construct
  source 1..19: mol_type = other RNA
aattccacgctgggttgtt

SEQ ID NO: 799 - RNA, 19 nt, synthetic construct
  source 1..19: mol_type = other RNA
tgtgaaagacatagctgca

SEQ ID NO: 800 - RNA, 19 nt, synthetic construct
  source 1..19: mol_type = other RNA
gtgtgaaagacatagctgc

SEQ ID NO: 801 - RNA, 19 nt, synthetic construct
  source 1..19: mol_type = other RNA
agtgtgaaagacatagctg

SEQ ID NO: 802 - RNA, 19 nt, synthetic construct
  source 1..19: mol_type = other RNA
cagtgtgaaagacatagct

SEQ ID NO: 803 - RNA, 19 nt, synthetic construct
  source 1..19: mol_type = other RNA
ccagtgtgaaagacatagc

SEQ ID NO: 804 - RNA, 19 nt, synthetic construct
  source 1..19: mol_type = other RNA
gccagtgtgaaagacatag

SEQ ID NO: 805 - RNA, 19 nt, synthetic construct
  source 1..19: mol_type = other RNA
agccagtgtgaaagacata

SEQ ID NO: 806 - RNA, 19 nt, synthetic construct
  source 1..19: mol_type = other RNA
aagccagtgtgaaagacat

SEQ ID NO: 807 - RNA, 19 nt, synthetic construct
  source 1..19: mol_type = other RNA
gaagccagtgtgaaagaca

SEQ ID NO: 808 - RNA, 19 nt, synthetic construct
  source 1..19: mol_type = other RNA
ctgctgtccacagtcaaca

SEQ ID NO: 809 - RNA, 19 nt, synthetic construct
  source 1..19: mol_type = other RNA
agctgctgtccacagtcaa

SEQ ID NO: 810 - RNA, 19 nt, synthetic construct
  source 1..19: mol_type = other RNA
gttcattctccaaatcccc

SEQ ID NO: 811 - RNA, 19 nt, synthetic construct
  source 1..19: mol_type = other RNA
ggttcattctccaaatccc

SEQ ID NO: 812 - RNA, 19 nt, synthetic construct
  source 1..19: mol_type = other RNA
tggttcattctccaaatcc

SEQ ID NO: 813 - RNA, 19 nt, synthetic construct
  source 1..19: mol_type = other RNA
ctggttcattctccaaatc

SEQ ID NO: 814 - RNA, 19 nt, synthetic construct
  source 1..19: mol_type = other RNA
tctggttcattctccaaat

SEQ ID NO: 815 - RNA, 19 nt, synthetic construct
  source 1..19: mol_type = other RNA
ttctggttcattctccaaa

SEQ ID NO: 816 - RNA, 19 nt, synthetic construct
  source 1..19: mol_type = other RNA
cttctggttcattctccaa

SEQ ID NO: 817 - RNA, 19 nt, synthetic construct
  source 1..19: mol_type = other RNA
cttcttctggttcattctc

SEQ ID NO: 818 - RNA, 19 nt, synthetic construct
  source 1..19: mol_type = other RNA
gcttcttctggttcattct

SEQ ID NO: 819 - RNA, 19 nt, synthetic construct
  source 1..19: mol_type = other RNA
tgcttcttctggttcattc

SEQ ID NO: 820 - RNA, 19 nt, synthetic construct
  source 1..19: mol_type = other RNA
ctgcttcttctggttcatt

SEQ ID NO: 821 - RNA, 19 nt, synthetic construct
  source 1..19: mol_type = other RNA
cctgcttcttctggttcat

SEQ ID NO: 822 - RNA, 19 nt, synthetic construct
  source 1..19: mol_type = other RNA
acctgcttcttctggttca

SEQ ID NO: 823 - RNA, 19 nt, synthetic construct
  source 1..19: mol_type = other RNA
cacctgcttcttctggttc

SEQ ID NO: 824 - RNA, 19 nt, synthetic construct
  source 1..19: mol_type = other RNA
cagctccgtctattccttt

SEQ ID NO: 825 - RNA, 19 nt, synthetic construct
  source 1..19: mol_type = other RNA
actccagctccgtctattc

SEQ ID NO: 826 - RNA, 19 nt, synthetic construct
  source 1..19: mol_type = other RNA
aactccagctccgtctatt

SEQ ID NO: 827 - RNA, 19 nt, synthetic construct
  source 1..19: mol_type = other RNA
agatgctcaaggagctcat

SEQ ID NO: 828 - RNA, 19 nt, synthetic construct
  source 1..19: mol_type = other RNA
aagatgctcaaggagctca

SEQ ID NO: 829 - RNA, 19 nt, synthetic construct
  source 1..19: mol_type = other RNA
gaagatgctcaaggagctc

SEQ ID NO: 830 - RNA, 19 nt, synthetic construct
  source 1..19: mol_type = other RNA
agaagatgctcaaggagct

SEQ ID NO: 831 - RNA, 19 nt, synthetic construct
  source 1..19: mol_type = other RNA
cagaagatgctcaaggagc

SEQ ID NO: 832 - RNA, 19 nt, synthetic construct
  source 1..19: mol_type = other RNA
ccagaagatgctcaaggag

SEQ ID NO: 833 - RNA, 19 nt, synthetic construct
  source 1..19: mol_type = other RNA
cccagaagatgctcaagga

SEQ ID NO: 834 - RNA, 19 nt, synthetic construct
  source 1..19: mol_type = other RNA
aagcccagaagatgctcaa

SEQ ID NO: 835 - RNA, 19 nt, synthetic construct
  source 1..19: mol_type = other RNA
aaagcccagaagatgctca

SEQ ID NO: 836 - RNA, 19 nt, synthetic construct
  source 1..19: mol_type = other RNA
gtcagccaggtgtactcag

SEQ ID NO: 837 - RNA, 19 nt, synthetic construct
  source 1..19: mol_type = other RNA
ggtcagccaggtgtactca

SEQ ID NO: 838 - RNA, 19 nt, synthetic construct
  source 1..19: mol_type = other RNA
actctggtatttcctaggg

SEQ ID NO: 839 - RNA, 19 nt, synthetic construct
  source 1..19: mol_type = other RNA
tactctggtatttcctagg

SEQ ID NO: 840 - RNA, 19 nt, synthetic construct
  source 1..19: mol_type = other RNA
ctactctggtatttcctag

SEQ ID NO: 841 - RNA, 19 nt, synthetic construct
  source 1..19: mol_type = other RNA
gctactctggtatttccta

SEQ ID NO: 842 - RNA, 19 nt, synthetic construct
  source 1..19: mol_type = other RNA
tgctactctggtatttcct

SEQ ID NO: 843 - RNA, 19 nt, synthetic construct
  source 1..19: mol_type = other RNA
gtgctactctggtatttcc

SEQ ID NO: 844 - RNA, 19 nt, synthetic construct
  source 1..19: mol_type = other RNA
ggtgctactctggtatttc

SEQ ID NO: 845 - RNA, 19 nt, synthetic construct
  source 1..19: mol_type = other RNA
gggtgctactctggtattt

SEQ ID NO: 846 - RNA, 19 nt, synthetic construct
  source 1..19: mol_type = other RNA
ggactccaagagaaaagcc

SEQ ID NO: 847 - RNA, 19 nt, synthetic construct
  source 1..19: mol_type = other RNA
aggactccaagagaaaagc

SEQ ID NO: 848 - RNA, 19 nt, synthetic construct
  source 1..19: mol_type = other RNA
tgaggactccaagagaaaa

SEQ ID NO: 849 - RNA, 19 nt, synthetic construct
  source 1..19: mol_type = other RNA
gtgaggactccaagagaaa

SEQ ID NO: 850 - RNA, 19 nt, synthetic construct
  source 1..19: mol_type = other RNA
tgtgaggactccaagagaa

SEQ ID NO: 851 - RNA, 19 nt, synthetic construct
  source 1..19: mol_type = other RNA
ctgtgaggactccaagaga

SEQ ID NO: 852 - RNA, 19 nt, synthetic construct
  source 1..19: mol_type = other RNA
cctgtgaggactccaagag

SEQ ID NO: 853 - DNA, 2475 bp, Homo sapiens
  source 1..2475: mol_type = other DNA
atgcccgtggccgaggccccccaggtggctggcgggcagggggacggaggtgatggcgaggaagcggagccggaggggatgttcaaggcctgtgaggactccaagagaaaagcccggggctacctccgcctggtgcccctgtttgtgctgctggccctgctcgtgctggcttcggcgggggtgctactctggtatttcctagggtacaaggcggaggtgatggtcagccaggtgtactcaggcagtctgcgtgtactcaatcgccacttctcccaggatcttacccgccgggaatctagtgccttccgcagtgaaaccgccaaagcccagaagatgctcaaggagctcatcaccagcacccgcctgggaacttactacaactccagctccgtctattcctttggggagggacccctcacctgcttcttctggttcattctccaaatccccgagcaccgccggctgatgctgagccccgaggtggtgcaggcactgctggtggaggagctgctgtccacagtcaacagctcggctgccgtcccctacagggccgagtacgaagtggaccccgagggcctagtgatcctggaagccagtgtgaaagacatagctgcattgaattccacgctgggttgttaccgctacagctacgtgggccagggccaggtcctccggctgaaggggcctgaccacctggcctccagctgcctgtggcacctgcagggccccaaggacctcatgctcaaactccggctggagtggacgctggcagagtgccgggaccgactggccatgtatgacgtggccgggcccctggagaagaggctcatcacctcggtgtacggctgcagccgccaggagcccgtggtggaggttctggcgtcgggggccatcatggcggtcgtctggaagaagggcctgcacagctactacgaccccttcgtgctctccgtgcagccggtggtcttccaggcctgtgaagtgaacctgacgctggacaacaggctcgactcccagggcgtcctcagcaccccgtacttccccagctactactcgccccaaacccactgctcctggcacctcacggtgccctctctggactacggcttggccctctggtttgatgcctatgcactgaggaggcagaagtatgatttgccgtgcacccagggccagtggacgatccagaacaggaggctgtgtggcttgcgcatcctgcagccctacgccgagaggatccccgtggtggccacggccgggatcaccatcaacttcacctcccagatctccctcaccgggcccggtgtgcgggtgcactatggcttgtacaaccagtcggacccctgccctggagagttcctctgttctgtgaatggactctgtgtccctgcctgtgatggggtcaaggactgccccaacggcctggatgagagaaactgcgtttgcagagccacattccagtgcaaagaggacagcacatgcatctcactgcccaaggtctgtgatgggcagcctgattgtctcaacggcagcgacgaagagcagtgccaggaaggggtgccatgtgggacattcaccttccagtgtgaggaccggagctgcgtgaagaagcccaacccgcagtgtgatgggcggcccgactgcagggacggctcggatgaggagcactgtgactgtggcctccagggcccctccagccgcattgttggtggagctgtgtcctccgagggtgagtggccatggcaggccagcctccaggttcggggtcgacacatctgtgggggggccctcatcgctgaccgctgggtgataacagctgcccactgcttccaggaggacagcatggcctccacggtgctgtggaccgtgttcctgggcaaggtgtggcagaactcgcgctggcctggagaggtgtccttcaaggtgagccgcctgctcctgcacccgtaccacgaagaggacagccatgactacgacgtggcgctgctgcagctcgaccacccggtggtgcgctcggccgccgtgcgccccgtctgcctgcccgcgcgctcccacttcttcgagcccggcctgcactgctggattacgggctggggcgccttgcgcgagggcgccctacgggcggatgctgtggccctattttatggatggagaaaccaaggctcagagacatgttgctgccccatcagcaacgctctgcagaaagtggatgtgcagttgatcccacaggacctgtgcagcgaggtctatcgctaccaggtgacgccacgcatgctgtgtgccggctaccgcaagggcaagaaggatgcctgtcagggtgactcaggtggtccgctggtgtgcaaggcactcagtggccgctggttcctggcggggctggtcagctggggcctgggctgtggccggcctaactacttcggcgtctacacccgcatcacaggtgtgatcagctggatccagcaagtggtgacctga

SEQ ID NO: 854 - DNA, 2400 bp, Mus musculus
  source 1..2400: mol_type = other DNA
atgcccaccaccgaggtcccccaagcggctgatggtcagggcgatgcgggtgatggagaggaagctgctgagccagaggggaagttcaagcccccaaaaaacaccaagagaaaaaaccgggactacgtccgcttcacgccactgttgctggtcttggctgcgctggtctcagcaggggtcatgctttggtatttcctagggtacaaagcggaagtgaccgtaagccaggtgtactctggcagcctccgggtgctcaaccgtcatttctcccaggacctgggccgacgggagtctattgctttccgcagtgaatctgccaaagcccagaagatgctccaagaactggttgccagcacccgcctgggtacttactacaactctagttctgtctactcctttggggagggacccctcacctgcttcttctggtttatccttgacatccctgagtaccagcgactgaccctgagccctgaagtagtgcgcgagctcctggtggatgagctactgtccaacagctcaaccctggcttcctataagaccgaatatgaggtggacccggaaggcctggtgatcctggaagccagtgtgaacgacatagtcgtactgaattccacgctgggctgttatcgctacagctatgtgaacccaggccaggtcctcccattgaaggggcctgaccagcagaccacaagctgcctgtggcatctgcaagggcccgaagacctcatgatcaaagtgcggctggagtggacccgggtcgattgcagagacagggtggcgatgtacgacgcagctgggcccctggagaagagacttatcacctcggtctatgggtgcagccgccaggaacctgtgatggaggtgctggcatcgggctccgtcatggccgtggtgtggaaaaagggcatgcatagctactatgaccctttcctgctctcagtgaagtctgtggccttccaggactgccaggtgaacctgacactggagggccggctggacacacagggcttcctccgtacaccctactaccccagttactactctcccagtacccactgctcctggcatctcacggtaccctctctggactacggcttggcgctctggttcgatgcctacgcactgaggaggcagaagtacaaccgactgtgtactcagggccagtggatgatccagaacaggaggctgtgtggcttccgtaccctgcagccatatgctgagaggatccccatggtggcctcagatggtgtcaccatcaacttcacctcccagatctccctcacaggcccgggtgtgcaagtgtactacagcttgtacaaccaatcagacccctgccctggtgagttcctctgctctgtgaatggactgtgtgtccctgcgtgtgacgggatcaaggactgccccaatggcctggatgagagaaactgtgtctgcagagccatgttccagtgccaagaggacagcacgtgcatttcactgcctagagtctgtgaccggcagcccgactgtctcaatggcagtgacgaagaacagtgccaagaaggagtgccctgtgggacattcactttccagtgtgaggaccggagctgtgtgaagaagcccaacccagagtgtgacggccagtcagattgcagagacggctcagatgagcaacactgtgactgtggcctccagggcctctccagccgtattgtgggcgggaccgtgtcctccgagggtgagtggccatggcaggccagcctccagattcggggtcgacacatctgtgggggggctctcatcgctgaccgctgggtcataacggccgcccactgcttccaggaggacagcatggcctccccgaagctgtggaccgtgttcctgggaaagatgcggcagaactcgcgctggccaggcgaggtgtccttcaaggtgagccgtctgttcctgcacccgtaccacgaggaggacagccatgactacgacgtggccctgctgcagctcgaccaccccgtggtgtactcggccactgtgcgccccgtctgcctgcctgcccgctcccacttctttgagccaggccagcactgctggatcacaggctggggagcccagcgagagggtggtccggtgagcaacaccctgcagaaggtggacgtacagctggtccctcaggacctctgcagtgaggcctaccgctaccaggtgtccccacgcatgctctgtgctggctaccgcaagggcaagaaagatgcctgccagggtgactctggaggcccactggtttgcagggagcccagtggccgctggttcctggcagggttggttagctggggcctgggctgtggccgacccaatttctttggcgtctacacccgtgtcacacgtgtgatcaactggatccagcaggtgctgacctga

SEQ ID NO: 855 - DNA, 2403 bp, Macaca mulatta
  source 1..2403: mol_type = other DNA
atgcctgtggccaaggccccccaggtggctggcgggcagggggacggaggtgatggcgaggaagcggagccagaggggatgttcgaggcccgtgaggactccaagagaaaagcccggggctacctccgcctggcgcccctgtggctgaccctggttgtgctgacttcagtgggggtgctactctggtatttcctagggtacaaggcggaggtgacggtcagccaggtgtactcaggcagccttcgcgtgctcaatcgccacttctcccaggatcttacccgccgggaatccagtgccttccgcagtgaaaccgccaaagcccagaagatgctcaaggagctcatcgccagcacccgcctgggaacttattacaactccagctccgtctattcctttggggagggaccgctcacctgcttcttctggttcattctccaaatccccgagcaccgccggccgatgctgagccccgaggtggtgcaggcactgctggtggaggagctgctgtccacagtcaacagctcggcggccgtcccctacagggccgagtacgaagtggaccccgagggcctagtgatcctagaagccagtgtgaaagacatagctgcactgaattccacgctgggttgttaccgctacagctacgtgggccagggtcaggtcctccggctgaagggacccgaccacctggcctccagctgcctgtggcacctgcagggccccgaagacctcatgctgaaactccggctggagtggacgctggccgagtgccgggaccgactggccatgtatgacgtggctgggcccctggagaagaggctcatcacctcggtgtatggctgcagccgccaggagcctgtggtggaagtcctggcatcgggggccatcatggcggtggtctggaagaagggcctgcacagctactacgacccctttatgctctccgtgcagtcggtggtcttccaggcctgcgaggtaaacctgacgctggatgacaggctggactcccagggcgtcctcagcaccccgtacttccccagctactactcgccccgaacccactgctcctggcacctcacggtgccctctctggactacggcttggccctctggtttgacgcctacgcactgcggaggcagaagtatgatttgccgtgcacccagggccagtggacgatccagaacaggaggctgtgtggcctgcgcatcctgcagccttacgccgagaggatccccgtggtggccacggccggcatcaccatcaatttcacctcccagatctccctcacagggcctggtgtgcgggtgcactatggcttgtacaaccagtcggacccctgccctggagagttcctctgctctgtgaacggactctgcgtccctgcctgtgatggggtcaaggactgccccaacggcctggatgagagaaactgcgtttgcagagccacattccagtgccaagaggacagcacgtgcatctcactgcttaaggtctgtgacgggcagcctgactgtctcaatggcagcgatgaagagcggtgccaggaaggggtgccctgcgggacattcaccttccagtgtgaggaccagagctgcgtgaagaagcccaacccacagtgtgatgggcggcccgactgcagggacggctcagacgagcagcactgtgactgtggcctccagggcccctccagtcgcattgttggtggggccgtgtcctccgagggtgagtggccatggcaggccagcctccaggttcggggtcgacacatctgtgggggcgccctcatcgctgaccgctgggtgataacagctgcccattgcttccaggaggacagcatggcctccccggcgctgtggacggtgttcctgggcaaggtgtggcagaactcgcgctggcctggagaggtgtccttcaaggtgagccgcctactcctgcatccgtatcacgaagaggacagccacgactacgacgtggcgctgttgcagctcgaccacccggtggtgcgctcggccgccgtgcgtcccgtctgcctgcccgcgcgctcccacttcttcgaacccggcctgcactgctggatcactggctggggcgccctgcgcgaaggcggccccaccagcaatgctctgcagaaagtggacgtgcagttgatcccacaggacctgtgcagcgaggcctatcgctaccaggtgacgccacgcatgctgtgtgccggctaccgcaagggcaagaaggatgcctgccagggtgactcgggtggtccgctggtatgcaaggcactcagtggccgctggttcctggcagggctggtcagctggggcctgggctgtggccggcctaactacttcggcgtctacacccgcatcacaggtgtgatcggctggatccagcaagtggtgacctga

SEQ ID NO: 856 - RNA, 16 nt, synthetic construct
  source 1..16: mol_type = other RNA
gcagccgaaaggctgc

SEQ ID NO: 857 - DNA, 19 bp, synthetic construct
  source 1..19: mol_type = other DNA
agcctgattgtctcaacgg

SEQ ID NO: 858 - DNA, 20 bp, synthetic construct
  source 1..20: mol_type = other DNA
ctggaaggtgaatgtcccac

SEQ ID NO: 859 - DNA, 22 bp, synthetic construct
  source 1..22: mol_type = other DNA
acgaagagcagtgccaggaagg

SEQ ID NO: 860 - DNA, 21 bp, synthetic construct
  source 1..21: mol_type = other DNA
gtactcaatcgccacttctcc

SEQ ID NO: 861 - DNA, 21 bp, synthetic construct
  source 1..21: mol_type = other DNA
gaatagacggagctggagttg

SEQ ID NO: 862 - DNA, 22 bp, synthetic construct
  source 1..22: mol_type = other DNA
cagtgaaaccgccaaagcccag

(19) United States
(12) Patent Application Publication
(10) Pub. No.: US 2024/0309388 A1
(43) Pub. Date: Sep. 19, 2024

(54) COMPOSITIONS AND METHODS FOR INHIBITING TRANSMEMBRANE SERINE PROTEASE 6 (TMPRSS6) EXPRESSION

(71) Applicants: Novo Nordisk A/S, Bagsvaerd (DK); Dicerna Pharmaceuticals, Inc., Lexington, MA (US)

(72) Inventors: Anna Linda Blois, Sudbury, MA (US); Christina Marie Priest, Lexington, MA (US); Jihye Park, Cambridge, MA (US); Henryk Dudek, Belmont, MA (US)

(21) Appl. No.: 18/441,001

(22) Filed: Feb. 14, 2024

Related U.S. Application Data
(63) Continuation of application No. 18/213,289, filed on Jun. 23, 2023, now Pat. No. 11,952,574.
(60) Provisional application No. 63/355,210, filed on Jun. 24, 2022.

(30) Foreign Application Priority Data
Nov. 23, 2022 (EP) 22209113.4

(51) Int. Cl. C12N 15/113; A61P 7/00

(52) U.S. Cl. CPC C12N 15/1138; A61P 7/00; C12N 2310/14; C12N 2310/315; C12N 2310/531

(57) ABSTRACT

Oligonucleotides are provided herein that inhibit TMPRSS6 expression. Also provided are compositions including the same and uses thereof, particularly uses relating to treating diseases, disorders and/or conditions associated with hepcidin deficiency or suppression.

### CROSS-REFERENCE TO RELATED APPLICATIONS

This application is a continuation of U.S. application Ser. No. 18/213,289, filed Jun. 23, 2023 which claims priority under 35 U.S.C. § 119 to U.S. Provisional Application No. 63/355,210, filed Jun. 24, 2022 and European Patent Application 22209113.4, filed Nov. 23, 2022; the contents of which are incorporated herein by reference.

### INCORPORATION-BY-REFERENCE OF THE SEQUENCE LISTING

The instant application contains a Sequence Listing which has been submitted in ASCII format via EFS-Web and is hereby incorporated by reference in its entirety. Said ASCII copy, created on Jan. 11, 2024, is named “210088US03”, and is 1,590 kilobytes in size.

### BACKGROUND

Iron is a micronutrient that is a biologically essential component of every living organism. It is required for an adequate erythropoietic function, oxidative metabolism, cellular immune response, and numerous other cellular processes. In its free form, iron is involved in oxidation-reduction reactions, leading to the formation of free radicals, oxidative stress which may lead to organ damage. Living organisms have developed protein systems to transport free iron through the cell membranes and biological fluids and store it in a non-toxic and readily mobilizable form to avoid iron toxicity. In the human body, iron mainly exists in complex forms bound to protein as heme compounds, heme enzymes, or nonheme compounds. Iron absorption occurs by the enterocytes by divalent metal transport 1 and takes place in the duodenum and upper jejunum. Iron is then transferred across the he duodenal mucosa into the blood, where it is transported by transferrin to the cells or the bone marrow for erythropoiesis. Since iron is required for a number of diverse cellular functions, a constant balance between iron uptake, transport, storage, and utilization is required to maintain iron homeostasis. Hepcidin, a circulating peptide hormone encoded by the HAMP gene, is secreted by the liver that plays a crucial role in maintaining iron homeostasis. Hepcidin expression is directly regulated by variations in iron intake and its repression leads to an increase in bioavailable serum iron level. Hepcidin blocks iron flux into the plasma by its direct binding to ferroportin, the principal iron exporter localized at the cell membrane of duodenal enterocytes, which absorb iron from the diet, and macrophages, which recycle iron from senescent erythrocytes. Hepcidin binding to ferroportin triggers its internalization and subsequent lysosomal degradation, thereby blocking iron export to the circulatory system. Hepatic hepcidin expression is regulated by three independent pathways, where inflammation (inflammatory cytokines) and high serum iron levels result in hepcidin upregulation, whereas it is downregulated by elevated erythropoiesis facilitating high iron demand for red blood cell production. Put differently, when hepcidin levels are high, serum iron levels decrease which can result in anemia. When hepcidin levels are low, in disease states such as hemochromatosis, iron levels rise and overload can occur.

In pathological situations, prolonged repression of hepcidin often leads to primary iron overload, due to its malfunctional expression as a result of loss of function in the plasma iron-sensing signalling pathway (e.g. hereditary hemochromatosis). In situations of elevated, but dysfunctional erythropoiesis (e.g. beta thalassemia), hepcidin is down regulated resulting in secondary iron overload. The pathophysiological consequences of primary and secondary iron overload are common resulting in built-up of free plasma iron, accumulation in organs causing damage at disease progression.

TMPRSS6 (transmembrane Protease, Serine 6) encodes a type II serine protease and is expressed mainly in the liver. TMPRSS6 participates in a transmembrane signaling pathway triggered by iron deficiency and suppresses diverse pathways of the HAMP activation, the gene that encodes hepcidin.

Studies show that upregulation of hepcidin ameliorate abnormal erythropoiesis and prevents or limits iron overload in mouse models of β-thalassemia intermedia and hereditary hemochromatosis (Gardenghi et al. J. Clin. Invest., 2010; Guo et al, J. Clin. Invest., 2013, Schmidt et al. Blood 2013; Casu et al. Blood 2016; Ramos et al. Blood 2012). HFE-associated hereditary hemochromatosis is the most common type of inherited iron overload disorder, with a high prevalence of the p.C282Y mutation in northern European populations (Alexander and Kowdley, Genetics in Medicine, 2009). It is thought that impaired hepcidin production in the liver leads to iron overload in HFE-associated hemochromatosis. Iron overload in this context often leads to complications such as liver cirrhosis and diabetes, as well as failure of other affected organs.

Consistent with its role in suppressing hepcidin expression, mutations in TMPRSS6 result in disorders such as iron-refractory, iron-deficient anemia. Hepcidin expression is significantly elevated in TMPRSS6−/− mice and reduction of TMPRSS6 in Hfe−/− mice could ameliorate the iron overload phenotype (Du et al. Science 2008; Folgueras et al. Blood 2008; Finberg K E et al., Blood, 2011).

Such evidence suggests a role for targeting TMPRSS6 in the treatment of diseases associated with iron overload. Current treatment options for such diseases are limited. Accordingly, methods for effective treatment of disorders associated with iron overload are currently needed and the present invention addresses this need.

### SUMMARY OF DISCLOSURE

The present disclosure is based, in part, on the discovery of oligonucleotides (e.g., RNAi oligonucleotides) that reduce TMPRSS6 expression in vitro and in vivo. Aberrant expression of TMPRSS6 has been shown to be associated with altered hepcidin expression and serum iron levels. As demonstrated herein, serum iron and serum iron saturation are reduced in vivo following administration of TMPRSS6 RNAi oligonucleotides. Without being bound by theory, inhibition of TMPRSS6 increases hepcidin expression and subsequently reduces serum iron levels preventing iron overload. Therefore, without wishing to be bound by theory, oligonucleotides targeting TMPRSS6 are useful for treating diseases or disorders associated with hepcidin deficiency or suppression.

Accordingly, in some aspects, the present disclosures provides an RNAi oligonucleotide for reducing transmembrane serine protease 6 (TMPRSS6) expression, the oligonucleotide comprising a sense strand and an antisense strand, wherein the sense strand and the antisense strand form a duplex region, wherein the antisense strand comprises a region of complementarity to a TMPRSS6 mRNA target sequence of any one of SEQ ID NOs: 661-852, and wherein the region of complementarity is at least 15 contiguous nucleotides in length.

In some embodiments the sense strand is 15 to 50 nucleotides in length. In some embodiments, the sense strand is 18 to 36 nucleotides in length.

In some embodiments, the antisense strand is 15 to 30 nucleotides in length.

In some embodiments, the antisense strand is 22 nucleotides in length and the antisense strand and the sense strand form a duplex region of at least 19 nucleotides in length, optionally at least 20 nucleotides in length. In some embodiments, the region of complementarity is at least 19 contiguous nucleotides in length. In some embodiments, the region of complementarity is at least 20 contiguous nucleotides in length.

In some aspects, the disclosure provides a double stranded RNAi oligonucleotide for reducing TMPRSS6 expression, the oligonucleotide comprising:
(i) an antisense strand of 19-30 nucleotides in length, wherein the antisense strand comprises a nucleotide sequence comprising a region of complementarity to a TMPRSS6 mRNA target sequence, wherein the region of complementarity is selected from SEQ ID NOs: 1-192, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some aspects, the disclosure provides a double stranded RNAi oligonucleotide for reducing TMPRSS6 expression, the oligonucleotide comprising:
(i) an antisense strand of 19-30 nucleotides in length, wherein the antisense strand comprises a nucleotide sequence selected from SEQ ID NOs: 1-192, and wherein the antisense strand is complementary a TMPRSS6 mRNA target sequence, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, the 3′ end of the sense strand comprises a stem-loop set forth as S1-Lp-S2, wherein S1 is complementary to S2, and wherein Lp forms a loop between S1 and S2 of 3-5 nucleotides in length. In some embodiments, Lp is a triloop or a tetraloop. In some embodiments, Lp is a tetraloop. In some embodiments, the tetraloop comprises the sequence 5′-GAAA-3′. In some embodiments, the S1 and S2 are 1-10 nucleotides in length and have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments, the stem-loop comprises the sequence 5′-GCAGCCGAAAGGCUGC-3′ (SEQ ID NO: 856).

In some embodiments, the sense strand, proximal to the 3′ end of the sense strand, comprises a stem-loop set forth as S1-Lp-S2, wherein S1 is complementary to S2, and wherein Lp forms a loop between S1 and S2 of 3-5 nucleotides in length. In some embodiments, Lp is a triloop or a tetraloop. In some embodiments, Lp is a tetraloop. In some embodiments, the tetraloop comprises the sequence 5′-GAAA-3′. In some embodiments, the S1 and S2 are 1-10 nucleotides in length and have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments, the stem-loop comprises the sequence 5′-GCAGCCGAAAGGCUGC-3′ (SEQ ID NO: 856).

In some embodiments, the antisense strand comprises a 3′ overhang sequence of one or more nucleotides in length. In some embodiments, the overhang comprises purine nucleotides. In some embodiments, the 3′ overhang sequence is 2 nucleotides in length. In some embodiments, the 3′ overhang is selected from AA, GG, AG, and GA. In some embodiments, the overhang is GG or AA. In some embodiments, the overhang is GG. 5 In some embodiments, the oligonucleotide comprises at least one modified nucleotide. In some embodiments, the modified nucleotide comprises a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the modification is a 2′-modification selected from 2′-fluoro and 2′-O-methyl. In 10 some embodiments, about 10-15%, 10%, 11%, 12%, 13%, 14% or 15% of the nucleotides of the sense strand comprise a 2′-fluoro modification. In some embodiments, about 25-35%, 25%, 26%, 27%, 28%, 29%, 30%, 31%, 32%, 33%, 34% or 35% of the nucleotides of the antisense strand comprise a 2′-fluoro modification. In some embodiments, about 25-35%, 25%, 26%, 27%, 28%, 29%, 30%, 31%, 32%, 33%, 34% or 35% of the nucleotides of the oligonucleotide comprise a 2′-fluoro modification. In some embodiments, the sense strand comprises 36 nucleotides with positions 1-36 from 5′ to 3′, wherein positions 8-11 comprise a 2′-fluoro modification. In some embodiments, the antisense strand comprises 22 nucleotides with positions 1-22 from 5′ to 3′, and wherein positions 2, 3, 4, 5, 7, 10 and 14 comprise a 2′-fluoro modification.

In some embodiments, the oligonucleotide comprises at least one modified internucleotide linkage. In some embodiments, the at least one modified internucleotide linkage is a phosphorothioate linkage. In some embodiments, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some embodiments, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some embodiments, the antisense strand is 22 nucleotides in length, and wherein the antisense strand comprises a phosphorothioate linkage between positions 1 and 2, between positions 2 and 3, positions 3 and 4, positions 20 and 21, and positions 21 and 22. In some embodiments, the sense strand comprises a phosphorothioate linkage between positions 1 and 2, wherein positions are numbered 5′ to 3′.

In some embodiments, the 4′-carbon of the sugar of the 5′-nucleotide of the antisense strand comprises a phosphate analog. In some embodiments, the phosphate analog is oxymethyl phosphonate, vinyl phosphonate or malonyl phosphonate, optionally wherein the phosphate analog is a 4′-phosphate analog comprising 4′-oxymethylphosphonate.

In some embodiments, at least one nucleotide of the oligonucleotide is conjugated to one or more targeting ligands. In some embodiments, each targeting ligand comprises a carbohydrate, amino sugar, cholesterol, or polypeptide. In some embodiments, the stem loop comprises one or more targeting ligands conjugated to one or more nucleotides of the stem loop. In some embodiments, the one or more targeting ligands is conjugated to one or more nucleotides of the loop. In some embodiments, the loop comprises 4 nucleotides numbered 1-4 from 5′ to 3′, wherein nucleotides at positions 2, 3, and 4 each comprise one or more targeting ligands, wherein the targeting ligands are the same or different. In some embodiments, each targeting ligand comprises a N-acetylgalactosamine (GalNAc) moiety. In some embodiments, the GalNac moiety is a monovalent GalNAc moiety, a bivalent GalNAc moiety, a trivalent GalNAc moiety or a tetravalent GalNAc moiety. In some embodiments, up to 4 nucleotides of Lp of the stem-loop are each conjugated to a monovalent GalNAc moiety.

In some embodiments, the region of complementarity is fully complementary to the mRNA target sequence. In some embodiments, the region of complementarity is partially complementary to the mRNA target sequence. In some embodiments, the region of complementarity comprises no more than four mismatches to the mRNA target sequence.

In some embodiments, the region of complementarity is fully complementary to the TMPRSS6 mRNA target sequence at nucleotide positions 2-8 of the antisense strand, wherein nucleotide positions are numbered 5′ to 3′. In some embodiments, the region of complementarity is fully complementary to the TMPRSS6 mRNA target sequence at nucleotide positions 2-11 of the antisense strand, wherein nucleotide positions are numbered 5′ to 3′.

In some embodiments, the sense strand comprises a nucleotide sequence of any one of SEQ ID NOs: 579-580, 585-587, 590 and 595-597. In some embodiments, the antisense strand comprises a nucleotide sequence of any one of SEQ ID NOs: 600-601, 606-608, 611 and 616-618.

In some embodiments, the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively.

In some embodiments, the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively.

In some embodiments, the sense strand comprises a nucleotide sequence as set forth in SEQ ID NO: 579, and the antisense strand comprises a nucleotide sequence as set forth in SEQ ID NO: 600. In some embodiments, the sense strand comprises a nucleotide sequence as set forth in SEQ ID NO: 580, and the antisense strand comprises a nucleotide sequence as set forth in SEQ ID NO: 601. In some embodiments, the sense strand comprises a nucleotide sequence as set forth in SEQ ID NO: 590, and the antisense strand comprises a nucleotide sequence as set forth in SEQ ID NO: 611. In some embodiments, the sense strand comprises a nucleotide sequence as set forth in SEQ ID NO: 597, and the antisense strand comprises a nucleotide sequence as set forth in SEQ ID NO: 618. In some embodiments, the sense strand comprises a nucleotide sequence as set forth in SEQ ID NO: 586, and the antisense strand comprises a nucleotide sequence as set forth in SEQ ID NO: 607.

In some embodiments, the antisense strand is 22 nucleotides in length.

In some embodiments, the antisense strand comprises a nucleotide sequence selected from SEQ ID NOs: 600-601, 606-608, 611, and 616-618.

In some embodiments, the sense strand is 36 nucleotides in length.

In some embodiments, the sense strand comprises a nucleotide sequence selected from SEQ ID NOs: 579-580, 585-587, 590, and 595-597.

In some embodiments, the sense strand and antisense strands comprise nucleotide sequences selected from the group consisting of:
a) SEQ ID NOs: 621 and 642, respectively;
b) SEQ ID NOs: 622 and 643, respectively;
c) SEQ ID NOs: 637 and 658, respectively;
d) SEQ ID NOs: 632 and 653, respectively;
e) SEQ ID NOs: 638 and 659, respectively;
f) SEQ ID NOs: 639 and 660, respectively;
g) SEQ ID NOs: 627 and 648, respectively;
h) SEQ ID NOs: 628 and 649, respectively; and,
i) SEQ ID NOs: 629 and 650, respectively.

In some embodiments, the sense strand and antisense strands comprise nucleotide sequences selected from the group consisting of:
a) SEQ ID NOs: 621 and 642, respectively;
b) SEQ ID NOs: 622 and 643, respectively;
c) SEQ ID NOs: 632 and 653, respectively;
d) SEQ ID NOs: 639 and 660, respectively; and,
e) SEQ ID NOs: 628 and 649, respectively.

In some embodiments, the sense strand comprises a nucleotide sequence as set forth in SEQ ID NO: 621, and the antisense strand comprises a nucleotide sequence as set forth in SEQ ID NO: 642. In some embodiments, the sense strand comprises a nucleotide sequence as set forth in SEQ ID NO: 622, and the antisense strand comprises a nucleotide sequence as set forth in SEQ ID NO: 643. In some embodiments, the sense strand comprises a nucleotide sequence as set forth in SEQ ID NO: 632, and the antisense strand comprises a nucleotide sequence as set forth in SEQ ID NO: 653. In some embodiments, the sense strand comprises a nucleotide sequence as set forth in SEQ ID NO: 639, and the antisense strand comprises a nucleotide sequence as set forth in SEQ ID NO: 660. In some embodiments, the sense strand comprises a nucleotide sequence as set forth in SEQ ID NO: 628, and the antisense strand comprises a nucleotide sequence as set forth in SEQ ID NO: 649.

In some aspects, the disclosure provides an RNAi oligonucleotide for reducing TMPRSS6 expression, the oligonucleotide comprising a sense strand and an antisense strand, wherein the sense strand comprises the sequence and all of the modifications of 5′-[mGs][mG][mU][mG][mC][mU][mA][fC][fU][fC][fU][mG][mG][mU][mA][mU][mU][mU][mC][mA][mG][mC][mA][mG][mC][mC][mG][ademA-GalNAc][ademA-GalNAc][ademA-GalNAc][mG][mG][mC][mU][mG][mC]-3′ (SEQ ID NO: 621), and wherein the antisense strand comprises the sequence and all of the modifications of 5′ [MePhosphonate-40-mUs][fGs][fAs][fA][fA][mU][fA][mC][mC][fA][mG][mA][mG][fU][mA][mG][mC][mA][mC][mCs][m Gs][mG]-3′ (SEQ ID NO: 642), wherein mC, mA, mG, mU=2′-OMe ribonucleosides; fA, fC, fG, fU=2′-F ribonucleosides; s=phosphorothioate, and wherein ademA-GalNAc=

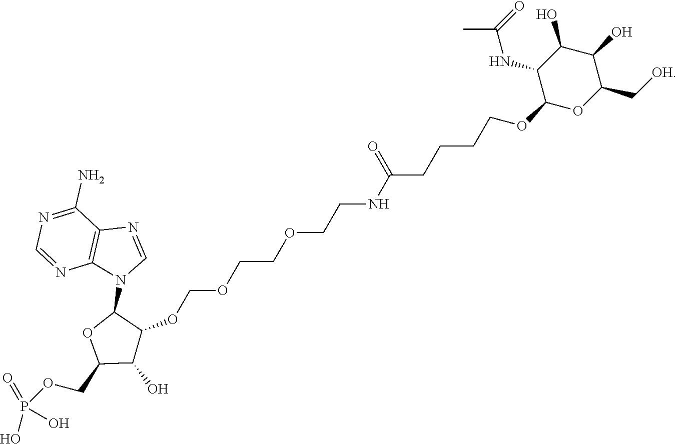 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the disclosure provides an RNAi oligonucleotide for reducing TMPRSS6 expression, the oligonucleotide comprising a sense strand and an antisense strand, wherein the sense strand comprises the sequence and all of the modifications of 5′ [mGs][mC][mU][mA][mC][mU][mC][fU][fG][fG][fU][mA][mU][mU][mU][mC][mC][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 622), and wherein the antisense strand comprises the sequence and all of the modifications of 5′ [MePhosphonate-40-mUs][fUs][fAs][fG][fG][mA][fA][mA][mU][fA][mC][mC][mA][fG][mA][mG][mU][mA][mG][mCs][m Gs][mG]-3′ (SEQ ID NO: 643), wherein mC, mA, mG, mU=2′-OMe ribonucleosides; fA, fC, fG, fU=2′-F ribonucleosides; s-phosphorothioate, and wherein ademA-GalNAc=

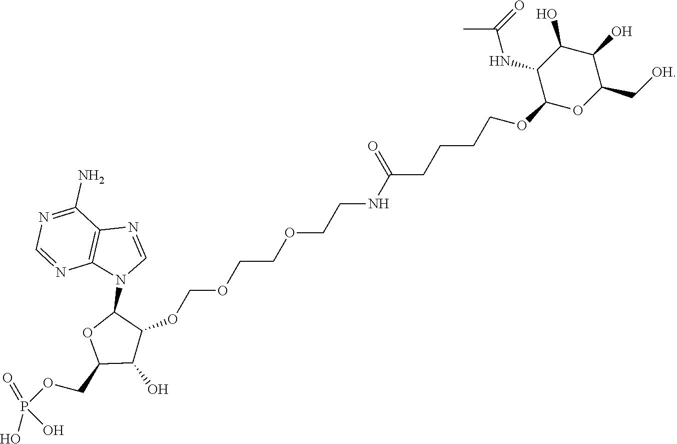 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the disclosure provides an RNAi oligonucleotide for reducing TMPRSS6 expression, the oligonucleotide comprising a sense strand and an antisense strand, wherein the sense strand comprises the sequence and all of the modifications of 5′-[mCs][mU][mC][mA][mC][mC][mU][fG][fC][fU][fU][mC][mU][mU][mC][mU][mG][mG][mU][mA][mG][mC][mA][mG][mC][mC][mG][ademA-GalNAc][ademA-GalNAc][ademA-GalNAc][mG][mG][mC][mU][mG][mC]-3′ (SEQ ID NO: 632), and wherein the antisense strand comprises the sequence and all of the modifications of 5′-[MePhosphonate-40-mUs][fAs][fCs][fC][fA][mG][fA][mA][mG][fA][mA][mG][mC][fA] [mG][mG][mU][mG][mA][mGs][mGs][mG]-3′ (SEQ ID NO: 653), wherein mC, mA, mG, mU=2′-OMe ribonucleosides; fA, fC, fG, fU=2′-F ribonucleosides; s=phosphorothioate, and wherein ademA-GalNAc=

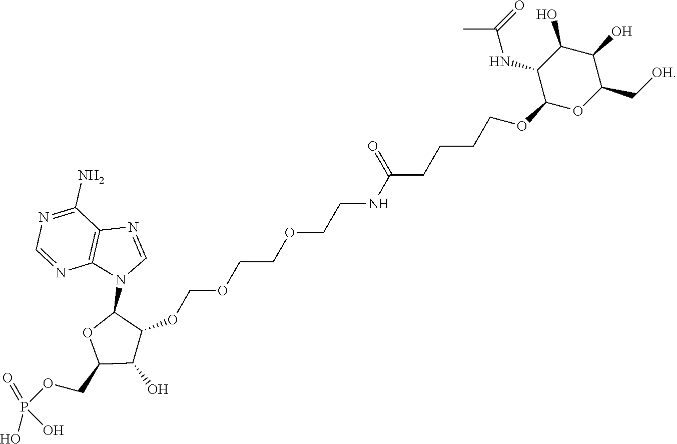 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the disclosure provides an RNAi oligonucleotide for reducing TMPRSS6 expression, the oligonucleotide comprising a sense strand and an antisense strand, wherein the sense strand comprises the sequence and all of the modifications of 5′-[mAs][mG][mU][mG][mU][mG][mA][fA][fA][fG][fA][mC][mA][mU][mA][mG][mC][mU][mG][mA][mG][mC][mA][mG][mC][mC][mG][ademA-GalNAc][ademA-GalNAc][ademA-GalNAc][mG][mG][mC][mU][mG][mC]3′ (SEQ ID NO: 639), and wherein the antisense strand comprises the sequence and all of the modifications of 5′-[MePhosphonate-40-mUs][fCs][fAs][G][fC][mU][fA][mU][mG][fU][mC][mU][mU][fU][mC][mA][mC][mA][mC][mUs][m Gs][mG]-3′ (SEQ ID NO: 660), wherein mC, mA, mG, mU=2′-OMe ribonucleosides; fA, fC, fG, fU=2′-F ribonucleosides; s-phosphorothioate, and wherein ademA-GalNAc=

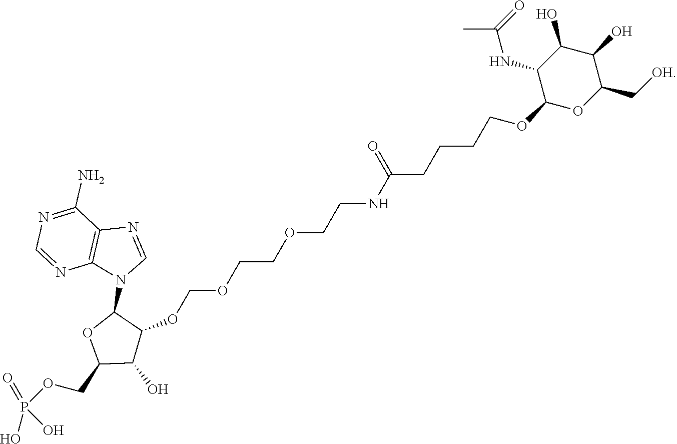 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the disclosure provides an RNAi oligonucleotide for reducing TMPRSS6 expression, the oligonucleotide comprising a sense strand and an antisense strand, wherein the sense strand comprises the sequence and all of the modifications of 5′-[mGs][mG][mG][mU][mG][mC][mA][fC][fU][fA][fU][mG][mG][mC][mU][mU][mG][mU][mA][mA][mG][mC][mA][mG][mC][mC][mG][ademA-GalNAc][ademA-GalNAc][ademA-GalNAc][mG][mG][mC][mU][mG][mC]3′ (SEQ ID NO: 628), and wherein the antisense strand comprises the sequence and all of the modifications of 5′-[MePhosphonate-40-mUs][fUs][fAs][fC][fA][mA][fG][mC][mC][fA][mU][mA][mG][fU][mG][mC][mA][mC][mC][mCs][m Gs][mG]-3′ (SEQ ID NO: 649), wherein mC, mA, mG, mU=2′-OMe ribonucleosides; fA, fC, fG, fU=2′-F ribonucleosides; s-phosphorothioate, and wherein ademA-GalNAc=

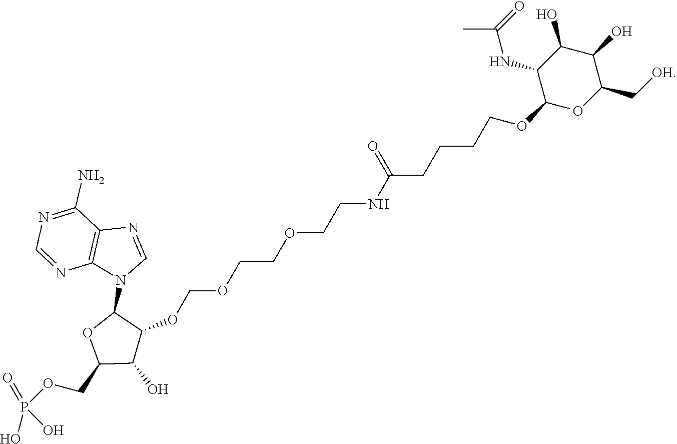 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the disclosure provides an RNAi oligonucleotide for reducing TMPRSS6 expression, the oligonucleotide comprising a sense strand and an antisense strand, wherein the sense strand comprises SEQ ID NO: 621 and the antisense strand comprises SEQ ID NO: 642, the antisense strand comprising a region of complementarity to a TMPRSS6 RNA transcript, wherein said RNAi is in the form of a conjugate having the structure as shown in FIG. 14A.

In some aspects, the disclosure provides an RNAi oligonucleotide for reducing TMPRSS6 expression, the oligonucleotide comprising a sense strand and an antisense strand, wherein the sense strand comprises SEQ ID NO: 632 and the antisense strand comprises SEQ ID NO: 653, the antisense strand comprising a region of complementarity to a TMPRSS6 RNA transcript, wherein said RNAi is in the form of a conjugate having the structure as shown in FIG. 14B.

In some aspects, the disclosure provides an RNAi oligonucleotide for reducing TMPRSS6 expression, the oligonucleotide comprising a sense strand and an antisense strand, wherein the sense strand comprises SEQ ID NO: 639 and the antisense strand comprises SEQ ID NO: 660, the antisense strand comprising a region of complementarity to a TMPRSS6 RNA transcript, wherein said RNAi is in the form of a conjugate having the structure as shown in FIG. 14C.

In some aspects, the disclosure provides an RNAi oligonucleotide for reducing TMPRSS6 expression, the oligonucleotide comprising a sense strand and an antisense strand, wherein the sense strand comprises SEQ ID NO: 628 and the antisense strand comprises SEQ ID NO: 649, the antisense strand comprising a region of complementarity to a TMPRSS6 RNA transcript, wherein said RNAi is in the form of a conjugate having the structure as shown in FIG. 14D.

In some aspects, the disclosure provides an RNAi oligonucleotide for reducing TMPRSS6 expression, the oligonucleotide comprising a sense strand and an antisense strand, wherein the sense strand comprises SEQ ID NO: 621 and the antisense strand comprises SEQ ID NO: 642, the antisense strand comprising a region of complementarity to a TMPRSS6 RNA transcript, wherein said RNAi is in the form of a conjugate having the structure as shown in FIGS. 10A-B.

In some aspects, the disclosure provides an RNAi oligonucleotide for reducing TMPRSS6 expression, the oligonucleotide comprising a sense strand and an antisense strand, wherein the sense strand comprises SEQ ID NO: 632 and the antisense strand comprises SEQ ID NO: 653, the antisense strand comprising a region of complementarity to a TMPRSS6 RNA transcript, wherein said RNAi is in the form of a conjugate having the structure as shown in FIGS. 11A-B.

In some aspects, the disclosure provides an RNAi oligonucleotide for reducing TMPRSS6 expression, the oligonucleotide comprising a sense strand and an antisense strand, wherein the sense strand comprises SEQ ID NO: 639 and the antisense strand comprises SEQ ID NO: 660, the antisense strand comprising a region of complementarity to a TMPRSS6 RNA transcript, wherein said RNAi is in the form of a conjugate having the structure as shown in FIGS. 12A-B.

In some aspects, the disclosure provides an RNAi oligonucleotide for reducing TMPRSS6 expression, the oligonucleotide comprising a sense strand and an antisense strand, wherein the sense strand comprises SEQ ID NO: 628 and the antisense strand comprises SEQ ID NO: 649, the antisense strand comprising a region of complementarity to a TMPRSS6 RNA transcript, wherein said RNAi is in the form of a conjugate having the structure as shown in FIGS. 13A-B.

In some embodiments, the disclosure provides a pharmaceutical composition comprising an RNAi oligonucleotide described herein, and a pharmaceutically acceptable carrier, delivery agent or excipient.

In some embodiments, the disclosure provides a method for treating a subject having a disease, disorder or condition associated with hepcidin deficiency or suppression, the method comprising administering to the subject a therapeutically effective amount of an RNAi oligonucleotide or pharmaceutical composition described herein, thereby treating the subject.

In some embodiments, hepcidin expression is increased after administering the RNAi oligonucleotide. In some embodiments, iron saturation levels in the serum are decreased after administering the RNAi oligonucleotide. In some embodiments, serum iron levels are decreased after administering the RNAi oligonucleotide.

In some embodiments, the disclosure provides a method of delivering an oligonucleotide to a subject, the method comprising administering a pharmaceutical composition described herein to the subject.

In some embodiments, the disclosure provides a method for reducing TMPRSS6 expression in a cell, a population of cells or a subject, the method comprising the step of:
i. contacting the cell or the population of cells with an RNAi oligonucleotide described herein, or a pharmaceutical composition described herein; or
ii. administering to the subject an RNAi oligonucleotide described herein, or a pharmaceutical composition described herein.

In some embodiments, reducing TMPRSS6 expression comprises reducing an amount or level of TMPRSS6 mRNA, an amount or level of matriptase-2 protein, or both. In some embodiments, reducing TMPRSS6 expression results in an increase in hepcidin production. In some embodiments, reducing TMPRSS6 expression results in a decrease in serum iron levels. In some embodiments, reducing TMPRSS6 expression results in a decrease in serum iron saturation.

In some embodiments, the subject has a disease, disorder or condition associated with hepcidin deficiency or suppression. In some embodiments, the disease, disorder or condition associated with hepcidin deficiency is hemochromatosis or beta-thalassemia. In some embodiments, the disease, disorder or condition associated with hepcidin suppression is polycythemia vera.

In some embodiments, the RNAi oligonucleotide, or pharmaceutical composition, is administered in combination with a second composition or therapeutic agent.

In some embodiments, the disclosure provides use of an RNAi oligonucleotide or pharmaceutical composition described herein, in the manufacture of a medicament for the treatment of a disease, disorder or condition associated with hepcidin deficiency or suppression, optionally for the treatment of hemochromatosis (e.g. hereditary hemochromatosis), polycythaemia vera or beta-thalassemia.

In some embodiments, the disclosure provides an RNAi oligonucleotide or pharmaceutical composition described herein, for use, or adaptable for use, in the treatment of a disease, disorder or condition associated with hepcidin deficiency or suppression, optionally for the treatment of hemochromatosis (e.g. hereditary hemochromatosis), polycythaemia vera or beta-thalassemia.

In some embodiments, the disclosure provides a kit comprising an RNAi oligonucleotide described herein, an optional pharmaceutically acceptable carrier, and a package insert comprising instructions for administration to a subject having a disease, disorder or condition associated with hepcidin deficiency or suppression.

In some embodiments, the disclosure provides an RNAi oligonucleotide or pharmaceutical composition for use, or adaptable for use, or a kit described herein, wherein the disease, disorder or condition associated with hepcidin deficiency or suppression is hemochromatosis (e.g. hereditary hemochromatosis), polycythaemia vera or beta-thalassemia.

### BRIEF DESCRIPTION OF DRAWINGS

FIGS. 1A and 1B provide graphs depicting the percent (%) of human TMPRSS6 mRNA remaining in the liver of mice exogenously expressing human TMPRSS6 (hydrodynamic injection model) after treatment with GalNAc-conjugated TMPRSS6 oligonucleotides. CD-1 mice were dosed subcutaneously with 1 mg/kg or 2 mg/kg of the indicated GalNAc-conjugated TMPRSS6 oligonucleotide formulated in PBS. Four days post-dose mice were hydrodynamically injected (HDI) with a DNA plasmid encoding human TMPRSS6. The level of human TMPRSS6 mRNA was determined from livers collected 18 hours later using a 3′ qPCR assay (FIG. 1A) and a 5′ qPCR assay (FIG. 1B). Hs/Mf=construct is human and monkey TMPRSS6 specific; Hs-Mf-Mm=construct is human, monkey, and murine TMPRSS6 specific.

FIG. 2 provides a graph depicting the percent (%) of murine TMPRSS6 mRNA remaining in the liver of mice of FIGS. 1A-1B.

FIGS. 3A-3D provide schematics depicting the modification patterns of GalNAc-conjugated TMPRSS6 oligonucleotides-0416 (FIG. 3A), -0651 (FIG. 3B), -0831 (FIG. 3C), and -1546 (FIG. 3D). The sense strand includes a tetraloop structure of nucleotides 27-30 of the 36-nucleotide strand. The antisense strand is complementary and includes a 2-nucleotide overhang.

FIGS. 4A-4F provide graphs depicting the percent (%) of endogenous monkey TMPRSS6 mRNA remaining in the liver of NHP (non-human primates) after treatment with GalNAc-conjugated TMPRSS6 oligonucleotides. Macaca fascicularis were dosed subcutaneously with 1 mg/kg or 4 mg/kg of the indicated GalNAc-conjugated TMPRSS6 oligonucleotide formulated in PBS at Day 0, 28, 56, 84, and 112. Liver samples were collected at Day-7 (1 week prior to administration) (FIG. 4A); Day 28 (FIG. 4B); Day 56 (FIG. 4C); Day 84 (FIG. 4D); Day 112 (FIG. 4E); and, Day 168 (FIG. 4F) and the level of monkey TMPRSS6 mRNA was determined using a qPCR assay relative to PBS treated animals.

FIGS. 5A-5F provide graphs depicting the percent (%) of monkey endogenous hepcidin mRNA remaining in the liver of NHP (non-human primate) after treatment with GalNAc-conjugated TMPRSS6 oligonucleotides. Macaca fascicularis were dosed subcutaneously with 1 mg/kg or 4 mg/kg of the indicated GalNAc-conjugated TMPRSS6 oligonucleotide formulated in PBS at Day 0, 28, 56, 84, and 112. Liver samples were collected at Day-7 (1 week prior to administration) (FIG. 5A); Day 28 (FIG. 5B); Day 56 (FIG. 5C); Day 84 (FIG. 5D); Day 112 (FIG. 5E); and, Day 168 (FIG. 5F) and the level of monkey hepcidin mRNA was determined using a qPCR assay relative to PBS treated animals.

FIGS. 6A-6F provide graphs depicting serum iron levels in monkeys after treatment with GalNAc-conjugated TMPRSS6 oligonucleotides. Macaca fascicularis were dosed subcutaneously with 1 mk/kg or 4 mg/kg of the indicated GalNAc-conjugated TMPRSS6 oligonucleotide formulated in PBS at Day 0, 28, 56, 84, and 112. Serum samples were collected at Day-7 (1 week prior to administration) (FIG. 6A); Day 28 (FIG. 6B); Day 56 (FIG. 6C); Day 84 (FIG. 6D); Day 112 (FIG. 6E); and, Day 168 (FIG. 6F) and the level of serum iron level was determined.

FIGS. 7A-7B provide graphs depicting the serum iron levels measured in FIGS. 6A-6F relative to PBS control treated animals for the doses of 1 mg/kg and 4 mg/kg, respectively.

FIGS. 8A-8F provide graphs depicting serum iron saturation levels in monkeys after treatment with GalNAc-conjugated TMPRSS6 oligonucleotides. Macaca fascicularis were dosed subcutaneously with 1 mk/kg or 4 mg/kg of the indicated GalNAc-conjugated TMPRSS6 oligonucleotide formulated in PBS at Day 0, 28, 56, 84, and 112. Serum samples were collected at Day-7 (1 week prior to administration) (FIG. 8A); Day 28 (FIG. 8B); Day 56 (FIG. 8C); Day 84 (FIG. 8D); Day 112 (FIG. 8E); and, Day 168 (FIG. 8F) and serum iron saturation was determined.

FIGS. 9A-9B provide graphs depicting the serum iron saturation measured in FIGS. 8A-8F relative to PBS control treated animals, for the doses of 1 mg/kg and 4 mg/kg, respectively.

FIGS. 10A-B provide a chemical drawing of GalNAc-conjugated TMPRSS6 oligonucleotide-0416, wherein A and B identify the bonds between FIGS. 10A and 10B.

FIGS. 11A-B provide a chemical drawing of GalNAc-conjugated TMPRSS6 oligonucleotide-0651, wherein A and B identify the bonds between FIGS. 11A and 11B.

FIGS. 12A-B provide a chemical drawing of GalNAc-conjugated TMPRSS6 oligonucleotide-0831, wherein A and B identify the bonds between FIGS. 12A and 12B.

FIGS. 13A-B provide a chemical drawing of GalNAc-conjugated TMPRSS6 oligonucleotide-1546, wherein A and B identify the bonds between FIGS. 13A and 13B.

FIGS. 14A-D provides provide chemical of GalNAc-conjugated TMPRSS6 oligonucleotides-0416 (FIG. 14A), -0651 (FIG. 14B), -0831 (FIG. 14C), and -1546 (FIG. 14D).

### DESCRIPTION

Transmembrane Serine Protease 6 (TMPRSS6) encodes the type II transmembrane serine proteinase, matriptase-2, located on the surface of cells. The matriptase-2 protein functions in iron homeostasis by regulating hepcidin.

According to some aspects, the disclosure provides oligonucleotides (e.g., RNAi oligonucleotides) that reduce TMPRSS6 expression in the liver. In some embodiments, the oligonucleotides provided herein are designed to treat diseases associated with hepcidin deficiency or suppression. In some respects, the disclosure provides methods of treating a disease associated with hepcidin deficiency or suppression by reducing TMPRSS6 expression in specific cells (e.g., cells of the liver) or in organs (e.g., liver).

### Oligonucleotide Inhibitors of TMPRSS6 Expression

### TMPRSS6 Target Sequences

In some embodiments, an oligonucleotide herein (e.g., an RNAi oligonucleotide) is targeted to a target sequence comprising a TMPRSS6 mRNA. In some embodiments, an oligonucleotide described herein is targeted to a target sequence within a TMPRSS6 mRNA sequence. In some embodiments, the oligonucleotide described herein corresponds to a target sequence within a TMPRSS6 mRNA sequence. In some embodiments, the oligonucleotide, or a portion, fragment, or strand thereof (e.g., an antisense strand or a guide strand of a double-stranded (ds) RNAi oligonucleotide) binds or anneals to a target sequence comprising TMPRSS6 mRNA, thereby inhibiting TMPRSS6 expression.

In some embodiments, the oligonucleotide is targeted to a TMPRSS6 target sequence for the purpose of inhibiting TMPRSS6 expression in vivo. In some embodiments, the amount or extent of inhibition of TMPRSS6 expression by an oligonucleotide targeted to a TMPRSS6 target sequence correlates with the potency of the oligonucleotide. In some embodiments, the amount or extent of inhibition of TMPRSS6 expression by an oligonucleotide targeted to a TMPRSS6 target sequence correlates with the amount or extent of therapeutic benefit in a subject or patient having a disease, disorder or condition associated with hepcidin deficiency or suppression treated with the oligonucleotide.

Through examination of the nucleotide sequence of mRNAs encoding TMPRSS6, including mRNAs of multiple different species (e.g., human, cynomolgus monkey, and mouse; see, e.g., Example 2) and as a result of in vitro and in vivo testing (see, e.g., Examples 2-5), it has been discovered that certain nucleotide sequences of TMPRSS6 mRNA are more amenable than others to oligonucleotide-based inhibition and are thus useful as target sequences for the oligonucleotides herein. In some embodiments, a TMPRSS6 target sequence comprises, or consists of, a sequence of any one of SEQ ID NOs: 661-852. In some embodiments, a TMPRSS6 target sequence comprises, or consists of, the sequence set forth in SEQ ID NO: 844, 841, 818, 794 or 762. In some embodiments, a TMPRSS6 target sequence comprises, or consists of, the sequence set forth in SEQ ID NO: 844. In some embodiments, a TMPRSS6 target sequence comprises, or consists of, the sequence set forth in SEQ ID NO: 841. In some embodiments, a TMPRSS6 target sequence comprises, or consists of, the sequence set forth in SEQ ID NO: 818. In some embodiments, a TMPRSS6 target sequence comprises, or consists of, the sequence set forth in SEQ ID NO: 794. In some embodiments, a TMPRSS6 target sequence comprises, or consists of, the sequence set forth in SEQ ID NO: 762.

### TMPRSS6 Targeting Sequences

In some embodiments, the oligonucleotides herein (e.g., RNAi oligonucleotides) have regions of complementarity to TMPRSS6 mRNA (e.g., within a target sequence of TMPRSS6 mRNA) for purposes of targeting the TMPRSS6 mRNA in cells and inhibiting and/or reducing TMPRSS6 expression. In some embodiments, the oligonucleotides herein comprise a TMPRSS6 targeting sequence (e.g., an antisense strand or a guide strand of an RNAi oligonucleotide) having a region of complementarity that binds or anneals to a TMPRSS6 target sequence by complementary (Watson-Crick) base pairing. The targeting sequence or region of complementarity is generally of a suitable length and base content to enable binding or annealing of the oligonucleotide (or a strand thereof) to a TMPRSS6 mRNA for purposes of inhibiting and/or reducing TMPRSS6 expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a targeting sequence or region of complementarity complementary to a sequence of any one of SEQ ID NOs: 661-852, and the targeting sequence or region of complementarity is 18 nucleotides in length. In some embodiments, an oligonucleotide comprises a targeting sequence or region of complementarity complementary to a sequence of any one of SEQ ID NOs: 661-852, and the targeting sequence or region of complementarity is 19 nucleotides in length. In some embodiments, an oligonucleotide comprises a targeting sequence or region of complementarity complementary to a sequence of any one of SEQ ID NOs: 193-384, and the targeting sequence or region of complementarity is 20 nucleotides in length. In some embodiments, an oligonucleotide comprises a targeting sequence or region of complementarity complementary to a sequence of any one of SEQ ID NOs: 193-384, and the targeting sequence or region of complementarity is 21 nucleotides in length. In some embodiments, an oligonucleotide comprises a targeting sequence or region of complementarity complementary to a sequence of any one of SEQ ID NOs: 193-384, and the targeting sequence or region of complementarity is 22 nucleotides in length. In some embodiments, an oligonucleotide comprises a targeting sequence or region of complementarity complementary to a sequence of any one of SEQ ID NOs: 193-384, and the targeting sequence or region of complementarity is 23 nucleotides in length. In some embodiments, an oligonucleotide comprises a targeting sequence or region of complementarity complementary to a sequence of any one of SEQ ID NOs: 193-384 and the targeting sequence or region of complementarity is 24 nucleotides in length.

In some embodiments, an oligonucleotide herein (e.g., an RNAi oligonucleotide) comprises a targeting sequence or a region of complementarity (e.g., an antisense strand or a guide strand of a double-stranded oligonucleotide) that is fully complementary to a TMPRSS6 target sequence. In some embodiments, the targeting sequence or region of complementarity is partially complementary to a TMPRSS6 target sequence. In some embodiments, the oligonucleotide comprises a targeting sequence or region of complementarity that is fully complementary to a TMPRSS6 target sequence. In some embodiments, the oligonucleotide comprises a targeting sequence or region of complementarity that is partially complementary to a TMPRSS6 target sequence.

In some embodiments, the oligonucleotide comprises a targeting sequence or region of complementarity that is fully complementary to a sequence of any one of SEQ ID NOs: 661-852. In some embodiments, the oligonucleotide comprises a targeting sequence or region of complementarity that is fully complementary to the sequence set forth in SEQ ID NOs: 844, 841, 818, 794, and 762. In some embodiments, the oligonucleotide comprises a targeting sequence or region of complementarity that is partially complementary to a sequence of any one of SEQ ID NOs: 661-852. In some embodiments, the oligonucleotide comprises a targeting sequence or region of complementarity that is partially complementary to the sequence set forth in SEQ ID NOs: 844, 841, 818, 794, and 762.

In some embodiments, an oligonucleotide herein (e.g., an RNAi oligonucleotide) comprises a targeting sequence or region of complementarity that is complementary to a contiguous sequence of nucleotides within a TMPRSS6 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides within a TMPRSS6 mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides within a TMPRSS6 mRNA, wherein the contiguous sequence of nucleotides is 19 nucleotides in length. In some embodiments, the oligonucleotide comprises a targeting sequence or region of complementarity that is complementary to a contiguous sequence of nucleotides within a TMPRSS6 mRNA, wherein the contiguous sequence of nucleotides is 20 nucleotides in length.

In some embodiments, an oligonucleotide herein (e.g., an RNAi oligonucleotide) comprises a targeting sequence or a region of complementary that is complementary to a contiguous sequence of nucleotides of any one of SEQ ID NOs: 661-852, optionally wherein the contiguous sequence of nucleotides is 19 nucleotides in length. In some embodiments, the oligonucleotide comprises a targeting sequence or a region of complementary that is complementary to a contiguous sequence of nucleotides of any one of SEQ ID NOs: 844, 841, 818, 794, and 762, wherein the contiguous sequence of nucleotides is 19 nucleotides in length. In some embodiments, the oligonucleotide comprises a targeting sequence or a region of complementary that is complementary to a contiguous sequence of nucleotides of any one of SEQ ID NOs: 376, 373, 350, 326, and 294, wherein the contiguous sequence of nucleotides is 20 nucleotides in length.

In some embodiments, an oligonucleotide herein (e.g., an RNAi oligonucleotide) comprises a targeting sequence or region of complementarity having one or more base pair (bp) mismatches with the corresponding TMPRSS6 target sequence. In some embodiments, the targeting sequence or region of complementarity may have up to about 1, up to about 2, up to about 3, up to about 4, up to about 5, etc. mismatches with the corresponding TMPRSS6 target sequence provided that the ability of the targeting sequence or region of complementarity to bind or anneal to the TMPRSS6 mRNA under appropriate hybridization conditions and/or the ability of the oligonucleotide to inhibit TMPRSS6 expression is maintained. Alternatively, the targeting sequence or region of complementarity may have no more than 1, no more than 2, no more than 3, no more than 4, or no more than 5 mismatches with the corresponding TMPRSS6 target sequence provided that the ability of the targeting sequence or region of complementarity to bind or anneal to the TMPRSS6 mRNA under appropriate hybridization conditions and/or the ability of the oligonucleotide to inhibit TMPRSS6 expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed throughout the targeting sequence or region of complementarity. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof. In some embodiments, the oligonucleotide comprises a targeting sequence or a region of complementary that is complementary to a contiguous sequence of nucleotides of any one of SEQ ID NOs: 661-852, wherein the targeting sequence or region of complementarity may have up to about 1, up to about 2, up to about 3, up to about 4, up to about 5, etc. mismatches with the corresponding TMPRSS6 target sequence. In some embodiments, the oligonucleotide comprises a targeting sequence or a region of complementary that is complementary to a contiguous sequence of nucleotides of any one of SEQ ID NOs: 661-852, wherein the targeting sequence or region of complementarity may have no more than 1, no more than 2, no more than 3, no more than 4, or no more than 5 mismatches with the corresponding TMPRSS6 target sequence. In some embodiments, the oligonucleotide comprises a targeting sequence or a region of complementary that is complementary to a contiguous sequence of nucleotides of any one of SEQ ID NOs: 661-852, wherein the targeting sequence or region of complementarity may have up to about 1, up to about 2, up to about 3, up to about 4, up to about 5, etc. mismatches with the corresponding TMPRSS6 target sequence. In some embodiments, the oligonucleotide comprises a targeting sequence or a region of complementary that is complementary to a contiguous sequence of nucleotides of any one of SEQ ID NOs: 844, 841, 818, 794, and 762, wherein the targeting sequence or region of complementarity may have no more than 1, no more than 2, no more than 3, no more than 4, or no more than 5 mismatches with the corresponding TMPRSS6 target sequence.

### Types of Oligonucleotides

A variety of oligonucleotide types and/or structures are useful for targeting TMPRSS6 in the methods herein including, but not limited to, RNAi oligonucleotides, antisense oligonucleotides (ASOs), miRNAs, etc. Any of the oligonucleotide types described herein or elsewhere are contemplated for use as a framework to incorporate a TMPRSS6 targeting sequence herein for the purposes of inhibiting TMPRSS6 expression.

In some embodiments, the oligonucleotides herein inhibit TMPRSS6 expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded (ss) extensions (on one or both sides of the molecule) as well as double-stranded (ds) extensions.

In some embodiments, the oligonucleotides herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotides in length capable of reducing TMPRSS6 expression are produced. In some embodiments, the oligonucleotide has an overhang (e.g., of 1, 2, or 3 nucleotides in length) in the 3′ end of the antisense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 36 (e.g., 17 to 36, 20 to 25 or 21-23) nucleotides in length. In some embodiments, the oligonucleotides described herein comprise an antisense strand of 19-30 nucleotides in length and a sense strand of 19-50 nucleotides in length, wherein the antisense and sense strands are separate strands which form an asymmetric duplex region having an overhang of 1-4 nucleotides at the 3′ terminus of the antisense strand. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., Nucleic Acids in Chemistry and Biology, Blackburn (ed.), ROYAL SOCIETY OF CHEMISTRY, 2006), shRNAs (e.g., having 19 bp or shorter stems; see, e.g., Moore et al. (2010) Methods Mol. Biol. 629:141-158), blunt siRNAs (e.g., of 19 bps in length; see, e.g., Kraynack & Baker (2006) RNA 12:163-176), asymmetrical siRNAs (aiRNA; see, e.g., Sun et al. (2008) Nat. Biotechnol. 26:1379-82), asymmetric shorter-duplex siRNA (see, e.g., Chang et al. (2009) Mol. Ther. 17:725-32), fork siRNAs (see, e.g., Hohjoh (2004) FEBS Lett. 557:193-98), ss siRNAs (Elsner (2012) Nat. Biotechnol. 30:1063), dumbbell-shaped circular siRNAs (see, e.g., Abe et al. (2007) J. Am. Chem. Soc. 129:15108-09), and small internally segmented interfering RNA (siRNA; see, e.g., Bramsen et al. (2007) Nucleic Acids Res. 35:5886-97). Further non-limiting examples of an oligonucleotide structures that may be used in some embodiments to reduce or inhibit the expression of TMPRSS6 are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting TMPRSS6 expression herein is single-stranded (ss). Such structures may include but are not limited to single-stranded RNAi molecules. Recent efforts have demonstrated the activity of ss RNAi molecules (see, e.g., Matsui et al. (2016) Mol. Ther. 24:946-55). However, in some embodiments, oligonucleotides herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) to induce RNaseH-mediated cleavage of its target RNA in cells or (e.g., as a mixmer) to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) Annu. Rev. Pharmacol. 57:81-105).

In some embodiments, the antisense oligonucleotide shares a region of complementarity with TMPRSS6 mRNA. In some embodiments, the antisense oligonucleotide targets various areas of the human TMPRSS6 gene identified as NM_001289000.2. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is complementary to any one of SEQ ID NOs: 661-852. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### Double-Stranded Oligonucleotides

In some aspects, the disclosure provides double-stranded (ds) RNAi oligonucleotides for targeting TMPRSS6 mRNA and inhibiting TMPRSS6 expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, a first region (R1) of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, the sense strand has a second region (R2), wherein R2 comprises a first subregion (S1), a loop (Lp), such as a tetraloop (tetraLp) or triloop (triLp), and a second subregion (S2), wherein Lp is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, an oligonucleotide provided herein comprises a sense strand comprising a sequence of any one of SEQ ID NOs: 193-384 and an antisense strand comprising a sequence of any one of SEQ ID NOs: 385-576. In some embodiments, an oligonucleotide provided herein comprises a sense strand comprising a sequence of any one of SEQ ID NOs: 661-852 and an antisense strand comprising a sequence of any one of SEQ ID NOs: 1-192.

In some embodiments, an oligonucleotide provided herein (e.g., an RNAi oligonucleotide) comprises a sense strand comprising a sequence of any one of SEQ ID NOs: 577-597 and an antisense strand comprising a sequence of any one of SEQ ID NOs: 598-618 as is arranged in Table 4.

In some embodiments, an oligonucleotide provided herein (e.g., an RNAi oligonucleotide) comprises a sense strand and an antisense strand comprising nucleotide sequences selected from:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively.

In some embodiments, an oligonucleotide provided herein (e.g., an RNAi oligonucleotide) comprises a sense strand and an antisense strand comprising nucleotide sequences selected from:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively.

In some embodiments, the sense strand comprises the sequence of SEQ ID NO: 579 and the antisense strand comprises the sequence of SEQ ID NO: 600. In some embodiments, the sense strand comprises the sequence of SEQ ID NO: 580 and the antisense strand comprises the sequence of SEQ ID NO: 601. In some embodiments, the sense strand comprises the sequence of SEQ ID NO: 590 and the antisense strand comprises the sequence of SEQ ID NO: 611. In some embodiments, the sense strand comprises the sequence of SEQ ID NO: 597 and the antisense strand comprises the sequence of SEQ ID NO: 618. In some embodiments, the sense strand comprises the sequence of SEQ ID NO: 586 and the antisense strand comprises the sequence of SEQ ID NO: 607.

It should be appreciated that, in some embodiments, sequences presented in the Sequence Listing may be referred to in describing the structure of an oligonucleotide (e.g., an RNAi oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, an oligonucleotide herein (e.g., an RNAi oligonucleotide) comprises a sense strand and an antisense strand that, when acted upon by a Dicer enzyme, results in an antisense strand that is incorporated into the mature RISC. In some embodiments, an oligonucleotide herein (e.g., an RNAi oligonucleotide) comprises a 25-nucleotide sense strand and a 27-nucleotide antisense strand that when acted upon by a Dicer enzyme results in an antisense strand that is incorporated into the mature RISC. In some embodiments, the 25-nucleotide sense strand comprises a sequence selected from SEQ ID NOs: 193-384. In some embodiments, the 27-nucleotide antisense strand comprises a sequence selected from SEQ ID NOs: 385-576. In some embodiments, the sense strand of the oligonucleotide is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide is longer than 25 nucleotides (e.g., 26, 27, 28, 29 or 30 nucleotides). In some embodiments, the sense strand of the oligonucleotide comprises a nucleotide sequence selected from SEQ ID NOs: 577-597, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide comprises a nucleotide sequence selected from SEQ ID NOs: 577-597, wherein the nucleotide sequence is longer than 25 nucleotides (e.g., 26, 27, 28, 29 or 30 nucleotides).

In some embodiments, oligonucleotides herein (e.g., RNAi oligonucleotides) have one 5′ end that is thermodynamically less stable when compared to the other 5′ end. In some embodiments, an asymmetric oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). In some embodiments, the oligonucleotide has an overhang comprising two (2) nucleotides on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. In some embodiments, the oligonucleotide comprises a targeting sequence or a region of complementary that is complementary to a contiguous sequence of nucleotides of any one of SEQ ID NOs: 661-852, and a 5′-overhang comprising a length of between 1 and 6 nucleotides. In some embodiments, the oligonucleotide comprises a sense strand comprising a nucleotide sequence selected from SEQ ID NOs: 577-597, wherein the oligonucleotide comprises a 5′-overhang comprising a length of between 1 and 6 nucleotides. In some embodiments, the oligonucleotide comprises an antisense strand comprising a nucleotide sequence selected from SEQ ID NOs: 598-618, wherein the oligonucleotide comprises a 5′-overhang comprising a length of between 1 and 6 nucleotides. In some embodiments, the oligonucleotide comprises a sense strand comprising a nucleotide sequence selected from SEQ ID NOs: 577-597 and antisense strand comprising a nucleotide sequence selected from SEQ ID NOs: 598-618, wherein the oligonucleotide comprises a 5′-overhang comprising a length of between 1 and 6 nucleotides.

In some embodiments, two (2) terminal nucleotides on the 3′ end of an antisense strand are modified. In some embodiments, the two (2) terminal nucleotides on the 3′ end of the antisense strand are complementary with the target mRNA (e.g., TMPRSS6 mRNA). In some embodiments, the two (2) terminal nucleotides on the 3′ end of the antisense strand are not complementary with the target mRNA. In some embodiments, the two (2) terminal nucleotides on the 3′ end of the antisense strand of an oligonucleotide herein are unpaired. In some embodiments, the two (2) terminal nucleotides on the 3′ end of the antisense strand of an oligonucleotide herein comprise an unpaired guanine (e.g., 5′-GG-3′). In some embodiments, the two (2) terminal nucleotides on the 3′ end of an antisense strand of an oligonucleotide herein are not complementary to the target mRNA. In some embodiments, two (2) terminal nucleotides on each 3′ end of an oligonucleotide are guanines (GG). In some embodiments, one or both of the two (2) terminal GG nucleotides on each 3′ end of an oligonucleotide herein is not complementary with the target mRNA. In some embodiments, the oligonucleotide comprises a targeting sequence or a region of complementary that is complementary to a contiguous sequence of nucleotides of any one of SEQ ID NOs: 661-852, wherein the two (2) terminal nucleotides on the 3′ end of the antisense strand of the oligonucleotide herein comprises an unpaired GG. In some embodiments, the oligonucleotide comprises an antisense strand comprising a nucleotide sequence selected from SEQ ID NOS: 1-192, wherein the two (2) terminal nucleotides on the 3′ end of the antisense strand of the oligonucleotide comprises an unpaired GG. In some embodiments, the oligonucleotide comprises a sense strand comprising a nucleotide sequence selected from SEQ ID NOs: 577-597 and antisense strand comprising a nucleotide sequence selected from SEQ ID NOs: 598-618, wherein the two (2) terminal nucleotides on the 3′ end of the antisense strand of the oligonucleotide comprises an unpaired GG.

In some embodiments, there is one or more (e.g., 1, 2, 3, 4 or 5) mismatch(s) between a sense and antisense strand comprising an oligonucleotide herein (e.g., an RNAi oligonucleotide). If there is more than one mismatch between a sense and antisense strand, they may be positioned consecutively (e.g., 2, 3 or more in a row), or interspersed throughout the region of complementarity. In some embodiments, the 3′ end of the sense strand comprises one or more mismatches. In some embodiments, two (2) mismatches are incorporated at the 3′ end of the sense strand. In some embodiments, base mismatches, or destabilization of segments at the 3′ end of the sense strand of an oligonucleotide herein improves or increases the potency of the oligonucleotide. In some embodiments, the sense and antisense strands of an oligonucleotide herein comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively, wherein there is one or more (e.g., 1, 2, 3, 4 or 5) mismatch(s) between the sense and antisense strands.

In some embodiments, an oligonucleotide provided herein (e.g., an RNAi oligonucleotide) comprises a sense strand and an antisense strand comprising nucleotide sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively, wherein there is one or more (e.g., 1, 2, 3, 4 or 5) mismatch(s) between the sense and antisense strands.

### Antisense Strands

In some embodiments, an antisense strand of an oligonucleotide herein (e.g., an RNAi oligonucleotide) is referred to as a “guide strand”. For example, an antisense strand that engages with RNA-induced silencing complex (RISC) and binds to an Argonaute protein such as Ago2, or engages with or binds to one or more similar factors, and directs silencing of a target gene, as the antisense strand is referred to as a guide strand. In some embodiments, a sense strand comprising a region of complementary to a guide strand is referred to herein as a “passenger strand.”

In some embodiments, an oligonucleotide herein (e.g., an RNAi oligonucleotide) comprises an antisense strand of up to about 50 nucleotides in length (e.g., up to 50, up to 40, up to 35, up to 30, up to 27, up to 25, up to 21, up to 19, up to 17 or up to 12 nucleotides in length). In some embodiments, an oligonucleotide comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide comprises an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide comprises antisense strand of 15 to 30 nucleotides in length. In some embodiments, an antisense strand of any one of the oligonucleotides disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises an antisense strand of 22 nucleotides in length.

In some embodiments, an oligonucleotide disclosed herein (e.g., an RNAi oligonucleotide) for targeting TMPRSS6 comprises an antisense strand comprising or consisting of a sequence as set forth in any one of SEQ ID NOs: 385-576. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 385-576. In some embodiments, an oligonucleotide disclosed herein for targeting TMPRSS6 comprises an antisense strand comprising or consisting of a sequence as set forth in any one of SEQ ID NOs: 598-618. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 598-618. In some embodiments, an oligonucleotide disclosed herein for targeting TMPRSS6 comprises an antisense strand comprising or consisting of a sequence as set forth in any one of SEQ ID NOs: 598-618. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 600, 601, 611, 618, and 607. In some embodiments, an oligonucleotide disclosed herein for targeting TMPRSS6 comprises an antisense strand comprising or consisting of a sequence as set forth in any one of SEQ ID NOs: 600, 601, 611, 618, and 607.

In some embodiments, an oligonucleotide herein comprises an antisense strand comprising a nucleotide sequence selected from SEQ ID NOs: 1-192. In some embodiments, an oligonucleotide herein comprises an antisense strand comprising a nucleotide sequence selected from SEQ ID NOs: 184, 181, 158, 134, and 102.

### Sense Strands

In some embodiments, an oligonucleotide disclosed herein (e.g., an RNAi oligonucleotide) for targeting TMPRSS6 mRNA and inhibiting TMPRSS6 expression comprises a sense strand sequence as set forth in any one of SEQ ID NOs: 661-852. In some embodiments, an oligonucleotide disclosed herein (e.g., an RNAi oligonucleotide) for targeting TMPRSS6 mRNA and inhibiting TMPRSS6 expression comprises a sense strand sequence as set forth in any one of SEQ ID NOs: 193-384. In some embodiments, an oligonucleotide herein has a sense strand comprised of at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 193-384. In some embodiments, an oligonucleotide herein has a sense strand comprised of at least about 12 (e.g., at least 13, at least 14, at least 15, at least 16, at least 17, at least 18, or at least 19) contiguous nucleotides of a sequence as set forth in in any one of SEQ ID NOs: 661-852. In some embodiments, an oligonucleotide disclosed herein for targeting TMPRSS6 mRNA and inhibiting TMPRSS6 expression comprises a sense strand sequence as set forth in any one of SEQ ID NOs: 577-597. In some embodiments, an oligonucleotide herein has a sense strand comprised of least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 577-597. In some embodiments, an oligonucleotide disclosed herein for targeting TMPRSS6 mRNA and inhibiting TMPRSS6 expression comprises a sense strand sequence as set forth in any one of SEQ ID NOs: 579, 580, 590, 597, and 586. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 579, 580, 590, 597, and 586. In some embodiments, an oligonucleotide disclosed herein for targeting TMPRSS6 mRNA and inhibiting TMPRSS6 expression comprises a sense strand sequence as set forth in any one of SEQ ID NOs: 844, 841, 818, 794, and 762. In some embodiments, an oligonucleotide herein has a sense strand that comprise at least about 12 (e.g., at least 13, at least 14, at least 15, at least 16, at least 17, at least 18, or at least 19) contiguous nucleotides of a sequence as set forth in any one of SEQ ID NOs: 844, 841, 818, 794, and 762.

In some embodiments, an oligonucleotide provided herein (e.g., an RNAi oligonucleotide) comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, an oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide herein comprises a sense strand of 15 to 50 nucleotides in length. In some embodiments, an oligonucleotide herein comprises a sense strand of 18 to 36 nucleotides in length. In some embodiments, an oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an oligonucleotide herein comprises a sense strand of 36 nucleotides in length.

In some embodiments, an oligonucleotide provided herein (e.g., an RNAi oligonucleotide) comprises a sense strand comprising a stem-loop structure proximal the 3′ end of the sense strand. In some embodiments, an oligonucleotide provided herein (e.g., an RNAi oligonucleotide) comprises a sense strand comprising a stem-loop structure at the 3′end of the first region (R1) of the sense strand. In some embodiments, the stem-loop is formed by intrastrand base pairing. In some embodiments, a sense strand comprises a stem-loop structure proximal its 5′ end. In some embodiments, a sense strand comprises a stem-loop structure at the 5′ end of the first region (R1). In some embodiments, the stem of the stem-loop comprises a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the oligonucleotide protection against degradation (e.g., enzymatic degradation), facilitates or improves targeting and/or delivery to a target cell, tissue, or organ (e.g., the liver), or both. For example, in some embodiments, the loop of a stem-loop is comprised of nucleotides comprising one or more modifications that facilitate, improve, or increase targeting to a target mRNA (e.g., a TMPRSS6 mRNA), inhibition of target gene expression (e.g., TMPRSS6 expression), and/or delivery, uptake, and/or penetrance into a target cell, tissue, or organ (e.g., the liver), or a combination thereof. In some embodiments, the stem-loop itself or modification(s) to the stem-loop do not affect or do not substantially affect the inherent gene expression inhibition activity of the oligonucleotide, but facilitates, improves, or increases stability (e.g., provides protection against degradation) and/or delivery, uptake, and/or penetrance of the oligonucleotide to a target cell, tissue, or organ (e.g., the liver). In certain embodiments, an oligonucleotide herein comprises a sense strand comprising (e.g., at its 3′ end, proximal its 3′ end and/or at the 3′ end of the first region (R1) of the sense strand) a stem-loop set forth as: S1-Lp-S2, in which S1 is complementary to S2, and in which Lp forms a single-stranded loop of linked nucleotides between S1 and S2 of up to about 10 nucleotides in length (e.g., 3, 4, 5, 6, 7, 8, 9 or 10 nucleotides in length). In some embodiments, the loop (Lp) is 3 nucleotides in length (referred to herein as “triloop”). In some embodiments, the loop (Lp) is 4 nucleotides in length (referred to herein as “tetraloop”). In some embodiments, the loop (Lp) is 5 nucleotides in length. In some embodiments, the loop (Lp) is 6 nucleotides in length. In some embodiments, the loop (Lp) is 7 nucleotides in length. In some embodiments, the loop (Lp) is 8 nucleotides in length. In some embodiments, the loop (Lp) is 9 nucleotides in length. In some embodiments, the loop (Lp) is 10 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 856).

In some embodiments, an oligonucleotide provided herein (e.g., an RNAi oligonucleotide) comprises a targeting sequence or a region of complementary that is complementary to a contiguous sequence of nucleotides of any one of SEQ ID NOs: 661-852, and the oligonucleotide comprises a sense strand comprising (e.g., at its 3′ end, proximal its 3′ end and/or at the 3′ end of the first region (R1) of the sense strand) a stem-loop set forth as: S1-Lp-S2, in which S1 is complementary to S2, and in which Lp forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of any one of SEQ ID NOs: 661-852, and the oligonucleotide comprises a sense strand comprising (e.g., at its 3′ end, proximal its 3′ end and/or at the 3′ end of the first region (R1) of the sense strand) a stem-loop set forth as: S1-Lp-S2, in which S1 is complementary to S2, and in which Lp forms a single-stranded loop between S1 and S2 of 4 nucleotides in length.

In some embodiments, a loop (Lp) of a stem-loop having the structure S1-Lp-S2 as described herein is a triloop (triLp). In some embodiments, the oligonucleotide comprises a targeting sequence or a region of complementary that is complementary to a contiguous sequence of nucleotides of any one of SEQ ID NOs: 661-852 and a triloop. In some embodiments, the triloop comprises ribonucleotides, deoxyribonucleotides, modified nucleotides, ligands (e.g., delivery ligands), and combinations thereof.

In some embodiments, a loop (Lp) of a stem-loop having the structure S1-Lp-S2 as described above is a tetraloop (tetraLp) as describe in U.S. Pat. No. 10,131,912, incorporated herein by reference. In some embodiments, an oligonucleotide herein comprises a targeting sequence or a region of complementary that is complementary to a contiguous sequence of nucleotides of any one of SEQ ID NOs: 661-852 and a tetraloop. In some embodiments, the tetraloop comprises ribonucleotides, deoxyribonucleotides, modified nucleotides, ligands (e.g., delivery ligands), and combinations thereof.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 12 nucleotides in length. In some embodiments, a duplex formed between a sense and antisense strand is 13 nucleotides in length. In some embodiments, a duplex formed between a sense and antisense strand is 14 nucleotides in length. In some embodiments, a duplex formed between a sense and antisense strand is 15 nucleotides in length. In some embodiments, a duplex formed between a sense and antisense strand is 16 nucleotides in length. In some embodiments, a duplex formed between a sense and antisense strand is 17 nucleotides in length. In some embodiments, a duplex formed between a sense and antisense strand is 18 nucleotides in length. In some embodiments, a duplex formed between a sense and antisense strand is 19 nucleotides in length. In some embodiments, a duplex formed between a sense and antisense strand is 20 nucleotides in length. In some embodiments, a duplex formed between a sense and antisense strand is 21 nucleotides in length. In some embodiments, a duplex formed between a sense and antisense strand is 22 nucleotides in length. In some embodiments, a duplex formed between a sense and antisense strand is 23 nucleotides in length. In some embodiments, a duplex formed between a sense and antisense strand is 24 nucleotides in length. In some embodiments, a duplex formed between a sense and antisense strand is 25 nucleotides in length. In some embodiments, a duplex formed between a sense and antisense strand is 26 nucleotides in length. In some embodiments, a duplex formed between a sense and antisense strand is 27 nucleotides in length. In some embodiments, a duplex formed between a sense and antisense strand is 28 nucleotides in length. In some embodiments, a duplex formed between a sense and antisense strand is 29 nucleotides in length. In some embodiments, a duplex formed between a sense and antisense strand is 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length)

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length)

### Oligonucleotide Termini

In some embodiments, an oligonucleotide disclosed herein (e.g., an RNAi oligonucleotide) comprises a sense strand and an antisense strand, wherein the termini of either or both strands comprise a blunt end. In some embodiments, an oligonucleotide herein comprises sense and antisense strands that are separate strands which form an asymmetric duplex region having an overhang at the 3′ terminus of the antisense strand. In some embodiments, an oligonucleotide herein comprises a sense strand and an antisense strand, wherein the termini of either or both strands comprise an overhang comprising one or more nucleotides. In some embodiments, the one or more nucleotides comprising the overhang are unpaired nucleotides. In some embodiments, an oligonucleotide herein comprises a sense strand and an antisense strand, wherein the 3′ termini of the sense strand and the 5′ termini of the antisense strand comprise a blunt end. In some embodiments, an oligonucleotide herein comprises a sense strand and an antisense strand, wherein the 5′ termini of the sense strand and the 3′ termini of the antisense strand comprise a blunt end.

In some embodiments, an oligonucleotide herein comprises a sense strand and an antisense strand, wherein the 3′ terminus of either or both strands comprise a 3′-overhang comprising one or more nucleotides. In some embodiments, an oligonucleotide herein comprises a sense strand and an antisense strand, wherein the sense strand comprises a 3′-overhang comprising one or more nucleotides. In some embodiments, an oligonucleotide herein comprises a sense strand and an antisense strand, wherein the antisense strand comprises a 3′-overhang comprising one or more nucleotides. In some embodiments, an oligonucleotide herein comprises a sense strand and an antisense strand, wherein both the sense strand and the antisense strand comprises a 3′-overhang comprising one or more nucleotides.

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

In some embodiments, an oligonucleotide disclosed herein (e.g., an RNAi oligonucleotide) comprises a sense strand and an antisense strand, wherein the sense and antisense strands of the oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide disclosed herein (e.g., an RNAi oligonucleotide) comprises a sense strand and an antisense strand, wherein the sense and antisense strands of the oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, an oligonucleotide disclosed herein (e.g., an RNAi oligonucleotide) comprises a sense strand and an antisense strand, wherein the sense and antisense strands of the oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
and wherein the antisense strand comprises a 5′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 5′-overhang is two (2) nucleotides in length.

In some embodiments, an oligonucleotide disclosed herein (e.g., an RNAi oligonucleotide) comprises a sense strand and an antisense strand, wherein the sense and antisense strands of the oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
and wherein the antisense strand comprises a 5′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 5′-overhang is two (2) nucleotides in length.

In some embodiments, one or more (e.g., 2, 3, 4, 5, or more) nucleotides comprising the 3′ terminus or 5′ terminus of a sense and/or antisense strand are modified. For example, in some embodiments, one or two terminal nucleotides of the 3′ terminus of the antisense strand are modified. In some embodiments, the last nucleotide at the 3′ terminus of an antisense strand is modified, such that it comprises 2′ modification, or it comprises, a 2′-O-methoxyethyl. In some embodiments, the last one or two terminal nucleotides at the 3′ terminus of an antisense strand are complementary with the target. In some embodiments, the last one or two nucleotides at the 3′ terminus of the antisense strand are not complementary with the target.

In some embodiments, an oligonucleotide disclosed herein (e.g., an RNAi oligonucleotide) comprises a sense strand and an antisense strand, wherein the 3′ terminus of the sense strand comprises a step-loop described herein and the 3′ terminus of the antisense strand comprises a 3′-overhang described herein. In some embodiments, an oligonucleotide herein (e.g., an RNAi oligonucleotide) comprises a sense strand and an antisense strand that form a nicked tetraloop structure described herein, wherein the 3′ terminus of the sense strand comprises a stem-loop, wherein the loop is a tetraloop described herein, and wherein the 3′ terminus of the antisense strand comprises a 3′-overhang described herein. In some embodiments, the 3′-overhang is two (2) nucleotides in length. In some embodiments, the two (2) nucleotides comprising the 3′-overhang both comprise guanine (G) nucleobases. One or both of the nucleotides comprising the 3′-overhang of the antisense strand may not be complementary with the target mRNA. Typically, one or both of the nucleotides comprising the 3′-overhang of the antisense strand are not complementary with the target mRNA.

### Oligonucleotide Modifications

In some embodiments, an oligonucleotide described herein (e.g., an RNAi oligonucleotide) comprises a modification. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic or research use.

In some embodiments, the modification is a modified sugar. In some embodiments, the modification is a 5′-terminal phosphate group. In some embodiments, the modification is a modified internucleotide linkage. In some embodiments, the modification is a modified base.

In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises at least one modified sugar, a 5′-terminal phosphate group, at least one modified internucleotide linkage, and at least one modified base.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises at least one modified sugar, a 5′-terminal phosphate group, at least one modified internucleotide linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all the nucleotides of an oligonucleotide are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, an oligonucleotide described herein (e.g., an RNAi oligonucleotide) comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) Tetrahedon 54:3607-30), unlocked nucleic acids (“UNA”; see, e.g., Snead et al. (2013) Mol. Ther-Nucl. Acids 2:e103) and bridged nucleic acids (“BNA”; see, e.g., Imanishi & Obika (2002) Chem Commun. (Camb) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar linked to a 1′-carbon or 4′-carbon of the sugar, or a 2′-oxygen linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, an oligonucleotide (e.g., an RNAi oligonucleotide) described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides oligonucleotides having different modification patterns. In some embodiments, an oligonucleotide herein comprises a sense strand having a modification pattern as set forth in the Examples and Sequence Listing and an antisense strand having a modification pattern as set forth in the Examples and Sequence Listing.

In some embodiments, an oligonucleotide disclosed herein (e.g., an RNAi oligonucleotide) comprises an antisense strand having nucleotides that are modified with 2′-F. In some embodiments, an oligonucleotide herein comprises an antisense strand comprising nucleotides that are modified with 2′-F and 2′-OMe. In some embodiments, an oligonucleotide disclosed herein comprises a sense strand having nucleotides that are modified with 2′-F. In some embodiments, an oligonucleotide disclosed herein comprises a sense strand comprises nucleotides that are modified with 2′-F and 2′-OMe.

In some embodiments, an oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2′-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification.

As used herein, oligonucleotide numbering of the sense and antisense strand, respectively, starts at the 5′ end with “position 1”.

In some embodiments, one or more of positions 8, 9, 10 or 11 of the sense strand is modified with a 2′-F group. In some embodiments, one or more of positions 3, 8, 9, 10, 12, 13 and 17 of the sense strand is modified with a 2′-F group. In some embodiments, one or more of positions 2, 3, 4, 5, 7, 10 and 14 of the antisense strand is modified with a 2′-F group. In some embodiments, one or more of positions 2, 3, 4, 5, 7, 8, 10, 14, 16 and 19 is modified with a 2′-F group. In some embodiments, the sugar moiety at each of nucleotides at positions 1-7 and 12-20 in the sense strand is modified with a 2′-OMe. In some embodiments, the sugar moiety at each of nucleotides at positions 1-7, 12-27 and 31-36 in the sense strand is modified with a 2′-OMe. In some embodiments, the sugar moiety at each of nucleotides at positions 6, 9, 11-13, 15, 17, 18 and 20-22 in the sense strand is modified with a 2′-OMe.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein one or more of positions 8, 9, 10 or 11 of the sense strand is modified with a 2′-F group.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein one or more of positions 8, 9, 10 or 11 of the sense strand is modified with a 2′-F group.

In some embodiments, an oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 4, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-3-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 8, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-fluoro (2′-F), 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-3-d-arabinonucleic acid (2′-FANA).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 3, 8, 9, 10, 12, 13 and 17 modified with 2′-F. In some embodiments, an oligonucleotide provided herein comprises a sense strand having the sugar moiety at positions 1-7 and 12-17 or 12-20 modified with 2′OMe. In some embodiments, an oligonucleotide provided herein comprises a sense strand having the sugar moiety at positions 1-7, 12-27 and 31-36 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, an oligonucleotide provided herein comprises a sense strand having the sugar moiety at positions 1-2, 4-7, 11, 14-16 and 18-20 modified with 2′OMe. In some embodiments, an oligonucleotide provided herein comprises a sense strand having the sugar moiety of each of the nucleotides at positions 1-2, 4-7, 11, 14-16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-fluoro (2′-F), 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, an oligonucleotide described herein (e.g., an RNAi oligonucleotide) comprises a sense strand and an antisense strand, wherein the antisense strand comprises a 5′-terminal phosphate. In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their performance and/or bioavailability in vivo. In some embodiments, an oligonucleotide herein includes analogs of 5′ phosphates that are resistant to such degradation. In some embodiments, the phosphate analog is oxymethyl phosphonate, vinyl phosphonate or malonyl phosphonate, or a combination thereof. In certain embodiments, the 5′ terminus of an oligonucleotide strand is attached to chemical moiety that mimics the electrostatic and steric properties of a natural 5′-phosphate group (“phosphate mimic”). In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises a 5′-terminal phosphate, optionally a 5′-terminal phosphate analog.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises a 5′-terminal phosphate, optionally a 5′-terminal phosphate analog.

In some embodiments, an oligonucleotide herein (e.g., an RNAi oligonucleotide) has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH₂—PO(OH)(R), in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃. In some embodiment, R is CH₃. In some embodiments, the 4′-phosphate analog is 4′-oxymethyl phosphonate.

In some embodiments, an oligonucleotide provided herein comprises an antisense strand comprising a 4′-phosphate analog at the 5′-terminal nucleotide, wherein 5′-terminal nucleotide comprises the following structure:

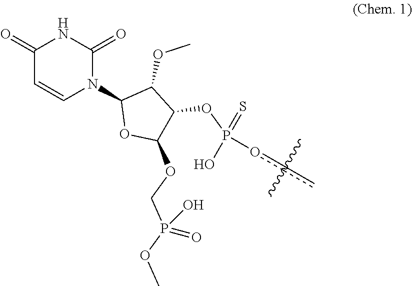

4′-O-monomethylphosphonate-2′-O-methyluridine phosphorothioate [MePhosphonate-40-mUs].

In some embodiments, an oligonucleotide provided herein comprises an antisense strand comprising a 4′-phosphate analog at the 5′-terminal nucleotide, wherein 5′-terminal nucleotide comprises the following structure:

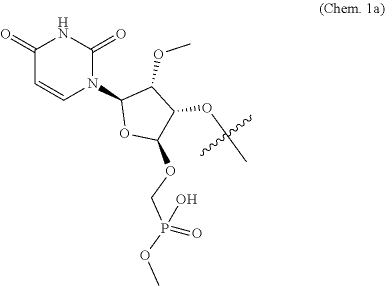 [SMILES: *O[C@@H]1[C@@H](OCP(=O)(O)OC)O[C@@H](n2ccc(=O)[nH]c2=O)[C@@H]1OC]

4′-O-monomethylphosphonate-2′-O-methyluridine [MePhosphonate-40-mU].

Chem. 1a may be referred to as 4′-O-monomethylphosphonate-2′-O-methyluridine phosphorothioate [MePhosphonate-40-mUs] when a phosphorothioate internucleotide linkage is provided.

### Modified Internucleotide Linkage

In some embodiments, an oligonucleotide provided herein (e.g., a RNAi oligonucleotide) comprises a modified internucleotide linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an oligonucleotide provided herein (e.g., a RNAi oligonucleotide) has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises a modified internucleotide linkage.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises a modified internucleotide linkage.

### Base Modifications

In some embodiments, an oligonucleotide provided herein (e.g., an RNAi oligonucleotides) comprises one or more modified nucleobases. In some embodiments, modified nucleobases (also referred to herein as base analogs) are linked at the 1′ position of a nucleotide sugar moiety. In certain embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic). In some embodiments, the sense and antisense strands of an oligonucleotide comprise nucleotides sequences selected from the group consisting of: a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises one or more modified nucleobases.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid (e.g., a TMPRSS6 mRNA), a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) Nucleic Acids Res. 23:4363-4370; Loakes et al. (1995) Nucleic Acids Res. 23:2361-66; and Loakes & Brown (1994) Nucleic Acids Res. 22:4039-43).

### Targeting Ligands

In some embodiments, it is desirable to target an oligonucleotide provided herein (e.g., an RNAi oligonucleotide) to one or more cells or cell type, tissues, organs, or anatomical regions or compartments. Such a strategy may help to avoid undesirable effects to the organism treated and/or to avoid undue loss of the oligonucleotide to cells, tissues, organs, or anatomical regions or compartments that would not benefit from the oligonucleotide or its effects (e.g., inhibition or reduction of TMPRSS6 expression). Accordingly, in some embodiments, oligonucleotides disclosed herein (e.g., RNAi oligonucleotides) are modified to facilitate targeting and/or delivery to particular cells or cell types, tissues, organs, or anatomical regions or compartments (e.g., to facilitate delivery of the oligonucleotide to the liver). In some embodiments, an oligonucleotide comprises at least one nucleotide (e.g., 1, 2, 3, 4, 5, 6 or more nucleotides) conjugated to one or more targeting ligand(s). In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises a targeting ligand conjugated to at least one nucleotide.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises a targeting ligand conjugated to at least one nucleotide.

In some embodiments, the targeting ligand comprises a carbohydrate, amino sugar, cholesterol, peptide, polypeptide, protein, or part of a protein (e.g., an antibody or antibody fragment), or lipid. In certain embodiments, the targeting ligand is a carbohydrate comprising at least one GalNAc moiety.

In some embodiments, 1 or more (e.g., 1, 2, 3, 4, 5 or 6) nucleotides of an oligonucleotide provided herein (e.g., an RNAi oligonucleotide) are each conjugated to a separate targeting ligand (e.g., a GalNAc moiety). In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ terminus of the sense or antisense strand) such that the targeting ligands resemble bristles of a toothbrush and the oligonucleotide resembles a toothbrush. For example, an oligonucleotide may comprise a stem-loop at either the 5′ or 3′ terminus of the sense strand and 1, 2, 3 or 4 nucleotides of the loop of the stem may be individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a triloop or a tetraloop, and wherein the 3 or 4 nucleotides comprising the triloop or tetraloop, respectively, are individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a tetraloop, and wherein 3 nucleotides of the tetraloop are individually conjugated to a targeting ligand.

GalNAc is a high affinity carbohydrate ligand for the asialoglycoprotein receptor (ASGPR), which is primarily expressed on the surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure (e.g., an RNAi oligonucleotide) is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure (e.g., an RNAi oligonucleotide) is conjugated directly or indirectly to a monovalent GalNAc moiety. In some embodiments, the oligonucleotide is conjugated directly or indirectly to more than one monovalent GalNAc (i.e., is conjugated to 2, 3 or 4 monovalent GalNAc moieties and is typically conjugated to 3 or 4 monovalent GalNAc moieties). In some embodiments, an oligonucleotide is conjugated to one or more bivalent GalNAc, trivalent GalNAc or tetravalent GalNAc moieties. In some embodiments, a bivalent, trivalent or tetravalent GalNAc moiety is conjugated to an oligonucleotide via a branched linker. In some embodiments, a monovalent GalNAc moiety is conjugated to a first nucleotide and a bivalent, trivalent, or tetravalent GalNAc moiety is conjugated to a second nucleotide via a branched linker.

In some embodiments, one (1) or more (e.g., 1, 2, 3, 4, 5 or 6) nucleotides of an oligonucleotide described herein (e.g., an RNAi oligonucleotide) are each conjugated to a GalNAc moiety. In some embodiments, two (2) to four (4) nucleotides of a tetraloop are each conjugated to a separate GalNAc moiety. In some embodiments, one (1) to three (3) nucleotides of a triloop are each conjugated to a separate GalNAc moiety. In some embodiments, targeting ligands are conjugated to two (2) to four (4) nucleotides at either ends of the sense or antisense strand (e.g., ligands are conjugated to a two (2) to four (4) nucleotide overhang or extension on the 5′ or 3′ terminus of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, three (3) or four (4) GalNAc moieties can be conjugated to nucleotides in the tetraloop of the sense strand where each GalNAc moiety is conjugated to one (1) nucleotide.

In some embodiments, an oligonucleotide described herein (e.g., an RNAi oligonucleotide) comprises a tetraloop, wherein the tetraloop (tetraLp) is any combination of adenine (A) and guanine (G) nucleotides. In some embodiments, the tetraloop (tetraLp) comprises a monovalent GalNAc moiety attached to any one or more guanine (G) nucleotides of the tetraloop via any linker described herein, as depicted below in Chem. 2 (X=heteroatom):

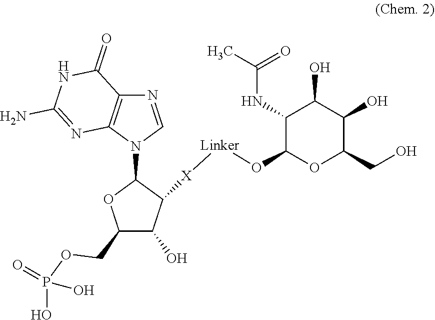

It is understood that when comprised in an oligonucleotide, Chem. 2 may be covalently linked to neighboring nucleotides. In some embodiments, the tetraloop (tetraLp) comprises a monovalent GalNAc moiety attached to any one or more guanine (G) nucleotides of the tetraloop via any linker described herein, as depicted below in Chem. 2a (X=heteroatom):

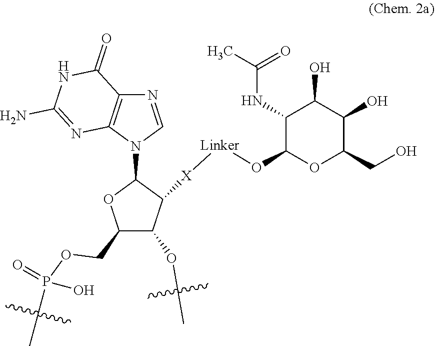

In some embodiments, the tetraloop (tetraLp) has a monovalent GalNAc attached to any one or more adenine (A) nucleotides of the tetraloop via any linker described herein, as depicted below Chem. 3 (X=heteroatom):

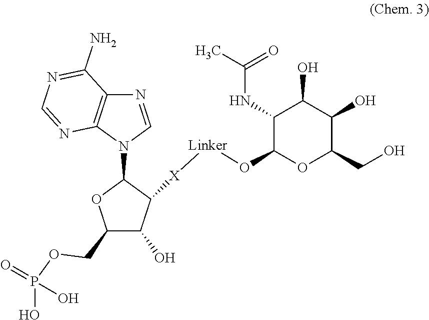

It is understood that when comprised in an oligonucleotide, Chem. 3 may be covalently linked to neighboring nucleotides. In some embodiments, the tetraloop (tetraLp) has a monovalent GalNAc attached to any one or more adenine (A) nucleotides of the tetraloop via any linker described herein, as depicted below Chem. 3a (X=heteroatom):

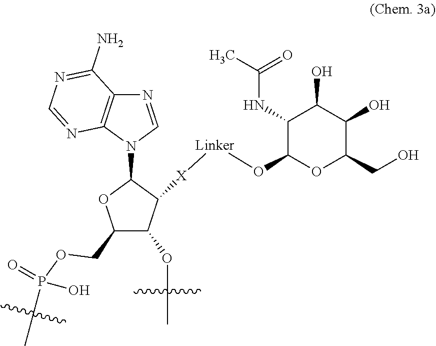

In some embodiments, an oligonucleotide herein (e.g., an RNAi oligonucleotide) comprises a monovalent GalNAc moiety attached to a guanine (G) nucleotide referred to as [ademG-GalNAc] or 2′-aminodiethoxymethanol-Guanine-GalNAc, as depicted below (Chem. 4):

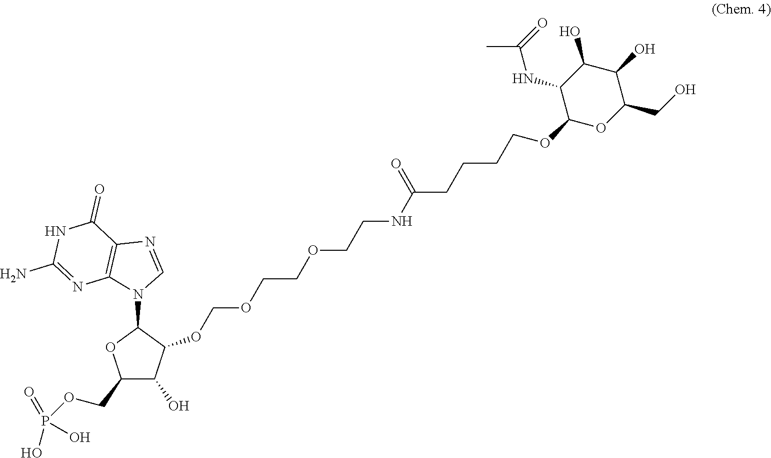 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

It is understood that when comprised in an oligonucleotide, Chem. 4 may be linked to neighboring nucleotides such as for instance shown in Chem 4a below:

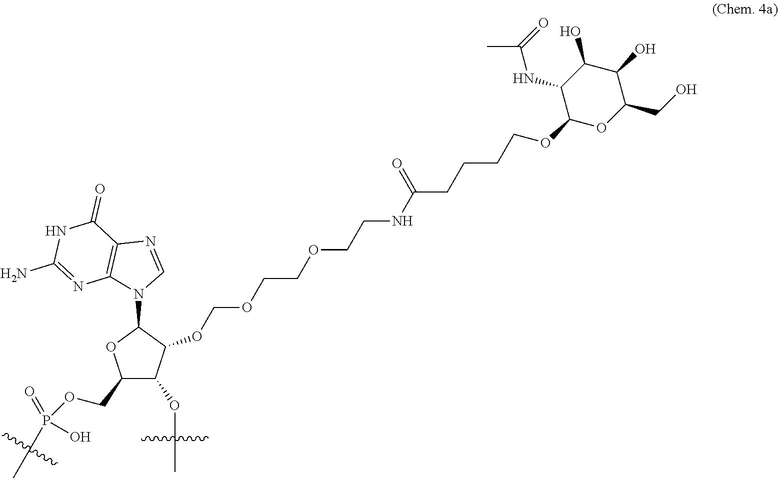 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(*)(=O)O]

In some embodiments, an oligonucleotide herein comprises a monovalent GalNAc moiety attached to an adenine nucleotide, referred to as [ademA-GalNAc] or 2′-aminodiethoxymethanol-Adenine-GalNAc, as depicted below (Chem. 5):

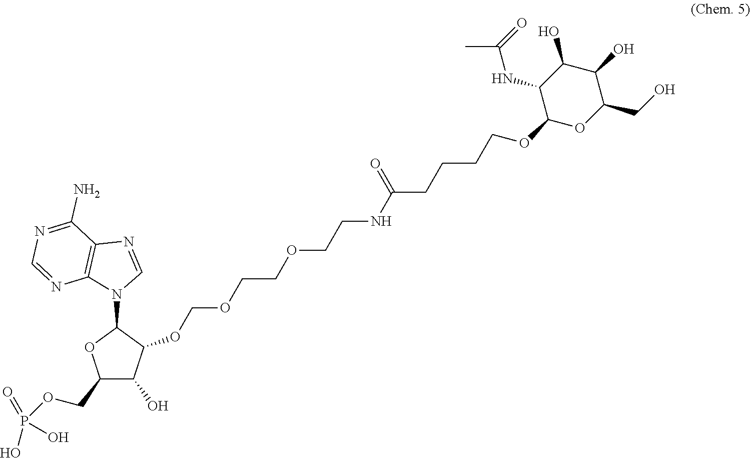 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

It is understood that when comprised in an oligonucleotide, Chem. 5 may be linked to neighboring nucleotides such as for instance shown in Chem 5a below:

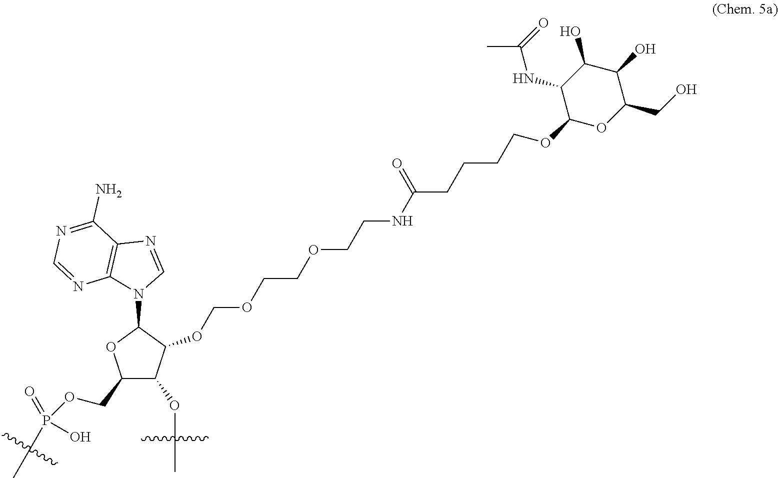 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

An example of such conjugation is shown below (Chem. 6) for a loop comprising from 5′ to 3′ the nucleotide sequence GAAA (L=linker, X=heteroatom). Such a loop may be present, for example, at positions 27-30 of a sense strand provided herein. In the chemical

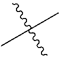

is used to describe an attachment point to the oligonucleotide strand (Chem. 6). 10 formula,

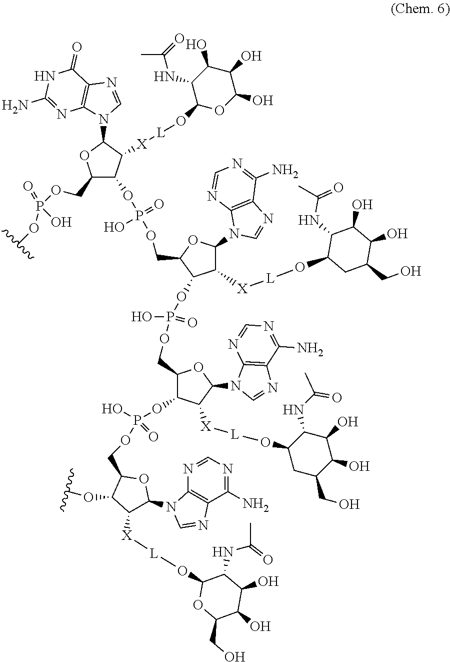

Another example of such conjugation is shown below (Chem. 6a) for a loop comprising from 5′ to 3′ the nucleotide sequence GAAA (L=linker, X=heteroatom). Such a loop may be present, for example, at positions 27-30 of a sense strand provided herein. In the chemical formula,

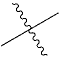

is used to describe an attachment point to the oligonucleotide strand.

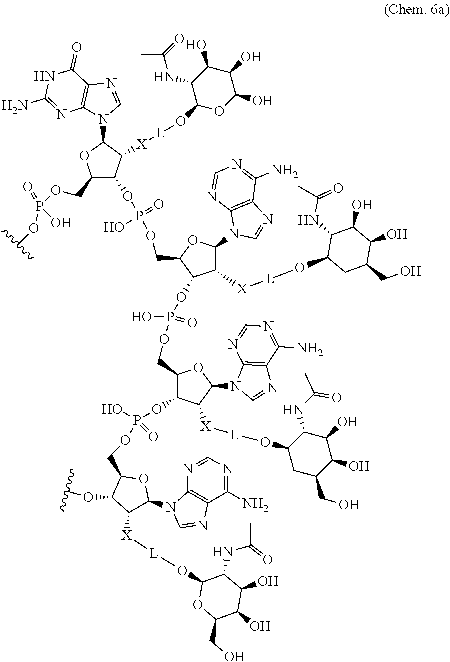

Appropriate methods or chemistry (e.g., click chemistry) can be used to link a targeting ligand to a nucleotide. In some embodiments, a targeting ligand is conjugated to a nucleotide comprising an oligonucleotide herein (e.g., an RNAi oligonucleotide) using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker (Chem. 7 and Chem. 8). Such a loop may be present, for example, at positions 27-30 of the any one of the sense strands. In the chemical formula,

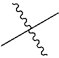

is an attachment point to the oligonucleotide strand (Chem. 7 and Chem. 8).

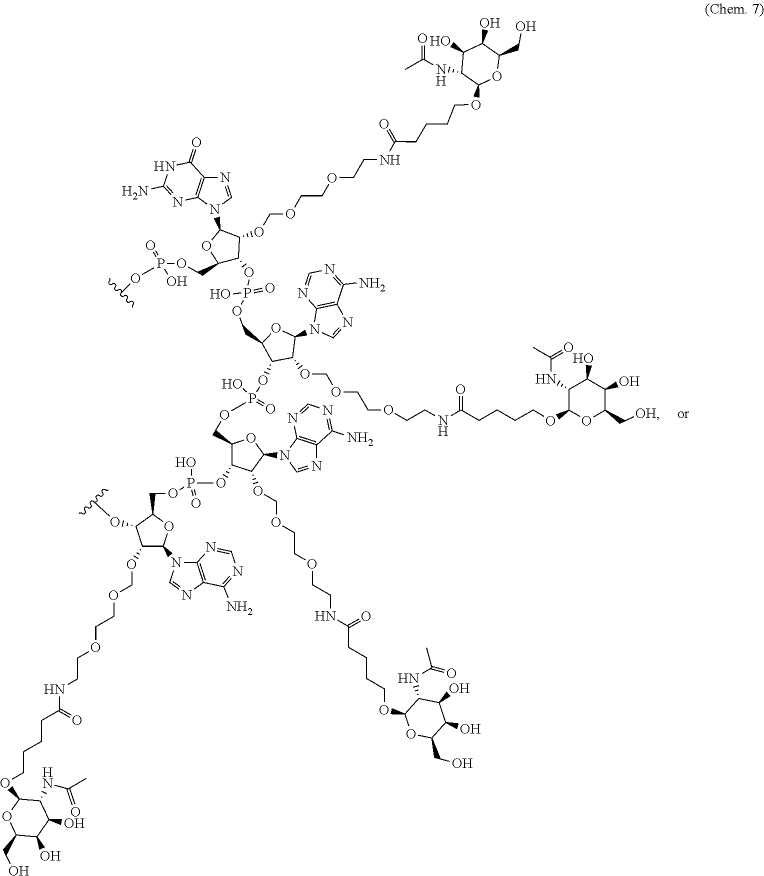 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

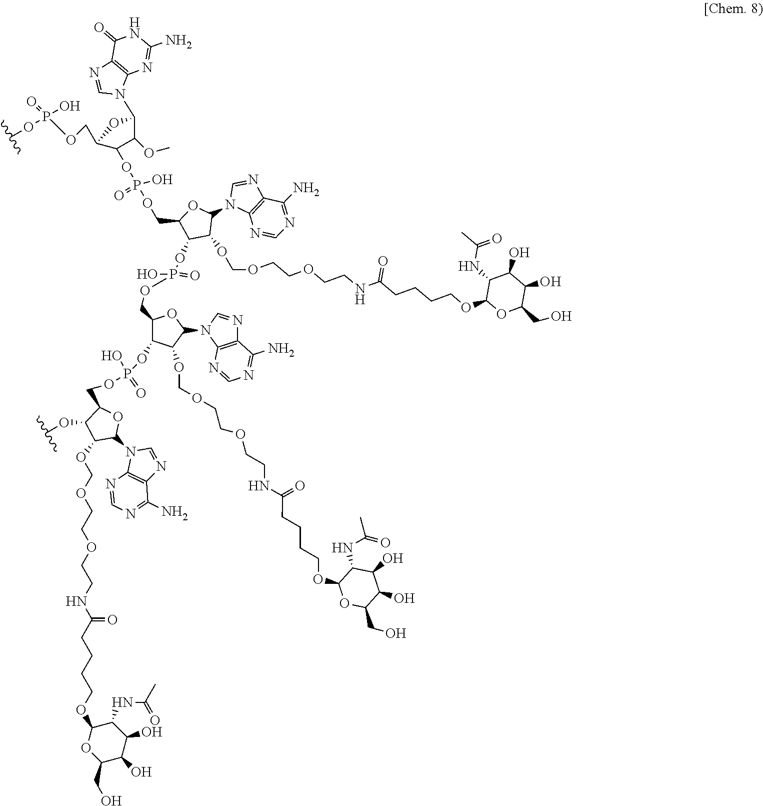

An example is shown below for a loop comprising from 5′ to 3′ the nucleotides GAAA, in which GalNAc moieties are attached to nucleotides of the loop using an acetal linker (Chem. 7a and Chem. 8a). Such a loop may be present, for example, at positions 27-30 of the any one of the sense strands. In the chemical formula,

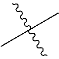

is an attachment point to the oligonucleotide strand (Chem. 7a and Chem. 8a):

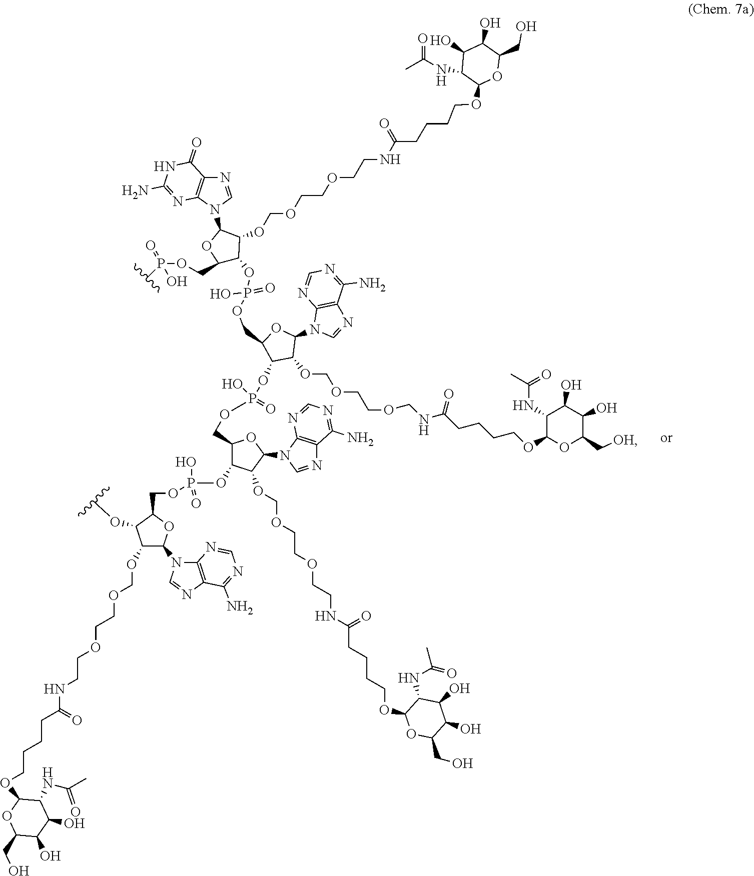 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(*)(=O)O]

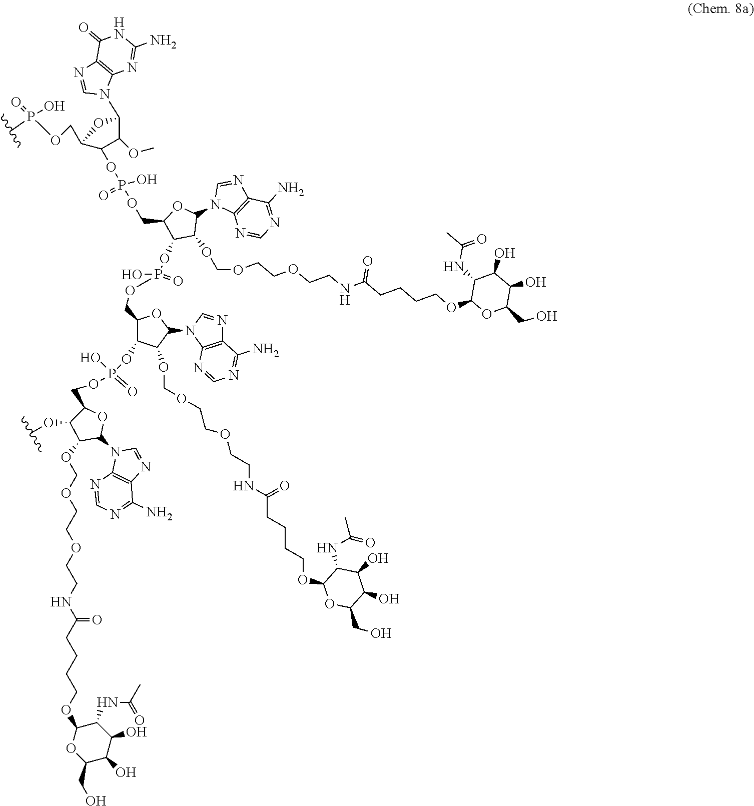

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, the oligonucleotides herein (e.g., RNAi oligonucleotides) do not have a GalNAc conjugated thereto.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively,
wherein the oligonucleotide comprises at least one GalNAc moiety conjugated to a nucleotide.

In some embodiments, the sense and antisense strands of an oligonucleotide comprise nucleotides sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively,
wherein the oligonucleotide comprises at least one GalNAc moiety conjugated to a nucleotide.

### Exemplary Oligonucleotides for Reducing TMPRSS6 Expression

In some embodiments, the TMPRSS6-targeting oligonucleotide (e.g., an RNAi oligonucleotide) for reducing TMPRSS6 expression provided by the disclosure comprise a sense strand and an antisense strand, wherein all nucleotides comprising the sense strand and antisense strand are modified, wherein the antisense strand comprises a region of complementarity to a TMPRSS6 mRNA target sequence of any one of SEQ ID NOs: 661-852 and wherein the region of complementarity is at least 15 contiguous nucleotides in length. In some embodiments, the 5′-terminal nucleotide of the antisense strand comprises 4′-O-monomethylphosphonate-2′-O-methyluridine [MePhosphonate-40-mU], as described herein. In some embodiments, the 5′-terminal nucleotide of the antisense strand comprises a phosphorothioate linkage. In some embodiments, the antisense strand and the sense strand comprise one or more 2′-fluoro (2′-F) and 2′-O-methyl (2′-OMe) modified nucleotides and at least one phosphorothioate linkage. In some embodiments, the antisense strand comprises four (4) phosphorothioate linkages and the sense strand comprises one (1) phosphorothioate linkage. In some embodiments, the antisense strand comprises five (5) phosphorothioate linkages and the sense strand comprises one (1) phosphorothioate linkage.

In some embodiments, an oligonucleotide provided herein (e.g., an RNAi oligonucleotide) comprises a sense strand having a sequence of any one of SEQ ID NOs: 193-384 and an antisense strand comprising a complementary sequence selected from SEQ ID NOs: 385-576.

In some embodiments, an oligonucleotide provided herein (e.g., an RNAi oligonucleotide) comprises a sense strand having a sequence of any one of SEQ ID NOs: 577-597 and an antisense strand comprising a complementary sequence selected from SEQ ID NOs: 598-618.

In some embodiments, an oligonucleotide provided herein (e.g., an RNAi oligonucleotide) comprises a sense strand having a sequence of any one of SEQ ID NOs: 619-639 and an antisense strand comprising a complementary sequence selected from SEQ ID NOs: 640-660.

In some embodiments, an oligonucleotide provided herein (e.g., and RNAi oligonucleotide) for reducing TMPRSS6 expression comprises:
a sense strand of 36 nucleotides comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand of 22 nucleotides comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively.

In some embodiments, the TMPRSS6-targeting oligonucleotide (e.g., an RNAi oligonucleotide) for reducing TMPRSS6 expression comprise:
a sense strand of 36 nucleotides comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand of 22 nucleotides comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively.

In some embodiments, a TMPRSS6-targeting oligonucleotide for reducing TMPRSS6 expression provided by the disclosure comprises a sense strand comprising the nucleotide sequence as set forth in SEQ ID NO: 579 and an antisense strand comprising the nucleotide sequence as set forth in SEQ ID NO: 600. In some embodiments, a TMPRSS6-targeting oligonucleotide for reducing TMPRSS6 expression provided by the disclosure comprises a sense strand comprising the nucleotide sequence as set forth in SEQ ID NO: 580 and an antisense strand comprising the nucleotide sequence as set forth in SEQ ID NO: 601. In some embodiments, a TMPRSS6-targeting oligonucleotide for reducing TMPRSS6 expression provided by the disclosure comprises a sense strand comprising the nucleotide sequence as set forth in SEQ ID NO: 590 and an antisense strand comprising the nucleotide sequence as set forth in SEQ ID NO: 611. In some embodiments, a TMPRSS6-targeting oligonucleotide for reducing TMPRSS6 expression provided by the disclosure comprises a sense strand comprising the nucleotide sequence as set forth in SEQ ID NO: 597 and an antisense strand comprising the nucleotide sequence as set forth in SEQ ID NO: 618. In some embodiments, a TMPRSS6-targeting oligonucleotide for reducing TMPRSS6 expression provided by the disclosure comprises a sense strand comprising the nucleotide sequence as set forth in SEQ ID NO: 586 and an antisense strand comprising the nucleotide sequence as set forth in SEQ ID NO: 607.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 184; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 181; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 158; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 134; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 102; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 184; and (ii) a sense strand of 19-50 nucleotides in length comprising a region of complementarity to the antisense strand and a stem-loop at the 3′ terminus, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 181; and (ii) a sense strand of 19-50 nucleotides in length comprising a region of complementarity to the antisense strand and a stem-loop at the 3′terminus, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 158; and (ii) a sense strand of 19-50 nucleotides in length comprising a region of complementarity to the antisense strand and a stem-loop at the 3′ terminus, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 134; and (ii) a sense strand of 19-50 nucleotides in length comprising a region of complementarity to the antisense strand and a stem-loop at the 3′terminus, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 102; and (ii) a sense strand of 19-50 nucleotides in length comprising a region of complementarity to the antisense strand and a stem-loop at the 3′ terminus, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 184; and (ii) a sense strand of 19-50 nucleotides in length comprising a region of complementarity to the antisense strand, wherein the region of complementarity to the antisense strand is set forth in SEQ ID NO: 844, wherein the antisense and sense strands are separate strands which form an asymmetric duplex region having an overhang of 1-4 nucleotides at the 3′ terminus of the antisense strand. In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 181; and (ii) a sense strand of 19-50 nucleotides in length comprising a region of complementarity to the antisense strand, wherein the region of complementarity to the antisense strand is set forth in SEQ ID NO: 841, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 158; and (ii) a sense strand of 19-50 nucleotides in length comprising a region of complementarity to the antisense strand, wherein the region of complementarity to the antisense strand is set forth in SEQ ID NO: 818, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 134; and (ii) a sense strand of 19-50 nucleotides in length comprising a region of complementarity to the antisense strand, wherein the region of complementarity to the antisense strand is set forth in SEQ ID NO: 794, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 102; and (ii) a sense strand of 19-50 nucleotides in length comprising a region of complementarity to the antisense strand, wherein the region of complementarity to the antisense strand is set forth in SEQ ID NO: 762, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 184; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 844, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 181; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 841, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 158; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 818, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 134; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 794, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a TMPRSS6-targeting oligonucleotide (e.g., an RNAi oligonucleotide) for reducing TMPRSS6 expression comprises (i) an antisense strand of 19-30 nucleotides in length, wherein the antisense strand comprises a nucleotide sequence comprising a region of complementarity to a TMPRSS6 mRNA target sequence, wherein the region of complementarity is set forth in SEQ ID NO: 102; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 762, wherein the stem-loop is set forth as S1-Lp-S2, wherein S1 is complementary to S2 and wherein Lp forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, the disclosure provides an oligonucleotide (e.g., an RNAi oligonucleotide) for reducing TMPRSS6 expression, wherein the oligonucleotide comprises a sense strand and an antisense strand according to:

Sense Strand:
5′-mX-S-mX-mX-mX-mX-mX-mX-fX-fX-fX-fX-mX-mX-mX-mX-
mX-mX-mX-mX-mX-mX-mX-mX-mX-mX-mX-mX-[ademX-
GalNAc]-[ademX-GalNAc]-[ademX-GalNAc]-mX-mX-mX-mX-
mX-mX-3′;

hybridized to:

Antisense Strand:
5′-[MePhosphonate-4O-mX]-S-fX-S-fX-S-fX-fX-mX-fX-
mX-mX-fX-mX-mX-mX-fX-mX-mX-mX-mX-mX-mX-S-mX-S-
mX-3′;

wherein mX=2′-O-methyl modified nucleotide,
fX=2′-fluoro modified nucleotide,
—S—=phosphorothioate linkage,
−=phosphodiester linkage,
[MePhosphonate-40-mX]=4′-O-monomethylphosphonate-2′-O-methyl modified nucleotide, and
ademX-GalNAc=GalNAc attached to a nucleotide.

In some embodiments, the disclosure provides an RNAi oligonucleotide for reducing TMPRSS6 expression, the oligonucleotide comprising a sense strand and an antisense strand, wherein the sense strand comprises the sequence and all of the modifications of 5′-[mGs][mG][mU][mG][mC][mU][mA][C][fU][fC][fU][mG][mG][mU][mA][mU][mU][mU][mC][mA][mG][mC][mA][mG][mC][mC][mG][ademA-GalNAc][ademA-GalNAc][ademA-GalNAc][mG][mG][mC][mU][mG][mC]-3′ (SEQ ID NO: 621), and wherein the antisense strand comprises the sequence and all of the modifications of 5′ [MePhosphonate-40-mUs][fGs][fAs][fA][fA][mU][fA][mC][mC][fA][mG][mA][mG][fU][mA][mG][mC][mA][mC][mCs][m Gs][mG]-3′ (SEQ ID NO: 642), wherein mC, mA, mG, mU=2′-OMe ribonucleosides; fA, fC, fG, fU=2′-F ribonucleosides; s=phosphorothioate, and wherein ademA-GalNAc=GalNAc modified adenine nucleotide.

In some embodiments, the disclosure provides an RNAi oligonucleotide for reducing TMPRSS6 expression, the oligonucleotide comprising a sense strand and an antisense strand, wherein the sense strand comprises the sequence and all of the modifications of 5′ [mGs][mC][mU][mA][mC][mU][mC][fU][fG][fG][fU][mA][mU][mU][mU][mC][mC][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 622), and wherein the antisense strand comprises the sequence and all of the modifications of 5′ [MePhosphonate-40-mUs][fUs][fAs][G][fG][mA][fA][mA][mU][fA][mC][mC][mA][fG][mA][mG][mU][mA][mG][mCs][m Gs][mG]-3′ (SEQ ID NO: 643), wherein mC, mA, mG, mU=2′-OMe ribonucleosides; fA, fC, fG, fU=2′-F ribonucleosides; s=phosphorothioate, and wherein ademA-GalNAc=GalNAc modified adenine nucleotide.

In some embodiments, the disclosure provides an RNAi oligonucleotide for reducing TMPRSS6 expression, the oligonucleotide comprising a sense strand and an antisense strand, wherein the sense strand comprises the sequence and all of the modifications of 5′-[mCs][mU][mC][mA][mC][mC][mU][fG][fC][fU][fU][mC][mU][mU][mC][mU][mG][mG][mU][mA][mG][mC][mA][mG][mC][mC][mG][ademA-GalNAc][ademA-GalNAc][ademA-GalNAc][mG][mG][mC][mU][mG][mC]-3′ (SEQ ID NO: 632), and wherein the antisense strand comprises the sequence and all of the modifications of 5′-[MePhosphonate-40-mUs][fAs][fCs][fC][fA][mG][fA][mA][mG][fA][mA][mG][mC][fA][mG][mG][mU][mG][mA][mGs][m Gs][mG]-3′ (SEQ ID NO: 653), wherein mC, mA, mG, mU=2′-OMe ribonucleosides; fA, fC, fG, fU=2′-F ribonucleosides; s=phosphorothioate, and wherein ademA-GalNAc=GalNAc modified adenine nucleotide.

In some embodiments, the disclosure provides, an RNAi oligonucleotide for reducing TMPRSS6 expression, the oligonucleotide comprising a sense strand and an antisense strand, wherein the sense strand comprises the sequence and all of the modifications of 5′-[mAs][mG][mU][mG][mU][mG][mA][fA][fA][fG][fA][mC][mA][mU][mA][mG][mC][mU][mG][mA][mG][mC][mA][mG][mC][mC][mG][ademA-GalNAc][ademA-GalNAc][ademA-GalNAc][mG][mG][mC][mU][mG][mC]3′ (SEQ ID NO: 639), and wherein the antisense strand comprises the sequence and all of the modifications of 5′-[MePhosphonate-40-mUs][fCs][fAs][fG][fC][mU][fA][mU][mG][fU][mC][mU][mU][fU][mC][mA][mC][mA][mC][mUs][m Gs][mG]-3′ (SEQ ID NO: 660), wherein mC, mA, mG, mU=2′-OMe ribonucleosides; fA, fC, fG, fU=2′-F ribonucleosides; s=phosphorothioate, and wherein ademA-GalNAc=GalNAc modified adenine nucleotide.

In some embodiments, the disclosure provides, an RNAi oligonucleotide for reducing TMPRSS6 expression, the oligonucleotide comprising a sense strand and an antisense strand, wherein the sense strand comprises the sequence and all of the modifications of 5′-[mGs][mG][mG][mU][mG][mC][mA][fC][fU][fA][fU][mG][mG][mC][mU][mU][mG][mU][mA][mA][mG][mC][mA][mG][mC][mC][mG][ademA-GalNAc][ademA-GalNAc][ademA-GalNAc][mG][mG][mC][mU][mG][mC]3′ (SEQ ID NO: 628), and wherein the antisense strand comprises the sequence and all of the modifications of 5′-[MePhosphonate-40-mUs][fUs][fAs][fC][fA][mA][fG][mC][mC][fA][mU][mA][mG][fU][mG][mC][mA][mC][mC][mCs][m Gs][mG]-3′ (SEQ ID NO: 649), wherein mC, mA, mG, mU=2′-OMe ribonucleosides; fA, fC, fG, fU=2′-F ribonucleosides; s=phosphorothioate, and wherein ademA-GalNAc=GalNAc modified adenine nucleotide.

In some embodiments, the disclosure provides an oligonucleotide (e.g., an RNAi oligonucleotide) for reducing TMPRSS6 expression, wherein the oligonucleotide comprises a sense strand and an antisense strand comprising nucleotide sequences selected from the group consisting of:
a) SEQ ID NOs: 621 and 642, respectively;
b) SEQ ID NOs: 622 and 643, respectively;
c) SEQ ID NOs: 637 and 658, respectively;
d) SEQ ID NOs: 632 and 653, respectively;
e) SEQ ID NOs: 638 and 659, respectively;
f) SEQ ID NOs: 639 and 660, respectively;
g) SEQ ID NOs: 627 and 648, respectively;
h) SEQ ID NOs: 628 and 649, respectively; and,
i) SEQ ID NOs: 629 and 650, respectively.

In some embodiments, a TMPRSS6-targeting oligonucleotide for reducing TMPRSS6 expression provided by the disclosure comprises a sense strand comprising the nucleotide sequence as set forth in SEQ ID NO: 621 and an antisense strand comprising the nucleotide sequence as set forth in SEQ ID NO: 642. In some embodiments, a TMPRSS6-targeting oligonucleotide for reducing TMPRSS6 expression provided by the disclosure comprises a sense strand comprising the nucleotide sequence as set forth in SEQ ID NO: 622 and an antisense strand comprising the nucleotide sequence as set forth in SEQ ID NO: 643. In some embodiments, a TMPRSS6-targeting oligonucleotide for reducing TMPRSS6 expression provided by the disclosure comprises a sense strand comprising the nucleotide sequence as set forth in SEQ ID NO: 632 and an antisense strand comprising the nucleotide sequence as set forth in SEQ ID NO: 653. In some embodiments, a TMPRSS6-targeting oligonucleotide for reducing TMPRSS6 expression provided by the disclosure comprises a sense strand comprising the nucleotide sequence as set forth in SEQ ID NO: 639 and an antisense strand comprising the nucleotide sequence as set forth in SEQ ID NO: 660. In some embodiments, a TMPRSS6-targeting oligonucleotide for reducing TMPRSS6 expression provided by the disclosure comprises a sense strand comprising the nucleotide sequence as set forth in SEQ ID NO: 628 and an antisense strand comprising the nucleotide sequence as set forth in SEQ ID NO: 649.

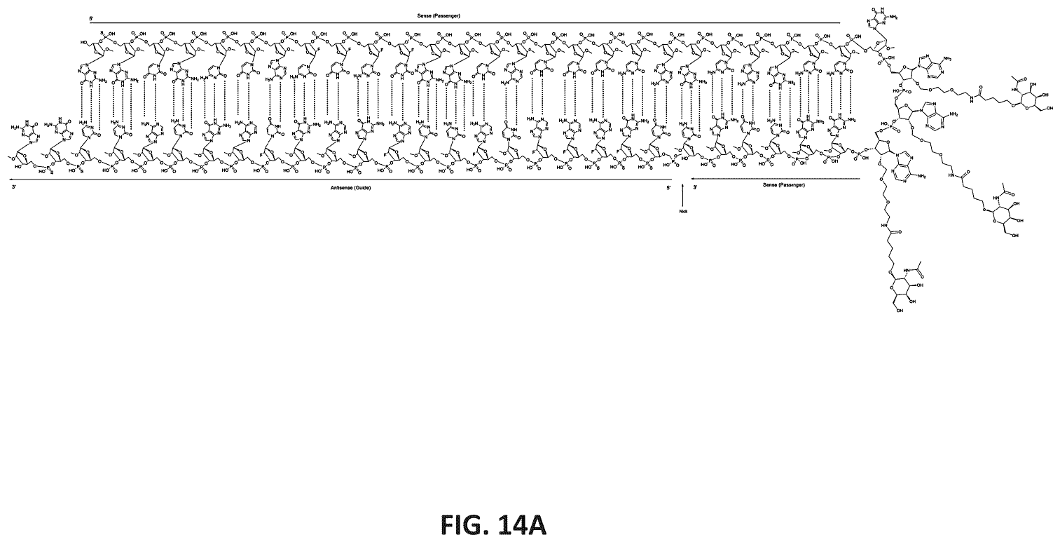

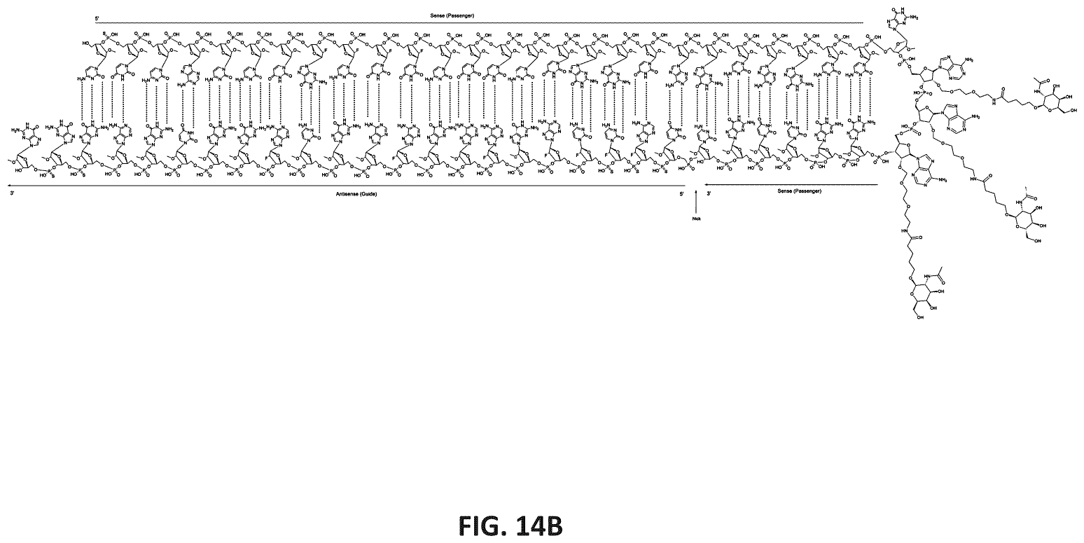

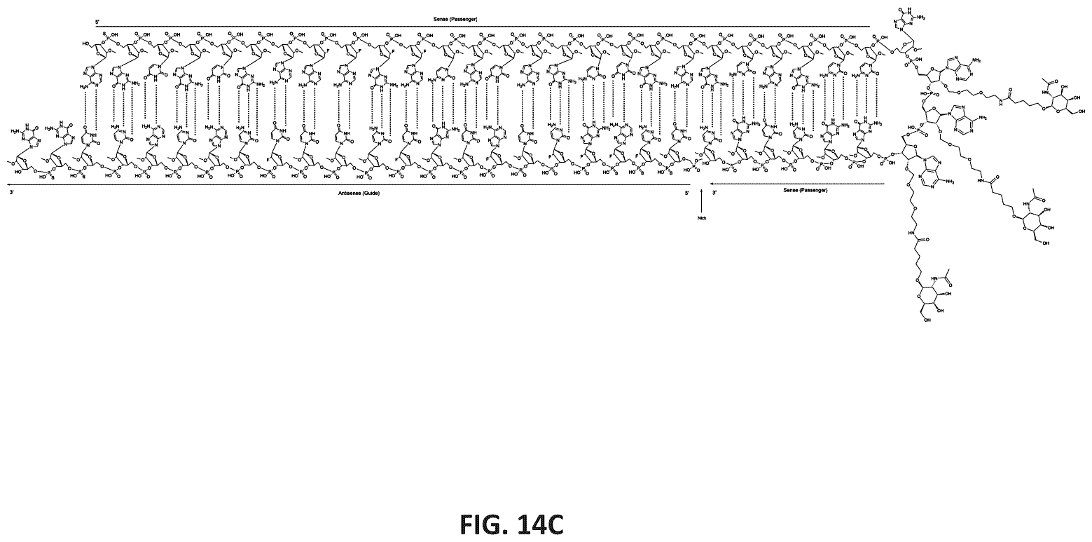

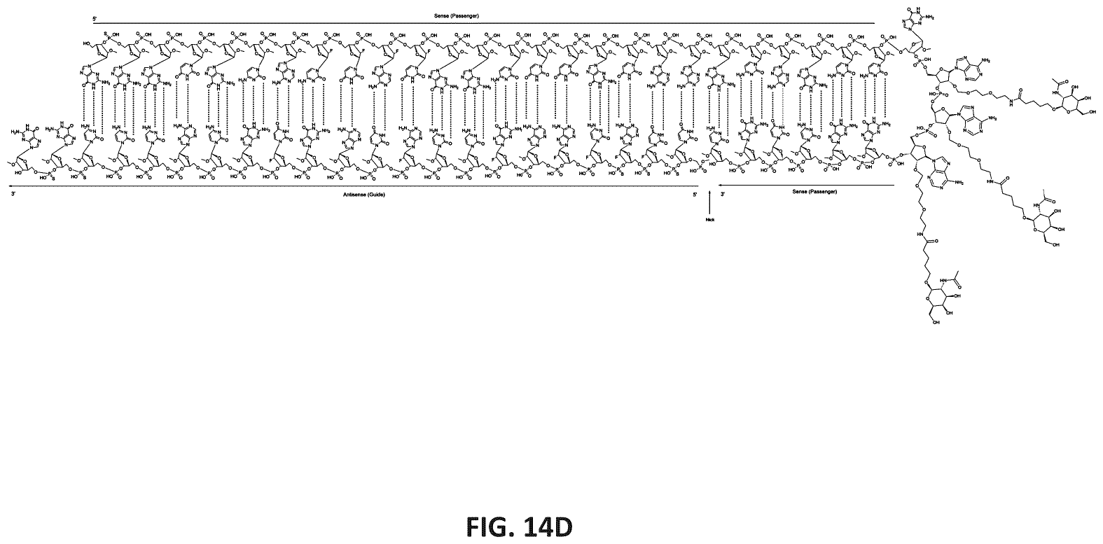

In some embodiments, a TMPRSS6-targeting oligonucleotide for reducing TMPRSS6 expression provided by the disclosure comprises a sense strand comprising the nucleotide sequence as set forth in SEQ ID NO: 621 and an antisense strand comprising the nucleotide sequence as set forth in SEQ ID NO: 642, the antisense strand comprising a region of complementarity to a TMPRSS6 RNA transcript, wherein said RNAi is in the form of a conjugate having the structure as shown in FIG. 14A.

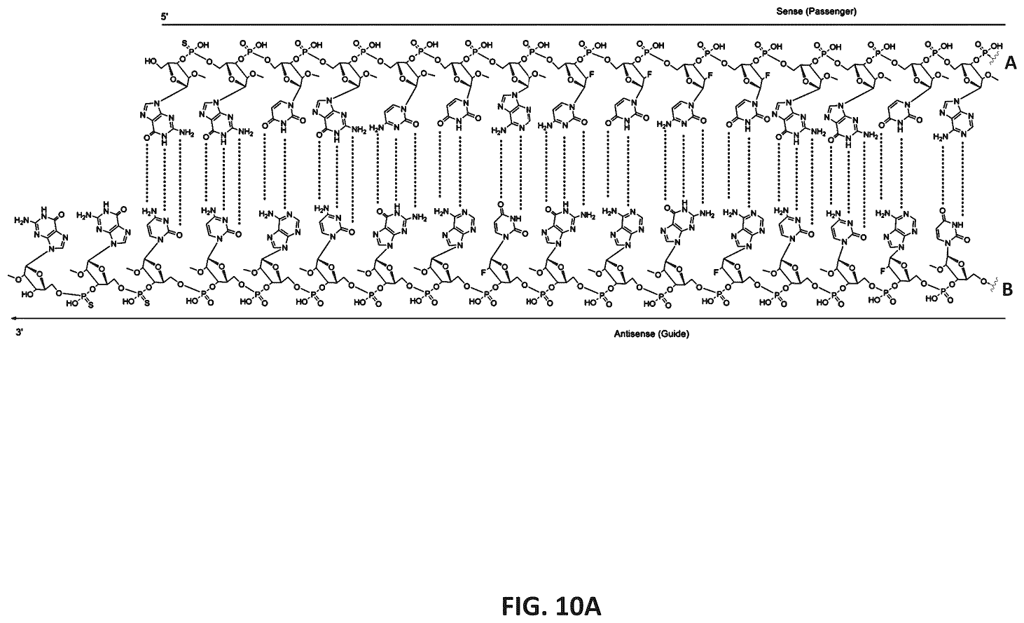

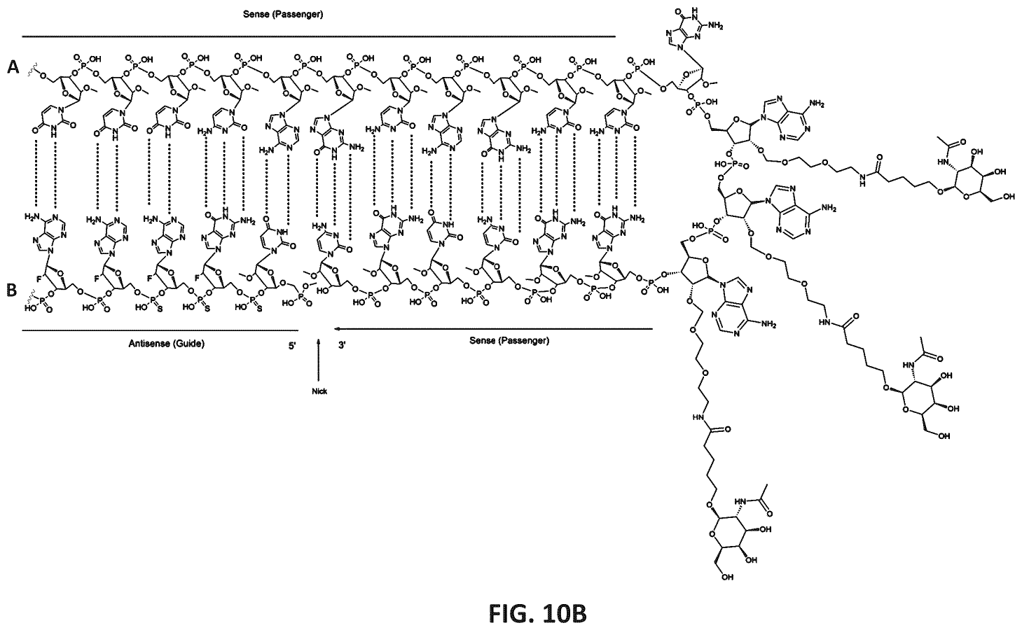

In some embodiments, a TMPRSS6-targeting oligonucleotide for reducing TMPRSS6 expression provided by the disclosure comprises a sense strand comprising the nucleotide sequence as set forth in SEQ ID NO: 621 and an antisense strand comprising the nucleotide sequence as set forth in SEQ ID NO: 642, the antisense strand comprising a region of complementarity to a TMPRSS6 RNA transcript, wherein said RNAi is in the form of a conjugate having the structure as shown in FIGS. 10A-B.

In some embodiments, a TMPRSS6-targeting oligonucleotide for reducing TMPRSS6 expression provided by the disclosure comprises a sense strand comprising the nucleotide sequence as set forth in SEQ ID NO: 632 and an antisense strand comprising the nucleotide sequence as set forth in SEQ ID NO: 653, the antisense strand comprising a region of complementarity to a TMPRSS6 RNA transcript, wherein said RNAi is in the form of a conjugate having the structure as shown in FIG. 14B.

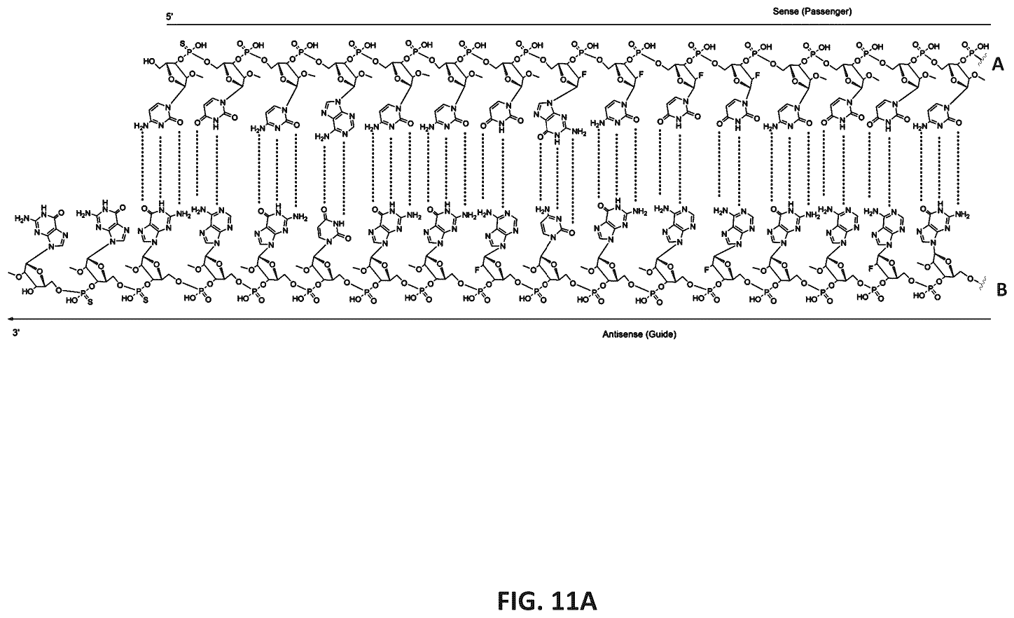

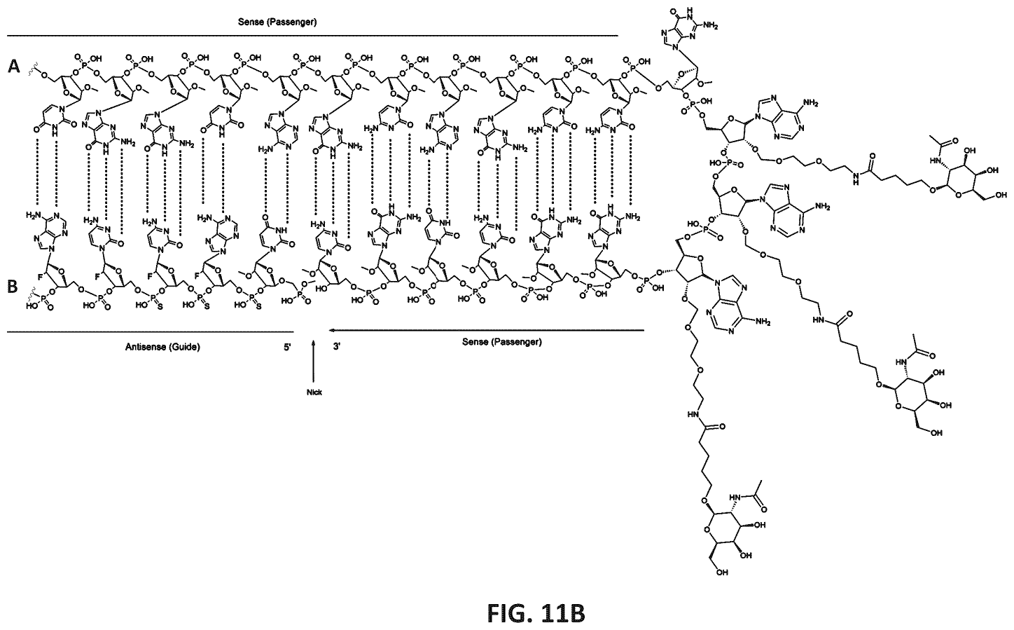

In some embodiments, a TMPRSS6-targeting oligonucleotide for reducing TMPRSS6 expression provided by the disclosure comprises a sense strand comprising the nucleotide sequence as set forth in SEQ ID NO: 632 and an antisense strand comprising the nucleotide sequence as set forth in SEQ ID NO: 653, the antisense strand comprising a region of complementarity to a TMPRSS6 RNA transcript, wherein said RNAi is in the form of a conjugate having the structure as shown in FIGS. 11A-B.

In some embodiments, a TMPRSS6-targeting oligonucleotide for reducing TMPRSS6 expression provided by the disclosure comprises a sense strand comprising the nucleotide sequence as set forth in SEQ ID NO: 639 and an antisense strand comprising the nucleotide sequence as set forth in SEQ ID NO: 660, the antisense strand comprising a region of complementarity to a TMPRSS6 RNA transcript, wherein said RNAi is in the form of a conjugate having the structure as shown in FIG. 14C.

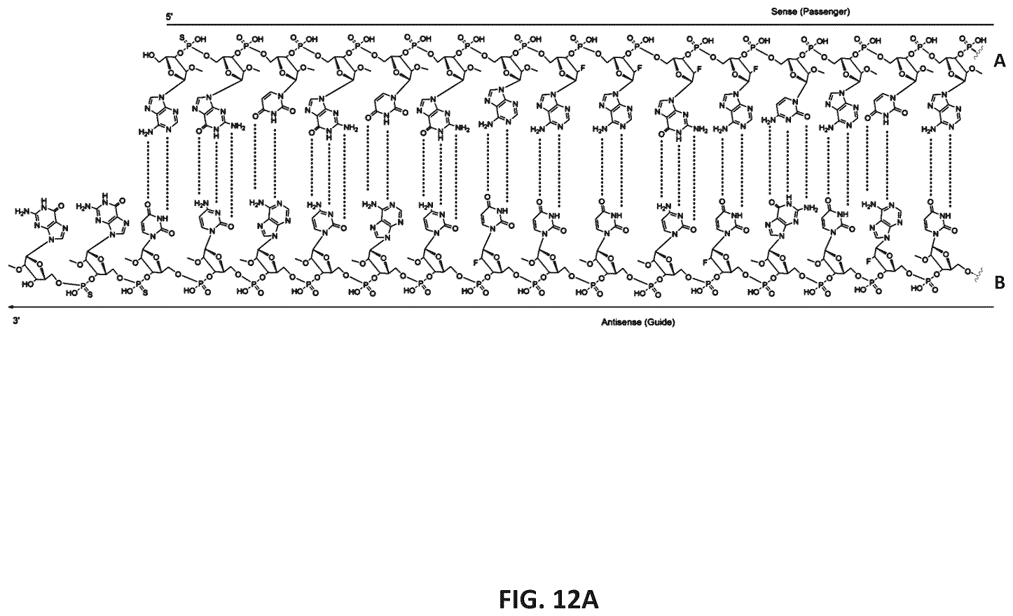

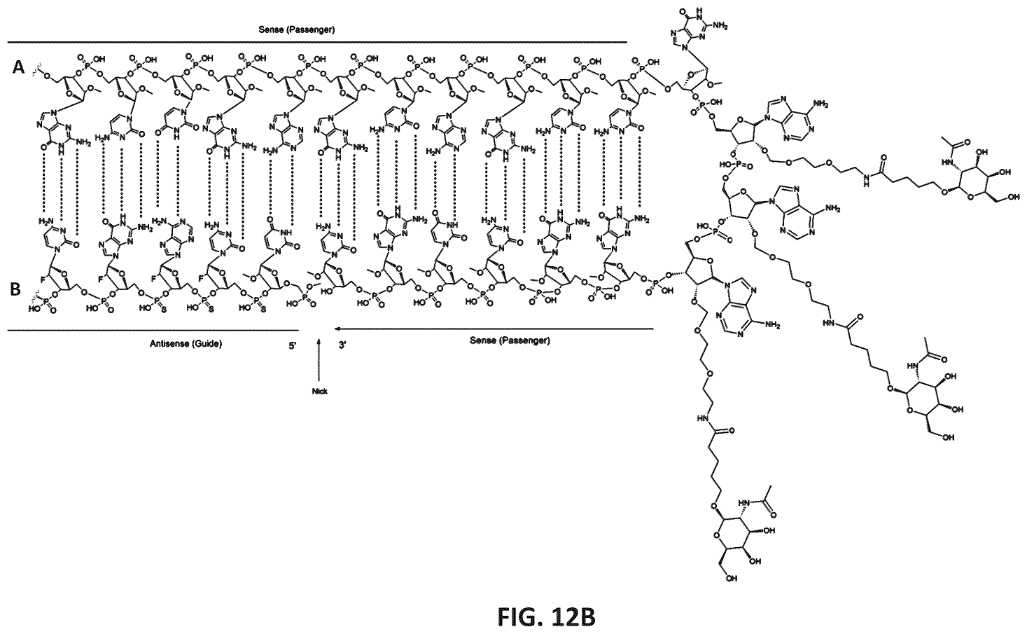

In some embodiments, a TMPRSS6-targeting oligonucleotide for reducing TMPRSS6 expression provided by the disclosure comprises a sense strand comprising the nucleotide sequence as set forth in SEQ ID NO: 639 and an antisense strand comprising the nucleotide sequence as set forth in SEQ ID NO: 660, the antisense strand comprising a region of complementarity to a TMPRSS6 RNA transcript, wherein said RNAi is in the form of a conjugate having the structure as shown in FIGS. 12A-B.

In some embodiments, a TMPRSS6-targeting oligonucleotide for reducing TMPRSS6 expression provided by the disclosure comprises a sense strand comprising the nucleotide sequence as set forth in SEQ ID NO: 628 and an antisense strand comprising the nucleotide sequence as set forth in SEQ ID NO: 649, the antisense strand comprising a region of complementarity to a TMPRSS6 RNA transcript, wherein said RNAi is in the form of a conjugate having the structure as shown in FIG. 14D.

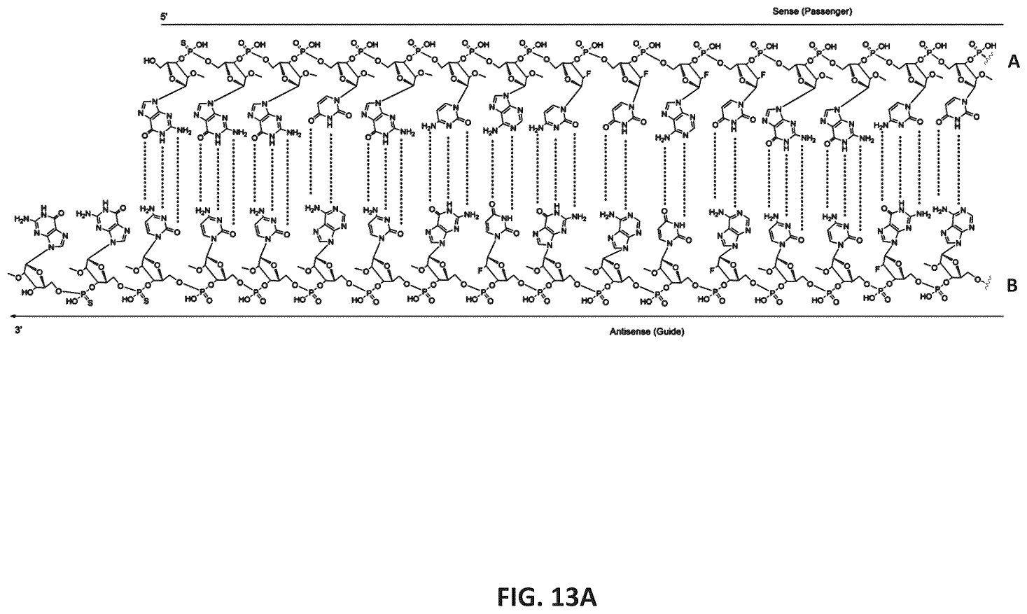

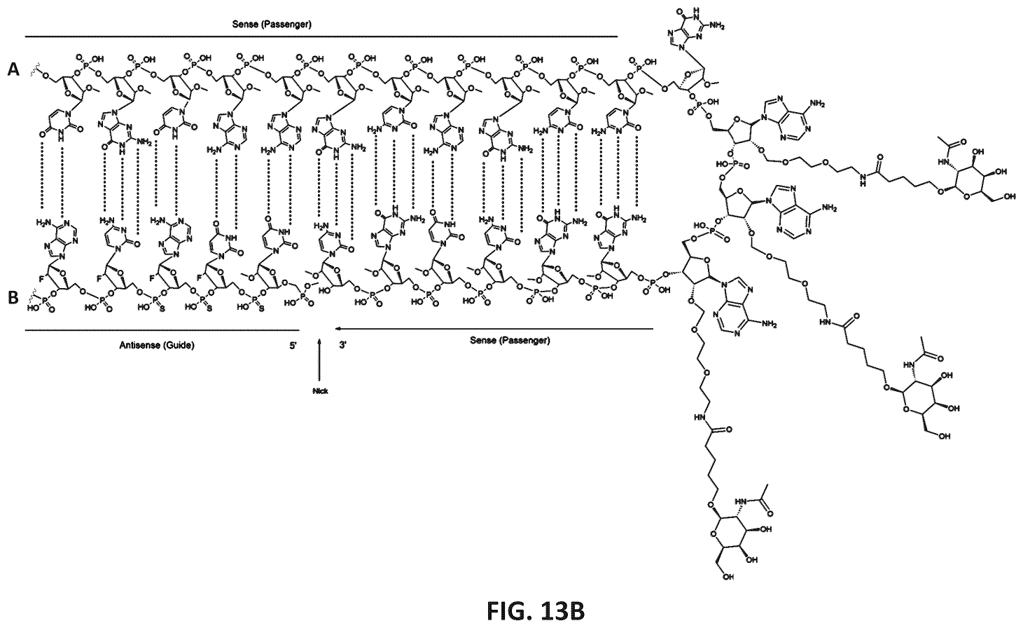

In some embodiments, a TMPRSS6-targeting oligonucleotide for reducing TMPRSS6 expression provided by the disclosure comprises a sense strand comprising the nucleotide sequence as set forth in SEQ ID NO: 628 and an antisense strand comprising the nucleotide sequence as set forth in SEQ ID NO: 649, the antisense strand comprising a region of complementarity to a TMPRSS6 RNA transcript, wherein said RNAi is in the form of a conjugate having the structure as shown in FIGS. 13A-B.

### Formulations

Various formulations (e.g., pharmaceutical formulations) have been developed for oligonucleotide use. For example, oligonucleotides (e.g., RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., RNAi oligonucleotides) reduce the expression of TMPRSS6. Such compositions can be suitably formulated such that when administered to a subject, either into the immediate environment of a target cell or systemically, a sufficient portion of the oligonucleotides enter the cell to reduce TMPRSS6 expression. Any variety of suitable oligonucleotide formulations can be used to deliver oligonucleotides for the reduction of TMPRSS6 as disclosed herein. In some embodiments, an oligonucleotide is formulated in buffer solutions such as phosphate buffered saline solutions, liposomes, micellar structures, and capsids. Any of the oligonucleotides described herein may be provided not only as nucleic acids, but also in the form of a pharmaceutically acceptable salt.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a RNAi oligonucleotide for reducing TMPRSS6 expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing TMPRSS6 Expression

In some embodiments, the disclosure provides methods for contacting or delivering to a cell or population of cells an effective amount of oligonucleotides provided herein (e.g., RNAi oligonucleotides) to reduce TMPRSS6 expression. In some embodiments, a reduction of TMPRSS6 expression is determined by measuring a reduction in the amount or level of TMPRSS6 mRNA, matriptase-2 protein, or matriptase-2 activity in a cell. The methods include those described herein and known to one of ordinary skill in the art.

Methods provided herein are useful in any appropriate cell type. In some embodiments, a cell is any cell that expresses TMPRSS6 mRNA (e.g., hepatocytes). In some embodiments, the cell is a primary cell obtained from a subject. In some embodiments, the primary cell has undergone a limited number of passages such that the cell substantially maintains its natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein (e.g., RNAi oligonucleotides) are delivered to a cell or population of cells using a nucleic acid delivery method known in the art including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other methods known in the art for delivering oligonucleotides to cells may be used, such as lipid-mediated carrier transport, chemical-mediated transport, and cationic liposome transfection such as calcium phosphate, and others.

In some embodiments, reduction of TMPRSS6 expression is determined by an assay or technique that evaluates one or more molecules, properties, or characteristics of a cell or population of cells associated with TMPRSS6 expression, or by an assay or technique that evaluates molecules that are directly indicative of TMPRSS6 expression in a cell or population of cells (e.g., TMPRSS6 mRNA or matriptase-2 protein). In some embodiments, the extent to which an oligonucleotide provided herein reduces TMPRSS6 expression is evaluated by comparing TMPRSS6 expression in a cell or population of cells contacted with the oligonucleotide to an appropriate control (e.g., an appropriate cell or population of cells not contacted with the oligonucleotide or contacted with a control oligonucleotide). In some embodiments, a control amount or level of TMPRSS6 expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide described herein (e.g., an RNAi oligonucleotide) to a cell or a population of cells results in a reduction in TMPRSS6 expression in a cell or population of cells not contacted with the oligonucleotide or contacted with a control oligonucleotide. In some embodiments, the reduction in TMPRSS6 expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of TMPRSS6 expression. In some embodiments, the control amount or level of TMPRSS6 expression is an amount or level of TMPRSS6 mRNA and/or matriptase-2 protein in a cell or population of cells that has not been contacted with an oligonucleotide herein. In some embodiments, TMPRSS6 mRNA expression is measured using methods known in the art. In some embodiments, TMPRSS6 mRNA expression is measured by qPCR. In some embodiments, TMPRSS6 protein expression is measured using methods known in the art. In some embodiments TMPRSS6 protein expression is measured by ELISA. In some embodiments, TMPRSS6 protein expression is measured by western blot. In some embodiments, the effect of delivery of an oligonucleotide herein to a cell or population of cells according to a method herein is assessed after any finite period or amount of time (e.g., minutes, hours, days, weeks, months). For example, in some embodiments, TMPRSS6 expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, TMPRSS6 expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide provided herein (e.g., an RNAi oligonucleotide) is delivered in the form of a transgene that is engineered to express in a cell the oligonucleotide or strands comprising the oligonucleotide (e.g., its sense and antisense strands). In some embodiments, an oligonucleotide herein is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

### Treatment Methods

The disclosure provides oligonucleotides (e.g., RNAi oligonucleotides) for use as a medicament, in particular for use in a method for the treatment of diseases, disorders, and conditions associated with hepcidin deficiency or suppression. The disclosure also provides oligonucleotides for use, or adaptable for use, to treat a subject (e.g., a human having a disease, disorder or condition associated with hepcidin deficiency or suppression) that would benefit from reducing TMPRSS6 expression. In some respects, the disclosure provides oligonucleotides for use, or adapted for use, to treat a subject having a disease, disorder or condition associated with hepcidin deficiency or suppression. In some embodiments, the subject in need of TMPRSS6 reduction has, or is at risk for, an iron accumulation disease, disorder or condition. In some embodiments, compositions, compounds and methods described herein are provided for use in reducing iron levels in an individual. In some embodiments, the iron accumulation is due to a disease, disorder, or condition in the subject. In some embodiments, the disease, disorder and/or condition is hemochromatosis such as hereditary hemochromatosis. In some embodiments, the disease, disorder and/or condition is β-thalassemia. In some embodiments, the disease, disorder and/or condition is polycythemia vera. The disclosure also provides oligonucleotides for use, or adaptable for use, in the manufacture of a medicament or pharmaceutical composition for treating a disease, disorder or condition associated with hepcidin deficiency or suppression. In some embodiments, the oligonucleotides for use, or adaptable for use, target TMPRSS6 mRNA and reduce TMPRSS6 expression (e.g., via the RNAi pathway). In some embodiments, the oligonucleotides for use, or adaptable for use, target TMPRSS6 mRNA and reduce the amount or level of TMPRSS6 mRNA, matriptase-2 protein and/or TMPRSS6 activity.

The disclosure also provides methods of treating a subject having, suspected of having, or at risk of developing a disease, disorder or condition associated with a hepcidin deficiency or suppression with an oligonucleotide provided herein. In some aspects, the disclosure provides methods of treating or attenuating the onset or progression of a disease, disorder or condition associated with hepcidin deficiency or suppression using the oligonucleotides herein. In other aspects, the disclosure provides methods to achieve one or more therapeutic benefits in a subject having a disease, disorder, or condition associated with hepcidin deficiency or suppression using the oligonucleotides provided herein. In some embodiments of the methods herein, the subject is treated by administering a therapeutically effective amount of any one or more of the oligonucleotides provided herein. In some embodiments, treatment comprises reducing TMPRSS6 expression. In some embodiments, the subject is treated therapeutically. In some embodiments, the subject is treated prophylactically.

In some embodiments of the methods herein, one or more oligonucleotides herein (e.g., RNAi oligonucleotides), or a pharmaceutical composition comprising one or more oligonucleotides, is administered to a subject having a disease, disorder or condition associated with hepcidin deficiency or suppression such that TMPRSS6 expression is reduced in the subject, thereby treating the subject. In some embodiments, an amount or level of TMPRSS6 mRNA is reduced in the subject. In some embodiments, an amount or level of matriptase-2 protein is reduced in the subject. In some embodiments, an amount or level of matriptase-2 activity is reduced in the subject.

In some embodiments of the methods herein, an oligonucleotide provided herein (e.g., an RNAi oligonucleotide), or a pharmaceutical composition comprising the oligonucleotide, is administered to a subject having a disease, disorder or condition associated with hepcidin deficiency or suppression such that TMPRSS6 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to TMPRSS6 expression prior to administration of one or more oligonucleotides or pharmaceutical composition. In some embodiments, TMPRSS6 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to TMPRSS6 expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein (e.g., RNAi oligonucleotides), or a pharmaceutical composition comprising the oligonucleotide or oligonucleotides, is administered to a subject having a disease, disorder or condition associated with hepcidin deficiency or suppression such that an amount or level of TMPRSS6 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of TMPRSS6 mRNA prior to administration of the oligonucleotide or pharmaceutical composition. In some embodiments, an amount or level of TMPRSS6 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of TMPRSS6 mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with hepcidin deficiency or suppression such that an amount or level of matriptase-2 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of matriptase-2 protein prior to administration of the oligonucleotide or pharmaceutical composition. In some embodiments, an amount or level of matriptase-2 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of matriptase-2 protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with hepcidin deficiency or suppression such that an amount or level of TMPRSS6 gene activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of TMPRSS6 activity prior to administration of the oligonucleotide or pharmaceutical composition. In some embodiments, an amount or level of TMPRSS6 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of TMPRSS6 activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide provided herein (e.g., an RNAi oligonucleotide), or a pharmaceutical composition comprising the oligonucleotide, is administered to a subject having a disease, disorder or condition associated with hepcidin deficiency or suppression such that hepcidin production is increased in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to hepcidin production prior to administration of one or more oligonucleotides or pharmaceutical composition. In some embodiments, hepcidin production is increased in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to hepcidin production in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide provided herein (e.g., an RNAi oligonucleotide), or a pharmaceutical composition comprising the oligonucleotide, is administered to a subject having a disease, disorder or condition associated with hepcidin deficiency or suppression such that serum iron is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to serum iron prior to administration of one or more oligonucleotides or pharmaceutical composition. In some embodiments, serum iron is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to serum iron in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide provided herein (e.g., an RNAi oligonucleotide), or a pharmaceutical composition comprising the oligonucleotide, is administered to a subject having a disease, disorder or condition associated with hepcidin deficiency or suppression such that serum iron saturation is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to serum iron saturation prior to administration of one or more oligonucleotides or pharmaceutical composition. In some embodiments, serum iron saturation is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to serum iron saturation in a subject (e.g., a reference or control subject) not receiving the oligonucleotide or oligonucleotides or pharmaceutical composition or receiving a control oligonucleotide or oligonucleotides, pharmaceutical composition or treatment.

Suitable methods for determining TMPRSS6 expression, the amount or level of TMPRSS6 mRNA, matriptase-2 protein, matriptase-2 activity, or a biomarker related to or affected by modulation of TMPRSS6 expression (e.g., a plasma biomarker), in the subject, or in a sample from the subject, are known in the art. Further, the Examples set forth herein illustrate methods for determining TMPRSS6 expression.

In some embodiments, TMPRSS6 expression, the amount or level of TMPRSS6 mRNA, matriptase-2 protein, matriptase-2 activity, or a biomarker related to or affected by modulation of TMPRSS6 expression, or any combination thereof, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other appropriate biological material obtained or isolated from the subject. In some embodiments, TMPRSS6 expression, the amount or level of TMPRSS6 mRNA, matriptase-2 protein, matriptase-2 activity, or a biomarker related to or affected by modulation of TMPRSS6 expression, or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), or more than one type of sample (e.g., a liver biopsy sample and one or more other type(s) of biopsy sample).

Because of their high specificity, the oligonucleotides provided herein (e.g., RNAi oligonucleotides) specifically target mRNA of target genes (e.g., TMPRSS6 mRNA) of cells and tissue(s), or organs(s) (e.g., in the liver). In preventing disease, the target gene may be one which is required for initiation or maintenance of the disease or which has been identified as being associated with a higher risk of contracting the disease. In treating disease, the oligonucleotide can be brought into contact with the cells, tissue(s), or organ(s) (e.g., liver) exhibiting or responsible for mediating the disease. For example, an oligonucleotide (e.g., an RNAi oligonucleotide) substantially identical to all or part of a wild-type (i.e., native) or mutated gene associated with a disorder or condition associated with hepcidin deficiency or suppression may be brought into contact with or introduced into a cell or tissue type of interest such as a hepatocyte or other liver cell.

Examples of a disease, disorder or condition associated with hepcidin deficiency or suppression include, but are not limited to hemochromatosis (e.g. hereditary hemochromatosis), beta-thalassemia, Polycythemia vera, iron-refractory iron deficiency anemia.

In some embodiments, the target gene may be a target gene from any mammal, such as a human target. Any target gene may be silenced according to the method described herein.

Methods described herein typically involve administering to a subject an effective amount of an oligonucleotide herein (e.g., a RNAi oligonucleotide), that is, an amount that produces or generates a desirable therapeutic result. A therapeutically acceptable amount may be an amount that therapeutically treats a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein (e.g., a composition comprising an RNAi oligonucleotide described herein) either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

In some embodiments, an oligonucleotide herein (e.g., an RNAi oligonucleotide), or a pharmaceutical composition comprising the oligonucleotide, is administered alone or in combination. In some embodiments, the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising an oligonucleotide herein (e.g., an RNAi oligonucleotide), and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein (e.g., an RNAi oligonucleotide), and a pharmaceutically acceptable carrier, or a pharmaceutical composition comprising the oligonucleotide and instructions for treating or delaying progression of a disease, disorder or condition associated with hepcidin deficiency or suppression in a subject in need thereof.

### Definitions

As used herein, the term “antisense oligonucleotide” encompasses a nucleic acid-based molecule which has a sequence complementary to all or part of the target mRNA, in particular seed sequence thereby capable of forming a duplex with a mRNA. Thus, the term “antisense oligonucleotide”, as used herein, may be referred to as a “complementary nucleic acid-based inhibitor” or “guide strand”.

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a disease, disorder, or condition in the subject).

As used herein, “attenuate,” “attenuating,” “attenuation” and the like refers to reducing or effectively halting. As a non-limiting example, one or more of the treatments herein may reduce or effectively halt the onset or progression of iron-refractory iron deficiency anemia, hemochromatosis or beta-thalassemia in a subject. This attenuation may be exemplified by, for example, a decrease in one or more aspects (e.g., symptoms, tissue characteristics, and cellular, inflammatory, or immunological activity, etc.) of iron-refractory iron deficiency anemia, hemochromatosis, or beta-thalassemia, no detectable progression (worsening) of one or more aspects of hemochromatosis or beta-thalassemia, or no detectable aspects of iron-refractory iron deficiency anemia, hemochromatosis, or beta-thalassemia in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “hepatocyte” or “hepatocytes” refers to cells of the parenchymal tissues of the liver. These cells make up about 70%-85% of the liver's mass and manufacture serum albumin, FBN and the prothrombin group of clotting factors (except for Factors 3 and 4). Markers for hepatocyte lineage cells include, but are not limited to, transthyretin (Ttr), glutamine synthetase (GluI), hepatocyte nuclear factor 1a (Hnf1a) and hepatocyte nuclear factor 4a (Hnf4a). Markers for mature hepatocytes may include, but are not limited to, cytochrome P450 (Cyp3a11), cytochrome P450 3A4 (CYP3A4), fumarylacetoacetate hydrolase (Fah), glucose 6-phosphate (G6p), albumin (Alb) and OC2-2F8. See, e.g., Huch et al. (2013) Nature 494:247-50.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal anti-inflammatory drugs (such as aspirin and phenylbutazone).

As used herein, the term “TMPRSS6” refers to transmembrane protease serine 6. The TMPRSS6 gene encodes the matriptase-2 protein which functions in a signaling pathway with hepcidin to regulate iron balance in the body. matriptase-2 As used herein, “labile linker” refers to a linker that can be cleaved (e.g., by acidic pH). A “fairly stable linker” refers to a linker that cannot be cleaved.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified internucleotide linkage may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. When referring to an oligonucleotide being modified, it may be understood to refer to an oligonucleotide that comprises a at least one modified nucleotide therein. When referring to an oligonucleotide being fully modified, it may be understood to refer to an oligonucleotide wherein all nucleotides therein are modified nucleotides. Said modified nucleotides need not comprise the same modifications.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand. The structure is said to be nicked by a discontinuity between the backbone of the sense and antisense strand, typically by a discontinuity between the pentose sugars of the adjacent nucleotides of the sense and antisense strands. A “nicked structure” may generally be referred to with this same definition when the loop may or may not be a tetraloop (e.g., one may refer to a nicked structure when the sense strand forms a triloop, tetraloop or other type of loop as disclosed herein).

As used herein, “oligonucleotide” refers to a short nucleic acid (e.g., less than about 100 nucleotides in length). An oligonucleotide may be single-stranded (ss) or double-stranded (ds). An oligonucleotide may comprise deoxyribonucleosides, ribonucleosides, or a combination of both. In some embodiments, a double-stranded oligonucleotide comprising ribonucleosides is referred to as “dsRNA”. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (DsiRNA), antisense oligonucleotide, short siRNA or ss siRNA.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at (or proximal to) the 5′ terminus or 3′ terminus of an oligonucleotide. In certain embodiments, the overhang is a 3′ or 5′ overhang on the antisense strand or sense strand of an oligonucleotide.

As used herein, features may be described as being provided “in”, “on” or “at” the 3′ end or 5′ end of a strand. Nucleotide features described at a given strand end, such as sugar modifications, internucleotide modifications, nucleotide mismatches and overhangs for instance, are understood to refer to the nucleotide(s) of that strand proximal to the defined strand end (e.g., at 3′ end of the sense strand).

As used herein, “phosphate analog” refers to a chemical moiety that mimics the electrostatic and/or steric properties of a phosphate group. In some embodiments, the phosphate analog mimics the electrostatic and/or steric properties of a phosphate group in biologic systems. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, e.g., US Patent Publication No. 2019-0177729. Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “reduced expression” of a gene (e.g., TMPRSS6) refers to a decrease in the amount or level of RNA transcript (e.g., TMPRSS6 mRNA) or protein encoded by the gene and/or a decrease in the amount or level of activity of the gene in a cell, a population of cells, a sample, or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising TMPRSS6 mRNA) may result in a decrease in the amount or level of TMPRSS6 mRNA, matriptase-2 protein and/or activity (e.g., via degradation of TMPRSS6mRNA by the RNAi pathway) when compared to a cell that is not treated with the oligonucleotide. Similarly, and as used herein, “reducing expression” refers to an act that results in reduced expression of a gene (e.g., TMPRSS6).

As used herein, “reduction of TMPRSS6 expression” refers to a decrease in the amount or level of TMPRSS6 mRNA, matriptase-2 protein and/or matriptase-2 activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., an oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence. A region of complementarity may be of a given length and be identified by a number of contiguous nucleotides (e.g., at least 15 contiguous nucleotides in length), referring to a number of nucleotides contiguously linked together through internucleotide linkages.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., TMPRSS6 mRNA) or (b) a single-stranded oligonucleotide having a single antisense strand, where that antisense strand (or part of that antisense strand) is used by the Ago2 endonuclease in the cleavage of a target mRNA (e.g., TMPRSS6 mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, non-human primates (NHP) and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide, or lipid, such as a GalNAc moiety for instance) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “target sequence” refers to a contiguous portion of the nucleotide sequence of an mRNA molecule formed during the transcription of a TMPRSS6 gene (e.g., SEQ ID NOs: 853, 854, and 855) including mRNA that is a product of RNA processing of a primary transcription product. In some embodiments, the target portion of the sequence will be at least long enough to serve as a substrate for RNAi-directed cleavage at or near that portion of the nucleotide sequence of an mRNA molecule formed during the transcription of a TMPRSS6 gene. In some embodiments, the target sequence is within the protein coding region of the TMPRSS6 gene. In some embodiments, the target sequence is within the 3′ UTR of the TMPRSS6 gene.

As used herein, “targeting sequence” refers to a nucleotide sequence that is fully or partially complementary to a target sequence.

As used herein, “loop”, “triloop”, or “tetraloop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”). A loop increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a loop (e.g., a tetraloop or triloop) can confer a Tₘ of at least about 50° C., at least about 55° C., at least about 56° C., at least about 58° C., at least about 60° C., at least about 65° C. or at least about 75° C. in 10 mM Na₂HPO₄ to a hairpin comprising a duplex of at least 2 base pairs (bp) in length. In some embodiments, a tetraloop can confer a Tₘ of at least about 50° C., at least about 55° C., at least about 56° C., at least about 58° C., at least about 60° C., at least about 65° C. or at least about 75° C. in 10 mM NaH₂PO₄ to a hairpin comprising a duplex of at least 2 base pairs (bp) in length. In some embodiments, a loop may stabilize a bp in an adjacent stem duplex by stacking interactions. In addition, interactions among the nucleotides in a loop include, but are not limited to, non-Watson-Crick base pairing, stacking interactions, hydrogen bonding and contact interactions (Cheong et al. (1990) Nature 346:680-82; Heus & Pardi (1991) Science 253:191-94). In some embodiments, a loop comprises or consists of 3 to 6 nucleotides and is typically 4 to 5 nucleotides. In certain embodiments, a loop comprises or consists of 3, 4, 5 or 6 nucleotides, which may or may not be modified (e.g., which may or may not be conjugated to a targeting moiety/targeting ligand). In one embodiment, a loop consisting of 3 nucleotides is a triloop. In one embodiment, a loop consisting of 4 nucleotides is a tetraloop. Any nucleotide may be used in the tetraloop and standard IUPAC-IUB symbols for such nucleotides may be used as described in Cornish-Bowden (1985) Nucleic Acids Res. 13:3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al. (1990) Proc. Natl. Acad. Sci. USA 87:8467-71; Antao et al. (1991) Nucleic Acids Res. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) Biochem. 41:14281-92; Shinji et al. (2000) Nippon Kagakkai Koen Yokoshu 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

“Pharmaceutically acceptable” indicates that the substance or composition must be chemically and/or toxicologically suitable for the treatment of mammals.

As used herein, the symbol

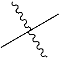

is used to describe an attachment point.

### List of Further Embodiments of the Invention

1. An RNAi oligonucleotide for reducing transmembrane serine protease 6 (TMPRSS6) expression, the oligonucleotide comprising a sense strand and an antisense strand, wherein the sense strand and the antisense strand form a duplex region, wherein the antisense strand comprises a region of complementarity to a TMPRSS6 mRNA target sequence of any one of SEQ ID NOs: 661-852, and wherein the region of complementarity is at least 15 contiguous nucleotides in length.

2. An RNAi oligonucleotide for reducing transmembrane serine protease 6 (TMPRSS6) expression, the oligonucleotide comprising a sense strand and an antisense strand forming a duplex region, wherein the antisense strand comprises a region of complementarity to a TMPRSS6 mRNA target sequence as set forth in any one of SEQ ID NOs: 661-852, and wherein the region of complementarity is at least 15 contiguous nucleotides in length.

3. The RNAi oligonucleotide according to embodiments 1 or 2, wherein the sense strand is 15 to 50 nucleotides in length.

4. The RNAi oligonucleotide according to any one of the preceding embodiments, wherein the sense strand is 18 to 36 nucleotides in length.

5. The RNAi oligonucleotide according to any one of the preceding embodiments, wherein the antisense strand is 15 to 30 nucleotides in length.

6. The RNAi oligonucleotide according to any one of the preceding embodiments, wherein the antisense strand is 22 nucleotides in length and wherein antisense strand and the sense strand form a duplex region of at least 19 nucleotides in length, optionally at least 20 nucleotides in length.

7. The RNAi oligonucleotide according to any one of the preceding embodiments, wherein the antisense strand is 22 nucleotides in length and wherein antisense strand and the sense strand form a duplex region of at least 20 nucleotides in length.

8. The RNAi oligonucleotide according to any one of the preceding embodiments, wherein the region of complementarity is at least 18 contiguous nucleotides in length, optionally the region of complementarity is at least 19 contiguous nucleotides in length.

9. The RNAi oligonucleotiode according to any one of the preceding embodiments, wherein the region of complementarity is at least 19 contiguous nucleotides in length.

10. The RNAi oligonucleotide according to any one of the preceding embodiments, wherein the region of complementarity is at least 19 contiguous nucleotides in length.

11. The RNAi oligonucleotide according to any one of the preceding embodiments, wherein the region of complementarity is at least 20 contiguous nucleotides in length.

12. A double stranded RNAi oligonucleotide for reducing TMPRSS6 expression, the oligonucleotide comprising:
(i) an antisense strand of 19-30 nucleotides in length, wherein the antisense strand comprises a nucleotide sequence comprising a region of complementarity to a TMPRSS6 mRNA target sequence, wherein the region of complementarity is selected from SEQ ID NOs: 1-192, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

13. The RNAi oligonucleotide according to any one of the preceding embodiments, wherein the 3′ end of the sense strand comprises a stem-loop set forth as S1-Lp-S2, wherein S1 is complementary to S2, and wherein Lp forms a loop between S1 and S2 of 3-5 nucleotides in length.

14. The RNAi oligonucleotide according to any one of embodiments 1-12, wherein the sense strand comprises a stem-loop set forth as S1-Lp-S2 proximal the 3′ end, wherein S1 is complementary to S2, and wherein Lp forms a loop of 3-5 nucleotides in length between S1 and S2.

15. The RNAi oligonucleotide according to any one of embodiments 1-12, wherein the sense strand proximal the 3′ end comprises a stem-loop set forth as S1-Lp-S2, wherein S1 is complementary to S2, and wherein Lp forms a loop of 3-5 nucleotides in length between S1 and S2.

16. The RNAi oligonucleotide according to any one of embodiments 13-15, wherein Lp is a triloop or a tetraloop.

17. The RNAi oligonucleotide according to any one of embodiments 13-16, wherein Lp is a tetraloop.

18. The RNAi oligonucleotide according to embodiment 17, wherein the tetraloop comprises the sequence 5′-GAAA-3′.

19. The RNAi oligonucleotide according to any one of embodiments 13-18, wherein the loop is a tetraloop comprising the sequence 5′-GAAA-3′.

20. The RNAi oligonucleotide according to any one of embodiments 13-19, wherein S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length.

21. The RNAi oligonucleotide according to any one of embodiments 13-19, wherein S1 and S2 are each 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length.

22. The RNAi oligonucleotide according to any one of embodiments 13-21, wherein the S1 and S2 are 1-10 nucleotides in length and have the same length.

23. The RNAi oligonucleotide according to any one of embodiments 13-22, wherein S1 and S2 are 6 nucleotides in length.

24. The RNAi oligonucleotide according to any one of embodiments 13-23, wherein the stem-loop comprises the sequence 5′-GCAGCCGAAAGGCUGC-3′ (SEQ ID NO: 856).

25. The RNAi oligonucleotide according to any one of embodiments 13-24, wherein a discontinuity is formed between the sense strand and antisense strand, forming a nicked structure.

26. The RNAi oligonucleotide according to any one of embodiments 13-25, wherein a discontinuity is formed between the sense strand and antisense strand, forming a nicked tetraloop structure.

27. The RNAi oligonucleotide according to any one of the preceding embodiments, wherein the antisense strand comprises a 3′ overhang sequence of one or more nucleotides in length.

28. The RNAi oligonucleotide according to embodiment 27, wherein the overhang comprises purine nucleotides.

29. The RNAi oligonucleotide according to any one of embodiments 27-28, wherein the 3′ overhang sequence is 2 nucleotides in length.

30. The RNAi oligonucleotide according to any one of embodiments 27-29, wherein the 3′ overhang is selected from AA, GG, AG, and GA.

31. The RNAi oligonucleotide according to embodiment 30, wherein the overhang is GG or AA.

32. The RNAi oligonucleotide according to any one of embodiments 30-31, wherein the overhang is GG.

33. The RNAi oligonucleotide according to any one of embodiments 27-29, wherein the 3′ overhang is selected from 5′-AA-3′, 5′-GG-3′, 5′-AG-3′, and 5′-GA-3′.

The RNAi oligonucleotide according to embodiment 33, wherein the overhang is 5′-34. GG-3′ or 5′-AA-3′.

35. The RNAi oligonucleotide according to any one of embodiments 33-34, wherein the overhang is 5′-GG-3′.

36. The RNAi oligonucleotide according to any one of the preceding embodiments, wherein the oligonucleotide comprises at least one modified nucleotide.

37. The RNAi oligonucleotide according to any one of the preceding embodiments, wherein the oligonucleotide is fully modified.

38. The RNAi oligonucleotide according to any one of the preceding embodiments, wherein the all the nucleotides of the oligonucleotide are modified nucleotides.

39. The RNAi oligonucleotide according to any one of embodiments 1-36, wherein the oligonucleotide is partially modified.

40. The RNAi oligonucleotide according to any one of embodiments 36-39, wherein the modified oligonucleotide comprises a targeting ligand conjugated nucleotide.

41. The RNAi oligonucleotide according to any one of embodiments 36-40, wherein the modified nucleotide comprises a targeting ligand conjugated nucleotide.

42. The RNAi oligonucleotide according to any one of embodiments 36-41, wherein the oligonucleotide comprises a 2′-modification.

43. The RNAi oligonucleotide according to any one of embodiment 36-42, wherein the modified nucleotide comprises a 2′-modification.

44. The RNAi oligonucleotide according to any one of embodiments 42-43, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

45. The RNAi oligonucleotide according to any one of embodiments 42-44, wherein the 2′-modification is selected from 2′-fluoro (2′-F) and 2′-O-methyl (2′-OMe).

46. The RNAi oligonucleotide according to any one of embodiments 36-45, wherein the modification is a 2′-modification selected from 2′-fluoro (2′-F) and 2′-O-methyl (2′-OMe).

47. The RNAi oligonucleotide according to any one of embodiments 36-46, wherein about 10-15%, 10%, 11%, 12%, 13%, 14% or 15% of the nucleotides of the sense strand comprise a 2′-fluoro (2′-F) modification.

48. The RNAi oligonucleotide according to any one of embodiments 36-47, wherein about 25-35%, 25%, 26%, 27%, 28%, 29%, 30%, 31%, 32%, 33%, 34% or 35% of the nucleotides of the antisense strand comprise a 2′-fluoro (2′-F) modification.

49. The RNAi oligonucleotide according to any one of embodiments 36-48, wherein about 25-35%, 25%, 26%, 27%, 28%, 29%, 30%, 31%, 32%, 33%, 34% or 35% of the nucleotides of the oligonucleotide comprise a 2′-fluoro (2′-F) modification.

50. The RNAi oligonucleotide according to any one of the preceding embodiments, wherein the sense strand comprises 36 nucleotides with positions 1-36 from 5′ to 3′, wherein positions 8-11 comprise a 2′-fluoro (2′-F) modification.

51. The RNAi oligonucleotide according to any one of the preceding embodiments, wherein the sense strand comprises 36 nucleotides, numbered 5′ to 3′; and all of positions 8-11 of the sense strand comprise a 2′-fluoro (2′-F) modification.

52. The RNAi oligonucleotide according to any one of the preceding embodiments, wherein the sense strand is 36 nucleotides in length, numbered 5′ to 3′; and all of positions 8-11 of the sense strand comprise a 2′-fluoro (2′-F) modification.

53. The RNAi oligonucleotide according to any one of the preceding embodiments, wherein the antisense strand comprises 22 nucleotides with positions 1-22 from 5′ to 3′, and wherein positions 2, 3, 4, 5, 7, 10 and 14 comprise a 2′-fluoro (2′-F) modification. 54. The RNAi oligonucleotide according to any one of the preceding embodiments, wherein the antisense strand comprises 22 nucleotides, numbered 5′ to 3′; and all of positions 2, 3, 4, 5, 7, 10 and 14 of the antisense strand comprise a 2′-fluoro (2′-F) modification.

55. The RNAi oligonucleotide according to any one of the preceding embodiments, wherein the antisense strand is 22 nucleotides in length, numbered 5′ to 3′; and all of positions 2, 3, 4, 5, 7, 10 and 14 of the antisense strand comprise a 2′-fluoro (2′-F) modification.

56. The RNAi oligonucleotide according to any one of the preceding embodiments, wherein the sense strand comprises 36 nucleotides with positions 1-36 from 5′ to 3′, and wherein positions 1-7, 12-27, and 31-36 comprise a 2′-O-methyl modification.

57. The RNAi oligonucleotide according to any one of the preceding embodiments, wherein the sense strand comprises 36 nucleotides, numbered 5′ to 3′; and all of positions 1-7, 12-27, and 31-36 of the sense strand comprise a 2′-O-methyl modification.

58. The RNAi oligonucleotide according to any one of the preceding embodiments, wherein the sense strand is 36 nucleotides in length, numbered 5′ to 3′; and all of positions 1-7, 12-27, and 31-36 of the sense strand comprise a 2′-O-methyl modification.

59. The RNAi oligonucleotide according to any one of the preceding embodiments, wherein the antisense strand comprises 22 nucleotides with positions 1-22 from 5′ to 3′, and wherein positions 1, 6, 8, 9, 11-13, and 15-22 comprise a 2′-O-methyl modification.

60. The RNAi oligonucleotide according to any one of the preceding embodiments, wherein the antisense strand comprises 22 nucleotides, numbered 5′ to 3′; and all of positions 1, 6, 8, 9, 11-13, and 15-22 of the antisense strand comprise a 2′-O-methyl modification.

61. The RNAi oligonucleotide according to any one of the preceding embodiments, wherein the antisense strand is 22 nucleotides in length, numbered 5′ to 3′; and all of positions 1, 6, 8, 9, 11-13, and 15-22 of the antisense strand comprise a 2′-O-methyl modification.

62. The RNAi oligonucleotide according to any one of the preceding embodiments, wherein the sense strand comprises 36 nucleotides and the antisense strand comprises 22 nucleotides, the nucleotides of each one of the strands being numbered 5′ to 3′; all of positions 1-7, 12-27, and 31-36 of the sense strand and positions 1, 6, 8, 9, 11-13, and 15-22 of the antisense strand comprise a 2′-O-methyl modification; and all of positions 8-11 of the sense strand and 2, 3, 4, 5, 7, 10 and 14 of the antisense strand comprise a 2′-fluoro modification.

63. The RNAi oligonucleotide according to any one of the preceding embodiments, wherein the oligonucleotide comprises at least one modified internucleotide linkage.

64. The RNAi oligonucleotide according to embodiment 63, wherein the at least one modified internucleotide linkage is a phosphorothioate linkage.

65. The RNAi oligonucleotide according to any one of the preceding embodiments, the oligonucleotide comprises at least one phosphorothioate linkage.

66. The RNAi oligonucleotide according to any one of the preceding embodiments, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.

67. The RNAi oligonucleotide according to any one of the preceding embodiments, wherein the antisense strand is 22 nucleotides in length, and wherein the antisense strand comprises a phosphorothioate linkage between positions 20 and 21 and between positions 21 and 22, wherein positions are numbered 1-22 from 5′ to 3′.

68. The RNAi oligonucleotide according to any one of the preceding embodiments, wherein the antisense strand comprises 22 nucleotides, numbered 5′ to 3′; and a phosphorothioate linkage is provided between positions 1 and 2, between positions 2 and 3, and between positions 3 and 4, between positions 20 and 21 and between positions 21 and 22 of the antisense strand.

69. The RNAi oligonucleotide according to any one of the preceding embodiments, wherein the sense strand comprises a phosphorothioate linkage between positions 1 and 2, wherein positions are numbered 5′ to 3′.

70. The RNAi oligonucleotide according to any one of the preceding embodiments, wherein
the antisense strand comprises 22 nucleotides;
the nucleotides of each one of the strands are numbered 5′ to 3′;
a phosphorothioate linkage is provided between positions 1 and 2 of the sense strand, and between positions 1 and 2, between positions 2 and 3, and between positions 3 and 4, between positions 20 and 21 and between positions 21 and 22 of the antisense strand.

71. The RNAi oligonucleotide according to any one of the preceding embodiments, wherein the 4′-carbon of the sugar of the 5′-nucleotide of the antisense strand comprises a phosphate analog.

72. The RNAi oligonucleotide according to embodiment 71, wherein the phosphate analog is oxymethyl phosphonate, vinyl phosphonate or malonyl phosphonate, optionally wherein the phosphate analog is a 4′-phosphate analog comprising 4′-oxymethylphosphonate.

73. The RNAi oligonucleotide according to any one of the preceding embodiments, wherein the 5′-terminal nucleotide of the antisense strand comprises a structure according to Chem. 1a (MePhosphonate-40-mU).

74. The RNAi oligonucleotide according to any one of the preceding embodiments, wherein at least one nucleotide of the oligonucleotide is conjugated to one or more targeting ligands.

75. The RNAi oligonucleotide according to embodiment 74, wherein each targeting ligand comprises a carbohydrate, amino sugar, cholesterol, or polypeptide.

76. The RNAi oligonucleotide according to any one of embodiments 13-75, wherein the stem-loop comprises one or more targeting ligands conjugated to one or more nucleotides of the stem-loop.

77. The RNAi oligonucleotide according to any one of embodiments 74-76, wherein the one or more targeting ligands is conjugated to one or more nucleotides of the loop.

78. The RNAi oligonucleotide according to any one of embodiments 13-77, wherein the loop (Lp) comprises 4 nucleotides numbered 1-4 from 5′ to 3′, wherein nucleotides at positions 2, 3, and 4 each comprise one or more targeting ligands, wherein the targeting ligands are the same or different.

79. The RNAi oligonucleotide according to any one of embodiments 74-78, wherein each targeting ligand comprises a N-acetylgalactosamine (GalNAc) moiety.

80. The RNAi oligonucleotide according to embodiment 79, wherein the GalNAc moiety is a monovalent GalNAc moiety, a bivalent GalNAc moiety, a trivalent GalNAc moiety or a tetravalent GalNAc moiety.

81. The RNAi oligonucleotide according to any one of embodiments 79-80, wherein the GalNAc moiety is a monovalent GalNAc moiety.

82. The RNAi oligonucleotide according to any one of embodiments 13-81, wherein up to 4 nucleotides of Lp of the stem-loop are each conjugated to a monovalent GalNAc moiety.

83. The RNAi oligonucleotide according to any one of embodiments 13-82, wherein the loop Lp is 4 nucleotides in length numbered 1-4 from 5′ to 3′, wherein the loop Lp nucleotides at positions 2, 3, and 4 are each conjugated to a monovalent N-acetylgalactosamine (GalNAc) moiety.

84. The RNAi oligonucleotide according to any one of embodiments 13-82, wherein the loop Lp is 4 nucleotides in length, and wherein the second, third and fourth nucleotides of the loop Lp from 5′ to 3′ are each conjugated to a monovalent N-acetylgalactosamine (GalNAc) moiety.

85. The RNAi oligonucleotide according to any one of the preceding embodiments, wherein the region of complementarity is fully complementary to the mRNA target sequence.

86. The RNAi oligonucleotide according to any one of embodiments 1-84, wherein the region of complementarity is partially complementary to the mRNA target sequence.

87. The RNAi oligonucleotide according to any one of embodiments 1-84 and 86, wherein the region of complementarity comprises no more than four mismatches to the mRNA target sequence.

88. The RNAi oligonucleotide according to any one of the preceding embodiments, wherein the region of complementarity is fully complementary to the TMPRSS6 mRNA target sequence at nucleotide positions 2-8 of the antisense strand, wherein nucleotide positions are numbered 5′ to 3′.

89. The RNAi oligonucleotide according to any one of the preceding embodiments, wherein the region of complementarity is fully complementary to the TMPRSS6 mRNA target sequence at nucleotide positions 2-11 of the antisense strand, wherein nucleotide positions are numbered 5′ to 3′.

90. The RNAi oligonucleotide according to any one of the preceding embodiments, wherein the TMPRSS6 mRNA target sequence is as set forth in SEQ ID NO: 844.

91. The RNAi oligonucleotide according to any one of the preceding embodiments, wherein the antisense strand comprises a nucleotide sequence as set forth in SEQ ID NO: 184, and optionally wherein the sense strand comprises a nucleotide sequence as set forth in SEQ ID NO: 844.

92. The RNAi oligonucleotide according to any one of the preceding embodiments, wherein the sense strand comprises a nucleotide sequence of any one of SEQ ID NOs: 579-580, 585-587, 590 and 595-597.

93. The RNAi oligonucleotide according to any one of the preceding embodiments, wherein the sense strand comprises a nucleotide sequence selected from SEQ ID NOs: 579-580, 585-587, 590, and 595-597.

94. The RNAi oligonucleotide according to any one of the preceding embodiments, wherein the antisense strand comprises a nucleotide sequence of any one of SEQ ID NOs: 600-601, 606-608, 611 and 616-618.

95. The RNAi oligonucleotide according to any one of the preceding embodiments, wherein the antisense strand comprises a nucleotide sequence selected from SEQ ID NOs: 600-601, 606-608, 611, and 616-618.

96. The RNAi oligonucleotide according to any one of the preceding embodiments, wherein the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 595 and 616, respectively;
d) SEQ ID NOs: 590 and 611, respectively;
e) SEQ ID NOs: 596 and 617, respectively;
f) SEQ ID NOs: 597 and 618, respectively;
g) SEQ ID NOs: 585 and 606, respectively;
h) SEQ ID NOs: 586 and 607, respectively; and,
i) SEQ ID NOs: 587 and 608, respectively.

97. The RNAi oligonucleotide according to any one of the preceding embodiments, wherein the sense strand and antisense strands comprise nucleotide sequences selected from the group consisting of:
a) SEQ ID NOs: 579 and 600, respectively;
b) SEQ ID NOs: 580 and 601, respectively;
c) SEQ ID NOs: 590 and 611, respectively;
d) SEQ ID NOs: 597 and 618, respectively; and,
e) SEQ ID NOs: 586 and 607, respectively.

98. The RNAi oligonucleotide according to any one of the preceding embodiments, wherein the antisense strand comprises a nucleotide sequence as set forth in SEQ ID NO: 600, and optionally wherein the sense strand comprises a nucleotide sequence as set forth in SEQ ID NO: 579.

99. The RNAi oligonucleotide according to any one of the preceding embodiments, wherein the sense strand comprises a nucleotide sequence as set forth in SEQ ID NO: 579, wherein the antisense strand comprises a nucleotide sequence as set forth in SEQ ID NO: 600.

100. The RNAi oligonucleotide according to any one of embodiments 1-97, wherein the sense strand comprises a nucleotide sequence as set forth in SEQ ID NO: 580, wherein the antisense strand comprises a nucleotide sequence as set forth in SEQ ID NO: 601.

101. The RNAi oligonucleotide according to any one of embodiments 1-97, wherein the sense strand comprises a nucleotide sequence as set forth in SEQ ID NO: 590, wherein the antisense strand comprises a nucleotide sequence as set forth in SEQ ID NO: 611.

102. The RNAi oligonucleotide according to any one of embodiments 1-97, wherein the sense strand comprises a nucleotide sequence as set forth in SEQ ID NO: 597, wherein the antisense strand comprises a nucleotide sequence as set forth in SEQ ID NO: 618.

103. The RNAi oligonucleotide according to any one of embodiments 1-97, wherein the sense strand comprises a nucleotide sequence as set forth in SEQ ID NO: 586, wherein the antisense strand comprises a nucleotide sequence as set forth in SEQ ID NO: 607.

104. The RNAi oligonucleotide according to any one of the preceding embodiments, wherein the antisense strand is 22 nucleotides in length.

105. The RNAi oligonucleotide according to any one of the preceding embodiments, wherein the sense strand is 36 nucleotides in length.

106. The RNAi oligonucleotide according to any one of the preceding embodiments, wherein the sense strand and antisense strands comprise nucleotide sequences selected from the group consisting of:
a) SEQ ID NOs: 621 and 642, respectively;
b) SEQ ID NOs: 622 and 643, respectively;
c) SEQ ID NOs: 637 and 658, respectively;
d) SEQ ID NOs: 632 and 653, respectively;
e) SEQ ID NOs: 638 and 659, respectively;
f) SEQ ID NOs: 639 and 660, respectively;
g) SEQ ID NOs: 627 and 648, respectively;
h) SEQ ID NOs: 628 and 649, respectively; and,
i) SEQ ID NOs: 629 and 650, respectively.

107. The RNAi oligonucleotide according to any one of the preceding embodiments, wherein the sense strand and antisense strands comprise nucleotide sequences selected from the group consisting of:
a) SEQ ID NOs: 621 and 642, respectively;
b) SEQ ID NOs: 622 and 643, respectively;
c) SEQ ID NOs: 632 and 653, respectively;
d) SEQ ID NOs: 639 and 660, respectively; and,
e) SEQ ID NOs: 628 and 649, respectively.

108. The RNAi oligonucleotide according to any one of the preceding embodiments, wherein the sense strand comprises a nucleotide sequence as set forth in SEQ ID NO: 621, wherein the antisense strand comprises a nucleotide sequence as set forth in SEQ ID NO: 642.

109. The RNAi oligonucleotide according to any one of embodiments 1-107, wherein the antisense strand comprises a nucleotide sequence as set forth in SEQ ID NO: 642, and optionally the sense strand comprises a nucleotide sequence as set forth in SEQ ID NO: 621.

110. The RNAi oligonucleotide according to any one of embodiments 1-107, wherein the sense strand comprises a nucleotide sequence as set forth in SEQ ID NO: 622, wherein the antisense strand comprises a nucleotide sequence as set forth in SEQ ID NO: 643.

111. The RNAi oligonucleotide according to any one of embodiments 1-107, wherein the sense strand comprises a nucleotide sequence as set forth in SEQ ID NO: 632, wherein the antisense strand comprises a nucleotide sequence as set forth in SEQ ID NO: 653.

112. The RNAi oligonucleotide according to any one of embodiments 1-107, wherein the sense strand comprises a nucleotide sequence as set forth in SEQ ID NO: 639, wherein the antisense strand comprises a nucleotide sequence as set forth in SEQ ID NO: 660.

113. The RNAi oligonucleotide according to any one of embodiments 1-107, wherein the sense strand comprises a nucleotide sequence as set forth in SEQ ID NO: 628, wherein the antisense strand comprises a nucleotide sequence as set forth in SEQ ID NO: 649.

114. An RNAi oligonucleotide for reducing transmembrane serine protease 6 (TMPRSS6) expression, the oligonucleotide comprising a sense strand and an antisense strand forming a duplex region, wherein
the antisense strand comprises 22 nucleotides and comprises a nucleotide sequence as set forth in SEQ ID NO: 600, and the sense strand comprises 36 nucleotides and comprises a nucleotide sequence as set forth in SEQ ID NO: 579, the nucleotides of each one of the strands being numbered 5′ to 3′;
all of positions 1-7, 12-27, and 31-36 of the sense strand and positions 1, 6, 8, 9, 11-13, and 15-22 of the antisense strand comprise a 2′-O-methyl (2′-OMe) modification, and all of positions 8-11 of the sense strand and 2, 3, 4, 5, 7, 10 and 14 of the antisense strand comprise a 2′-Fluoro (2′-F) modification;
a phosphorothioate linkage is provided between positions 1 and 2 of the sense strand, and between positions 1 and 2, between positions 2 and 3, between positions 3 and 4, between positions 20 and 21 and between positions 21 and 22 of the antisense strand;
the sense strand proximal the 3′ end comprises a stem-loop set forth as S1-Lp-S2, S1 being complementary to S2, Lp forming a loop of 4 nucleotides in length, and wherein the second, third and fourth nucleotides of the loop Lp from 5′ to 3′ are each conjugated to a monovalent N-acetylgalactosamine (GalNAc) moiety; and
the 5′-terminal nucleotide of the antisense strand comprises a structure according to Chem. 1a (MePhosphonate-40-mU):

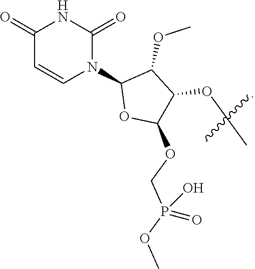 [SMILES: *O[C@@H]1[C@@H](OCP(=O)(O)OC)O[C@@H](n2ccc(=O)[nH]c2=O)[C@@H]1OC]

115. The RNAi oligonucleotide according to embodiment 114, wherein the antisense strand comprises a 3′ overhang of one or more purine nucleotides in length, and optionally wherein the 3′ overhang is 5′-GG-3′.

116. The RNAi oligonucleotide according to any one of embodiments 114-115, wherein the nucleotides of the loop Lp conjugated to the monovalent N-acetylgalactosamine (GalNAc) moiety each comprise a structure according to Chem. 5a:

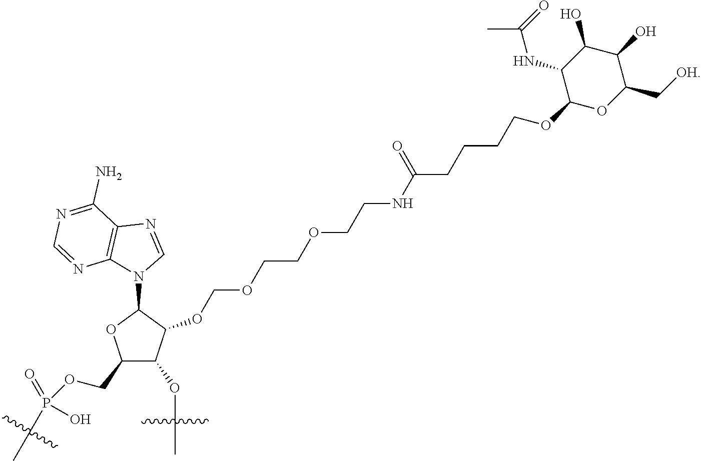 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

117. An RNAi oligonucleotide for reducing TMPRSS6 expression, the oligonucleotide comprising a sense strand and an antisense strand, wherein the sense strand comprises the sequence of the modifications of 5′- and all [mGs][mG][mU][mG][mC][mU][mA][fC][fU][fC][fU][mG][mG][mU][mA][mU][mU][mU][mC][mA][mG][mC][mA][mG][mC][mC][mG][ademA-GalNAc][ademA-GalNAc][ademA-GalNAc][mG][mG][mC][mU][mG][mC]-3′ (SEQ ID NO: 621), and wherein the antisense strand comprises the sequence and all of the modifications of 5′-[MePhosphonate-40-mUs][fGs][fAs][fA][fA][mU][fA][mC][mC][fA][mG][mA][mG][fU][mA][mG][mC][mA][mC][mCs][m Gs][mG]-3′ (SEQ ID NO: 642), wherein mC, mA, mG, mU=2′-OMe ribonucleosides; fA, fC, fG, fU=2′-F ribonucleosides; s=phosphorothioate, and wherein ademA-GalNAc=

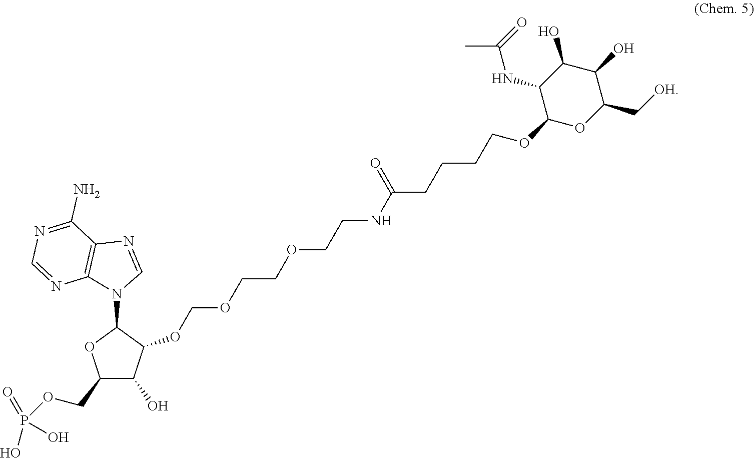 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

118. An RNAi oligonucleotide for reducing TMPRSS6 expression, the oligonucleotide comprising a sense strand and an antisense strand, wherein
the sense strand comprises the sequence and all of the modifications of 5′-[mGs][mG][mU][mG][mC][mU][mA][fC][fU][fC][fU][mG][mG][mU][mA][mU][mU][mU][mC][mA][mG][mC][mA][mG][mC][mC][mG][ademA-GalNAc][ademA-GalNAc][ademA-GalNAc][mG][mG][mC][mU][mG][mC]-3′ (SEQ ID NO: 621);
the antisense strand comprises the sequence and all of the modifications of 5′-[MePhosphonate-40-mUs][fGs][fAs][fA][fA][mU][fA][mC][mC][fA][mG][mA][mG][fU][mA][mG][mC][mA][mC][mCs][m Gs][mG]-3′ (SEQ ID NO: 642);
mC, mA, mG, mU indicate 2′-O-methyl (2′-OMe) modified nucleotides;
fA, fC, fG, fU indicate 2′-Fluoro (2′-F) modified nucleotides;
s indicates a phosphorothioate internucleotide linkage; and
ademA-GalNAc comprises a structure according to Chem. 5a:

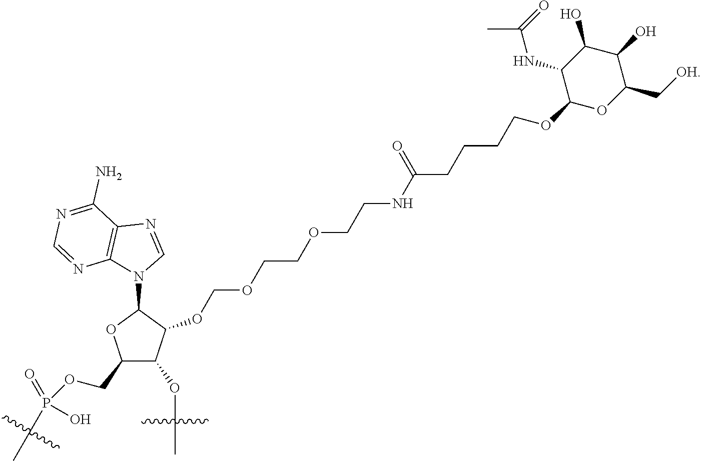 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

119. An RNAi oligonucleotide for reducing TMPRSS6 expression, the oligonucleotide comprising a sense strand and an antisense strand, wherein
the sense strand comprises the sequence and all of the modifications of 5′-[mGs][mG][mU][mG][mC][mU][mA][fC][fU][C][fU][mG][mG][mU][mA][mU][mU][mU][mC][mA][mG][mC][mA][mG][mC][mC][mG][ademA-GalNAc][ademA-GalNAc][ademA-GalNAc][mG][mG][mC][mU][mG][mC]-3′ (SEQ ID NO: 621);
the antisense strand comprises the sequence and all of the modifications of 5′-[MePhosphonate-40-mUs][fGs][fAs][fA][fA][mU][fA][mC][mC][fA][mG][mA][mG][fU][mA][mG][mC][mA][mC][mCs][m Gs][mG]-3′ (SEQ ID NO: 642);
mC, mA, mG, mU indicate 2′-O-methyl (2′-OMe) modified nucleotides;
fA, fC, fG, fU indicate 2′-fluoro (2′-F) modified nucleotides;
s indicates a phosphorothioate internucleotide linkage;
ademA-GalNAc comprises a structure according to Chem. 5a:

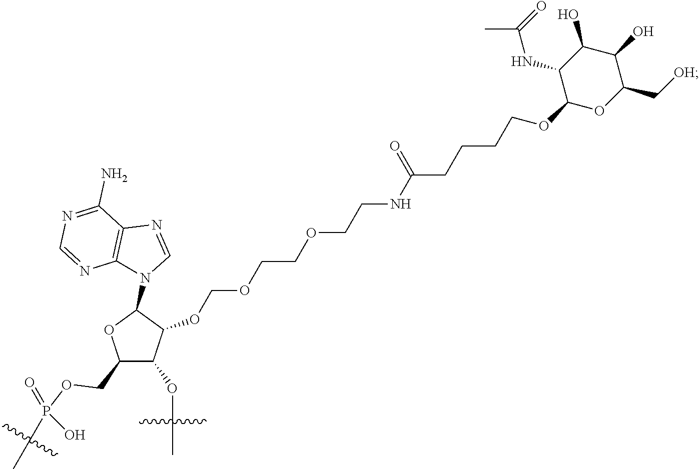 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

and
MePhosphonate-40-mU comprises a structure according to Chem. 1a:

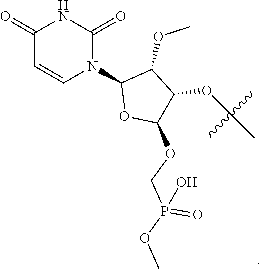 [SMILES: *O[C@@H]1[C@@H](OCP(=O)(O)OC)O[C@@H](n2ccc(=O)[nH]c2=O)[C@@H]1OC]

120. An RNAi oligonucleotide for reducing TMPRSS6 expression, the oligonucleotide comprising a sense strand and an antisense strand, wherein the sense strand comprises the sequence and all of the modifications of 5′-[mGs][mC][mU][mA][mC][mU][mC][fU][fG][fG][fU][mA][mU][mU][mU][mC][mC][mU][mA][mA][mG][mC][mA][mG][mC][mC][mG][ademA-GalNAc][ademA-GalNAc][ademA-10 GalNAc][mG][mG][mC][mU][mG][C]-3′ (SEQ ID NO: 622), and wherein the antisense strand comprises the sequence and all of the modifications of 5′-[MePhosphonate-40-mUs][fUs][fAs][fG][fG][mA][fA][mA][mU][fA][mC][mC][mA][G][mA][mG][mU][mA][mG][mCs][m Gs][mG]-3′ (SEQ ID NO: 643), wherein mC, mA, mG, mU-2′-OMe ribonucleosides; fA, fC, fG, fU=2′-F ribonucleosides; s=phosphorothioate, and wherein ademA-GalNAc=

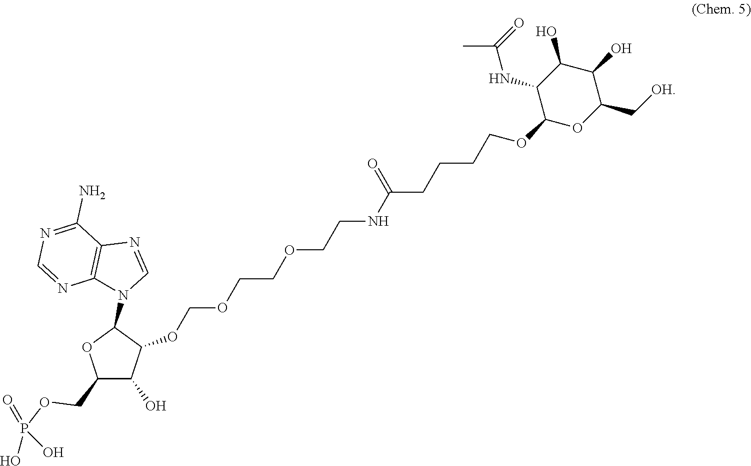 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

121. An RNAi oligonucleotide for reducing TMPRSS6 expression, the oligonucleotide comprising a sense strand and an antisense strand, wherein
the sense strand comprises the sequence and all of the modifications of 5′-[mGs][mC][mU][mA][mC][mU][mC][fU][fG][fG][fU][mA][mU][mU][mU][mC][mC][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 622);
the antisense strand comprises the sequence and all of the modifications of 5′-[MePhosphonate-40-mUs][fUs][fAs][fG][fG][mA][fA][mA][mU][fA][mC][mC][mA][fG][mA][mG][mU][mA][mG][mCs][m Gs][mG]-3′ (SEQ ID NO: 643)
mC, mA, mG, mU indicate 2′-O-methyl (2′-OMe) modified nucleotides;
fA, fC, fG, fU indicate 2′-Fluoro (2′-F) modified nucleotides;
s indicates a phosphorothioate internucleotide linkage; and
ademA-GalNAc comprises a structure according to Chem 5a:

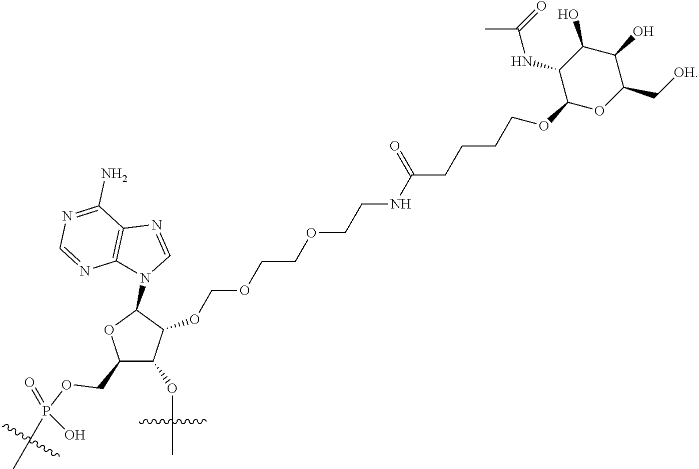 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

122. An RNAi oligonucleotide for reducing TMPRSS6 expression, the oligonucleotide comprising a sense strand and an antisense strand, wherein the sense strand comprises the sequence the of and all of modifications 5′-[mCs][mU][mC][mA][mC][mC][mU][fG][fC][fU][fU][mC][mU][mU][mC][mU][mG][mG][mU][mA][mG][mC][mA][mG][mC][mC][mG][ademA-GalNAc][ademA-GalNAc][ademA-GalNAc][mG][mG][mC][mU][mG][mC]-3′ (SEQ ID NO: 632), and wherein the antisense strand comprises the sequence and all of the modifications of 5′-[MePhosphonate-40-mUs][fAs][fCs][fC][fA][mG][fA][mA][mG][fA][mA][mG][mC][fA][mG][mG][mU][mG][mA][mGs][m Gs][mG]-3′ (SEQ ID NO: 653), wherein mC, mA, mG, mU=2′-OMe ribonucleosides; fA, fC, fG, fU=2′-F ribonucleosides; s=phosphorothioate, and wherein ademA-GalNAc=

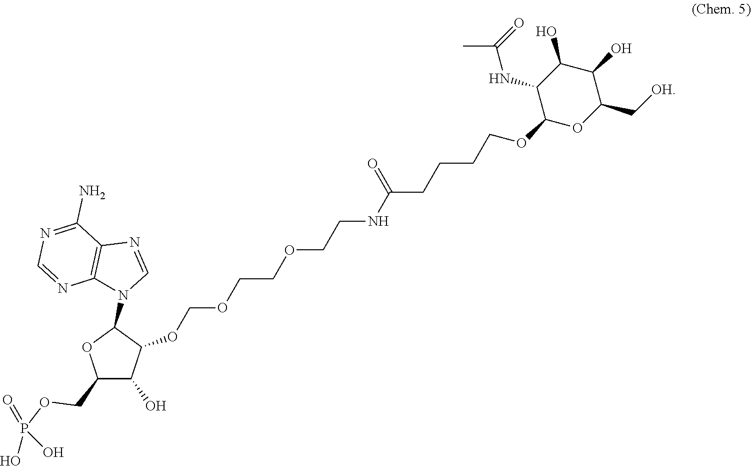 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

123. An RNAi oligonucleotide for reducing TMPRSS6 expression, the oligonucleotide comprising a sense strand and an antisense strand, wherein
the sense strand comprises the sequence and all of the modifications of 5′-[mCs][mU][mC][mA][mC][mC][mU][fG][fC][fU][fU][mC][mU][mU][mC][mU][mG][mG][mU][mA][mG][mC][mA][mG][mC][mC][mG][ademA-GalNAc][ademA-GalNAc][ademA-GalNAc][mG][mG][mC][mU][mG][mC]-3′ (SEQ ID NO: 632);
the antisense strand comprises the sequence and all of the modifications of 5′-[MePhosphonate-40-mUs][fAs][fCs][fC][fA][mG][fA][mA][mG][fA][mA][mG][mC][fA][G][mG][mU][mG][mA][mGs][m Gs][mG]-3′ (SEQ ID NO: 653);
mC, mA, mG, mU indicate 2′-O-methyl (2′-OMe) modified nucleotides;
fA, fC, fG, fU indicate 2′-Fluoro (2′-F) modified nucleotides;
s indicates a phosphorothioate internucleotide linkage; and
ademA-GalNAc comprises a structure according to Chem. 5a:

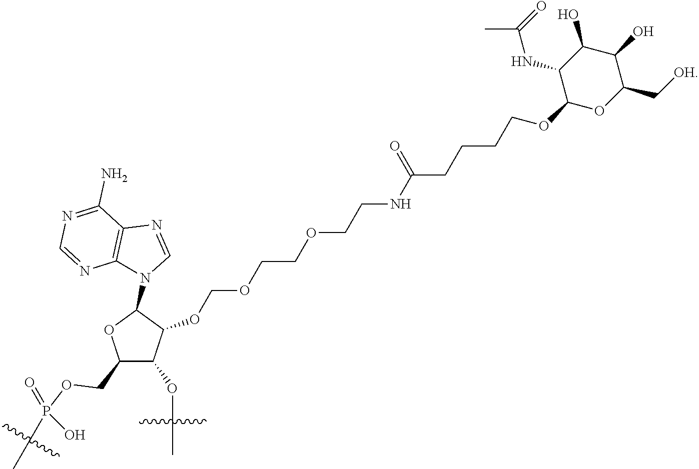 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

124. An RNAi oligonucleotide for reducing TMPRSS6 expression, the oligonucleotide comprising a sense strand and an antisense strand, wherein the sense strand comprises the sequence and all the of modifications of 5′-[mAs][mG][mU][mG][mU][mG][mA][fA][fA][fG][fA][mC][mA][mU][mA][mG][mC][mU][mG][mA][mG][mC][mA][mG][mC][mC][mG][ademA-GalNAc][ademA-GalNAc][ademA-GalNAc][mG][mG][mC][mU][mG][mC]-3′ (SEQ ID NO: 639), and wherein the antisense strand comprises the sequence and all of the modifications of 5′-[MePhosphonate-40-mUs][fCs][fAs][fG][fC][mU][fA][mU][mG][fU][mC][mU][mU][fU][mC][mA][mC][mA][mC][mUs][m Gs][mG]-3′ (SEQ ID NO: 660), wherein mC, mA, mG, mU=2′-Ome ribonucleosides; fA, fC, fG, fU=2′-F ribonucleosides; s=phosphorothioate, and wherein ademA-GalNAc=

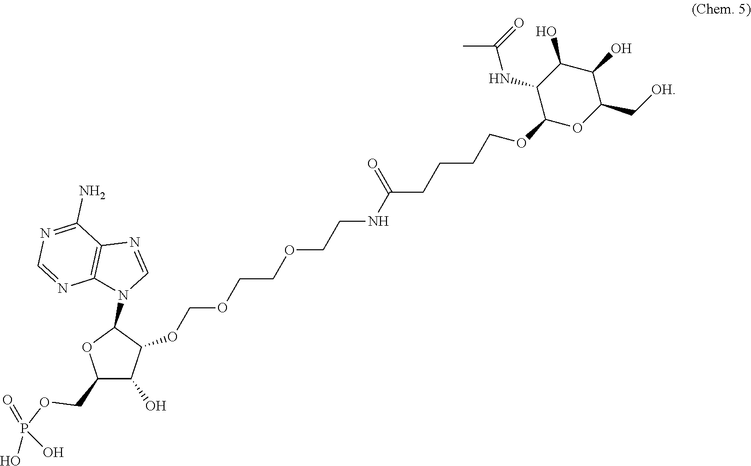 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

125. An RNAi oligonucleotide for reducing TMPRSS6 expression, the oligonucleotide comprising a sense strand and an antisense strand, wherein
the sense strand comprises the sequence and all of the modifications of 5′-[mAs][mG][mU][mG][mU][mG][mA][fA][fA][fG][fA][mC][mA][mU][mA][mG][mC][mU][mG][mA][mG][mC][mA][mG][mC][mC][mG][ademA-GalNAc][ademA-GalNAc][ademA-GalNAc][mG][mG][mC][mU][mG][mC]-3′ (SEQ ID NO: 639);
the antisense strand comprises the sequence and all of the modifications of 5′-[MePhosphonate-40-mUs][fCs][fAs][fG][fC][mU][fA][mU][mG][fU][mC][mU][mU][fU][mC][mA][mC][mA][mC][mUs][m Gs][mG]-3′ (SEQ ID NO: 660);
mC, mA, mG, mU indicate 2′-O-methyl (2′-OMe) modified nucleotides;
fA, fC, fG, fU indicate 2′-Fluoro (2′-F) modified nucleotides;
s indicates a phosphorothioate internucleotide linkage; and
ademA-GalNAc comprises a structure according to Chem. 5a:

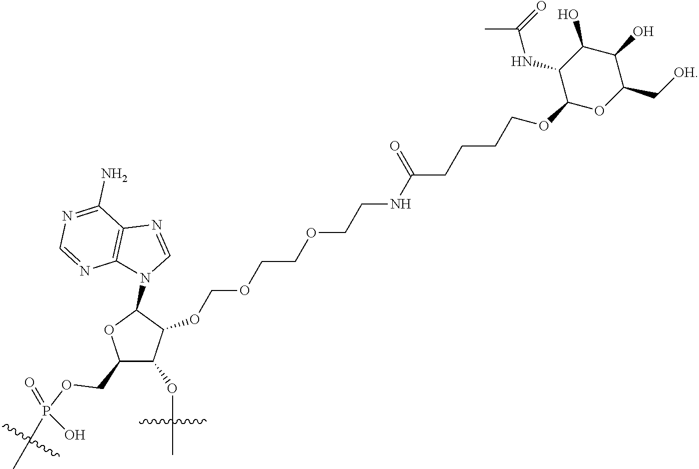 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

126. An RNAi oligonucleotide for reducing TMPRSS6 expression, the oligonucleotide comprising a sense strand and an antisense strand, wherein the sense strand comprises the sequence and all of the modifications of 5′-[mGs][mG][mG][mU][mG][mC][mA][fC][fU][fA][fU][mG][mG][mC][mU][mU][mG][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 628), and wherein the antisense strand comprises the sequence and all of the modifications of 5′-[MePhosphonate-40-mUs][fUs][fAs][fC][fA][mA][fG][mC][mC][fA][mU][mA][mG][fU][mG][mC][mA][mC][mC][mCs][m Gs][mG]-3′ (SEQ ID NO: 649), wherein mC, mA, mG, mU=2′-OMe ribonucleosides; fA, fC, fG, fU=2′-F ribonucleosides; s=phosphorothioate, and wherein ademA-GalNAc=

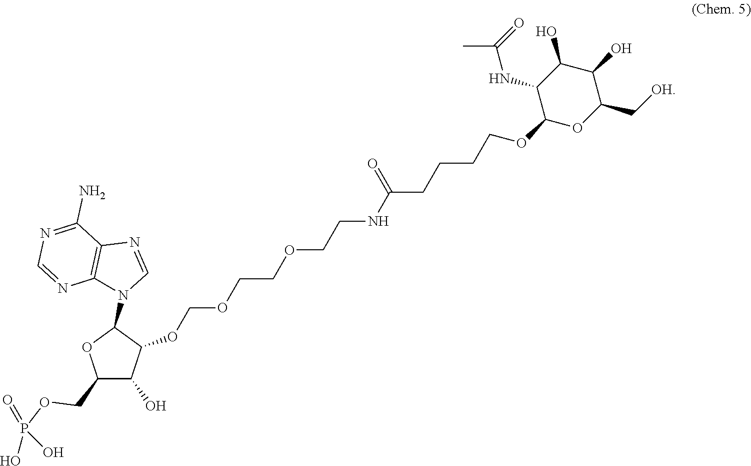 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

127. An RNAi oligonucleotide for reducing TMPRSS6 expression, the oligonucleotide comprising a sense strand and an antisense strand, wherein
the sense strand comprises the sequence and all of the modifications of 5′-[mGs][mG][mG][mU][mG][mC][mA][fC][fU][fA][fU][mG][mG][mC][mU][mU][mG][mU][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 628);
the antisense strand comprises the sequence and all of the modifications of 5′-[MePhosphonate-40-mUs][fUs][fAs][fC][fA][mA][fG][mC][mC][fA][mU][mA][mG][fU][mG][mC][mA][mC][mC][mCs][m Gs][mG]-3′ (SEQ ID NO: 649);
mC, mA, mG, mU indicate 2′-O-methyl (2′-OMe) modified nucleotides;
fA, fC, fG, fU indicate 2′-Fluoro (2′-F) modified nucleotides;
s indicates a phosphorothioate internucleotide linkage; and
ademA-GalNAc comprises a structure according to Chem. 5a:

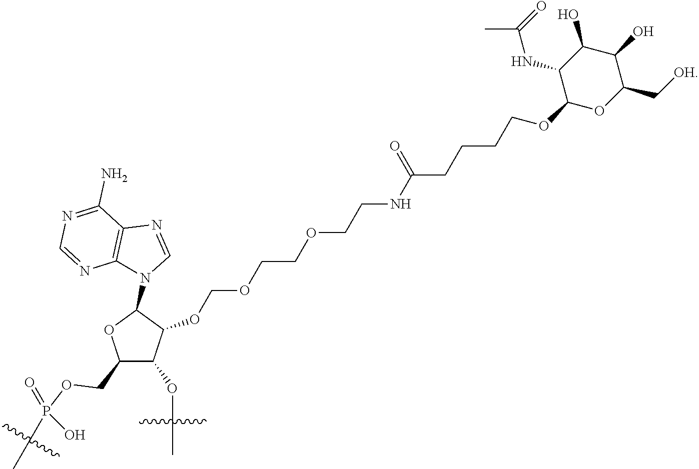 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(*)(=O)O]

128. The RNAi oligonucleotide according to any one of embodiments 116-127, wherein MePhosphonate-40-mUs comprises a structure according to Chem. 1:

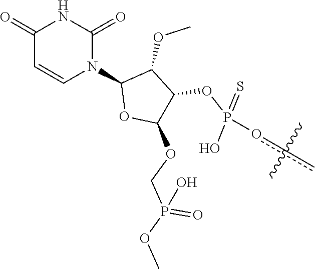

129. The RNAi oligonucleotide according to any one of embodiments 116-127, wherein MePhosphonate-40-mU comprises a structure according to Chem. 1a:

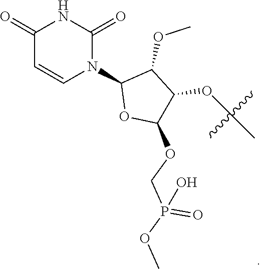 [SMILES: *O[C@@H]1[C@@H](OCP(=O)(O)OC)O[C@@H](n2ccc(=O)[nH]c2=O)[C@@H]1OC]

130. An RNAi oligonucleotide for reducing TMPRSS6 expression comprising a sense strand and an antisense strand, wherein the sense strand comprises SEQ ID NO: 621 and the antisense strand comprises SEQ ID NO: 642, wherein the antisense strand comprises a region of complementarity to a TMPRSS6 RNA transcript, and wherein the oligonucleotide is in the form of a conjugate having the structure as shown in FIGS. 10A-B.

131. An RNAi oligonucleotide for reducing TMPRSS6 expression comprising a sense strand and an antisense strand, wherein the sense strand comprises SEQ ID NO: 621 and the antisense strand comprises SEQ ID NO: 642, wherein the antisense strand comprises a region of complementarity to a TMPRSS6 RNA transcript, and wherein the oligonucleotide is in the form of a conjugate having the structure as shown in FIG. 14A.

132. An RNAi oligonucleotide for reducing TMPRSS6 expression comprising a sense strand and an antisense strand, wherein the sense strand comprises SEQ ID NO: 632 and the antisense strand comprises SEQ ID NO: 653, wherein the antisense strand comprises a region of complementarity to a TMPRSS6 RNA transcript, and wherein the oligonucleotide is in the form of a conjugate having the structure as shown in FIGS. 11A-B.

133. An RNAi oligonucleotide for reducing TMPRSS6 expression comprising a sense strand and an antisense strand, wherein the sense strand comprises SEQ ID NO: 632 and the antisense strand comprises SEQ ID NO: 653, wherein the antisense strand comprises a region of complementarity to a TMPRSS6 RNA transcript, and wherein the oligonucleotide is in the form of a conjugate having the structure as shown in FIG. 14B.

134. An RNAi oligonucleotide for reducing TMPRSS6 expression comprising a sense strand and an antisense strand, wherein the sense strand comprises SEQ ID NO: 639 and the antisense strand comprises SEQ ID NO: 660, wherein the antisense strand comprises a region of complementarity to a TMPRSS6 RNA transcript, and wherein the oligonucleotide is in the form of a conjugate having the structure as shown in FIGS. 12A-B.

135. An RNAi oligonucleotide for reducing TMPRSS6 expression comprising a sense strand and an antisense strand, wherein the sense strand comprises SEQ ID NO: 639 and the antisense strand comprises SEQ ID NO: 660, wherein the antisense strand comprises a region of complementarity to a TMPRSS6 RNA transcript, and wherein the oligonucleotide is in the form of a conjugate having the structure as shown in FIG. 14C.

136. An RNAi oligonucleotide for reducing TMPRSS6 expression comprising a sense strand and an antisense strand, wherein the sense strand comprises SEQ ID NO: 628 and the antisense strand comprises SEQ ID NO: 649, wherein the antisense strand comprises a region of complementarity to a TMPRSS6 RNA transcript, and wherein the oligonucleotide is in the form of a conjugate having the structure as shown in FIG. 13A-B.

137. An RNAi oligonucleotide for reducing TMPRSS6 expression comprising a sense strand and an antisense strand, wherein the sense strand comprises SEQ ID NO: 628 and the antisense strand comprises SEQ ID NO: 649, wherein the antisense strand comprises a region of complementarity to a TMPRSS6 RNA transcript, and wherein the oligonucleotide is in the form of a conjugate having the structure as shown in FIG. 14D.

138. A pharmaceutical composition comprising the RNAi oligonucleotide according to any one of the preceding embodiments, and a pharmaceutically acceptable carrier, delivery agent or excipient.

139. A method for treating a subject having a disease, disorder or condition associated with hepcidin deficiency or suppression, the method comprising administering to the subject a therapeutically effective amount of the RNAi oligonucleotide according to any one of embodiments 1-137, or the pharmaceutical composition according to embodiment 138, thereby treating the subject.

140. The method according to embodiment 139, wherein (i) hepcidin expression is increased; (ii) serum iron levels are decreased; (iii) serum iron saturation is decreased; or (iv) any combination of (i)-(iii), after administering the RNAi oligonucleotide.

141. A method of delivering an oligonucleotide to a subject, the method comprising administering pharmaceutical composition of embodiment 138 to the subject.

142. A method for reducing TMPRSS6 expression in a cell, a population of cells or a subject, the method comprising the step of:
i) contacting the cell or the population of cells with the RNAi oligonucleotide according to any one of embodiments 1-137, or the pharmaceutical composition of embodiment 138; or
ii) administering to the subject the RNAi oligonucleotide according to any one of embodiments 1-137, or the pharmaceutical composition of embodiment 138.

143. The method according to embodiment 142, wherein reducing TMPRSS6 expression comprises reducing an amount or level of TMPRSS6 mRNA, an amount or level of matriptase-2 protein, or both.

144. The method according to embodiment 142 or 143, wherein reducing TMPRSS6 expression results in (i) an increase in hepcidin production; (ii) a decrease in serum iron saturation; (iii) a decrease in serum iron; or (iv) any combination of (i)-(iii).

145. The method according to any one of embodiments 142-144, wherein the subject has a disease, disorder or condition associated with hepcidin deficiency or suppression.

146. The method according to any one of embodiments 139-140 and 145, wherein the disease, disorder or condition associated with hepcidin deficiency is hemochromatosis such hereditary hemochromatosis.

147. The method according to any one of embodiments 139-140 and 145, wherein the disease, disorder or condition associated with hepcidin deficiency is beta-thalassemia.

148. The method according to any one of embodiments 139-140 and 145, wherein the disease, disorder or condition associated with hepcidin suppression is polycythaemia vera.

149. The method according to any one of embodiments 139-148, wherein the RNAi oligonucleotide, or pharmaceutical composition, is administered in combination with a second composition or therapeutic agent.

150. Use of the RNAi oligonucleotide according to any one of embodiments 1-137, or the pharmaceutical composition according to embodiment 138, in the manufacture of a medicament for the treatment of a disease, disorder or condition associated with hepcidin deficiency or suppression, optionally for the treatment of hemochromatosis such as hereditary hemochromatosis, polycythaemia vera, or beta-thalassemia.

151. The RNAi oligonucleotide according to any one of embodiments 1-137, or the pharmaceutical composition according to embodiment 138, for use as a medicament.

152. The RNAi oligonucleotide according to any one of embodiments 1-137, or the pharmaceutical composition according to embodiment 138, for use as a medicament.

153. The RNAi oligonucleotide according to any one of embodiments 1-137, or the pharmaceutical composition of embodiment 138, for use, or adaptable for use, in the treatment of a disease, disorder or condition associated with hepcidin deficiency or suppression, optionally for the treatment of hemochromatosis such as hereditary hemochromatosis, polycythaemia vera, or beta-thalassemia.

154. The RNAi oligonucleotide according to any one of embodiments 1-137, or the pharmaceutical composition according to 138, for use in the treatment of hemochromatosis such as hereditary hemochromatosis.

155. The RNAi oligonucleotide according to any one of embodiments 1-137, or the pharmaceutical composition according to 138, for use in the treatment of polycythaemia vera.

156. The RNAi oligonucleotide according to any one of embodiments 1-137, or the pharmaceutical composition according to 138, for use in the treatment of beta-thalassemia.

157. The RNAi oligonucleotide according to any one of embodiments 1-137, or the pharmaceutical composition according to 138, for use in the treatment of hemochromatosis such as hereditary hemochromatosis or beta-thalassemia.

158. A kit comprising the RNAi oligonucleotide according to any one of embodiments 1-137, an optional pharmaceutically acceptable carrier, and a package insert comprising instructions for administration to a subject having a disease, disorder or condition associated with hepcidin deficiency or suppression.

159. A kit comprising the RNAi oligonucleotide according to any one of embodiments 1-137, or the pharmaceutical composition of embodiment 138, and a package insert comprising instructions for administration to a subject having a disease, disorder or condition associated with hepcidin deficiency or suppression.

160. The use of embodiment 150, the RNAi oligonucleotide or pharmaceutical composition for use, or adaptable for use, according to any one of embodiments 151-157, or the kit according to any one of embodiments 158-159, wherein the disease, disorder or condition associated with hepcidin deficiency or suppression is hemochromatosis such as hereditary hemochromatosis, polycythaemia vera, or beta-thalassemia.

### EXAMPLES

### Example 1: Preparation of RNAi Oligonucleotides

### Oligonucleotide Synthesis and Purification

The oligonucleotides (RNAi oligonucleotides) described in the foregoing Examples were chemically synthesized using methods described herein. Generally, RNAi oligonucleotides are synthesized using solid phase oligonucleotide synthesis methods as described for 19-23mer siRNAs (see, e.g., Scaringe et al. (1990) Nucleic Acids Res. 18:5433-5441 and Usman et al. (1987) J. Am. Chem. Soc. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) Cold Spring Harb Perspect Biol. 9(1):a023812; Beaucage S. L., Caruthers M. H. Studies on Nucleotide Chemistry V: Deoxynucleoside Phosphoramidites—A New Class of Key Intermediates for Deoxypolynucleotide Synthesis, Tetrahedron Lett. 1981; 22:1859-62. Doi: 10.1016/S0040-4039(01)90461-7). dsRNAi oligonucleotides having a 19mer core sequence were formatted into constructs having a 25mer sense strand and a 27mer antisense strand to allow for processing by the RNAi machinery. The 19mer core sequence is complementary to a region in the TMPRSS6 mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) Methods Mol. Biol. 20:81-114; Wincott et al. (1995) Nucleic Acids Res. 23:2677-84). The oligomers were purified using ion-exchange high performance liquid chromatography (IE-HPLC) on an Amersham Source 15Q column (1.0 cmx25 cm; Amersham Pharmacia Biotech) using a 15 min. step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectrometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 UM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5 minutes in RNA buffer (IDT) and were allowed to cool to room temperature before use. The RNAi oligonucleotides were stored at −20° C. Single strand RNA oligomers were stored lyophilized or in nuclease-free water at −80° C.

### Example 2: Generation of TMPRSS6-Targeting GalNAc-Conjugated RNAi Oligonucleotides

Transmembrane protease, serine 6 (TMPRSS6) is a type II transmembrane serine protease found on the cell surface. The protein functions in a signaling pathway with hepcidin to regulate iron balance in the body.

Identification of TMPRSS6 mRNA Target Sequences

To generate TMPRSS6 RNAi oligonucleotides, a computer-based algorithm was used to computationally identify TMPRSS6 mRNA target sequences suitable for assaying inhibition of TMPRSS6 expression by the RNAi pathway. The algorithm provided RNAi oligonucleotide guide (antisense) strand sequences each having a region of complementarity to a suitable TMPRSS6 mRNA target sequence of human (Hs) or murine (Mm) mRNA (e.g., SEQ ID NOS: 853 and 854, respectively; Table 1). Due to sequence conservation across species, some of the TMPRSS6 mRNA target sequences identified for human TMPRSS6 mRNA are homologous to the corresponding TMPRSS6 mRNA target sequence of murine (Mm) TMPRSS6 mRNA (SEQ ID NO:854; Table 1) and/or cynomolgus monkey (Mf) TMPRSS6 mRNA (SEQ ID NO:855; Table 1). TMPRSS6 RNAi oligonucleotides comprising a region of complementarity to homologous TMPRSS6 mRNA target sequences with nucleotide sequence similarity are predicted to have the ability to target homologous TMPRSS6 mRNAs (e.g., human TMPRSS6 and monkey TMPRSS6 mRNAs).

TABLE 1
Exemplary Human TMPRSS6, Monkey TMPRSS6,
and Murine TMPRSS6 mRNA Sequences

| Species | GenBank Ref Seq # | SEQ ID NO |
|---|---|---|
| Human (Hs) | NM_001289000.2 | 853 |
| Murine (Mm) | NM_001355601.1 | 854 |
| Cynomolgus monkey (Mf) | XM_005567384.2 | 855 |

Specifically, oligonucleotides synthesized as described in Example 1 were used to generate double-stranded RNAi oligonucleotides comprising a nicked tetraloop GalNAc-conjugated structure (referred to herein as “GalNAc-conjugated TMPRSS6 oligonucleotides” or “GalNAc-TMPRSS6 oligonucleotides”) having a 36-mer passenger strand and a 22-mer guide strand. Further, the nucleotide sequences comprising the passenger strand and guide strand have a distinct pattern of modified nucleotides and phosphorothioate linkages. Three of the nucleotides comprising the tetraloop were each conjugated to a GalNAc moiety (CAS #14131-60-3). The modification pattern of each strand is illustrated below:

Sense Strand:
5′-[mXs][mX][mX][mX][mX][mX][mX][fX][fX][fX][fX]
[mX][mX][mX][mX][mX][mX][mX][mX][mX][mX][mX][mX]
[mX][mX][mX][mX][ademX-GalNAc][ademX-GalNAc]
[ademX-GalNAc][mX][mX][mX][mX][mX][mX]-3′

### Hybridized to:

Antisense Strand:
5′-[MePhosphonate-4O-mXs][fXs][fXs][fX][fX][mX]
[fX][mX][mX][fX][mX][mX][mX][fX][mX][mX][mX]
[mX][mX][mXs][mXs][mX]-3′

TABLE 2
Modification key

| Symbol | Modification/linkage |
|---|---|
| [MePhosphonate-4O- | 4′-O-monomethylphosphonate-2′-O-methyl modified |
| mXs] | nucleotide with a phosphorothioate linkage to the |
| | neighboring nucleotide |
| [ademX-GalNAc] | GalNAc attached to a nucleotide |
| [mXs] | 2′-O-methyl modified nucleotide with a |
| | phosphorothioate linkage to the neighboring nucleotide |
| [fXs] | 2′-fluoro modified nucleotide with a phosphorothioate |
| | linkage to the neighboring nucleotide |
| [mX] | 2′-O-methyl modified nucleotide with phosphodiester |
| | linkages to neighboring nucleotides |
| [fX] | 2′-fluoro modified nucleotide with phosphodiester |
| | linkages to neighboring nucleotides |

It is understood that “X” in the above modification patterns and modification key refers to nucleobases.

### In Vitro Cell-Based Assays

The ability of GalNAc-conjugated TMPRSS6 oligonucleotides to reduce TMPRSS6 mRNA was measured using in vitro cell-based assays. Briefly, human Hep3B cells expressing endogenous human TMPRSS6 gene were transfected with GalNAc-conjugated TMPRSS6 oligonucleotides at 1 nM in separate wells of a multi-well cell-culture plate. Cells were maintained for 24 hours following transfection with the modified GalNAc-conjugated TMPRSS6 oligonucleotides and then the amount of remaining TMPRSS6 mRNA from the transfected cells was determined using TAQMAN®-based qPCR assays. Two qPCR assays, a 3′ assay and a 5′ assay, were used to determine TMPRSS6 mRNA levels as measured using PCR probes conjugated to 6-carboxy-fluorescein (FAM). These assays are identified in Table 3 below. Primer pairs were assayed for percent (%) remaining mRNA.

TABLE 3
qPCR Assays

| | | | |
|---|---|---|---|
| 3′ | Forward-1652 | AGCCTGATTGTCTCAACGG | SEQ ID |
| Assay | (F1652) | | NO: 857 |
| | Reverse-1725 | CTGGAAGGTGAATGTCCCAC | SEQ ID |
| | | | NO: 858 |
| | Probe-1676 | ACGAAGAGCAGTGCCAGGAAGG | SEQ ID |
| | | | NO: 859 |
| 5′ | Forward-394 | GTACTCAATCGCCACTTCTCC | SEQ ID |
| Assay | (F394) | | NO: 860 |
| | Reverse-539 | GAATAGACGGAGCTGGAGTTG | SEQ ID |
| | | | NO: 861 |
| | Probe-450 | CAGTGAAACCGCCAAAGCCCAG | SEQ ID |
| | | | NO: 862 |

The results indicated that GalNAc-conjugated TMPRSS6 oligonucleotides designed to target TMPRSS6 mRNA inhibited TMPRSS6 expression in cells, as determined by a reduced amount of TMPRSS6 mRNA in GalNAc-conjugated TMPRSS6 oligonucleotide-transfected cells relative to control cells, and indicated that the nucleotide sequences comprising the GalXC-conjugated oligonucleotides are useful for generating RNAi oligonucleotides to inhibit TMPRSS6 expression.

### Example 3: GalNAc-Conjugated TMPRSS6 RNAi Oligonucleotides Inhibit Human and Murine TMPRSS6 Expression In Vivo

To evaluate the ability of RNAi oligonucleotides to reduce TMPRSS6 expression in vivo, an HDI mouse model was used.

Nine GalNAc-conjugated TMPRSS6 oligonucleotides shown in Table 4 were evaluated. The oligonucleotides selected were based on the nucleotide sequences that were used to generate RNAi oligonucleotides to inhibit TMPRSS6 expression in the screen of Example 2.

Oligonucleotides were evaluated in mice engineered to transiently express human TMPRSS6 mRNA in hepatocytes of the mouse liver. Briefly, 6-8 week-old female CD-1 mice (n=5) were subcutaneously administered the indicated GalNAc-conjugated TMPRSS6 oligonucleotides at a concentration of 1 mg/kg or 2 mg/kg formulated in PBS. A control group of mice (n=5) were administered only PBS. Three days later (72 hours), the mice were hydrodynamically injected (HDI) with 25 μg of DNA plasmid encoding the open reading frame (ORF) human TMPRSS6 gene (pCMV6_TMPRSS6 containing NM_153609 (Cat #: SC306623, Origene)) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine TMPRSS6 mRNA levels. Specifically, RNA was extracted from liver tissue to determine human and endogenous murine TMPRSS6 mRNA levels by qPCR (normalized to the neoR gene). The levels of human TMPRSS6 mRNA were determined using 5′ and 3′ PrimeTime™ qPCR Probe Assays (IDT), which consisted of a primer pair and fluorescently labeled probe specific to human TMPRSS6 mRNA. The levels of murine TMPRSS6 were similarly measured. The percentage of human and endogenous murine TMPRSS6 mRNA remaining in the samples from treated mice was determined using the 2-44Ct (“delta-delta Ct”) method (Livak and Schmittgen (2001) Methods 25:402-408). The values were normalized for transfection efficiency using the NeoR gene included on the DNA plasmid.

TABLE 4
GalNAc-Conjugated Human TMPRSS6 RNAi
Oligonucleotides for HDI screen

| | | Unmodified | Unmodified | Modified | Modified |
|---|---|---|---|---|---|
| | | Sense | Antisense | Sense | Antisense |
| | | Strand | strand | Strand | strand |
| | Species | (SEQ | (SEQ | (SEQ | (SEQ |
| | Targets | ID NO) | ID NO) | ID NO) | ID NO) |
| TMPRSS6- | Hs/Mf | 579 | 600 | 621 | 642 |
| 416 | | | | | |
| TMPRSS6- | Hs/Mf | 580 | 601 | 622 | 643 |
| 0419 | | | | | |
| TMPRSS6- | Hs/Mf | 595 | 616 | 637 | 658 |
| 0615 | | | | | |
| TMPRSS6- | Hs/Mf/ | 590 | 611 | 632 | 653 |
| 0651 | Mm | | | | |
| TMPRSS6- | Hs/Mf | 596 | 617 | 638 | 659 |
| 0654 | | | | | |
| TMPRSS6- | Hs/Mf | 597 | 618 | 639 | 660 |
| 0831 | | | | | |
| TMPRSS6- | Hs/Mf | 585 | 606 | 627 | 648 |
| 1375 | | | | | |
| TMPRSS6- | Hs/Mf | 586 | 607 | 628 | 649 |
| 1546 | | | | | |
| TMPRSS6- | Hs/Mf | 587 | 608 | 629 | 650 |
| 1550 | | | | | |

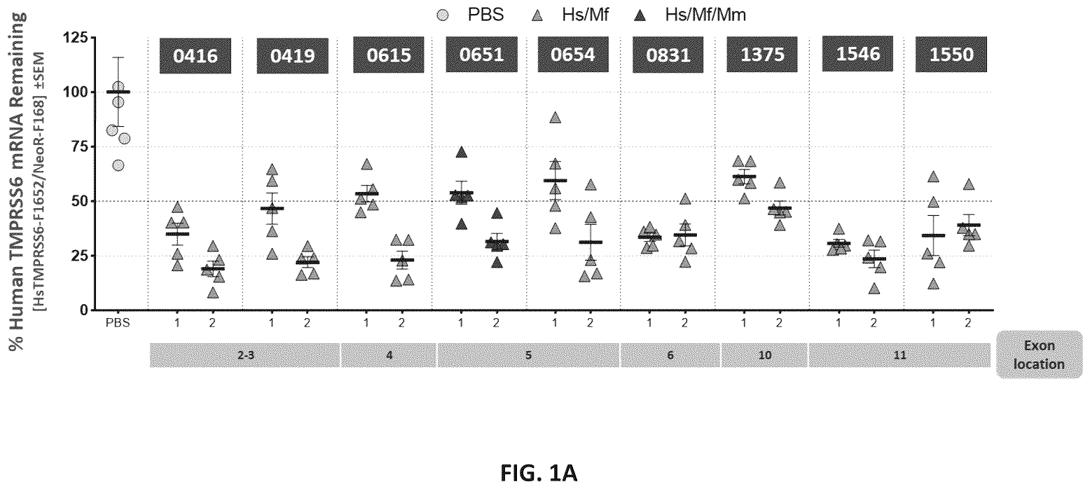

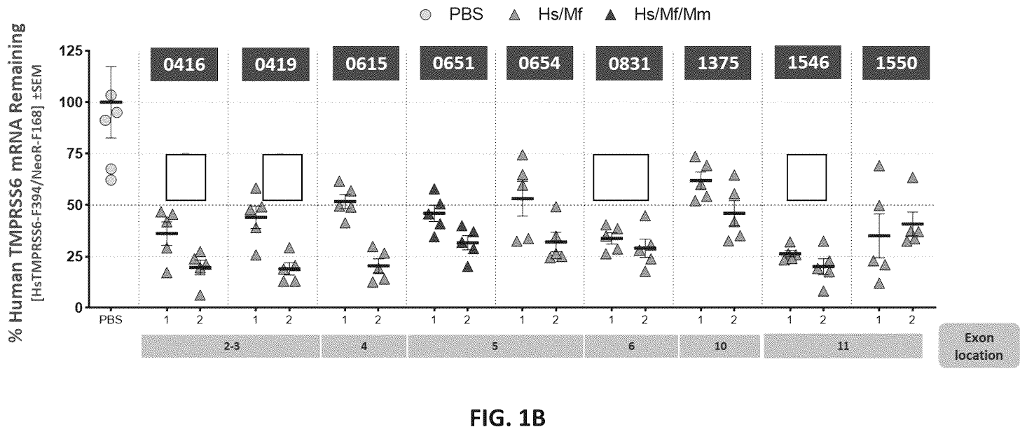

As shown in the 5′ and 3′ qPCR assays in FIGS. 1A and 1B, and Table 5, each oligonucleotide reduced TMPRSS6 expression by at least 50% at 2 mg/kg. TMPRSS6-0416, -0831, and -1546 were the most potent oligonucleotides and reduced TMPRSS6 expression by more than 50% at the lower 1 mg/kg concentration. This data demonstrated that GalNAc-modified oligonucleotides successfully reduced human TMPRSS6 expression in vivo.

TABLE 5
Summary of 5′ and 3′ assays from FIGS. 1A and 1B

| | Hs TMPRSS6-5′/F394 | | | | Hs TMPRSS6-3′/F1652 | | | |
|---|---|---|---|---|---|---|---|---|
| | 1 mg/kg | | 2 mg/kg | | 1 mg/kg | | 2 mg/kg | |
| Construct | % remaining | SEM | % remaining | SEM | % remaining | SEM | % remaining | SEM |
| TMPRSS6-416 | 36.1 | 5.7 | 19.7 | 3.6 | 34.9 | 5.0 | 18.9 | 3.6 |
| TMPRSS6-0419 | 44.0 | 5.5 | 19.0 | 3.0 | 46.6 | 7.2 | 22.0 | 2.5 |
| TMPRSS6-0615 | 51.7 | 3.5 | 20.5 | 3.4 | 53.4 | 3.8 | 23.0 | 4.1 |
| TMPRSS6-0651 | 46.0 | 4.0 | 31.7 | 3.5 | 53.9 | 5.3 | 31.5 | 3.7 |
| TMPRSS6-0654 | 53.1 | 8.5 | 32.0 | 4.7 | 59.4 | 8.7 | 31.2 | 8.2 |
| TMPRSS6-0831 | 33.8 | 2.7 | 29.1 | 4.5 | 33.4 | 1.8 | 34.5 | 5.0 |
| TMPRSS6-1375 | 61.9 | 4.2 | 46.0 | 6.1 | 61.2 | 3.3 | 46.8 | 3.2 |
| TMPRSS6-1546 | 26.4 | 1.5 | 26.1 | 3.9 | 30.6 | 1.7 | 23.5 | 4.1 |
| TMPRSS6-1550 | 35.0 | 10.7 | 40.8 | 5.7 | 34.2 | 9.2 | 39.0 | 4.9 |

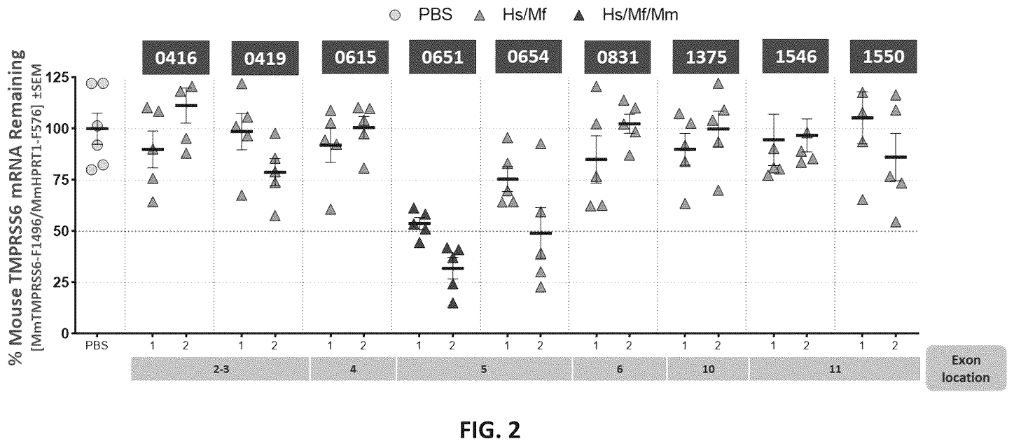

As shown in FIG. 2, GalNAc-conjugated TMPRSS6 RNAi oligonucleotide TMPRSS6-0651, having specificity for murine TMPRSS6 in addition to human TMPRSS6, reduced endogenous murine TMPRSS6 expression by at least 50% at 2 mg/kg. This data demonstrated that GalNAc-modified oligonucleotides successfully reduced endogenous murine TMPRSS6 expression in vivo.

### Example 4: GalNAc-Conjugated TMPRSS6 RNAi Oligonucleotides Inhibit Monkey TMPRSS6 Expression In Vivo

To evaluate the ability of RNAi oligonucleotides to reduce TMPRSS6 expression in vivo, a monkey model was used (Cynomolgus macaques).

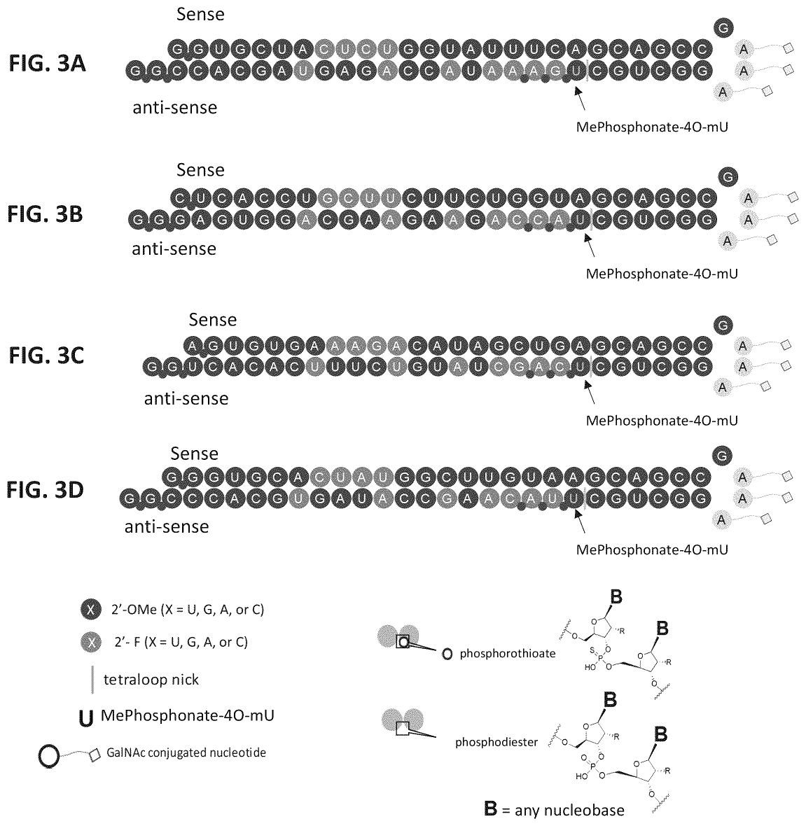

Four modified GalNAc-conjugated TMPRSS6 oligonucleotides shown in Table 6 and depicted in FIGS. 3A-3D were evaluated. The oligonucleotides selected were based on the screen in Example 2 and results of Example 3. Briefly, cynomolgus monkeys, generally referred to herein as monkeys, were subcutaneously administered the indicated GalNAc-conjugated TMPRSS6 oligonucleotides at a concentration of 1 mg/kg or 4 mg/kg formulated in PBS at Day 0, 28, 56, 84, and 112. A control group of monkeys (n=5) were administered only PBS. Total RNA derived from these monkeys were subjected to qRT-PCR analysis to determine TMPRSS6 mRNA levels. Specifically, RNA was extracted from liver tissue to determine monkey TMPRSS6 mRNA levels by qPCR (normalized to the B2M gene) at Day −7, 28, 56, 84, 112, and 168. The levels of monkey TMPRSS6 mRNA were determined using a PrimeTime™ qPCR Probe Assay (IDT), which consisted of a primer pair and fluorescently labeled probe specific to monkey TMPRSS6 mRNA. The percentage of monkey TMPRSS6 mRNA remaining in the samples from treated monkeys was determined using the 2-44Ct (“delta-delta Ct”) method (Livak and Schmittgen (2001) Methods 25:402-408).

TABLE 6
GalNAc-Conjugated Monkey TMPRSS6 RNAi
Oligonucleotides for NHP screen

| | | Unmodified | Unmodified | Modified | Modified |
|---|---|---|---|---|---|
| | | Sense | Antisense | Sense | Antisense |
| | | Strand | strand | Strand | strand |
| | Species | (SEQ | (SEQ | (SEQ | (SEQ |
| | Targets | ID NO) | ID NO) | ID NO) | ID NO) |
| TMPRSS6- | Hs/Mf | 579 | 600 | 621 | 642 |
| 416 | | | | | |
| TMPRSS6- | Hs/Mf/ | 590 | 611 | 632 | 653 |
| 0651 | Mm | | | | |
| TMPRSS6- | Hs/Mf | 597 | 618 | 639 | 660 |
| 0831 | | | | | |
| TMPRSS6- | Hs/Mf | 586 | 607 | 628 | 649 |
| 1546 | | | | | |

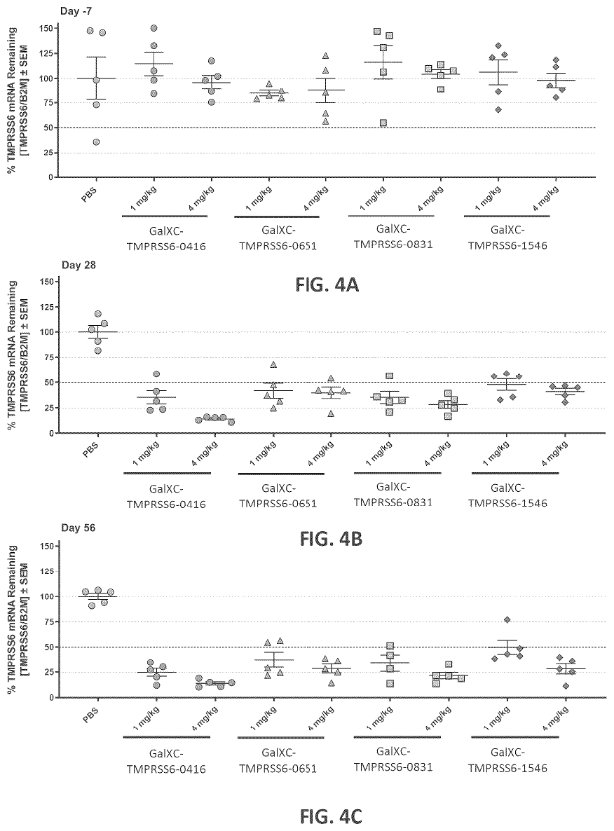

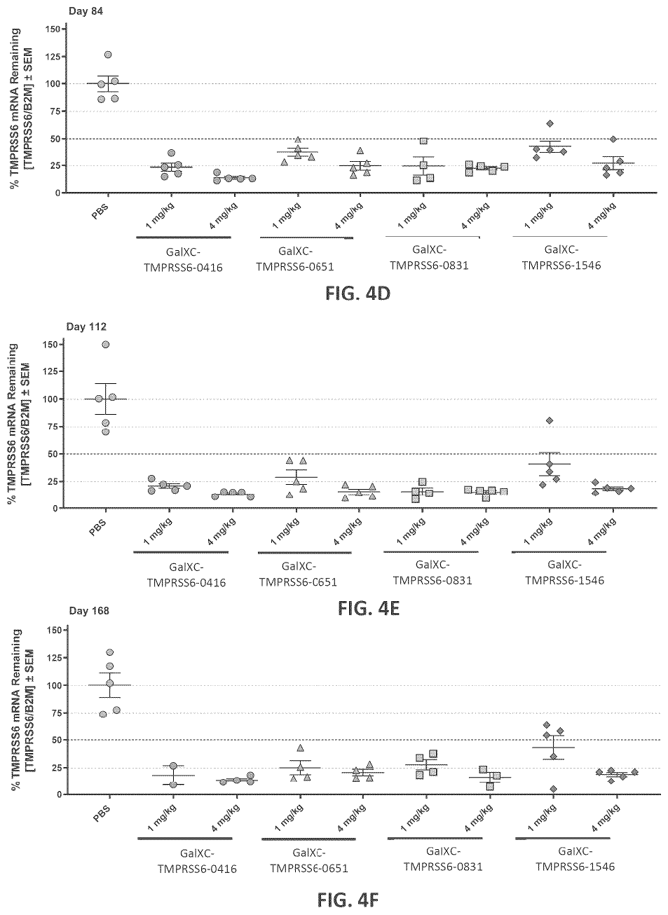

As shown in FIGS. 4A-4F, corresponding to each one of the samples at days-7, 28, 56, 84, 112, and 168 respectively, each oligonucleotide reduced TMPRSS6 expression by at least 50% at both 1 mg/kg and 4 mg/kg throughout the course of the study (i.e., out to 168 days). This data demonstrated that the GalNAc-modified oligonucleotides successfully reduced monkey TMPRSS6 expression in vivo.

### Example 5: GalNAc-Conjugated TMPRSS6 RNAi Oligonucleotides Modulate Iron Homeostasis In Vivo

Loss of function mutations in TMPRSS6 result in elevated hepcidin plasma levels and lead to severe disorders including iron-refractory, iron-deficient anemia. Hepcidin protein is a regulator of iron homeostasis and directs entry of iron into circulation. When levels of hepcidin are high, serum iron levels decrease which can result in anemia. When hepcidin levels are low, in disease states such as hemochromatosis, iron levels rise and overload can occur. TMPRSS6 is known to suppress hepcidin expression and thus inhibition of TMPRSS6 may modulate hepcidin expression and alter serum iron levels and saturation.

To evaluate changes in iron homeostasis, levels of hepcidin, serum iron, and serum iron saturation were measured after inhibition of TMPRSS6. The four modified GalNAc-conjugated TMPRSS6 oligonucleotides shown in Table 6 and depicted in FIGS. 3A-3D were administered to cynomolgus monkeys as described in Example 4. Total RNA derived from these monkeys were subjected to qRT-PCR analysis to determine hepcidin mRNA levels. Specifically, RNA was extracted from liver tissue to determine monkey hepcidin mRNA levels by qPCR (normalized to the B2M gene) at Day-7, 28, 56, 84, 112, and 168. The levels of monkey hepcidin mRNA were determined using a PrimeTime™ qPCR Probe Assay (IDT), which consisted of a primer pair and fluorescently labeled probe specific to monkey TMPRSS6 mRNA. The percentage of monkey hepcidin mRNA remaining in the samples from treated monkeys was determined using the 2-44Ct (“delta-delta Ct”) method (Livak and Schmittgen (2001) Methods 25:402-408).

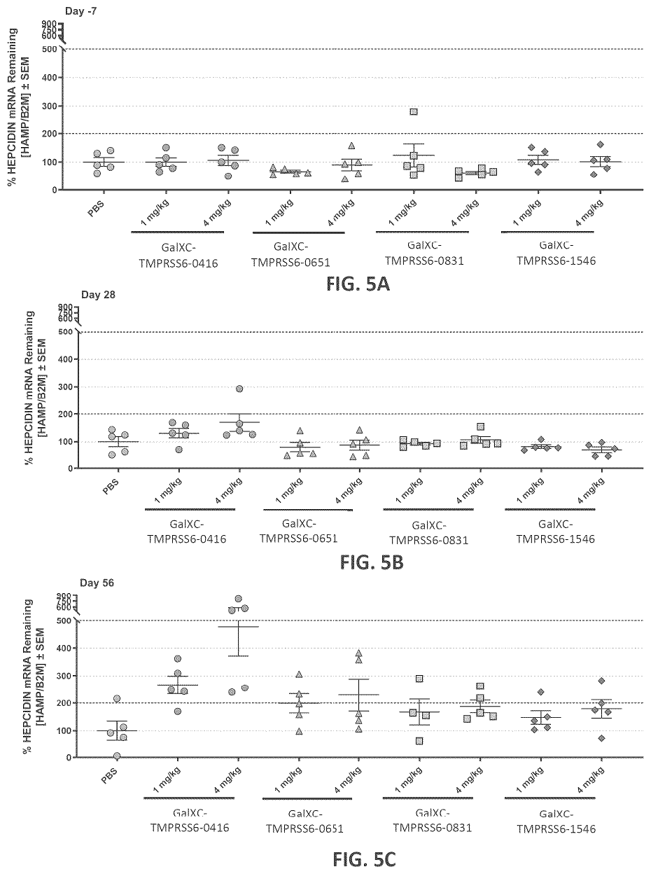

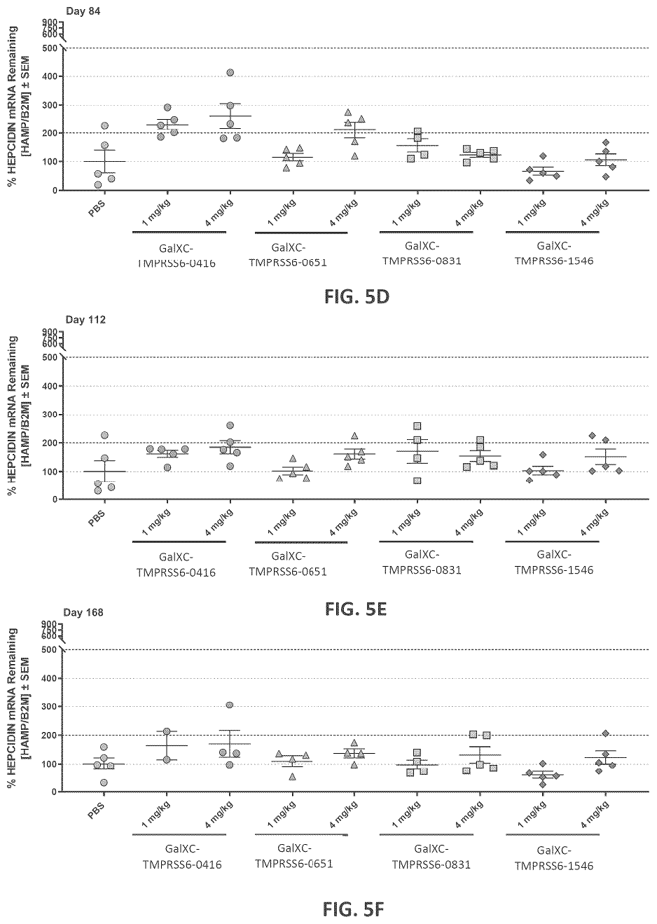

As shown in FIGS. 5A-5F, corresponding to each one of the samples at days-7, 28, 56, 84, 112, and 168 respectively, by Day 56 (FIG. 5C) a significant upregulation in hepcidin was observed for all oligonucleotides. Upregulation of hepcidin was observed for the remainder of the study to Day 168. This data demonstrates that GalNAc-modified oligonucleotides for inhibiting TMPRSS6 successfully reduce increase hepcidin expression in vivo.

To measure serum iron and serum iron saturation, serum was collected from the animals at Day-7, 28, 56, 84, 112, and 168. The levels of iron were measured using an iron panel analysis conducted at Cornell Veterinary Diagnostic Lab.

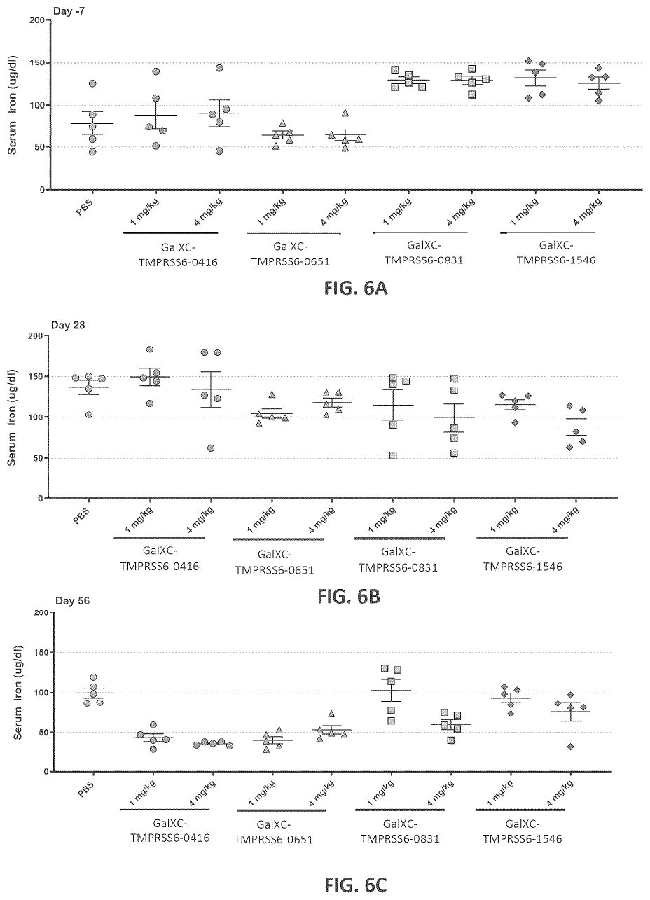

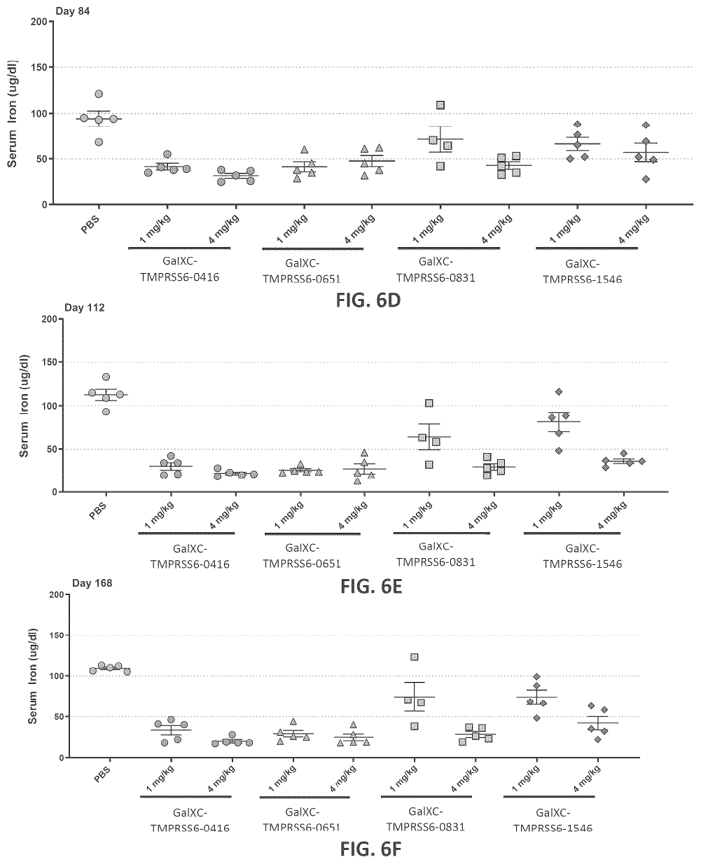

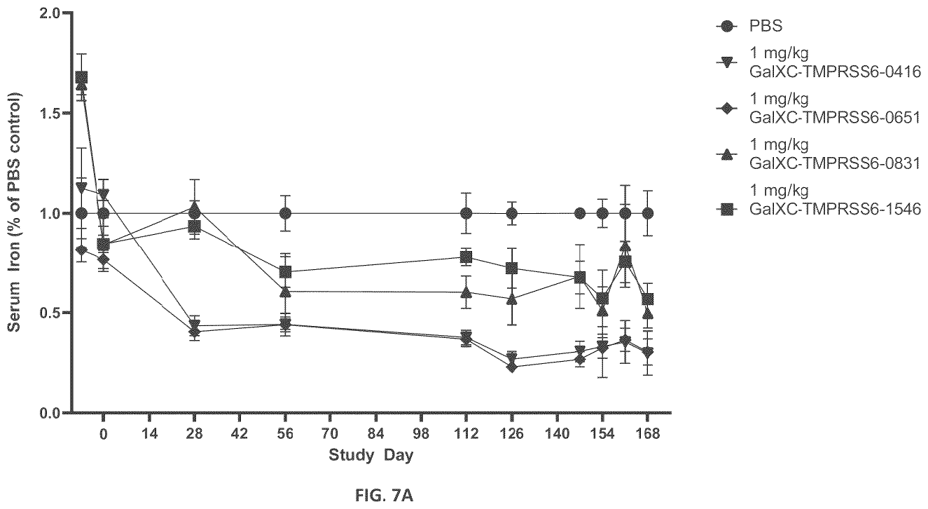

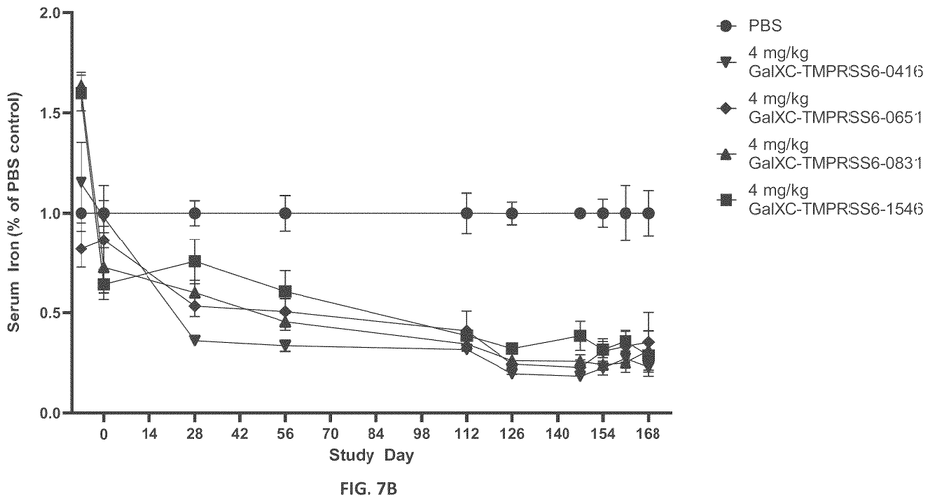

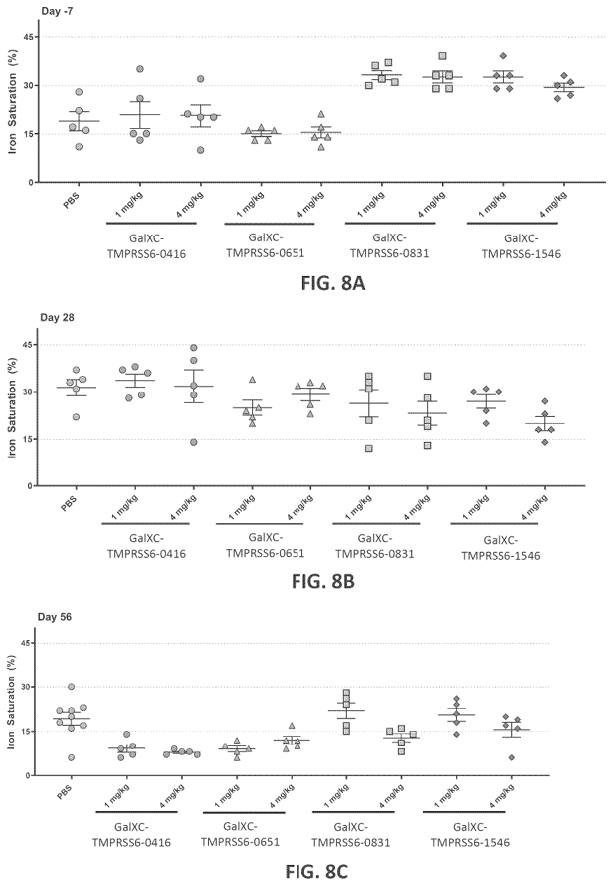

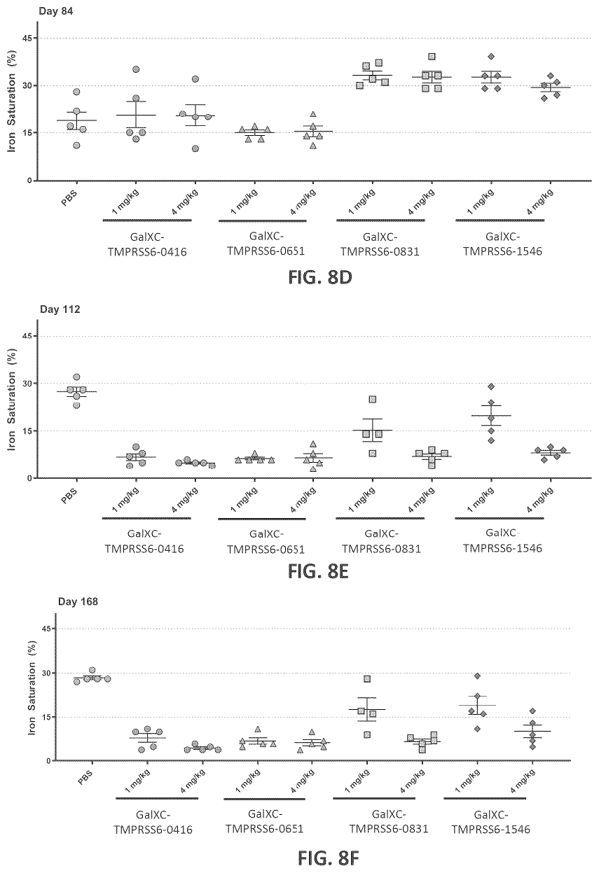

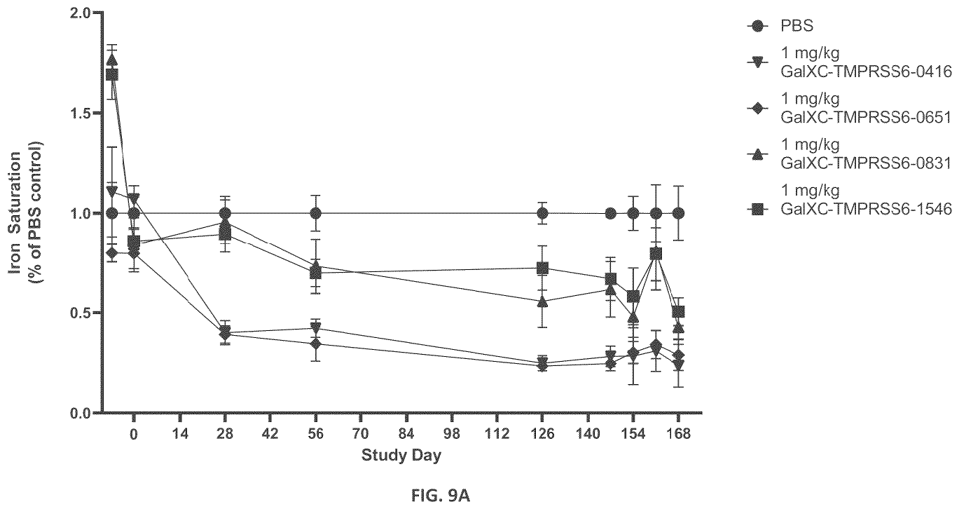

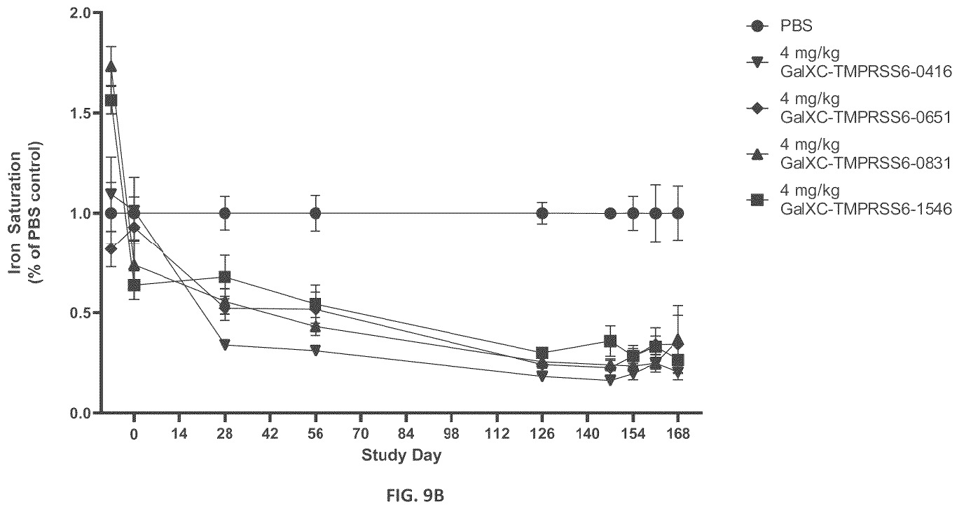

As shown in FIGS. 6A-6F and FIGS. 7A-7B, a dose dependent reduction of serum iron was observed after Day 28 and generally throughout the 168-day study for each oligonucleotide. Similarly, a dose dependent reduction of serum iron saturation was observed after Day 28 and generally throughout the 168-day study for each oligonucleotide, as shown in FIGS. 8A-8F and FIGS. 9A-9B. Together, this data demonstrated that GalNAc-modified oligonucleotides for inhibiting TMPRSS6 successfully reduced serum iron and serum iron saturation in vivo. Subsequently, reducing free iron may benefit patients with iron overload diseases, like hereditary haemochromatosis (primary iron overload) and beta-thalassemia (secondary iron overload) and would limit miss-regulated and elevated erythropoiesis as observed in polycythemia vera.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An RNAi oligonucleotide for reducing transmembrane serine protease 6 (TMPRSS6) expression, the oligonucleotide comprising a sense strand and an antisense strand, wherein the sense strand and the antisense strand form a duplex region, wherein the antisense strand comprises at least 20 contiguous nucleotides of a nucleotide sequence as set forth in SEQ ID NO: 600, and wherein the antisense strand comprises a region of complementarity to a TMPRSS6 mRNA target sequence, the region of complementarity being at least 18 contiguous nucleotides.

2. The RNAi oligonucleotide according to claim 1, wherein the region of complementarity is at least 19 contiguous nucleotides.

3. The RNAi oligonucleotide according to claim 1, wherein the sense strand comprises a nucleotide sequence as set forth in SEQ ID NO: 844.

4. The RNAi oligonucleotide according to claim 1, wherein the sense strand comprises a nucleotide sequence as set forth in SEQ ID NO: 579.

5. The RNAi oligonucleotide according to claim 1, wherein the antisense strand comprises at least 21 contiguous nucleotides of the nucleotide sequence set forth in SEQ ID NO: 600.

6. The RNAi oligonucleotide according to claim 1, wherein the oligonucleotide comprises at least one modified nucleotide.

7. The RNAi oligonucleotide according to claim 6, wherein all nucleotides of the oligonucleotide are modified nucleotides.

8. The RNAi oligonucleotide according to claim 1, wherein
the sense strand comprises 36 nucleotides and the antisense strand comprises 22 nucleotides, the nucleotides of each one of the strands being numbered 5′ to 3′;
all of positions 1-7, 12-27, and 31-36 of the sense strand and positions 1, 6, 8, 9, 11-13, and 15-22 of the antisense strand comprise a 2′-O-methyl (2′-OMe) modification; and
all of positions 8-11 of the sense strand and 2, 3, 4, 5, 7, 10 and 14 of the antisense strand comprise a 2′-fluoro (2′-F) modification.

9. The RNAi oligonucleotide according to claim 1, wherein
the antisense strand comprises 22 nucleotides;
the nucleotides of each one of the strands are numbered 5′ to 3′; and
a phosphorothioate linkage is provided between positions 1 and 2 of the sense strand, and between positions 1 and 2, between positions 2 and 3, between positions 3 and 4, between positions 20 and 21 and between positions 21 and 22 of the antisense strand.

10. The RNAi oligonucleotide according to claim 8, wherein
the antisense strand comprises 22 nucleotides;
the nucleotides of each one of the strands are numbered 5′ to 3′; and
a phosphorothioate linkage is provided between positions 1 and 2 of the sense strand, and between positions 1 and 2, between positions 2 and 3, between positions 3 and 4, between positions 20 and 21 and between positions 21 and 22 of the antisense strand.

11. The RNAi oligonucleotide according to claim 1, wherein a 5′-terminal nucleotide of the antisense strand comprises a structure according to Chem. 1a (MePhosphonate-40-mU):

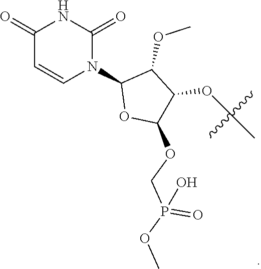 [SMILES: *O[C@@H]1[C@@H](OCP(=O)(O)OC)O[C@@H](n2ccc(=O)[nH]c2=O)[C@@H]1OC]

12. The RNAi oligonucleotide according to claim 10, wherein a 5′-terminal nucleotide of the antisense strand comprises a structure according to Chem. 1a (MePhosphonate-40-mU):

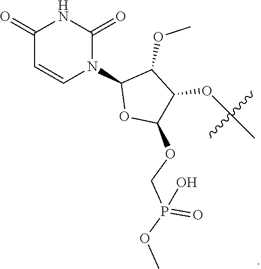 [SMILES: *O[C@@H]1[C@@H](OCP(=O)(O)OC)O[C@@H](n2ccc(=O)[nH]c2=O)[C@@H]1OC]

13. The RNAi oligonucleotide according to claim 1, wherein the sense strand proximal the 3′ end comprises a stem-loop set forth as S1-Lp-S2, wherein S1 is complementary to S2, and wherein Lp forms a loop of 3-5 nucleotides in length between S1 and S2.

14. The RNAi oligonucleotide according to claim 13, wherein the loop Lp is 4 nucleotides in length, and wherein the second, third and fourth nucleotides of the loop Lp from 5′ to 3′ are each conjugated to a monovalent N-acetylgalactosamine (GalNAc) moiety.

15. The RNAi oligonucleotide according to claim 12, wherein the sense strand proximal the 3′ end comprises a stem-loop set forth as S1-Lp-S2, wherein S1 is complementary to S2, and wherein Lp forms a loop of 3-5 nucleotides in length between S1 and S2.

16. The RNAi oligonucleotide according to claim 15, wherein the loop Lp is 4 nucleotides in length, and wherein the second, third and fourth nucleotides of the loop Lp from 5′ to 3′ are each conjugated to a monovalent N-acetylgalactosamine (GalNAc) moiety.

17. The RNAi oligonucleotide according to claim 1, wherein the antisense strand comprises a 3′ overhang of one or more purine nucleotides in length, and optionally wherein the 3′ overhang is 5′-GG-3′.

18. The RNAi oligonucleotide according to claim 16, wherein the antisense strand comprises a 3′ overhang of one or more purine nucleotides in length, and optionally wherein the 3′ overhang is 5′-GG-3′.

19. A pharmaceutical composition comprising the RNAi oligonucleotide according to claim 1, and a pharmaceutically acceptable carrier, delivery agent or excipient.

20. A pharmaceutical composition comprising the RNAi oligonucleotide according to claim 18, and a pharmaceutically acceptable carrier, delivery agent or excipient.

21. A method of treating hemochromatosis, comprising administering to a patient in need thereof the pharmaceutical composition of claim 19.

22. The method of claim 21, wherein the hemochromatosis is hereditary hemochromatosis or beta-thalassemia.
